SEQUENCE LISTING (120 sequences)

SEQ ID NO: 1 - DNA, 601 bp, Homo sapiens
actcgggtctgtgggtaatccaaggctgccccgcttacccttcaaatgctctttccccctccggaagccctcgcgtcctcatccttaccccacctcttgttccccaagcgtggccagggctaaggctccagggatgcgccaagcacccttcggttttcccggggagaattttccccggcccggggactagggtctggcgctggggcgcccctcggacctgcgggatcgcccctacactctggcgcgctgagggcggtgagcgagggcgccaaggcacaggtggggcgggagtcgagcgcggaggctcggggggcgggacgcggggcctgggagcggccagggaccgcggcagcgcctcagtgccagcctggcgcccgcgactgcctgccccagcccctcagtggcggcttgctctcttctctcgctccgaaccagacacagccgctgccgctgccgtccggcgcgctacagactcccgagaacagccctggctgtcagcgagcaccagccgcttcctgtccccatcgcggagactggaggggcgcaccacggccatggagccagaggcgcttcaggaggcaagagaagtccccgcgcgctc

SEQ ID NO: 2 - DNA, 603 bp, Homo sapiens
gcggagagcagagaagtaaaggaataaaaccaacttcgctggcaacttcaataaatccggtaactggaggcaaaaaatagactccgagggctgggtctggtccggatgaaacgctagggctccttcccttccttgaatcttgggcgctggaagccagccacgggcgtcttgccacgcgagtgcccctagacagcaacacacccactggaaacgcacgtgaacaaagctctcgcccccgggagccgctgcctgcggtttcctagtcgatcccagcttctctagggagtgtcaggcgcacacagggttaagttagttccctccctggtaggagggagaggaggaggaggggaaaagcagcatactgtctcaggctgggtaccttgtagttagttgtacgttcgaaacctgtcgccgtcacttgcgcgtttggcattatccattgtcaccgcggaggaacgagcgctcgagatatcatcagtgcccgcaaatctccgcgccaaggcgctgagctactcctttccgaggtgcgcctctggtcctccgtccctggtgcccagcagcggcgaggcggcatctccgctcccgccgccgtgtccaccgagc

SEQ ID NO: 3 - DNA, 601 bp, Homo sapiens
tcgccgcccgcggctcggaagctggagagtcagcgacggggcccgactgcgggaccgagggctgcaagaagaagcgaacaaatagtccccagcgcctcctctggatgcggtcgcgtctgtggtcctggcagccgctgggcgggccaggccaggtcgggccgggccgagccgggcacatggacctgggcctgcgggctctaattgcggcgcttatgttgatgattttttttttaatcacagcagcccccagtttagcggactgatttactcccggtattggtaaatatgatcacgtgggccgcgcgaccaatggtggaggctgcagcctgcgaactagtcggtggctcgggcgccggcggggagctgctcggcggcggacagtgtaatgttgggtgggagtgcgggacgcctcaaaatgtcttccagtggcaccctcagcaactactacgtggactcgcttataggccatgagggcgacgaggtgttcgcggcgcgcttcgggccgccggggccaggcgcgcagggccggcctgcaggtgtggctgatggcccggccgccaccgccgccgagttcgcctcgtgtagttttgcccccagatcg

SEQ ID NO: 4 - DNA, 601 bp, Homo sapiens
gctctgcactacgccgatccggggacaggagcaggaggctgtggctgcactcagacttcgggacaggccgagctgaaaaccgtgagaggggtggggtggaggcgaccgaaacgccaaggctgggttcccggaacgcgcggggactagggtggaaggcaacttcggggaaactgggaaaggcgaccgggacctcggggacgccccgtaccccgggcgtaaactcactcccgcgttagcggccccaaagcggggagggggtggtcccgtggtccgcacccaggggagctcagtggactgtgcgccttgcctttctgctgcgcaaagcccagtccaggtcatcacctcgggcggggcggactcggctgggcggactcagcggggcgggcgcaggcgcagggcgggtcctttgcgtccggccctctttcccctgaccataaaagcagccgctggctgctgggccctaccaagccttccacgtgcgccttatagcctctcaacttcttgcttgggatctccaacctcaccgcggctcgaaatggaccccaactgctcctgcgccactggtaagggatgctaggtttctggtccttaggatacctat

SEQ ID NO: 5 - DNA, 601 bp, Homo sapiens
ccgtcaagccccgggcccgggcacggcgcgactgccggaccctcgcagccagatccctgtcctgggccagcgggcgccggcagctcggcctccaagcccagcaggtccgtgatggcgaagccccgggggcgcgagcccctaggggaaccgccaggcaccagcgccctgctgctagtgcgcccgtcggaaagcgagtcccggccggtcatggttccttagcaagcaaggcgcgagcctctctggatcccgtttgcggagggcccagcttagaggaagctttataggattgggctgccggtcgggcgcgggaggcctgaagggctccaatcagctgggcgcgggcgcgtcgccgttccggaccccgcgagatggggacgccaccagttcccctcccctcggttaccgcccacccagctgcagtcccctcagtcacctccttctcctcctctcttctgtccagcccaccccaccccaccccacccccattctaggagggaggggaggaaagaaggtaagggacagtgactccgctgtctgcctgtccctcgaaaagtcgcaagttctacatcctggcgtggccttctccttccttccagactct

SEQ ID NO: 6 - DNA, 601 bp, Homo sapiens
cagaaagcctgcggaaaacgaatcgcaaagccaatcacgaccaagaagagtcccaggggacacttgggcagagtcaccctcttgcccgatgtccccagctgctgaagccgggcctggaaacccgcagacagttagtcttcgctcaacctgatttggctctgctggcagcctcgtccttcgccatcgaacattgcgggtgttatcataatactctgaagggggggaaaacgggtcggggggatgtaggcggtgctgaaatgaccggctttgaagaacctgcaggcaaagtttcgtccaatcgtctgagcctgtcctcttattcccggttgtaactaaatactgttgcgagcgcagccgaagccctttgttggagatgtgtgagcgcagtctctacagagcgggctatgtgggctcgcttctgaatctgcagtcgccagactctttctacttctccaacctgaggccgaatggcggccagttggccgcgcttccccctatctcctacccgcgcggcgcgctgccctgggccgccacgcccgcctcctgcgcccccgcgcagcctgcgggcgccactgccttcggcggcttctcgcagccctac

SEQ ID NO: 7 - DNA, 601 bp, Homo sapiens
tggtcagtaaatgagaaacaataaatagcctttgcacaaactgaaccacgcagcgcagcctctgctactgcacacaataacaggagttcggtcaaggcccaaaaggaaagttccagaagtgagcgcagacctttggacctcagtctgtacggagatcgagtcaccgtctattcaatccaggttttgttccacttgctgatgattagacatcaaattatgcaatctgcaaggaaaaggagagagaacgattttaccgccgatttaagcgttctcctttttccccttcgtggccgttttattcggcactgaaatgctgtttaacctcagcaagagtaggcgtgggaaggtggcgctcttccccacagcacaccaggcaccccccagctgctcagcggaagtgggcagaggcaagaggctagcggctggaccacttgtgctggagtggtaaagaactatcatgaatccatttactgaaagtgtccatttctgaactcaccctaaagaggacaaacaccgcaaagtagttaaaagtcaggcattcgcgtcggacgtctgggtttgaattctgccctggcttggtaaaatagggctttggataaat

SEQ ID NO: 8 - DNA, 601 bp, Homo sapiens
ccgggtggaggtcggaagggaggttcgagcgccaggctccgaggacccgaggccagctctgagcgcccgagagtccgcccgccccgtcgcggccgcactcagcccgggtgcaacgcaagtgcagccagcccggcccgcgcgccttttaacgcgcaggaccccgcggaggggggcggggcggggcgagctccgaaggggcggggcctaggaggcccagacaccgcccaaagccgccccgacccccaccccgaagggtccccgccgccgtcttagggctcagaggtgtggagctgggatgcgcgggaccctatccccggagtccctatcctgccccaatccggaccagaccttcggtctctacacagcgtccgaggccagagggtcccgacccttttcccgggcctccccacctgcggccccgcggaagggaggggaggcaggcctggcggcgctcgtgtgaagaagttaattcaattcgctctgataaccgagttattccgatctggcccctcagccccgcccccagcgaggtcccatcccggcggagactgcggaggcggcccaggaagggggatggagaccgggaagagacggagcctgg

SEQ ID NO: 9 - DNA, 601 bp, Homo sapiens
gtctctaatcgctcagcgaaaagttggtttcgggaagggcagcgccggggtgcgggagggcaaaggaggaagaggaatgcatatgactgtgagaaggaaaagtaatcgtggcaaaaaaaaaaaaaaaaaaaaaaaaagaaagaaagaaaagaaaaaagaaaaaaaatttccgtgagaagagggaaaaaatttggctaaaaaaaaagttgctactcctggcagccctggtttgtcaaaaggggatgtcaagcgctttacaatacctgggattgatgaggcgggcgggccaatgagctgcgcgcggcgcctcggcgcgccctccgttggcgcggcggctgagggcggggggaatgcgggcgcaccaatgggcgccagcgtcggcagcacgtgacacctcccccctgctcccattcatcaagggggggacggtgtcgtcctttcaattcatttatctgcaggaatgattgctgctatcagtctcgcgctcaccgcccggctgaggaggtgaaagtttctccccaggaagataaaccgcaaaagacaatattgtgcatgatttgcgccttttctttggctttttctttctttcttcaccccccca

SEQ ID NO: 10 - DNA, 600 bp, Homo sapiens
cctccctaaccaattgtgtcgcttcctgcgctttctccacggtcacttcacagctaagatttctttctttccgagctgtagaaggcagaacgctctcgggaggacgaagtgatccgaagggatgtggcaagcgcactttccgatggagatgcagaccggctgaggcttgggatgcttctataagtaggtggtgctgtggtccaaggcatctaagtctaggctgcctcggagttcctgggtcctaaagccagaaacgtcccggtttccctgaggtcctaaagaacgttgacagccaacagcgcgcctaacttggagcgaatcctcttcgggctttccagagtgcgggggatagataaagagtagctggggagacgccccctgaccttgctgggtcccagaacccggctgctcacccccaaggggtcctctccagccctctagggtcagttccaacctcagcgccaacctttctgcagggagatctgactgaaggactgatgactccctttaaacttttgcaatatgccacaactctgccactttttaatttacatcttgtctgtgttttgcaggaaggtatgttagccggctgaaattatt

SEQ ID NO: 11 - DNA, 961 bp, Homo sapiens
ggaaaggaaacaggagacgcggaagagttttatttgttgacagtgcccgagacaatgtggaaccgaagtccccagtctagagctagcctctctgctctagccctctctctgcctctgtccctcacacctctccccgcccccaccagtcgcgctctctgtttcttgcaccggcgtgttggcaggtgcgccccctcggcttctctgctgctgattggcgcccaggttggggagtcgccgccccgcgcttatgtcagagtgcgtgcagtcccggcccacctcgcctttgaacttcgccgcttttggctctcgttcaccagcctccccgcattctcagccaggtgcagcgctaggcgcggcagctccgggaaagccacgggaagggggagccacgactggaaacgcaagaggagagaagggaactagagaaggcaaaggtggagagggggagaaggaggagcttcttgccctggctaaaaaattacctggccacttaatcccggagactccgatacttgggtcccagcccctggtgcctgaagccccccggccgatcgccctctcctcccccgccctttctgcccccactcgctcccccggggctgcctgggcgctgggactggcatgatcagctgaactttgtggggtcagcgcttctctctggcttcccctcagcggcgccaaggcgaggggagcgcagaaccccggctcaggacggacagacagacggccagccgcgcccaggctcgtctgcagaacccttgcactccctacccccacccacctagccgccgggaccatgtccaaaccttcagaccacatcaagcggcccatgaacgccttcatggtatggtcccggggccagcggcgcaagatggcccaggaaaaccccaagatgcacaactcggagatcagcaaacgcctaggtgccgaatggaagcttctgtccgaggcagagaagcgg

SEQ ID NO: 12 - DNA, 600 bp, Homo sapiens
ctaaatgaaagaaaattcggtttgtggttaaaaagggggcttcttgcattataatgtcctttagagagacggggaggtgcttaacagttgaatgaacccgtgagcagctcggcgaggggaccaatcaggagggcagcctgcaaatgtcagcgcccggagagtcccccactccgcccgcccgctcgcctgcgcccgcctgaaggaggcggcggcggcagccctggcagctccagcccggagcaggagctaaagcagcctcagccgcctcggcctcgccctgagcttctgggcccggctccctctagttcttccctgcgattttctccaggctcccctttccttccccttggccggcgctgcgcgcttcgaaggcgtagctctgtgcctctgagctaacatcttcctagggcaggggcagagcaggggtcagagcgggccatgaccctggcgtgacctgagggtcgcccccacctgcgctctctgggcctgccgctcatattggcccgccagcccagggcgcgacgagggcacaccgcggccttcagacaacgcctggacttcgccgcggctcagtaggcctgcgcctagggtccaggcgcc

SEQ ID NO: 13 - DNA, 600 bp, Homo sapiens
tctgcgacgagtggcgagactcgctgataaagtttcaaacgatgggcccagcgagcgataaaggccagcccggaccgcctcgcccacagcccaattccccaggcgcagcgagcgcctgcagcccgaaagctctgcgcgcctcccgcgcgccccgtccgctctgctcccctccgggcgcagccgagggagggggtgctgcgcctggcgggggtggggagaaacgcggggttttagacacgggtgcatgaaatgatggcaagagatgtctggggatgaggtgaggcggggaagggggaacatcgctgcatggttacgagcacacgcgaatacaggaacctttagggaagctccaggccaaagagcatccgtgggctctcggagcgtgcgttccggattgccgaggccatccgctggcaaacaagtccctgagcgtgcaatgtgaggaaacgaggaagggggccaggggataaggagattactatgcatgggatacaagaatccagagatgattctcaactcattgcttcgcttctctccctgaagctgggatgaagagctagcacttgtaaatttggagctgccgcgtctatgcagggcaag

SEQ ID NO: 14 - DNA, 601 bp, Homo sapiens
atagtctcataaccctggggcctccggacgccgcgcctcctgcgtgggcgaattcctcgtgtcgtggccgcgcgggcgctcaggttatatagcccagttagtgatgctaagcgcgggcggggccgctagctgaggggctagcaggtctatgcgcctgacgcctgcgcacgcgtccaggctgtgcgctccccgttctcccctcctccccacttctccccacgccttgctcgtctcccgccctcctccgacaaccgctcccctcaccctccacccctacccccgcccctcctccttcctccccggcatgcgccatatggtcttcccggtccagccaagagcctggaaccacgtgacctgcccatttgtatgccgcggagcgctccattccggcccctttgtggccagaagaaagtggcccatctgtcgccagttagagactccgcggacctgtttttacccgcaggagagattaaccctttcaggcggcagaagggacggggatagaggggggaggggatttgagggaaggaagggaaaagctaatgttaactaggtctagatcagagcgagtggcctgctttcgcgccggaagtaggacaga

SEQ ID NO: 15 - DNA, 1001 bp, Homo sapiens
agcggctagccaggtccctcgcaccgaaagcgcggattcgcaggatcaggtccagggcgccgggcgcagcgttcagggcggctggtccggcggggtcctctcctgcctggccgcccgcccccagtccctgcgcacaactttctcgtccctcgtgcagcccggagagcgcgaagcgggcacacgcgctctatttataggagcgcagcgtccggctgggttggcttatcgcgcaacccgcgggctccgagaaaagggggagaaggcgtctggtgaccccatctgagcagctctctcctgacgtttaacgcaccccaaagcaagcggggcgcacagcactggtgattggcaaggttcgaggcccctcagcccctgaaagcccgtggcgtaaaggatgcgtgcccagagaggagcccacggccctgcgctggccccagctccgcgcccactctctcctgctcgatctgagtccgcagaggaggcggctggtcccctcacccctcccagcgcggaactctgccgccgagtgccctccaagcggagcgcggcgctgccaggccgggtcgcgaaaaaggataagccgccgcggacccgcgctgcgcgctgccccgaacctggggaccggcgggcgtcccgaccgcgcctggccgggagcccgccccacacagccctggggcctggcgagctcagggaagccttggggcggaccagagcccgtgggggcggctgggaggaggtgcctgattccttccttccgctccgggaagatgagcctcagaagccgcaggggcgcgccttccccccacaccgcgaatcctccccaggcatggccaaaaaaaagagtgggagagggagttaactcagcaagaataagtcctccgaaaccgtgccaacaacaataaaaaaataaaaataaaaataaaaataaaagacaatcccactcctaggaatacagcatttttctcccctttggatgcgctttccgcccccgtgccactccactg

SEQ ID NO: 16 - DNA, 601 bp, Homo sapiens
cgctgcctgtaaggcagcgcacatcggagagctccatggctctgggccgcagggaaggaggcccagaaccttggggaggcagccggaggaggggttcctaaagactcaaaggcggcctggtaggagcttggcaaggatcctttttatcctaggctgccccgttgcaggagccctgcgctagggctccctgtaggtcgggtcatactgtgcccaggggcaaagggcacctggaagatcgcggcggacctcggcgtccttgcgtgctgctctcccgccagccggcgcagcttgggtacgcagcggcagtggctatttgtcctcaggagccgcagcgctgtgagccacgcagccccttcctgggctcaggctccttggttgattctaggctcagcgttttggcgcagcctttgctctccaagagacgcctaagcgcctgggcgccgtgcttctgctgcgcgtcgggcgggtcctggctgctgcactgggctttggacgcccactgctccgcggctgcagggggcctggccggcggtgaccggcggcagcgatgggtgcgaagttagcagatgtggctggaatcatcgcccttcggagaactcca

SEQ ID NO: 17 - DNA, 601 bp, Homo sapiens
caatttttaagcaaaaaccccaggattcctccaagactgactgtctggaagggtagggatgtgagaatctgaagtccacatacatgctaggtaggtacaccttgataaatgcaggacccatgcctgcttcagtcatcgcagagctgtggggctatgcgcagaccctcaacctctttcggagtaatattttcggaatccctgaagataaggggggacagcaatggaatctagtgaaagggaaagaatagacagtgatttaaccccctccttcctcctctacagcccgatccactctgttggcggctgcgccttgccttgggtgcagtgggacgtgtccctcccatgtttcccttaattaggaggaggcaaacttctgtcttcccctcctgccggtttcggtttagtaatcagaccacatgagtcacgccaaacacgcagccccctcccgctggagtgacaactggccagcatactctaggctgttgtccctttaaaacttgaatccaagggggtaatgatttatcaaacttgtattatcaagaaaatgtcaaaccaagggcaccttgctttgcactgacgcaaacccggcctttcccaagga

SEQ ID NO: 18 - DNA, 601 bp, Homo sapiens
cgcaccccagagaactcaccaaaccgccaggcacaggaagcgcaggagggcgaaactgaccccggcggacagagcgatcgcgggtaccttcttggcagaaaccatctttgcaacatgccatacttcaagcctgttcacgtttccaggctcttcagatgccctgagcagggtgcgaggagagggcagagaggctcccgcggcgtgcgcacactcgcgctcacactcgcccgcgctcagccagcccgagccgcggtgggcgtgtgtgtgcacggggccaggggaggcggaacctgccttcctcgccccctcctttctcgctcagcgctcaatgtgtatgtagagctatgtataccgctccacaccctttcgtgcccgcgcgctgaaggttctggggttcgtatccgcgcgcttgcgctgcaagactcggcaagtttgttccgactgtaactccggggatgaggaacggggtctctcgcccctcctccggcttcgcagcccccgaccccaagtgggctccccgcccacagcagtgttcgtcctcctacctcctggccccctcccttccgctgggatttggatctaaccaatgaagctcctctgt

SEQ ID NO: 19 - DNA, 600 bp, Homo sapiens
gagggggtggggagggggaaagggagggagagagagaaaaaaacgcggattcgccggctcctagtcaccctcgccaggaaaggagagggaaagagaggagggaaaaaaagaaaagaaagaaaaggcgaagaagatctcgaagccgacacactttttgtggtttccaggaatagaaaaggacagagaagcctccaaaagtctaagcttgcatggcagccgcggctcgctcgccctccccccaccccccaccccccaaacaaaaatataccaccacttaaagaggtcctcgctttacatttcgaagaagggatgccagttcataaacagttttcggtgatttacttccctgcaaatgagttgtttcatattttgcactgtcttttcatgatcatttgcatccattagagaccccgcatcctattggcttcttcgtactcctcccggacagaacgcagagcgagggtgagagcgagagagagcgagcgagagagagagctagagcgagagagcgccagggagtgaggagagagcggaaaaaaaaaaaggaggggaagaaaaaaacacccagggagagagagggggagagggggagagagaggg

SEQ ID NO: 20 - DNA, 600 bp, Homo sapiens
cgctgcagcagcgagctcgggttcgcctgctggtctgagagcgtaccctgtgcggcgcgccgcttctccgagtgcacgatcagcagcatgtcgatagatacagcgttgatgtccttcttcagctccatgatgccgcctcctttccgacctgggcgccgcggccgcacgctaggctgcttgcgctgcaaatggccccgtgcgcgcagctgccccacacgggagagggcgagggcgcggaagagggcgctcccccgccgctggagccgcagggcgctgcttttcgctgactctgccaaacacgccattagggcccgcctgggagggcgctaccctaatgaagcggctctaagtcacctttgaaaggaaatccccgggcccaagccttgacctgcaccggggccatagtcagcaggggcggccggtccagagcctccctccctgcgcccctgcctcccgcccggtgcacttgaagcgctggtcctgggctccagggcttctctggctgagcgctgcctcatccccacggtctccggagggttctcgagagggctggagccgacctcccacgagaagcccctggcaaatacagaaatcgcacag

SEQ ID NO: 21 - DNA, 600 bp, Homo sapiens
cctgaccgccagatagcacttacccgcggctccgaaccacaccagcagctgtccccagcagcccatccctgttgggtccacccggcaacaaaagcccttgcggtcgcggccacacccctgtcaacctaaagttgacagctcattttgccacggcagccctggccaccacttacaggggtgtgctgccgctggcgagacactccccggacctaaagctggtctggccacgcccacctcagaccccaggacgccagaggaggaaagggaggggcgtgtgggcagcgaggtccctacgcttccgggaggagccttaaggagtccccacccaggggttctagtttccagccgtcagatgtgccgcagcctgctgggagctccggtaggtttgaggccggagaggaggtgccccacgcttaaagtgggagccacacctcggacaccaggtgggggcttccaggttcctgaggacaggagcagtggagtgtgcacttcatttgtggcagtggtggtggtggtgagtgtgtggcatgcgtcaccacacgtgggaagtactctaaactgctccaagtaaggcctcattacctgccattacttgtgcct

SEQ ID NO: 22 - DNA, 600 bp, Homo sapiens
accggcgcccagggcgcaggggccagagcgagcctggcgttccgccgcagccggctgagactcggcgacgcgggggctgtacctgtggctgcggggccgacggccggctgcagggcggctggctctcccgcctcgagactaggcgcactcccatccccgccgcatgttctccacgcgggctccagcgcgctcaccaccgccaccgccgtcgtctcggctttatttacccagcccggcgcgcgccgcccgggaacaggaatagcgaggccttctcatgtttcctgactgccggtcccagccggcgaacatcctgcgggcgcggtatccacgttcccgggcgggtggagaggaagcgtccgcgcccgcccggccgcgggggctgctgttaacccctagcagaggagccgctgccggggagccccgggcccgccgagtcctccggagccgggcgcggctcccagatccgccggccttgcagtcgtggagcagacggggcttttcccttcgcactttaggagagaaattggagaagctgccgggagcgctcggctccggctccagaagctgagaggctgcggtgcaaatcccgtgtcttagctg

SEQ ID NO: 23 - DNA, 600 bp, Homo sapiens
cctgcggagcgtgcacctacgcctcgggctcctttcctcctccactcccctttcctgctgggcaccctgctttccctctcccagagagggtttgcaacttttctcccaggctggggctcgccctgcttggctaacccccaagagccactgccgtcccgcagcgcccctgcccccgagttgcctgccccgctgggcccccgggaggagcggagcgcgctcacccttcgcccggggctgggagggcggcgagtcgggcgcacgcgcaccccctgcccgcccctggcgcccctccccgcgggcggtgcagctacccctgcagcgcctcccctagctagaagggagcgggagggggctccgggcgccgcgcagcagacctgctccggccgcgcgcctcgccgctgtcctccgggagcggcagcagtagcccgggcggcgagggctgggggttcctcgagactctcagaggggcgcctcccatcggcgcccaccaccccaacctgttcctcgcgcgccactgcgctgcgccccaggacccgctgcccaacatggattttctcctggcgctggtgctggtatcctcgctctacctgcaggcggccg

SEQ ID NO: 24 - DNA, 600 bp, Homo sapiens
tgtcccgactcctctcctgccgcggggactccaagcgccggacacgcgggagcgagcgctcagcagccccggatcctgcacgccggggacggtgagcctcgctcgcggctccctcgctgggtctgaaccaggagcagctgagaacgtgccggggagaggccgagctccgactcactcatcccttagcaccgagccgcctacttattttaatccaccaccctccctcctccaccacccccacccccgccccttgcctcaggattcctcccctccctcccctgccccccaaccctgccctgcgcctgcgcaatgaaagacaaagtttttgctgacccgggcagcagactttggcagctgctctcgcttggacccggcctctcccaacctgcgtcccctcctcttcagtcccgcgcgacccctgctgcctttttaggtccagagcggcacgcctggcgcactggaggaaacggtccctagcagatgagagcgggctggcaccgcacgtttcgaggtcctatacttgggctcctaggacacagcccgtgctctttgggaggagggagggaggggcagtagctctgtgcaccgggatccccacac

SEQ ID NO: 25 - DNA, 600 bp, Homo sapiens
ggcggccccgccgcgggctgctgctgctgctcctgctgctgccaggcgctccctctgaccgcgcagagcgccgcgcgccggcctcgcggtcccgcttctccgacagctctagccccgcgcgccgacgggatgcactcctctaacacctgagccccacttctcgcggccgtcccggggagctctcgcgagagctcgcggccccaccgcgctgacaggcatcagctgtctccgtctgtctgacgcgcgctctccttctcggcggccccgtgcgtctgcgcgcgcgcgcgctcgtccggccgcgaccagcgcgtgggagccgctcttatagtgaccaccagcaaggacagcgcaggtgatgcacctgtagctacccgggcatgcgcactgcccgcctcacctttcccaccgtggtctggaaagatcagaaggtccagctattgtctaagggtgggcaccttttagaataaaaaagacgagatgcgcgcacgcccctctatgtgtgtgtgtgtgtgtgtgtgtgcgtgcgtgtgtgtgtatgtgtgtatgtttcttctccctgatagcaagccttgaatatatgagcattacttaggcattcct

SEQ ID NO: 26 - DNA, 600 bp, Homo sapiens
gcagagccccgctctttttttccttcttctttttttactgctccagcccccccaataataacacatcaatgggacaggaggtgggggaggagagggaggaggagaaagaagaagaaaaagaggagaagaaagaagaaagaggaggagaagtagaaggagaaaaagaagtaagaagaggaggaggaggaagaagaggaagagggggagaagggtgaagaggagagggaggaggaggaggagaagagaaggggcgggggctgccggagggaaatgggaactgatgcttccctgccggagcgcgcttgttcaatgttaagatctttgacaattcttcctgcggagagttcagatctgatagcgacgctgcgcttgaagtcccactatttatacttcctaacggcggcctcttgttttgagtaaattacctcccctcgcaactcggagtgagcccccttcccgggtagcagtcgcgcctctaccgggggttggggaggggaaaaggagagaaacagagagagagagagagagagagagagagagagagagagagagagagagaagaggagagagagagattggagttcgggcgggctggggctg

SEQ ID NO: 27 - DNA, 600 bp, Homo sapiens
attacattctactttaagactgaaaatcatgcaaaaaattgtttgcaaattcaagccagatatcgtgtcctgcttactagagaatcgctcatattaagtcaagcacccctcttagggctgcaggctataggaacagtttctgccatgagcgtgcatcgatgaggttatccacccgccccctccactttgtgtgtctgggtctccagatttcattataaagaggcagagcccatcagagaacttgatcggcagccaggaacggttttttcctgctctggtcctttgtcaggtctttgttccgcccctggctacactcgataacagaaccacttactggcgattcacaggaggggaccgggcggcagggcggggcggggcctgcggggcgcggggccgcggcgcggagccggcgcagcgattgggttcacccggggggccgcgctgcgcggagtgccaggctgcgggcggctgcagacctgggagcggagaccggcccgccgcccccgacgccgccgagcacgtcagcggcgcgcagccggggctcggagaccgacgggcagaacgacgggcggcgactgcggcgaccgcgggacggcgaga

SEQ ID NO: 28 - DNA, 600 bp, Homo sapiens
ctggcacatcaccttgttgtgagccagcactgtgctcgcagctcctgctggaaatgcagccctgctctgccaactcgctgctttctggagctgctgcacgcgccgccgtctcgagccgtccactcagctccctgcgcccggcgcgctccccccatctccaccctcccttcgcgcgcggatctccagcttactcccctccgttccccctccctccccgacctccgtttttttcctcttccacctcctcctatcccaccttcctctcgctggatcccagctacagttggctccggataggacgcttggctggagccagaggacgaggtagctcctagcctaggaatccttatctgctttgtaacctctcccctccagaagaggttctcatgttcgcgagacagcctgctcattctctgctcctggatttaacacaaagtgtactatctctcgcagttgcctctgctccacaatttccagggttgtcaggcagacgtgagaaacaagatctgaggaaaactttatgcattaataaataagaatacctggacttcctaccctgtaaagctcctgaactcagctcctccctaaagaaaggccatc

SEQ ID NO: 29 - DNA, 600 bp, Homo sapiens
tcgcccgcagacctttcctcatcgtagacagaggtgtgccggggggaggggagggggacagagagacgcgcaaacagctgaaagccccactgggcagaagctgaatccgtgcaccgcgaagcagtccgaagaaataacgacaaccaagcgccacactccactttctccaaaggcaggggccaaaagggggaggagaggagacgccgaggaggagtttgaaggtccctctttcccaatgccttctaactgacgtctcccgcgagcgactgctctgcccccacttgggaagtccagggaggcgcaaggcggccacctccgccgctccccccagcagcaggagcagccgcagctccgccgcacgccgaagtctggactgctctccgggtcccctcgcctgtgaggaacacacacagacaccgggggtgggggtgggggtgggggttgaagtggggggagttggaggaggagggaacggagggaggaagcttgggcaccgtcaggcgctctggtctccaatctcagtgttctcacgccggcggagagatgtcccggctgtcgctgctgggtggccaaggaggttcacatcctagagtagggcgc

SEQ ID NO: 30 - DNA, 122 bp, Homo sapiens
  misc_feature (61)..(62): /note=“This region represents a CpG dinucleotide”
  misc_feature (61)..(61): /note=“Nucleotide may be methylated to form 5-methylcytosine”
accccaggacgccagaggaggaaagggaggggcgtgtgggcagcgaggtccctacgcttccgggaggagccttaaggagtccccacccaggggttctagtttccagccgtcagatgtgccgc

SEQ ID NO: 31 - DNA, 122 bp, Homo sapiens
  misc_feature (61)..(62): /note=“This region represents a CpG dinucleotide”
  misc_feature (61)..(61): /note=“Nucleotide may be methylated to form 5-methylcytosine”
ccccaaagcggggagggggtggtcccgtggtccgcacccaggggagctcagtggactgtgcgccttgcctttctgctgcgcaaagcccagtccaggtcatcacctcgggcggggcggactcg

SEQ ID NO: 32 - DNA, 122 bp, Homo sapiens
  misc_feature (61)..(62): /note=“This region represents a CpG dinucleotide”
  misc_feature (61)..(61): /note=“Nucleotide may be methylated to form 5-methylcytosine”
acttgatcggcagccaggaacggttttttcctgctctggtcctttgtcaggtctttgttccgcccctggctacactcgataacagaaccacttactggcgattcacaggaggggaccgggcg

SEQ ID NO: 33 - DNA, 122 bp, Homo sapiens
  misc_feature (61)..(62): /note=“This region represents a CpG dinucleotide”
  misc_feature (61)..(61): /note=“Nucleotide may be methylated to form 5-methylcytosine”
ctggcgcgctgagggcggtgagcgagggcgccaaggcacaggtggggcgggagtcgagcgcggaggctcggggggcgggacgcggggcctgggagcggccagggaccgcggcagcgcctcag

SEQ ID NO: 34 - DNA, 122 bp, Homo sapiens
  misc_feature (61)..(62): /note=“This region represents a CpG dinucleotide”
  misc_feature (61)..(61): /note=“Nucleotide may be methylated to form 5-methylcytosine”
gcagcccccagtttagcggactgatttactcccggtattggtaaatatgatcacgtgggccgcgcgaccaatggtggaggctgcagcctgcgaactagtcggtggctcgggcgccggcgggg

SEQ ID NO: 35 - DNA, 122 bp, Homo sapiens
  misc_feature (61)..(62): /note=“This region represents a CpG dinucleotide”
  misc_feature (61)..(61): /note=“Nucleotide may be methylated to form 5-methylcytosine”
ctctggcgcgctgagggcggtgagcgagggcgccaaggcacaggtggggcgggagtcgagcgcggaggctcggggggcgggacgcggggcctgggagcggccagggaccgcggcagcgcctc

SEQ ID NO: 36 - DNA, 122 bp, Homo sapiens
  misc_feature (61)..(62): /note=“This region represents a CpG dinucleotide”
  misc_feature (61)..(61): /note=“Nucleotide may be methylated to form 5-methylcytosine”
gggatgtcaagcgctttacaatacctgggattgatgaggcgggcgggccaatgagctgcgcgcggcgcctcggcgcgccctccgttggcgcggcggctgagggcggggggaatgcgggcgca

SEQ ID NO: 37 - DNA, 122 bp, Homo sapiens
  misc_feature (61)..(62): /note=“This region represents a CpG dinucleotide”
  misc_feature (61)..(61): /note=“Nucleotide may be methylated to form 5-methylcytosine”
gaagagaaggggcgggggctgccggagggaaatgggaactgatgcttccctgccggagcgcgcttgttcaatgttaagatctttgacaattcttcctgcggagagttcagatctgatagcga

SEQ ID NO: 38 - DNA, 122 bp, Homo sapiens
  misc_feature (61)..(62): /note=“This region represents a CpG dinucleotide”
  misc_feature (61)..(61): /note=“Nucleotide may be methylated to form 5-methylcytosine”
gatgtaggcggtgctgaaatgaccggctttgaagaacctgcaggcaaagtttcgtccaatcgtctgagcctgtcctcttattcccggttgtaactaaatactgttgcgagcgcagccgaagc

SEQ ID NO: 39 - DNA, 122 bp, Homo sapiens
  misc_feature (61)..(62): /note=“This region represents a CpG dinucleotide”
  misc_feature (61)..(61): /note=“Nucleotide may be methylated to form 5-methylcytosine”
ctggatcccgtttgcggagggcccagcttagaggaagctttataggattgggctgccggtcgggcgcgggaggcctgaagggctccaatcagctgggcgcgggcgcgtcgccgttccggacc

SEQ ID NO: 40 - DNA, 122 bp, Homo sapiens
  misc_feature (61)..(62): /note=“This region represents a CpG dinucleotide”
  misc_feature (61)..(61): /note=“Nucleotide may be methylated to form 5-methylcytosine”
atgtataccgctccacaccctttcgtgcccgcgcgctgaaggttctggggttcgtatccgcgcgcttgcgctgcaagactcggcaagtttgttccgactgtaactccggggatgaggaacgg

SEQ ID NO: 41 - DNA, 122 bp, Homo sapiens
  misc_feature (61)..(62): /note=“This region represents a CpG dinucleotide”
  misc_feature (61)..(61): /note=“Nucleotide may be methylated to form 5-methylcytosine”
cccggagagtcccccactccgcccgcccgctcgcctgcgcccgcctgaaggaggcggcggcggcagccctggcagctccagcccggagcaggagctaaagcagcctcagccgcctcggcctc

SEQ ID NO: 42 - DNA, 122 bp, Homo sapiens
  misc_feature (61)..(62): /note=“This region represents a CpG dinucleotide”
  misc_feature (61)..(61): /note=“Nucleotide may be methylated to form 5-methylcytosine”
ggggcggactcggctgggcggactcagcggggcgggcgcaggcgcagggcgggtcctttgcgtccggccctctttcccctgaccataaaagcagccgctggctgctgggccctaccaagcct

SEQ ID NO: 43 - DNA, 122 bp, Homo sapiens
  misc_feature (61)..(62): /note=“This region represents a CpG dinucleotide”
  misc_feature (61)..(61): /note=“Nucleotide may be methylated to form 5-methylcytosine”
gcaacacacccactggaaacgcacgtgaacaaagctctcgcccccgggagccgctgcctgcggtttcctagtcgatcccagcttctctagggagtgtcaggcgcacacagggttaagttagt

SEQ ID NO: 44 - DNA, 122 bp, Homo sapiens
  misc_feature (61)..(62): /note=“This region represents a CpG dinucleotide”
  misc_feature (61)..(61): /note=“Nucleotide may be methylated to form 5-methylcytosine”
gagaagagaaggggcgggggctgccggagggaaatgggaactgatgcttccctgccggagcgcgcttgttcaatgttaagatctttgacaattcttcctgcggagagttcagatctgatagc

SEQ ID NO: 45 - DNA, 122 bp, Homo sapiens
  misc_feature (61)..(62): /note=“This region represents a CpG dinucleotide”
  misc_feature (61)..(61): /note=“Nucleotide may be methylated to form 5-methylcytosine”
gggagtcgagcgcggaggctcggggggcgggacgcggggcctgggagcggccagggaccgcggcagcgcctcagtgccagcctggcgcccgcgactgcctgccccagcccctcagtggcggc

SEQ ID NO: 46 - DNA, 122 bp, Homo sapiens
  misc_feature (61)..(62): /note=“This region represents a CpG dinucleotide”
  misc_feature (61)..(61): /note=“Nucleotide may be methylated to form 5-methylcytosine”
ccccaccccgaagggtccccgccgccgtcttagggctcagaggtgtggagctgggatgcgcgggaccctatccccggagtccctatcctgccccaatccggaccagaccttcggtctctaca

SEQ ID NO: 47 - DNA, 122 bp, Homo sapiens
  misc_feature (61)..(62): /note=“This region represents a CpG dinucleotide”
  misc_feature (61)..(61): /note=“Nucleotide may be methylated to form 5-methylcytosine”
gagaacgattttaccgccgatttaagcgttctcctttttccccttcgtggccgttttattcggcactgaaatgctgtttaacctcagcaagagtaggcgtgggaaggtggcgctcttcccca

SEQ ID NO: 48 - DNA, 122 bp, Homo sapiens
  misc_feature (61)..(62): /note=“This region represents a CpG dinucleotide”
  misc_feature (61)..(61): /note=“Nucleotide may be methylated to form 5-methylcytosine”
tctcccgccagccggcgcagcttgggtacgcagcggcagtggctatttgtcctcaggagccgcagcgctgtgagccacgcagccccttcctgggctcaggctccttggttgattctaggctc

SEQ ID NO: 49 - DNA, 122 bp, Homo sapiens
  misc_feature (61)..(62): /note=“This region represents a CpG dinucleotide”
  misc_feature (61)..(61): /note=“Nucleotide may be methylated to form 5-methylcytosine”
actggcatgatcagctgaactttgtggggtcagcgcttctctctggcttcccctcagcggcgccaaggcgaggggagcgcagaaccccggctcaggacggacagacagacggccagccgcgc

SEQ ID NO: 50 - DNA, 122 bp, Homo sapiens
  misc_feature (61)..(62): /note=“This region represents a CpG dinucleotide”
  misc_feature (61)..(61): /note=“Nucleotide may be methylated to form 5-methylcytosine”
ctggtgaccccatctgagcagctctctcctgacgtttaacgcaccccaaagcaagcggggcgcacagcactggtgattggcaaggttcgaggcccctcagcccctgaaagcccgtggcgtaa

SEQ ID NO: 51 - DNA, 122 bp, Homo sapiens
  misc_feature (61)..(62): /note=“This region represents a CpG dinucleotide”
  misc_feature (61)..(61): /note=“Nucleotide may be methylated to form 5-methylcytosine”
gcgccgcccgggaacaggaatagcgaggccttctcatgtttcctgactgccggtcccagccggcgaacatcctgcgggcgcggtatccacgttcccgggcgggtggagaggaagcgtccgcg

SEQ ID NO: 52 - DNA, 122 bp, Homo sapiens
  misc_feature (61)..(62): /note=“This region represents a CpG dinucleotide”
  misc_feature (61)..(61): /note=“Nucleotide may be methylated to form 5-methylcytosine”
ctttataggattgggctgccggtcgggcgcgggaggcctgaagggctccaatcagctgggcgcgggcgcgtcgccgttccggaccccgcgagatggggacgccaccagttcccctcccctcg

SEQ ID NO: 53 - DNA, 122 bp, Homo sapiens
  misc_feature (61)..(62): /note=“This region represents a CpG dinucleotide”
  misc_feature (61)..(61): /note=“Nucleotide may be methylated to form 5-methylcytosine”
tcttcccggtccagccaagagcctggaaccacgtgacctgcccatttgtatgccgcggagcgctccattccggcccctttgtggccagaagaaagtggcccatctgtcgccagttagagact

SEQ ID NO: 54 - DNA, 122 bp, Homo sapiens
  misc_feature (61)..(62): /note=“This region represents a CpG dinucleotide”
  misc_feature (61)..(61): /note=“Nucleotide may be methylated to form 5-methylcytosine”
tggcaagagatgtctggggatgaggtgaggcggggaagggggaacatcgctgcatggttacgagcacacgcgaatacaggaacctttagggaagctccaggccaaagagcatccgtgggctc

SEQ ID NO: 55 - DNA, 122 bp, Homo sapiens
  misc_feature (61)..(62): /note=“This region represents a CpG dinucleotide”
  misc_feature (61)..(61): /note=“Nucleotide may be methylated to form 5-methylcytosine”
agtcccggccggtcatggttccttagcaagcaaggcgcgagcctctctggatcccgtttgcggagggcccagcttagaggaagctttataggattgggctgccggtcgggcgcgggaggcct

SEQ ID NO: 56 - DNA, 122 bp, Homo sapiens
  misc_feature (61)..(62): /note=“This region represents a CpG dinucleotide”
  misc_feature (61)..(61): /note=“Nucleotide may be methylated to form 5-methylcytosine”
tcctaaagccagaaacgtcccggtttccctgaggtcctaaagaacgttgacagccaacagcgcgcctaacttggagcgaatcctcttcgggctttccagagtgcgggggatagataaagagt

SEQ ID NO: 57 - DNA, 122 bp, Homo sapiens
  misc_feature (61)..(62): /note=“This region represents a CpG dinucleotide”
  misc_feature (61)..(61): /note=“Nucleotide may be methylated to form 5-methylcytosine”
aatcacagcagcccccagtttagcggactgatttactcccggtattggtaaatatgatcacgtgggccgcgcgaccaatggtggaggctgcagcctgcgaactagtcggtggctcgggcgcc

SEQ ID NO: 58 - DNA, 122 bp, Homo sapiens
  misc_feature (61)..(62): /note=“This region represents a CpG dinucleotide”
  misc_feature (61)..(61): /note=“Nucleotide may be methylated to form 5-methylcytosine”
acgcgcgctctccttctcggcggccccgtgcgtctgcgcgcgcgcgcgctcgtccggccgcgaccagcgcgtgggagccgctcttatagtgaccaccagcaaggacagcgcaggtgatgcac

SEQ ID NO: 59 - DNA, 122 bp, Homo sapiens
  misc_feature (61)..(62): /note=“This region represents a CpG dinucleotide”
  misc_feature (61)..(61): /note=“Nucleotide may be methylated to form 5-methylcytosine”
acctcctcctatcccaccttcctctcgctggatcccagctacagttggctccggataggacgcttggctggagccagaggacgaggtagctcctagcctaggaatccttatctgctttgtaa

SEQ ID NO: 60 - DNA, 122 bp, Homo sapiens
  misc_feature (61)..(62): /note=“This region represents a CpG dinucleotide”
  misc_feature (61)..(61): /note=“Nucleotide may be methylated to form 5-methylcytosine”
acccccgccccttgcctcaggattcctcccctccctcccctgccccccaaccctgccctgcgcctgcgcaatgaaagacaaagtttttgctgacccgggcagcagactttggcagctgctct

SEQ ID NO: 61 - DNA, 122 bp, Homo sapiens
  misc_feature (61)..(62): /note=“This region represents a CpG dinucleotide”
  misc_feature (61)..(61): /note=“Nucleotide may be methylated to form 5-methylcytosine”
aagaatagacagtgatttaaccccctccttcctcctctacagcccgatccactctgttggcggctgcgccttgccttgggtgcagtgggacgtgtccctcccatgtttcccttaattaggag

SEQ ID NO: 62 - DNA, 122 bp, Homo sapiens
  misc_feature (61)..(62): /note=“This region represents a CpG dinucleotide”
  misc_feature (61)..(61): /note=“Nucleotide may be methylated to form 5-methylcytosine”
agggcggcgagtcgggcgcacgcgcaccccctgcccgcccctggcgcccctccccgcgggcggtgcagctacccctgcagcgcctcccctagctagaagggagcgggagggggctccgggcg

SEQ ID NO: 63 - DNA, 122 bp, Homo sapiens
  misc_feature (61)..(62): /note=“This region represents a CpG dinucleotide”
  misc_feature (61)..(61): /note=“Nucleotide may be methylated to form 5-methylcytosine”
ctggaaacgcacgtgaacaaagctctcgcccccgggagccgctgcctgcggtttcctagtcgatcccagcttctctagggagtgtcaggcgcacacagggttaagttagttccctccctggt

SEQ ID NO: 64 - DNA, 122 bp, Homo sapiens
  misc_feature (61)..(62): /note=“This region represents a CpG dinucleotide”
  misc_feature (61)..(61): /note=“Nucleotide may be methylated to form 5-methylcytosine”
gagggcgctcccccgccgctggagccgcagggcgctgcttttcgctgactctgccaaacacgccattagggcccgcctgggagggcgctaccctaatgaagcggctctaagtcacctttgaa

SEQ ID NO: 65 - DNA, 122 bp, Homo sapiens
  misc_feature (61)..(62): /note=“This region represents a CpG dinucleotide”
  misc_feature (61)..(61): /note=“Nucleotide may be methylated to form 5-methylcytosine”
caccccccaccccccaaacaaaaatataccaccacttaaagaggtcctcgctttacatttcgaagaagggatgccagttcataaacagttttcggtgatttacttccctgcaaatgagttgt

SEQ ID NO: 66 - DNA, 122 bp, Homo sapiens
  misc_feature (61)..(62): /note=“This region represents a CpG dinucleotide”
  misc_feature (61)..(61): /note=“Nucleotide may be methylated to form 5-methylcytosine”
cttctaactgacgtctcccgcgagcgactgctctgcccccacttgggaagtccagggaggcgcaaggcggccacctccgccgctccccccagcagcaggagcagccgcagctccgccgcacg

SEQ ID NO: 67 - DNA, 23 bp, Artificial Sequence
  source: /note=“Description of Artificial Sequence Synthetic probe”
aggcgttgtcgcggtttttggtc

SEQ ID NO: 68 - DNA, 28 bp, Artificial Sequence
  source: /note=“Description of Artificial Sequence Synthetic probe”
agttttcgttttcgggagtcttgtttgc

SEQ ID NO: 69 - DNA, 27 bp, Artificial Sequence
  source: /note=“Description of Artificial Sequence Synthetic probe”
aaatatgattacgtgggtcgcgcgatt

SEQ ID NO: 70 - DNA, 23 bp, Artificial Sequence
  source: /note=“Description of Artificial Sequence Synthetic probe”
tagggcgggttttttgcgttcgg

SEQ ID NO: 71 - DNA, 25 bp, Artificial Sequence
  source: /note=“Description of Artificial Sequence Synthetic probe”
cgcgttcgcgtttagttgattggag

SEQ ID NO: 72 - DNA, 33 bp, Artificial Sequence
  source: /note=“Description of Artificial Sequence Synthetic probe”
tcgggaataagaggataggtttagacgattgga

SEQ ID NO: 73 - DNA, 29 bp, Artificial Sequence
  source: /note=“Description of Artificial Sequence Synthetic probe”
ttagtaagagtaggcgtgggaaggtggcg

SEQ ID NO: 74 - DNA, 30 bp, Artificial Sequence
  source: /note=“Description of Artificial Sequence Synthetic probe”
tatatttttgagttttaagacggcggcggg

SEQ ID NO: 75 - DNA, 22 bp, Artificial Sequence
  source: /note=“Description of Artificial Sequence Synthetic probe”
cgggcgggttaatgagttgcgc

SEQ ID NO: 76 - DNA, 33 bp, Artificial Sequence
  source: /note=“Description of Artificial Sequence Synthetic probe”
tttggagcgaatttttttcgggttttttagagt

SEQ ID NO: 77 - DNA, 25 bp, Artificial Sequence
  source: /note=“Description of Artificial Sequence Synthetic probe”
ttttttagcggcgttaaggcgaggg

SEQ ID NO: 78 - DNA, 26 bp, Artificial Sequence
  source: /note=“Description of Artificial Sequence Synthetic probe”
ttgtcgtcgtcgttttttttaggcgg

SEQ ID NO: 79 - DNA, 30 bp, Artificial Sequence
  source: /note=“Description of Artificial Sequence Synthetic probe”
ttaaagagtattcgtgggttttcggagcgt

SEQ ID NO: 80 - DNA, 34 bp, Artificial Sequence
  source: /note=“Description of Artificial Sequence Synthetic probe”
taggttacgtggttttaggtttttggttggatcg

SEQ ID NO: 81 - DNA, 30 bp, Artificial Sequence
  source: /note=“Description of Artificial Sequence Synthetic probe”
cgcgttttatttataggagcgtagcgttcg

SEQ ID NO: 82 - DNA, 25 bp, Artificial Sequence
  source: /note=“Description of Artificial Sequence Synthetic probe”
cggcgtagtttgggtacgtagcggt

SEQ ID NO: 83 - DNA, 35 bp, Artificial Sequence
  source: /note=“Description of Artificial Sequence Synthetic probe”
taaggtaaggcgtagtcgttaatagagtggatcgg

SEQ ID NO: 84 - DNA, 31 bp, Artificial Sequence
  source: /note=“Description of Artificial Sequence Synthetic probe”
taaatttgtcgagttttgtagcgtaagcgcg

SEQ ID NO: 85 - DNA, 20 bp, Artificial Sequence
  source: /note=“Description of Artificial Sequence Synthetic forward primer”
gcgggcgttaggttggtatt

SEQ ID NO: 86 - DNA, 22 bp, Artificial Sequence
  source: /note=“Description of Artificial Sequence Synthetic forward primer”
tgggcgttggaagttagttacg

SEQ ID NO: 87 - DNA, 24 bp, Artificial Sequence
  source: /note=“Description of Artificial Sequence Synthetic forward primer”
gcggattgatttattttcggtatt

SEQ ID NO: 88 - DNA, 18 bp, Artificial Sequence
  source: /note=“Description of Artificial Sequence Synthetic forward primer”
cggttgggcggatttagc

SEQ ID NO: 89 - DNA, 20 bp, Artificial Sequence
  source: /note=“Description of Artificial Sequence Synthetic forward primer”
gggaattggtggcgttttta

SEQ ID NO: 90 - DNA, 26 bp, Artificial Sequence
  source: /note=“Description of Artificial Sequence Synthetic forward primer”
cggttgcgttcgtaatagtatttagt

SEQ ID NO: 91 - DNA, 24 bp, Artificial Sequence
  source: /note=“Description of Artificial Sequence Synthetic forward primer”
tggtcgttttattcggtattgaaa

SEQ ID NO: 92 - DNA, 25 bp, Artificial Sequence
  source: /note=“Description of Artificial Sequence Synthetic forward primer”
ggatagggtttcgcgtattttagtt

SEQ ID NO: 93 - DNA, 26 bp, Artificial Sequence
  source: /note=“Description of Artificial Sequence Synthetic forward primer”
gcgttttataatatttgggattgatg

SEQ ID NO: 94 - DNA, 23 bp, Artificial Sequence
  source: /note=“Description of Artificial Sequence Synthetic forward primer”
cgtttcggtttttttgaggtttt

SEQ ID NO: 95 - DNA, 24 bp, Artificial Sequence
  source: /note=“Description of Artificial Sequence Synthetic forward primer”
cgttgggattggtatgattagttg

SEQ ID NO: 96 - DNA, 22 bp, Artificial Sequence
  source: /note=“Description of Artificial Sequence Synthetic forward primer”
gtttttgtttcgggttggagtt

SEQ ID NO: 97 - DNA, 27 bp, Artificial Sequence
  source: /note=“Description of Artificial Sequence Synthetic forward primer”
tgtatggttacgagtatacgcgaatat

SEQ ID NO: 98 - DNA, 23 bp, Artificial Sequence
  source: /note=“Description of Artificial Sequence Synthetic forward primer”
gagcgtttcgcggtatataaatg

SEQ ID NO: 99 - DNA, 17 bp, Artificial Sequence
  source: /note=“Description of Artificial Sequence Synthetic forward primer”
gagcgcgaagcgggtat

SEQ ID NO: 100 - DNA, 22 bp, Artificial Sequence
  source: /note=“Description of Artificial Sequence Synthetic forward primer”
ttttgcgtgttgttttttcgtt

SEQ ID NO: 101 - DNA, 23 bp, Artificial Sequence
  source: /note=“Description of Artificial Sequence Synthetic forward primer”
ggaaatatgggagggatacgttt

SEQ ID NO: 102 - DNA, 18 bp, Artificial Sequence
  source: /note=“Description of Artificial Sequence Synthetic forward primer”
gcgcgttgaaggttttgg

SEQ ID NO: 103 - DNA, 23 bp, Artificial Sequence
  source: /note=“Description of Artificial Sequence Synthetic reverse primer”
ccctacactctaacgcgctaaaa

SEQ ID NO: 104 - DNA, 29 bp, Artificial Sequence
  source: /note=“Description of Artificial Sequence Synthetic reverse primer”
cactcctaaaaaaactaaaatcgactaaa

SEQ ID NO: 105 - DNA, 23 bp, Artificial Sequence
  source: /note=“Description of Artificial Sequence Synthetic reverse primer”
caccgactaattcgcaaactaca

SEQ ID NO: 106 - DNA, 21 bp, Artificial Sequence
  source: /note=“Description of Artificial Sequence Synthetic reverse primer”
cccaacaaccaacgactactt

SEQ ID NO: 107 - DNA, 20 bp, Artificial Sequence
  source: /note=“Description of Artificial Sequence Synthetic reverse primer”
actaccgatcgaacgcgaaa

SEQ ID NO: 108 - DNA, 26 bp, Artificial Sequence
  source: /note=“Description of Artificial Sequence Synthetic reverse primer”
ccgactttaaaaaacctacaaacaaa

SEQ ID NO: 109 - DNA, 29 bp, Artificial Sequence
  source: /note=“Description of Artificial Sequence Synthetic reverse primer”
cttccgctaaacaactaaaaaatacctaa

SEQ ID NO: 110 - DNA, 17 bp, Artificial Sequence
  source: /note=“Description of Artificial Sequence Synthetic reverse primer”
cccaaacaccgcccaaa

SEQ ID NO: 111 - DNA, 20 bp, Artificial Sequence
  source: /note=“Description of Artificial Sequence Synthetic reverse primer”
cattaatacgcccgcattcc

SEQ ID NO: 112 - DNA, 27 bp, Artificial Sequence
  source: /note=“Description of Artificial Sequence Synthetic reverse primer”
cgtctccccaactactctttatctatc

SEQ ID NO: 113 - DNA, 24 bp, Artificial Sequence
  source: /note=“Description of Artificial Sequence Synthetic reverse primer”
ccgtctatctatccgtcctaaacc

SEQ ID NO: 114 - DNA, 19 bp, Artificial Sequence
  source: /note=“Description of Artificial Sequence Synthetic reverse primer”
atcaacgcccgaaaaatcc

SEQ ID NO: 115 - DNA, 20 bp, Artificial Sequence
  source: /note=“Description of Artificial Sequence Synthetic reverse primer”
aacctcgacaatccgaaacg

SEQ ID NO: 116 - DNA, 23 bp, Artificial Sequence
  source: /note=“Description of Artificial Sequence Synthetic reverse primer”
cccgacatacgccatataatctt

SEQ ID NO: 117 - DNA, 20 bp, Artificial Sequence
  source: /note=“Description of Artificial Sequence Synthetic reverse primer”
gcgaattacgcataaaccaa

SEQ ID NO: 118 - DNA, 27 bp, Artificial Sequence
  source: /note=“Description of Artificial Sequence Synthetic reverse primer”
gctacgactcctaaaaacaaataacca

SEQ ID NO: 119 - DNA, 22 bp, Artificial Sequence
  source: /note=“Description of Artificial Sequence Synthetic reverse primer”
taaccccctccttcctcctcta

SEQ ID NO: 120 - DNA, 22 bp, Artificial Sequence
  source: /note=“Description of Artificial Sequence Synthetic reverse primer”
ccccgaaattacaatcgaaaca

(19) United States
(12) Patent Application Publication
(10) Pub. No.: US 2024/0043933 A1
(43) Pub. Date: Feb. 8, 2024

(54) METHODS FOR DETECTING AND PREDICTING BREAST CANCER

(71) Applicant: UCL Business Ltd, London (GB)

(72) Inventors: Martin Widschwendter, London (GB); Allison Jones, London (GB); Iona Evans, London (GB)

(21) Appl. No.: 18/009,959

(22) PCT Filed: Jun. 17, 2021
(86) PCT No.: PCT/GB2021/051539

§ 371 Date: Dec. 12, 2022

(30) Foreign Application Priority Data
Jun. 17, 2020 (GB) 2009217.7

(51) Int. Cl. C12Q 1/6886

(52) U.S. Cl. CPC C12Q 1/6886; C12Q 2600/118; C12Q 2600/154

(57) ABSTRACT

The present invention relates to assays for predicting the presence, absence or development of breast cancer in an individual, by determining the methyl ati on status of certain CpGs in a population of DNA molecules in a sample which has been taken from the individual, deriving an index value based on the methylation status of the certain CpGs, and predicting the presence, absence or development of breast cancer in the individual based on the breast cancer index value. The invention further relates to a method of treating and/or preventing breast cancer in an individual, the method comprising assessing the presence, absence or development of breast cancer in an individual by performing the assays of the invention, followed by administering one or more therapeutic or preventative treatments or measures to the individual based on the assessment. The invention further provides a method of monitoring the breast cancer status of an individual according to changes in the individual's breast cancer index value over the course of time. The invention further relates to arrays which are suitable for performing the assays of the invention.

### SEQUENCE LISTING

The instant application contains a Sequence Listing which has been submitted electronically in ASCII format and is hereby incorporated by reference in its entirety. Said ASCII copy, created on Jun. 9, 2021, is named ‘Sequence listing as filed 17 Jun. 2021 N417983WO MGW JAS.txt’ and is 59,899 bytes in size.

### FIELD OF THE INVENTION

The present invention relates to assays for predicting the presence, absence or development of breast cancer in an individual, by determining the methylation status of certain CpGs in a population of DNA molecules in a sample which has been taken from the individual, deriving an index value based on the methylation status of the certain CpGs, and predicting the presence, absence or development of breast cancer in the individual based on the breast cancer index value. The invention further relates to a method of treating and/or preventing breast cancer in an individual, the method comprising assessing the presence, absence or development of breast cancer in an individual by performing the assays of the invention, followed by administering one or more therapeutic or preventative treatments or measures to the individual based on the assessment. The invention further provides a method of monitoring the breast cancer status of an individual according to changes in the individual's breast cancer index value over the course of time. The invention further relates to arrays which are suitable for performing the assays of the invention.

The project leading to this application has received funding from the European Research Council (ERC) under the European Union's Horizon 2020 research and innovation programme (grant agreement No 742432—BRCA-ERC).

### BACKGROUND TO THE INVENTION

Breast cancer is by far the most common cancer in women. Eight in ten women will be diagnosed with this disease at least once in their lifetime.

To date, effective non-surgical primary breast cancer prevention has not materialized owing to two main issues. The first is that only selective estrogen receptor modulators, having been demonstrated to reduce contralateral breast cancer in adjuvant treatment trials (i.e. tamoxifen and aromatase-inhibitors), have being used so far in large prospective primary prevention trials. However, not one of these drugs has led to a reduction in estrogen receptor negative cancers. Specifically, there was no reduction in triple negative breast cancers (TNBC), which are highly prevalent in BRCA1/2 mutation carriers. Triple negative breast cancers, for which luminal progenitor cells are deemed to serve as cells-of-origin, lack the estrogen receptor (ER), the progesterone receptor (PR) and the human epidermal growth factor receptor 2 (HER2), have a particularly poor prognosis and are regarded as being aggressive forms of breast cancer. The second main issue which has hampered effective non-surgical primary breast cancer prevention is that no molecular marker(s) yet exists that are capable of monitoring the efficacy of cancer-preventive strategies.

Progesterone plays an essential role in the formation of aggressive breast cancers. A recent meta-analysis showed that women taking a progesterone-containing menopausal hormone treatment not only had a higher breast cancer incidence, but additional cancers spread beyond the breast. Furthermore, these women were more likely to die from the breast cancer compared to women who only took estrogens.

Additional evidence for the role of progesterone in breast carcinogenesis comes from the observation that compared to non-mutation carriers, BRCA1/2 germline mutation carriers show elevated levels of luteal phase progesterone. This leads to an increase in RANKL levels in the breast and to reduced levels of the physiological RANKL-antagonist Osteoprotegerin, thus leading to an increase and expansion of estrogen- and progesterone-receptor negative mammary stem cells and eventual breast cancer formation. Interfering with the progesterone pathway in rodents showed that BRCA-mediated breast cancer formation can be prevented by the anti-progesterone compound Mifepristone. Recently, it was also suggested that moderate folic acid- and vitamin B12-containing supplement use may be protective for BRCA-associated breast cancer.

Thus, whilst there is evidence that progesterone antagonists may be efficacious in breast cancer treatment, there remains a need for (i) one or more markers for indicating the risk of breast cancer development to thus provide clinicians with an indication of a suitable preventative treatment plan or screening regimen; and (ii) one or more markers for monitoring the efficacy of preventative breast cancer treatments or for monitoring the efficacy of treatments administered to individuals that harbour breast cancer.

### SUMMARY OF THE INVENTION

The current inventors set out to understand whether DNAme (DNA methylation) profiles may be used to detect the presence or absence of breast cancer. The inventors also set out to understand whether said DNAme profiles may be associated with the development of breast cancer, and therefore whether such profiles may be capable of functioning as surrogate markers for individual stratification purposes in connection with breast cancer.

In this regard the inventors have succeeded in developing assays involving “breast cancer index values” which are derived from and associated with DNAme profiles established from breast tissue from a given individual, and which values can be used to stratify the individual in connection with breast cancer.

The breast cancer index value is determined from data relating to the methylation status of one or more CpGs in a panel of CpGs as further defined and described herein. CpGs of the panel are methylation sites in DNA from cells derived from breast tissue.

For the purposes of the present invention, the breast cancer index value may be used interchangeably herein with “WID-Breast”, “WID-Breast-Index”, “breast cancer index”, “index” or “index value” (WID=women's risk identification for breast cancer index). Furthermore, any reference to a cancer index value in the context of the present invention, may be equally used for the assessment of the presence, absence or development of endometrial cancer and/or ovarian cancer in an individual.

Based on studies with patients known to be free of breast cancer, the inventors have established breast cancer index values, using specific panels of CpGs, which have been determined to be associated with/characteristic of breast tissue which is negative for breast cancer, i.e. normal breast tissue which is free of breast cancer. Based on studies with patients known to possess breast cancer, the inventors have established breast cancer index values which have been determined to be associated with/characteristic of breast tissue which is positive for breast cancer. Based on studies with patients known to possess triple negative breast cancer, the inventors have established breast cancer index values which have been determined to be associated with/characteristic of breast tissue which is positive for triple negative breast cancer. Based on studies with patients known to possess non-triple negative breast cancer, the inventors have established breast cancer index values which have been determined to be associated with/characteristic of breast tissue which is positive for non-triple negative breast cancer.

Thus, the inventors have been able to establish a breast cancer index value continuum, using specific panels of CpGs, which at one end can be defined as characterizing normal breast tissue which is free of breast cancer and which at the opposite end can be defined as characterizing breast tissue harboring triple negative breast cancer, a highly aggressive form of breast cancer.

The inventors have been able to establish breast cancer index values which can be defined as characterizing breast tissue harboring non-triple negative breast cancer. The inventors have established that breast cancer index values which characterize breast tissue as positive for non-triple negative breast cancer fall, on the above-described index value continuum, between breast cancer index values which characterize breast tissue which is negative for breast cancer and breast cancer index values which characterize breast tissue which is positive for triple negative breast cancer.

By determining the methylation profile-based breast cancer index value from breast tissue derived from the individual, the individual may be seen to possess a breast cancer index value which correlates with those possessed by individuals which are known, via the inventor's studies described herein, to fall within one of these groups. Such correlations have been determined with a high degree of statistical accuracy, particularly with respect to parameters relevant to biological assays such as receiver operating characteristics (ROC) sensitivity and specificity, as well as area under the curve (AUC). Accordingly, by determining the breast cancer index value from breast tissue from a given individual, the individual may be determined to possess breast tissue which is positive for breast cancer, i.e. the individual is diagnosed as having breast cancer. Conversely, by determining the breast cancer index value from breast tissue from a given individual, the individual may be determined to possess breast tissue which is negative for breast cancer, i.e. the individual is diagnosed as not having breast cancer. By determining the methylation profile-based breast cancer index value from breast tissue derived from the individual, the individual may be seen to possess a breast cancer index value which is intermediate between these groups. As such, the breast cancer index value may identify that the individual has a breast tissue-based methylation profile which is more characteristic of e.g. breast tissue which is negative for breast cancer, or which is more characteristic of e.g. breast tissue which is positive for breast cancer. The inventors have observed that such intermediate breast cancer index values may be possessed by individuals harboring certain BRCA mutations, but who have not yet developed breast cancer. Furthermore, as described in more detail herein, the inventors have studied women who have been administered pharmaceutical compounds known to possess breast cancer preventative properties, such as mifepristone. Remarkably, the methylation profile-based breast cancer index value from breast tissue derived from certain of these individuals was shown to change over the course of treatment with the compound, so as to be more characteristic of breast tissue which is negative for breast cancer. These observations surprisingly demonstrate that over the course of treatment the methylation profile of DNA from breast tissue from these individuals, as assessed by the breast cancer index value, was subject to alteration, and changed from a profile more characteristic of breast tissue which is positive for breast cancer to a profile more characteristic of breast tissue which is negative for breast cancer. These observations from the current inventors establish that the breast cancer index value, as further described and defined herein, is dynamic and can change according to genetic and environmental conditions. Consequently the breast cancer index value can be used as a means for assessing the status of a given individual on a breast cancer index value continuum in connection with breast cancer. It further follows that the breast cancer index value can be used as a means for monitoring the status of a given individual on a breast cancer index value-based continuum in connection with breast cancer, for example before, during and after one or more breast cancer preventative or therapeutic treatments.

Accordingly, in the context of the present invention, by determining the breast cancer index value from breast tissue from a given individual it is possible to assess the presence, absence or development of breast cancer in an individual, or in other words to stratify the individual for breast cancer. In the context of the present invention, stratification for breast cancer is the process of categorizing the individual as being a member of a group of individuals who possess a phenotype in connection with breast cancer, including the presence or absence of breast cancer in the individual, or the development of breast cancer, i.e. by having breast tissue which is more characteristic of breast tissue which is breast cancer positive than breast tissue which is breast cancer negative.

As explained herein, the assays methods of the invention are based on a breast cancer index value derived from a methylation profile from DNA originating from breast tissue. Accordingly, the assays provide means for correlating a breast tissue-derived DNA methylation profile with a status connected with breast cancer ranging from the originating breast tissue being breast cancer negative, to the originating breast tissue being breast cancer positive, and even to triple negative breast cancer positive. Because the assays of the invention provide a correlation between the methylation profile and the disease status, the skilled person will appreciate that as part of the stratification process and outcome, disease status is assigned on the basis of a likelihood. As such, the methods of the invention provide assays which are predictive of an individual's status with respect to breast cancer. The assays of the invention accordingly provide means for predicting the presence or absence of breast cancer in an individual. The assays of the invention accordingly also provide means for predicting the development of breast cancer in an individual. The assays of the invention can provide means for predicting the development of breast cancer in an individual since the inventors have demonstrated that specific breast cancer index values can define breast tissue which is breast cancer negative, whilst others can define breast tissue which is breast cancer positive, and since the specific breast cancer index values may be dynamic and may be subject to change along a continuum from breast cancer negative to breast cancer positive.

Whilst disease status may be assigned on the basis of a likelihood, the inventors have demonstrated herein that correlations between a breast tissue-derived DNA methylation profile and breast cancer disease status using breast cancer index values can be achieved with a very high degree of statistical accuracy using parameters relevant to biological assays, as described further herein. As such, the assays of the invention provide means for predicting the presence or absence of breast cancer in an individual and for predicting the development of breast cancer in an individual, and for stratifying an individual for breast cancer, and wherein the prediction/stratification can be defined to be statistically highly reliable and robust. This in turn means that the prediction/stratification can be made with a high level of confidence. The assays of the invention can be defined to be statistically accurate by means known in the art, as further described and defined herein. The assays of the invention can be defined according to parameters relating to their statistical specificity and sensitivity. These parameters define the likelihood of false positive and false negative test results. The lower the proportion of false positive and false negative test results the more statistically accurate the assay becomes. In this regard the inventors have established CpG panels, as described and defined further herein, wherein the methylation status of CpGs in the panel can be used to establish breast cancer index values such that the assays produce statistically accurate predictions of breast cancer disease status. Accordingly, the inventors have determined that the assays described herein may be defined according to statistical parameters such as percentage specificity and sensitivity and also by receiver operating characteristics (ROC) area under the curve (AUC). All such means are known in the art and are known to be defined measures of statistical accuracy for biological assays such as those described and defined herein.

Thus the methods of the invention provide assays which can be used, with a high degree of statistical accuracy, to predict the presence, absence or development of breast cancer. The methods of the invention provide assays which can be used, with a high degree of statistical accuracy, to stratify an individual with respect to breast cancer status. Accordingly the methods of the invention provide useful information to individuals and their physicians concerning patient breast cancer status. This information may help inform actual therapeutic treatment measures if the presence of breast cancer is identified in the individual. The information may help to monitor the progress of therapeutic treatment measures in the individual by monitoring changes in the breast cancer index value over the course of a period of time. The information may help to monitor the progress of prophylactic or preventative treatment measures in the individual by monitoring changes in the breast cancer index value over the course of a period of time. As such the methods of the invention offer significant advantages in the personalized treatment, management and prevention of breast cancer in individuals.

Accordingly, the invention provides the following.

The invention provides an assay for assessing the presence, absence or development of breast cancer in an individual, the assay comprising:
a. providing a sample which has been taken from the individual, the sample comprising a population of DNA molecules;
b. determining in the population of DNA molecules in the sample the methylation status of a panel of one or more CpGs selected from within a panel of one or more Differentially Methylated Regions (DMRs) defined by SEQ ID NOs 1 to 29, wherein selected CpGs in each DMR are denoted by CG;
c. deriving a breast cancer index value (WID-Breast-Index) based on the methylation status of the one or more CpGs in the panel; and
d. assessing the presence, absence or development of breast cancer in the individual based on the breast cancer index value.

Any assay of the invention may be defined as an assay for stratifying an individual for the presence, absence or development of breast cancer, the assay comprising:
a. providing a sample which has been taken from the individual, the sample comprising a population of DNA molecules;
b. determining in the population of DNA molecules in the sample the methylation status of a panel of one or more CpGs selected from within a panel of one or more Differentially Methylated Regions (DMRs) defined by SEQ ID NOs 1 to 29, wherein selected CpGs in each DMR are denoted by CG;
c. deriving a breast cancer index value (WID-Breast-Index) based on the methylation status of the one or more CpGs in the panel; and
d. stratifying the individual for the presence, absence or development of breast cancer based on the breast cancer index value.

Any assay of the invention may be defined as an assay for stratifying an individual for breast cancer, the assay comprising:
a. providing a sample which has been taken from the individual, the sample comprising a population of DNA molecules;
b. determining in the population of DNA molecules in the sample the methylation status of a panel of one or more CpGs selected from within a panel of one or more Differentially Methylated Regions (DMRs) defined by SEQ ID NOs 1 to 29, wherein selected CpGs in each DMR are denoted by CG;
c. deriving a breast cancer index value (WID-Breast-Index) based on the methylation status of the one or more CpGs in the panel; and
d. stratifying the individual for breast cancer based on the breast cancer index value.

Any of the aforementioned assays may be performed in accordance with any of the assay methods disclosed and defined herein.

The invention further provides a method of treating and/or preventing breast cancer in an individual, the method comprising:
a. determining the breast cancer status of the individual by assessing the presence, absence or development of breast cancer in the individual by performing any the assays disclosed and defined herein; and
b. administering one or more therapeutic or preventative treatments or measures to the individual based on the assessment.

The invention further provides a method of monitoring the breast cancer status of an individual according to the individual's breast cancer index value, the method comprising: (a) assessing the presence, absence or development of breast cancer in an individual by performing any the assays disclosed and defined herein at a first time point; (b) assessing the presence, absence or development of breast cancer in the individual by performing any the assays disclosed and defined herein at one or more further time points; and (c) monitoring any change in the breast cancer status of the individual, preferably wherein the assays of (a) and (b) are performed using the same assays.

The invention further provides an array capable of discriminating between methylated and non-methylated forms of CpGs; the array comprising oligonucleotide probes specific for a methylated form of each CpG in a CpG panel and oligonucleotide probes specific for a non-methylated form of each CpG in the panel; wherein the panel consists of any combination of CpGs disclosed in connection with any the assays described and defined herein.

The invention further provides a hybridized array, wherein the array is obtainable by hybridizing to an array of the invention, as described above, any group of oligonucleotides comprising any set or combination of CpGs disclosed in connection with any the assays described and defined herein.

The invention further provides a process for making a hybridized array, comprising contacting an array of the invention, as described above, with a group of oligonucleotides comprising or corresponding with any set or combination of CpGs disclosed in connection with any the assays described and defined herein.

### BRIEF DESCRIPTION OF THE FIGURES

FIG. 1 shows the WID-Breast29 increased in triple negative breast cancer (TNBC) and in the surrounding tissue.

The WID-Breast29 (definition shown in Panel A) was significantly greater in normal breast tissue surrounding TNBC (n=14) when compared to normal tissue from cancer-free women (n=14), (Panel B); significance was assessed with the t-test, and all enrolled volunteers were included in the analysis. Subsequently, the WID-Breast29 increased significantly from normal surrounding tissue to TNBC in the same volunteers (n=14), (Panel C); significance was assessed with the paired-sample t-test, and all enrolled volunteers were included in the analysis.

FIG. 2 shows the WID-Breast29 increased consistently from healthy cancer-free to late-stage breast-cancer.

The WID-Breast29 increased significantly from normal breast tissue from cancer-free women to normal tissue surrounding breast cancer, to early-stage, and to late-stage breast cancer, with increasing magnitude corresponding to the progressively worsening prognosis of these groups (Panel A); significance was assessed with the t-test, and nine tumour samples were excluded from the analysis owing to missing data on disease stage. The WID-Breast29 also increased significantly from normal surrounding tissue to cancer in the same volunteers (n=42), (Panel B); significance was assessed with the paired-sample t-test, and all enrolled volunteers were included in the analysis.

FIG. 3 shows that mifepristone treatment reduced the WID-Breast29.

Treatment with vitamins did not lead to a significant change in the WID-Breast29 (panel A), whereas treatment with mifepristone significantly reduced the WID-Breast29 (panel B). Significance was assessed with the paired-sample t-test.

FIG. 4 shows enrolment and randomisation in the mifepristone clinical trials.

The flow-chart describes the two trials that recruited healthy women (Trial 1) and BRCA mutation carriers (Trial 2) and randomised them into vitamin and mifepristone arms. Numbers in brackets represent the total number of women recruited.

FIG. 5 shows expression levels of WID-Breast genes in basal stem-cells and luminal progenitors.

The WID-Breast genes were defined as all 29 genes mapped by any WID-Breast CpG, according to the DNAme array information provided by Illumina for the EPIC DNAme array, and which appears on the Agilent SurePrint G3 gene expression microarray. Significances compare mean expression across all genes as defined above for all samples of the two different types (basal stem-cell and luminal progenitor), and were calculated with the Wilcox-test.

FIG. 6 shows the epithelial cell fraction did not increase in the surrounding tissue or in the triple negative breast cancer (TNBC).

The epithelial cell fraction did not significantly change between normal breast tissue surrounding TNBC (n=14) compared to normal tissue from cancer-free women (n=14), (Panel A); significance was assessed with the t-test, and all enrolled volunteers were included in the analysis. The epithelial cell fraction did not significantly change between normal tissue surrounding TNBC compared to TNBC in the same volunteers (n=14), (Panel B); significance was assessed with the paired-sample t-test, and all enrolled volunteers were included in the analysis

FIG. 7 shows a boxplot of WID-Breast for Ki67<14% and Ki67>14%.

Significances were calculated with the Fisher's exact test, with samples partitioned into two groups by the median of their WID-Breast, as in FIG. 12. Samples with missing data (113 in total) were excluded from the analyses.

FIG. 8 shows the association of the WID-Breast with chronological age.

Significances were calculated with the t-test, based on Fisher-transformed product-moment correlation coefficients between the variables. All enrolled volunteers for which we had normal breast tissue samples available in Set 1 (Panels A and B) and Set 2 (Panels C and D) were included in the analyses.

FIG. 9 shows that the WID-Breast5 increased in the normal breast tissue surrounding triple negative breast cancer compared to normal breast tissue form cancer-free women.

Significances were calculated with the t-test, based on Fisher-transformed product-moment correlation coefficients between the variables.

FIG. 10 shows that the WID-Breast5 increased in the triple negative breast cancer compared to the matched normal breast tissue surrounding the cancer.

Significances were calculated with the t-test, based on Fisher-transformed product-moment correlation coefficients between the variables.

FIG. 11 shows that mifepristone treatment reduced the WID-Breast5.

Treatment with vitamins did not lead to a significant change in the WID-Breast5 (panel A), whereas treatment with mifepristone significantly reduced the WID-Breast5 (panel B). Significance was assessed with the paired-sample t-test.

FIG. 12 the association of the WID-Breast29 with various clinical factors.

Samples were partitioned into two groups by the median of their WID-Breast29, 0.303. Based on this, 2×2 contingency tables were produced and significance was assessed by Fisher's exact test. The number of samples that were missing data for each clinical factor (and therefore excluded from significance calculations) are also provided.

FIG. 13 shows the panel of 37 WID-Breast CpGs.

For deriving the breast cancer index of the invention, the methylation status of each CpG in a set of test CpGs selected from the panel of CpGs identified in FIG. 10 may be derived.

FIG. 14 shows forward and reverse primers, and probes designed for the methylated version of the regions, of the 18 genes for the Methylight regions.

### DETAILED DESCRIPTION OF THE INVENTION

### Identification of CpGs

It is understood that the mitotic age of normal stem cells is strongly associated with cancer risk. The inventors have previously demonstrated that DNA methylation of polycomb-group target genes (PCGTs) is highly prevalent in cancer, and increases with increasing age. Furthermore, the inventors previously identified CpG sites within these PCGTs by comparing stem cells in fetal and adult tissues that are capable of defining a “tick rate” that correlates with the estimated number of accumulated stem cell divisions in normal tissues. The inventors called this stem cell “mitotic clock” pcgtAge.

The inventors sought to extend previous studies to breast cancer and to develop assays capable of assessing the presence, absence or development of breast cancer in an individual.

The inventors compared CpGs methylation levels from within the collection of CpGs that make up pcgtAge. This led to the surprising establishment of a “breast cancer index” (used interchangeably herein with “WID-Breast” or “WID-Breast-Index” or “breast cancer index value” or “index” or “index value”) based on a comparison of CpG methylation levels in non-cancerous breast tissue samples derived from control individuals relative to non-cancerous breast tissue derived from, as yet, cancer-free women with a BRCA1/2 germline mutation (see Examples for further details).

A CpG as defined herein refers to the CG dinucleotide motif identified in relation to each SEQ ID NO. and chromosome position of the sequence as set out below (SEQ ID NOs 1 to 29), wherein the cytosine base of the dinucleotide identified in square brackets may (or may not) be modified. Thus by determining the methylation status of any panel of one or more CpGs defined by or identified in a given SEQ ID NO, it is meant that a determination is made as to the methylation status of the cytosine of the CG dinucleotide motif identified in square brackets in the panel of one or more CpGs in each sequence shown below, accepting that variations in the sequence upstream and downstream of any given CpG may exist due to sequencing errors or variation between individuals.

As explained in the examples below, the inventors identified 29 differentially methylated regions (DMRs) with relevance to human breast cancer. The nucleotide sequences of the 29 DMRs are defined respectively by the nucleotide sequences of SEQ TD NO: 1 to 29 as set out in Table 1 below, accepting that variation in the nucleotide sequence of any given DMR may exist due to sequencing errors and/or variation between individuals. In each sequence shown below the cytosine of the CG dinucleotide motif identified in square brackets is a cytosine of a CpG which may be included in a panel of CpGs when performing the assays of the invention. Any CG dinucleotide present within the sequence of SEQ ID NO: 1 to 29 may be included in a panel of CpGs when performing the assays of the invention. The CG dinucleotide motifs identified in square brackets, underlined and in bold correspond with the additional 37 CpGs defined according to SEQ TD NOS: 30 to 66 as set out further herein (e.g. see Table 2 below).

TABLE 1
Differentially methylated regions (DMRs) harbouring CpGs for use in assays
defined herein.

DRD5 (SEQ ID NO: 1)
>hg19_dna range = chr4: 9782900-9783500 5′pad = 0 3′pad = 0 strand = +
repeatMasking = none
ACT[CG]GGTCTGTGGGTAATCCAAGGCTGCCC[CG]CTTACCCTTCAAATGCTCTTTCCCCCTC[CG]
GAAGCCCT[CG][CG]TCCTCATCCTTACCCCACCTCTTGTTCCCCAAG[CG]TGGCCAGGGCTAAG
GCTCCAGGGATG[CG]CCAAGCACCCTT[CG]GTTTTCC[CG]GGGAGAATTTTCCC[CG]GCC[CG]
GGGACTAGGGTCTGG[CG]CTGGGG[CG]CCCCT[CG]GACCTG[CG]GGAT[CG]CCCCTACACTCT
GG[CG][CG]CTGAGGG[CG]GTGAG[CG]AGGG[CG]CCAAGGCACAGGTGGGG[CG]GGAGT[CG]
AG[CG][CG]GAGGCT[CG]GGGGG[CG]GGA[CG][CG]GGGCCTGGGAG[CG]GCCAGGGAC[CG]
[CG]GCAG[CG]CCTCAGTGCCAGCCTGG[CG]CC[CG][CG]ACTGCCTGCCCCAGCCCCTCAGTGG
[CG]GCTTGCTCTCTTCTCT[CG]CTC[CG]AACCAGACACAGC[CG]CTGC[CG]CTGC[CG]TC[C
G]G[CG][CG]CTACAGACTCC[CG]AGAACAGCCCTGGCTGTCAG[CG]AGCACCAGC[CG]CTTCC
TGTCCCCAT[CG][CG]GAGACTGGAGGGG[CG]CACCA[CG]GCCATGGAGCCAGAGG[CG]CTTCA
GGAGGCAAGAGAAGTCCC[CG][CG][CG]CTC
FLRT2 (SEQ ID NO: 2)
>hg19_dna range = chr14: 85996100-85996702 5′pad = 0 3′pad = 0 strand = +
repeatMasking = none
G[CG]GAGAGCAGAGAAGTAAAGGAATAAAACCAACTT[CG]CTGGCAACTTCAATAAATC[CG]GTA
ACTGGAGGCAAAAAATAGACTC[CG]AGGGCTGGGTCTGGTC[CG]GATGAAA[CG]CTAGGGCTCCT
TCCCTTCCTTGAATCTTGGG[CG]CTGGAAGCCAGCCA[CG]GG[CG]TCTTGCCA[CG][CG]AGTG
CCCCTAGACAGCAACACACCCACTGGAAA[CG]CA[CG]TGAACAAAGCTCT[CG]CCCC[CG]GGAG
C[CG]CTGCCTG[CG]GTTTCCTAGT[CG]ATCCCAGCTTCTCTAGGGAGTGTCAGG[CG]CACACAG
GGTTAAGTTAGTTCCCTCCCTGGTAGGAGGGAGAGGAGGAGGAGGGGAAAAGCAGCATACTGTCTCAG
GCTGGGTACCTTGTAGTTAGTTGTA[CG]TT[CG]AAACCTGT[CG]C[CG]TCACTTG[CG][CG]T
TTGGCATTATCCATTGTCAC[CG][CG]GAGGAA[CG]AG[CG]CT[CG]AGATATCATCAGTGCC[CG]
CAAATCTC[CG][CG]CCAAGG[CG]CTGAGCTACTCCTTTC[CG]AGGTG[CG]CCTCTGGTCCT
C[CG]TCCCTGGTGCCCAGCAG[CG]G[CG]AGG[CG]GCATCTC[CG]CTCC[CG]C[CG]C[CG]T
GTCCAC[CG]AGC
HOXD9 (SEQ ID NO: 3)
>hg19_dna range = chr2: 176987112-176987712 5′pad = 0 3′pad = 0 strand = +
repeatMasking = none
T[CG]C[CG]CC[CG][CG]GCT[CG]GAAGCTGGAGAGTCAG[CG]A[CG]GGGCC[CG]ACTG[CG]
GGAC[CG]AGGGCTGCAAGAAGAAG[CG]AACAAATAGTCCCCAG[CG]CCTCCTCTGGATG[CG]G
T[CG][CG]TCTGTGGTCCTGGCAGC[CG]CTGGG[CG]GGCCAGGCCAGGT[CG]GGC[CG]GGC[CG]
AGC[CG]GGCACATGGACCTGGGCCTG[CG]GGCTCTAATTG[CG]G[CG]CTTATGTTGATGATT
TTTTTTTTAATCACAGCAGCCCCCAGTTTAG[CG]GACTGATTTACTCC[CG]GTATTGGTAAATATG
ATCA[CG]TGGGC[CG][CG][CG]ACCAATGGTGGAGGCTGCAGCCTG[CG]AACTAGT[CG]GTGG
CT[CG]GG[CG]C[CG]G[CG]GGGAGCTGCT[CG]G[CG]G[CG]GACAGTGTAATGTTGGGTGGGA
GTG[CG]GGA[CG]CCTCAAAATGTCTTCCAGTGGCACCCTCAGCAACTACTA[CG]TGGACT[CG]C
TTATAGGCCATGAGGG[CG]A[CG]AGGTGTT[CG][CG]G[CG][CG]CTT[CG]GGC[CG]C[CG]
GGGCCAGG[CG][CG]CAGGGC[CG]GCCTGCAGGTGTGGCTGATGGCC[CG]GC[CG]CCAC[CG]C
[CG]C[CG]AGTT[CG]CCT[CG]TGTAGTTTTGCCCCCAGAT[CG]
MTA1 (SEQ ID NO: 4)
>hg19_dna range = chr16: 56672116-56672716 5′pad = 0 3′pad = 0 strand = +
repeatMasking = none
GCTCTGCACTA[CG]C[CG]ATC[CG]GGGACAGGAGCAGGAGGCTGTGGCTGCACTCAGACTT[CG]
GGACAGGC[CG]AGCTGAAAAC[CG]TGAGAGGGGTGGGGTGGAGG[CG]AC[CG]AAA[CG]CCAAG
GCTGGGTTCC[CG]GAA[CG][CG][CG]GGGACTAGGGTGGAAGGCAACTT[CG]GGGAAACTGGGA
AAGG[CG]AC[CG]GGACCT[CG]GGGA[CG]CCC[CG]TACCC[CG]GG[CG]TAAACTCACTCC[CG]
[CG]TTAG[CG]GCCCCAAAG[CG]GGGAGGGGGTGGTCC[CG]TGGTC[CG]CACCCAGGGGAGC
TCAGTGGACTGTG[CG]CCTTGCCTTTCTGCTG[CG]CAAAGCCCAGTCCAGGTCATCACCT[CG]GG
[CG]GGG[CG]GACT[CG]GCTGGG[CG]GACTCAG[CG]GGG[CG]GG[CG]CAGG[CG]CAGGG[CG]
GGTCCTTTG[CG]TC[CG]GCCCTCTTTCCCCTGACCATAAAAGCAGC[CG]CTGGCTGCTGGGCC
CTACCAAGCCTTCCA[CG]TG[CG]CCTTATAGCCTCTCAACTTCTTGCTTGGGATCTCCAACCTCAC
[CG][CG]GCT[CG]AAATGGACCCCAACTGCTCCTG[CG]CCACTGGTAAGGGATGCTAGGTTTCTG
GTCCTTAGGATACCTAT
VSX1 (SEQ ID NO: 5)
>hg19_dna range = chr20:25062524-25063124 5′pad = 0 3′pad = 0 strand = +
repeatMasking = none
C[CG]TCAAGCCC[CG]GGCC[CG]GGCA[CG]G[CG][CG]ACTGC[CG]GACCCT[CG]CAGCCAG
ATCCCTGTCCTGGGCCAG[CG]GG[CG]C[CG]GCAGCT[CG]GCCTCCAAGCCCAGCAGGTC[CG]T
GATGG[CG]AAGCCC[CG]GGGG[CG][CG]AGCCCCTAGGGGAAC[CG]CCAGGCACCAG[CG]CCC
TGCTGCTAGTG[CG]CC[CG]T[CG]GAAAG[CG]AGTCC[CG]GC[CG]GTCATGGTTCCTTAGCAA
GCAAGG[CG][CG]AGCCTCTCTGGATCC[CG]TTTG[CG]GAGGGCCCAGCTTAGAGGAAGCTTTAT
AGGATTGGGCTGC[CG]GT[CG]GG[CG][CG]GGAGGCCTGAAGGGCTCCAATCAGCTGGG[CG][CG]
GG[CG][CG]T[CG]C[CG]TTC[CG]GACCC[CG][CG]AGATGGGGA[CG]CCACCAGTTCCCC
TCCCCT[CG]GTTAC[CG]CCCACCCAGCTGCAGTCCCCTCAGTCACCTCCTTCTCCTCCTCTCTTCT
GTCCAGCCCACCCCACCCCACCCCACCCCCATTCTAGGAGGGAGGGGAGGAAAGAAGGTAAGGGACAG
TGACTC[CG]CTGTCTGCCTGTCCCT[CG]AAAAGT[CG]CAAGTTCTACATCCTGG[CG]TGGCCTT
CTCCTTCCTTCCAGACTCT
HOXD12 (SEQ ID NO: 6)
>hg19_dna range = chr2: 176964157-176964757 5′pad = 0 3′pad = 0 strand = +
repeatMasking = none
CAGAAAGCCTG[CG]GAAAA[CG]AAT[CG]CAAAGCCAATCA[CG]ACCAAGAAGAGTCCCAGGGGA
CACTTGGGCAGAGTCACCCTCTTGCC[CG]ATGTCCCCAGCTGCTGAAGC[CG]GGCCTGGAAACC[CG]
CAGACAGTTAGTCTT[CG]CTCAACCTGATTTGGCTCTGCTGGCAGCCT[CG]TCCTT[CG]CCAT
[CG]AACATTG[CG]GGTGTTATCATAATACTCTGAAGGGGGGGAAAA[CG]GGT[CG]GGGGGATGT
AGG[CG]GTGCTGAAATGAC[CG]GCTTTGAAGAACCTGCAGGCAAAGTTT[CG]TCCAAT[CG]TCT
GAGCCTGTCCTCTTATTCC[CG]GTTGTAACTAAATACTGTTG[CG]AG[CG]CAGC[CG]AAGCCCT
TTGTTGGAGATGTGTGAG[CG]CAGTCTCTACAGAG[CG]GGCTATGTGGGCT[CG]CTTCTGAATCT
GCAGT[CG]CCAGACTCTTTCTACTTCTCCAACCTGAGGC[CG]AATGG[CG]GCCAGTTGGC[CG]
[CG]CTTCCCCCTATCTCCTACC[CG][CG][CG]G[CG][CG]CTGCCCTGGGC[CG]CCA[CG]CC
[CG]CCTCCTG[CG]CCCC[CG][CG]CAGCCTG[CG]GG[CG]CCACTGCCTT[CG]G[CG]GCTTCT
[CG]CAGCCCTAC
LHX8 (SEQ ID NO: 7)
>hg19_dna range = chr1: 75593716-75594316 5′pad = 0 3′pad = 0 strand = +
repeatMasking = none
TGGTCAGTAAATGAGAAACAATAAATAGCCTTTGCACAAACTGAACCA[CG]CAG[CG]CAGCCTCTG
CTACTGCACACAATAACAGGAGTT[CG]GTCAAGGCCCAAAAGGAAAGTTCCAGAAGTGAG[CG]CAG
ACCTTTGGACCTCAGTCTGTA[CG]GAGAT[CG]AGTCAC[CG]TCTATTCAATCCAGGTTTTGTTCC
ACTTGCTGATGATTAGACATCAAATTATGCAATCTGCAAGGAAAAGGAGAGAGAA[CG]ATTTTAC[CG]
C[CG]ATTTAAG[CG]TTCTCCTTTTTCCCCTT[CG]TGGC[CG]TTTTATT[CG]GCACTGAAAT
GCTGTTTAACCTCAGCAAGAGTAGG[CG]TGGGAAGGTGG[CG]CTCTTCCCCACAGCACACCAGGCA
CCCCCCAGCTGCTCAG[CG]GAAGTGGGCAGAGGCAAGAGGCTAG[CG]GCTGGACCACTTGTGCTGG
AGTGGTAAAGAACTATCATGAATCCATTTACTGAAAGTGTCCATTTCTGAACTCACCCTAAAGAGGAC
AAACAC[CG]CAAAGTAGTTAAAAGTCAGGCATT[CG][CG]T[CG]GA[CG]TCTGGGTTTGAATTC
TGCCCTGGCTTGGTAAAATAGGGCTTTGGATAAAT
PHOX2A (SEQ ID NO: 8)
>hg19_dna range = chr11: 71955101-71955701 5′pad = 0 3′pad = 0 strand = +
repeatMasking = none
C[CG]GGTGGAGGT[CG]GAAGGGAGGTT[CG]AG[CG]CCAGGCTC[CG]AGGACC[CG]AGGCCAG
CTCTGAG[CG]CC[CG]AGAGTC[CG]CC[CG]CCC[CG]T[CG][CG]GC[CG]CACTCAGCC[CG]
GGTGCAA[CG]CAAGTGCAGCCAGCC[CG]GCC[CG][CG][CG]CCTTTTAA[CG][CG]CAGGACC
C[CG][CG]GAGGGGGG[CG]GGG[CG]GGG[CG]AGCTC[CG]AAGGGG[CG]GGGCCTAGGAGGCC
CAGACAC[CG]CCCAAAGC[CG]CCC[CG]ACCCCCACCC[CG]AAGGGTCCC[CG]C[CG]C[CG]T
CTTAGGGCTCAGAGGTGTGGAGCTGGGATG[CG][CG]GGACCCTATCCC[CG]GAGTCCCTATCCTG
CCCCAATC[CG]GACCAGACCTT[CG]GTCTCTACACAG[CG]TC[CG]AGGCCAGAGGGTCC[CG]A
CCCTTTTCC[CG]GGCCTCCCCACCTG[CG]GCCC[CG][CG]GAAGGGAGGGGAGGCAGGCCTGG[CG]
G[CG]CT[CG]TGTGAAGAAGTTAATTCAATT[CG]CTCTGATAAC[CG]AGTTATTC[CG]ATCT
GGCCCCTCAGCCC[CG]CCCCCAG[CG]AGGTCCCATCC[CG]G[CG]GAGACTG[CG]GAGG[CG]G
CCCAGGAAGGGGGATGGAGAC[CG]GGAAGAGA[CG]GAGCCTGG
ZIC1 (SEQ ID NO: 9)
>hg19_dna range = chr3: 147126711-147127311 5′pad = 0 3′pad = 0 strand = +
repeatMasking = none
GTCTCTAAT[CG]CTCAG[CG]AAAAGTTGGTTT[CG]GGAAGGGCAG[CG]C[CG]GGGTG[CG]GG
AGGGCAAAGGAGGAAGAGGAATGCATATGACTGTGAGAAGGAAAAGTAAT[CG]TGGCAAAAAAAAAA
AAAAAAAAAAAAAAAGAAAGAAAGAAAAGAAAAAAGAAAAAAAATTTC[CG]TGAGAAGAGGGAAAAA
ATTTGGCTAAAAAAAAAGTTGCTACTCCTGGCAGCCCTGGTTTGTCAAAAGGGGATGTCAAG[CG]CT
TTACAATACCTGGGATTGATGAGG[CG]GG[CG]GGCCAATGAGCTG[CG][CG][CG]G[CG]CCT
[CG]G[CG][CG]CCCTC[CG]TTGG[CG][CG]G[CG]GCTGAGGG[CG]GGGGGAATG[CG]GG[CG]
CACCAATGGG[CG]CCAG[CG]T[CG]GCAGCA[CG]TGACACCTCCCCCCTGCTCCCATTCATCAA
GGGGGGGA[CG]GTGT[CG]TCCTTTCAATTCATTTATCTGCAGGAATGATTGCTGCTATCAGTCT
[CG][CG]CTCAC[CG]CC[CG]GCTGAGGAGGTGAAAGTTTCTCCCCAGGAAGATAAAC[CG]CAAAAG
ACAATATTGTGCATGATTTG[CG]CCTTTTCTTTGGCTTTTTCTTTCTTTCTTCACCCCCCCA
RSPO2 (SEQ ID NO: 10)
>hg19_dna range = chr8: 109095692-109096291 5′pad = 0 3′pad = 0 strand = +
repeatMasking = none
CCTCCCTAACCAATTGTGT[CG]CTTCCTG[CG]CTTTCTCCA[CG]GTCACTTCACAGCTAAGATTT
CTTTCTTTC[CG]AGCTGTAGAAGGCAGAA[CG]CTCT[CG]GGAGGA[CG]AAGTGATC[CG]AAGG
GATGTGGCAAG[CG]CACTTTC[CG]ATGGAGATGCAGAC[CG]GCTGAGGCTTGGGATGCTTCTATA
AGTAGGTGGTGCTGTGGTCCAAGGCATCTAAGTCTAGGCTGCCT[CG]GAGTTCCTGGGTCCTAAAGC
CAGAAA[CG]TCC[CG]GTTTCCCTGAGGTCCTAAAGAA[CG]TTGACAGCCAACAG[CG][CG]CCT
AACTTGGAG[CG]AATCCTCTT[CG]GGCTTTCCAGAGTG[CG]GGGGATAGATAAAGAGTAGCTGGG
GAGA[CG]CCCCCTGACCTTGCTGGGTCCCAGAACC[CG]GCTGCTCACCCCCAAGGGGTCCTCTCCA
GCCCTCTAGGGTCAGTTCCAACCTCAG[CG]CCAACCTTTCTGCAGGGAGATCTGACTGAAGGACTGA
TGACTCCCTTTAAACTTTTGCAATATGCCACAACTCTGCCACTTTTTAATTTACATCTTGTCTGTGTT
TTGCAGGAAGGTATGTTAGC[CG]GCTGAAATTATT
SOX14 (SEQ ID NO: 11)
>hg19_dna range = chr3:137482831-137483791 5′pad = 0 3′pad = 0 strand = +
repeatMasking = none
GGAAAGGAAACAGGAGA[CG][CG]GAAGAGTTTTATTTGTTGACAGTGCC[CG]AGACAATGTGGAA
C[CG]AAGTCCCCAGTCTAGAGCTAGCCTCTCTGCTCTAGCCCTCTCTCTGCCTCTGTCCCTCACACC
TCTCCC[CG]CCCCCACCAGT[CG][CG]CTCTCTGTTTCTTGCAC[CG]G[CG]TGTTGGCAGGTG
[CG]CCCCCT[CG]GCTTCTCTGCTGCTGATTGG[CG]CCCAGGTTGGGGAGT[CG]C[CG]CCC[CG]
[CG]CTTATGTCAGAGTG[CG]TGCAGTCC[CG]GCCCACCT[CG]CCTTTGAACTT[CG]C[CG]CT
TTTGGCTCT[CG]TTCACCAGCCTCCC[CG]CATTCTCAGCCAGGTGCAG[CG]CTAGG[CG][CG]G
CAGCTC[CG]GGAAAGCCA[CG]GGAAGGGGGAGCCA[CG]ACTGGAAA[CG]CAAGAGGAGAGAAGG
GAACTAGAGAAGGCAAAGGTGGAGAGGGGGAGAAGGAGGAGCTTCTTGCCCTGGCTAAAAAATTACCT
GGCCACTTAATCC[CG]GAGACTC[CG]ATACTTGGGTCCCAGCCCCTGGTGCCTGAAGCCCCC[CG]
GC[CG]AT[CG]CCCTCTCCTCCCC[CG]CCCTTTCTGCCCCCACT[CG]CTCCCC[CG]GGGCTGCC
TGGG[CG]CTGGGACTGGCATGATCAGCTGAACTTTGTGGGGTCAG[CG]CTTCTCTCTGGCTTCCCC
TCAG[CG]G[CG]CCAAGG[CG]AGGGGAG[CG]CAGAACCC[CG]GCTCAGGA[CG]GACAGACAGA
[CG]GCCAGC[CG][CG]CCCAGGCT[CG]TCTGCAGAACCCTTGCACTCCCTACCCCCACCCACCTA
GC[CG]C[CG]GGACCATGTCCAAACCTTCAGACCACATCAAG[CG]GCCCATGAA[CG]CCTTCATG
GTATGGTCC[CG]GGGCCAG[CG]G[CG]CAAGATGGCCCAGGAAAACCCCAAGATGCACAACT[CG]
GAGATCAGCAAA[CG]CCTAGGTGC[CG]AATGGAAGCTTCTGTC[CG]AGGCAGAGAAG[CG]G
OTP (SEQ ID NO: 12)
>hg19_dna range = chr5: 76934484-76935083 5′pad = 0 3′pad = 0 strand = +
repeatMasking = none
CTAAATGAAAGAAAATT[CG]GTTTGTGGTTAAAAAGGGGGCTTCTTGCATTATAATGTCCTTTAGAG
AGA[CG]GGGAGGTGCTTAACAGTTGAATGAACC[CG]TGAGCAGCT[CG]G[CG]AGGGGACCAATC
AGGAGGGCAGCCTGCAAATGTCAG[CG]CC[CG]GAGAGTCCCCCACTC[CG]CC[CG]CC[CG]CT
[CG]CCTG[CG]CC[CG]CCTGAAGGAGG[CG]G[CG]G[CG]GCAGCCCTGGCAGCTCCAGCC[CG]G
AGCAGGAGCTAAAGCAGCCTCAGC[CG]CCT[CG]GCCT[CG]CCCTGAGCTTCTGGGCC[CG]GCTC
CCTCTAGTTCTTCCCTG[CG]ATTTTCTCCAGGCTCCCCTTTCCTTCCCCTTGGC[CG]G[CG]CTG
[CG][CG]CTT[CG]AAGG[CG]TAGCTCTGTGCCTCTGAGCTAACATCTTCCTAGGGCAGGGGCAGAG
CAGGGGTCAGAG[CG]GGCCATGACCCTGG[CG]TGACCTGAGGGT[CG]CCCCCACCTG[CG]CTCT
CTGGGCCTGC[CG]CTCATATTGGCC[CG]CCAGCCCAGGG[CG][CG]A[CG]AGGGCACAC[CG]
[CG]GCCTTCAGACAA[CG]CCTGGACTT[CG]C[CG][CG]GCTCAGTAGGCCTG[CG]CCTAGGGTC
CAGG[CG]CC
WNT2 (SEQ ID NO: 13)
>hg19_dna range = chr7: 116963179-116963779 5′pad = 0 3′pad = 0 strand = +
repeatMasking = none
TCTG[CG]A[CG]AGTGG[CG]AGACT[CG]CTGATAAAGTTTCAAA[CG]ATGGGCCCAG[CG]AG
[CG]ATAAAGGCCAGCC[CG]GAC[CG]CCT[CG]CCCACAGCCCAATTCCCCAGG[CG]CAG[CG]AG
[CG]CCTGCAGCC[CG]AAAGCTCTG[CG][CG]CCTCC[CG][CG][CG]CCC[CG]TC[CG]CTCT
GCTCCCCTC[CG]GG[CG]CAGC[CG]AGGGAGGGGGTGCTG[CG]CCTGG[CG]GGGGTGGGGAGAA
A[CG][CG]GGGTTTTAGACA[CG]GGTGCATGAAATGATGGCAAGAGATGTCTGGGGATGAGGTGAG
G[CG]GGGAAGGGGGAACAT[CG]CTGCATGGTTA[CG]AGCACA[CG][CG]AATACAGGAACCTTT
AGGGAAGCTCCAGGCCAAAGAGCATC[CG]TGGGCTCT[CG]GAG[CG]TG[CG]TTC[CG]GATTGC
[CG]AGGCCATC[CG]CTGGCAAACAAGTCCCTGAG[CG]TGCAATGTGAGGAAA[CG]AGGAAGGGG
GCCAGGGGATAAGGAGATTACTATGCATGGGATACAAGAATCCAGAGATGATTCTCAACTCATTGCTT
[CG]CTTCTCTCCCTGAAGCTGGGATGAAGAGCTAGCACTTGTAAATTTGGAGCTGC[CG][CG]TCT
ATGCAGGGCAAG
NEUROD1 (SEQ ID NO: 14)
>hg19_dna range = chr2: 182545185-182545785 5′pad = 0 3′pad = 0 strand = +
repeatMasking = none
ATAGTCTCATAACCCTGGGGCCTC[CG]GA[CG]C[CG][CG]CCTCCTG[CG]TGGG[CG]AATTCC
T[CG]TGT[CG]TGGC[CG][CG][CG]GG[CG]CTCAGGTTATATAGCCCAGTTAGTGATGCTAAG
[CG][CG]GG[CG]GGGC[CG]CTAGCTGAGGGGCTAGCAGGTCTATG[CG]CCTGA[CG]CCTG[CG]
CA[CG][CG]TCCAGGCTGTG[CG]CTCCC[CG]TTCTCCCCTCCTCCCCACTTCTCCCCA[CG]CCT
TGCT[CG]TCTCC[CG]CCCTCCTC[CG]ACAAC[CG]CTCCCCTCACCCTCCACCCCTACCCC[CG]
CCCCTCCTCCTTCCTCCC[CG]GCATG[CG]CCATATGGTCTTCC[CG]GTCCAGCCAAGAGCCTGGA
ACCA[CG]TGACCTGCCCATTTGTATGC[CG][CG]GAG[CG]CTCCATTC[CG]GCCCCTTTGTGGC
CAGAAGAAAGTGGCCCATCTGT[CG]CCAGTTAGAGACTC[CG][CG]GACCTGTTTTTACC[CG]CA
GGAGAGATTAACCCTTTCAGG[CG]GCAGAAGGGA[CG]GGGATAGAGGGGGGAGGGGATTTGAGGGA
AGGAAGGGAAAAGCTAATGTTAACTAGGTCTAGATCAGAG[CG]AGTGGCCTGCTTT[CG][CG]C
[CG]GAAGTAGGACAGA
CBLN4 (SEQ ID NO: 15)
>hg19_dna range = chr20: 54579870-54580870 5′pad = 0 3′pad = 0 strand = +
repeatMasking = none
AG[CG]GCTAGCCAGGTCCCT[CG]CAC[CG]AAAG[CG][CG]GATT[CG]CAGGATCAGGTCCAGG
G[CG]C[CG]GG[CG]CAG[CG]TTCAGGG[CG]GCTGGTC[CG]G[CG]GGGTCCTCTCCTGCCTGG
C[CG]CC[CG]CCCCCAGTCCCTG[CG]CACAACTTTCT[CG]TCCCT[CG]TGCAGCC[CG]GAGAG
[CG][CG]AAG[CG]GGCACA[CG][CG]CTCTATTTATAGGAG[CG]CAG[CG]TC[CG]GCTGGGT
TGGCTTAT[CG][CG]CAACC[CG][CG]GGCTC[CG]AGAAAAGGGGGAGAAGG[CG]TCTGGTGAC
CCCATCTGAGCAGCTCTCTCCTGA[CG]TTTAA[CG]CACCCCAAAGCAAG[CG]GGG[CG]CACAGC
ACTGGTGATTGGCAAGGTT[CG]AGGCCCCTCAGCCCCTGAAAGCC[CG]TGG[CG]TAAAGGATG
[CG]TGCCCAGAGAGGAGCCCA[CG]GCCCTG[CG]CTGGCCCCAGCTC[CG][CG]CCCACTCTCTCCT
GCT[CG]ATCTGAGTC[CG]CAGAGGAGG[CG]GCTGGTCCCCTCACCCCTCCCAG[CG][CG]GAAC
TCTGC[CG]C[CG]AGTGCCCTCCAAG[CG]GAG[CG][CG]G[CG]CTGCCAGGC[CG]GGT[CG]
[CG]AAAAAGGATAAGC[CG]C[CG][CG]GACC[CG][CG]CTG[CG][CG]CTGCCC[CG]AACCTG
GGGAC[CG]G[CG]GG[CG]TCC[CG]AC[CG][CG]CCTGGC[CG]GGAGCC[CG]CCCCACACAGC
CCTGGGGCCTGG[CG]AGCTCAGGGAAGCCTTGGGG[CG]GACCAGAGCC[CG]TGGGGG[CG]GCTG
GGAGGAGGTGCCTGATTCCTTCCTTC[CG]CTC[CG]GGAAGATGAGCCTCAGAAGC[CG]CAGGGG
[CG][CG]CCTTCCCCCCACAC[CG][CG]AATCCTCCCCAGGCATGGCCAAAAAAAAGAGTGGGAGAG
GGAGTTAACTCAGCAAGAATAAGTCCTC[CG]AAAC[CG]TGCCAACAACAATAAAAAAATAAAAATA
AAAATAAAAATAAAAGACAATCCCACTCCTAGGAATACAGCATTTTTCTCCCCTTTGGATG[CG]CTT
TC[CG]CCCC[CG]TGCCACTCCACTG
ADCY8 (SEQ ID NO: 16)
>hg19_dna range = chr8: 132052543-132053143 5′pad = 0 3′pad = 0 strand = +
repeatMasking = none
[CG]CTGCCTGTAAGGCAG[CG]CACAT[CG]GAGAGCTCCATGGCTCTGGGC[CG]CAGGGAAGGAG
GCCCAGAACCTTGGGGAGGCAGC[CG]GAGGAGGGGTTCCTAAAGACTCAAAGG[CG]GCCTGGTAGG
AGCTTGGCAAGGATCCTTTTTATCCTAGGCTGCCC[CG]TTGCAGGAGCCCTG[CG]CTAGGGCTCCC
TGTAGGT[CG]GGTCATACTGTGCCCAGGGGCAAAGGGCACCTGGAAGAT[CG][CG]G[CG]GACCT
[CG]G[CG]TCCTTG[CG]TGCTGCTCTCC[CG]CCAGC[CG]G[CG]CAGCTTGGGTA[CG]CAG
[CG]GCAGTGGCTATTTGTCCTCAGGAGC[CG]CAG[CG]CTGTGAGCCA[CG]CAGCCCCTTCCTGGGC
TCAGGCTCCTTGGTTGATTCTAGGCTCAG[CG]TTTTGG[CG]CAGCCTTTGCTCTCCAAGAGA[CG]
CCTAAG[CG]CCTGGG[CG]C[CG]TGCTTCTGCTG[CG][CG]T[CG]GG[CG]GGTCCTGGCTGCT
GCACTGGGCTTTGGA[CG]CCCACTGCTC[CG][CG]GCTGCAGGGGGCCTGGC[CG]G[CG]GTGAC
[CG]G[CG]GCAG[CG]ATGGGTG[CG]AAGTTAGCAGATGTGGCTGGAATCAT[CG]CCCTT[CG]G
AGAACTCCA
SLC6A5 (SEQ ID NO: 17)
>hg19_dna range = chr11:20620524-20621124 5′pad = 0 3′pad = 0 strand = +
repeatMasking = none
CAATTTTTAAGCAAAAACCCCAGGATTCCTCCAAGACTGACTGTCTGGAAGGGTAGGGATGTGAGAAT
CTGAAGTCCACATACATGCTAGGTAGGTACACCTTGATAAATGCAGGACCCATGCCTGCTTCAGTCAT
[CG]CAGAGCTGTGGGGCTATG[CG]CAGACCCTCAACCTCTTT[CG]GAGTAATATTTT[CG]GAAT
CCCTGAAGATAAGGGGGGACAGCAATGGAATCTAGTGAAAGGGAAAGAATAGACAGTGATTTAACCCC
CTCCTTCCTCCTCTACAGCC[CG]ATCCACTCTGTTGG[CG]GCTG[CG]CCTTGCCTTGGGTGCAGT
GGGA[CG]TGTCCCTCCCATGTTTCCCTTAATTAGGAGGAGGCAAACTTCTGTCTTCCCCTCCTGC
[CG]GTTT[CG]GTTTAGTAATCAGACCACATGAGTCA[CG]CCAAACA[CG]CAGCCCCCTCC[CG]CT
GGAGTGACAACTGGCCAGCATACTCTAGGCTGTTGTCCCTTTAAAACTTGAATCCAAGGGGGTAATGA
TTTATCAAACTTGTATTATCAAGAAAATGTCAAACCAAGGGCACCTTGCTTTGCACTGA[CG]CAAAC
C[CG]GCCTTTCCCAAGGA
GABRA4 (SEQ ID NO: 18)
>hg19_dna range = chr4: 46995337-46995937 5′pad = 0 3′pad = 0 strand = +
repeatMasking = none
[CG]CACCCCAGAGAACTCACCAAAC[CG]CCAGGCACAGGAAG[CG]CAGGAGGG[CG]AAACTGAC
CC[CG]G[CG]GACAGAG[CG]AT[CG][CG]GGTACCTTCTTGGCAGAAACCATCTTTGCAACATGC
CATACTTCAAGCCTGTTCA[CG]TTTCCAGGCTCTTCAGATGCCCTGAGCAGGGTG[CG]AGGAGAGG
GCAGAGAGGCTCC[CG][CG]G[CG]TG[CG]CACACT[CG][CG]CTCACACT[CG]CC[CG][CG]
CTCAGCCAGCC[CG]AGC[CG][CG]GTGGG[CG]TGTGTGTGCA[CG]GGGCCAGGGGAGG[CG]GA
ACCTGCCTTCCT[CG]CCCCCTCCTTTCT[CG]CTCAG[CG]CTCAATGTGTATGTAGAGCTATGTAT
AC[CG]CTCCACACCCTTT[CG]TGCC[CG][CG][CG]CTGAAGGTTCTGGGGTT[CG]TATC[CG]
[CG][CG]CTTG[CG]CTGCAAGACT[CG]GCAAGTTTGTTC[CG]ACTGTAACTC[CG]GGGATGAG
GAA[CG]GGGTCTCT[CG]CCCCTCCTC[CG]GCTT[CG]CAGCCCC[CG]ACCCCAAGTGGGCTCCC
[CG]CCCACAGCAGTGTT[CG]TCCTCCTACCTCCTGGCCCCCTCCCTTC[CG]CTGGGATTTGGATC
TAACCAATGAAGCTCCTCTGT
HOXB8 (SEQ ID NO: 19)
>hg19_dna range = chr17: 46692123-46692722 5′pad = 0 3′pad = 0 strand = +
repeatMasking = none
GAGGGGGTGGGGAGGGGGAAAGGGAGGGAGAGAGAGAAAAAAA[CG][CG]GATT[CG]C[CG]GCTC
CTAGTCACCCT[CG]CCAGGAAAGGAGAGGGAAAGAGAGGAGGGAAAAAAAGAAAAGAAAGAAAAGG
[CG]AAGAAGATCT[CG]AAGC[CG]ACACACTTTTTGTGGTTTCCAGGAATAGAAAAGGACAGAGAAG
CCTCCAAAAGTCTAAGCTTGCATGGCAGC[CG][CG]GCT[CG]CT[CG]CCCTCCCCCCACCCCCCA
CCCCCCAAACAAAAATATACCACCACTTAAAGAGGTCCT[CG]CTTTACATTT[CG]AAGAAGGGATG
CCAGTTCATAAACAGTTTT[CG]GTGATTTACTTCCCTGCAAATGAGTTGTTTCATATTTTGCACTGT
CTTTTCATGATCATTTGCATCCATTAGAGACCC[CG]CATCCTATTGGCTTCTT[CG]TACTCCTCC
[CG]GACAGAA[CG]CAGAG[CG]AGGGTGAGAG[CG]AGAGAGAG[CG]AG[CG]AGAGAGAGAGCTA
GAG[CG]AGAGAG[CG]CCAGGGAGTGAGGAGAGAG[CG]GAAAAAAAAAAAGGAGGGGAAGAAAAAA
ACACCCAGGGAGAGAGAGGGGGAGAGGGGGAGAGAGAGGG
KY (SEQ ID NO: 20)
>hg19_dna range = chr3: 134369675-134370274 5′pad = 0 3′pad = 0 strand = +
repeatMasking = none
[CG]CTGCAGCAG[CG]AGCT[CG]GGTT[CG]CCTGCTGGTCTGAGAG[CG]TACCCTGTG[CG]G
[CG][CG]C[CG]CTTCTC[CG]AGTGCA[CG]ATCAGCAGCATGT[CG]ATAGATACAG[CG]TTGAT
GTCCTTCTTCAGCTCCATGATGC[CG]CCTCCTTTC[CG]ACCTGGG[CG]C[CG][CG]GC[CG]CA
[CG]CTAGGCTGCTTG[CG]CTGCAAATGGCCC[CG]TG[CG][CG]CAGCTGCCCCACA[CG]GGAG
AGGG[CG]AGGG[CG][CG]GAAGAGGG[CG]CTCCCC[CG]C[CG]CTGGAGC[CG]CAGGG[CG]C
TGCTTTT[CG]CTGACTCTGCCAAACA[CG]CCATTAGGGCC[CG]CCTGGGAGGG[CG]CTACCCTA
ATGAAG[CG]GCTCTAAGTCACCTTTGAAAGGAAATCCC[CG]GGCCCAAGCCTTGACCTGCAC[CG]
GGGCCATAGTCAGCAGGGG[CG]GC[CG]GTCCAGAGCCTCCCTCCCTG[CG]CCCCTGCCTCC[CG]
CC[CG]GTGCACTTGAAG[CG]CTGGTCCTGGGCTCCAGGGCTTCTCTGGCTGAG[CG]CTGCCTCAT
CCCCA[CG]GTCTC[CG]GAGGGTTCT[CG]AGAGGGCTGGAGC[CG]ACCTCCCA[CG]AGAAGCCC
CTGGCAAATACAGAAAT[CG]CACAG
LTK (SEQ ID NO: 21)
>hg19_dna range = chr15: 41805842-41806441 5′pad = 0 3′pad = 0 strand = +
repeatMasking = none
CCTGAC[CG]CCAGATAGCACTTACC[CG][CG]GCTC[CG]AACCACACCAGCAGCTGTCCCCAGCA
GCCCATCCCTGTTGGGTCCACC[CG]GCAACAAAAGCCCTTG[CG]GT[CG][CG]GCCACACCCCTG
TCAACCTAAAGTTGACAGCTCATTTTGCCA[CG]GCAGCCCTGGCCACCACTTACAGGGGTGTGCTGC
[CG]CTGG[CG]AGACACTCCC[CG]GACCTAAAGCTGGTCTGGCCA[CG]CCCACCTCAGACCCCAG
GA[CG]CCAGAGGAGGAAAGGGAGGGG[CG]TGTGGGCAG[CG]AGGTCCCTA[CG]CTTC[CG]GGA
GGAGCCTTAAGGAGTCCCCACCCAGGGGTTCTAGTTTCCAGC[CG]TCAGATGTGC[CG]CAGCCTGC
TGGGAGCTC[CG]GTAGGTTTGAGGC[CG]GAGAGGAGGTGCCCCA[CG]CTTAAAGTGGGAGCCACA
CCT[CG]GACACCAGGTGGGGGCTTCCAGGTTCCTGAGGACAGGAGCAGTGGAGTGTGCACTTCATTT
GTGGCAGTGGTGGTGGTGGTGAGTGTGTGGCATG[CG]TCACCACA[CG]TGGGAAGTACTCTAAACT
GCTCCAAGTAAGGCCTCATTACCTGCCATTACTTGTGCCT
MKX (SEQ ID NO: 22)
>hg19_dna range = chr10:28034554-28035153 5′pad = 0 3′pad = 0 strand = +
repeatMasking = none
AC[CG]G[CG]CCCAGGG[CG]CAGGGGCCAGAG[CG]AGCCTGG[CG]TTC[CG]C[CG]CAGC[CG]
GCTGAGACT[CG]G[CG]A[CG][CG]GGGGCTGTACCTGTGGCTG[CG]GGGC[CG]A[CG]GC
[CG]GCTGCAGGG[CG]GCTGGCTCTCC[CG]CCT[CG]AGACTAGG[CG]CACTCCCATCCC[CG]C
[CG]CATGTTCTCCA[CG][CG]GGCTCCAG[CG][CG]CTCACCAC[CG]CCAC[CG]C[CG]T[CG]T
CT[CG]GCTTTATTTACCCAGCC[CG]G[CG][CG][CG]C[CG]CC[CG]GGAACAGGAATAG[CG]
AGGCCTTCTCATGTTTCCTGACTGC[CG]GTCCCAGC[CG]G[CG]AACATCCTG[CG]GG[CG]
[CG]GTATCCA[CG]TTCC[CG]GG[CG]GGTGGAGAGGAAG[CG]TC[CG][CG]CC[CG]CC[CG]GC
[CG][CG]GGGGCTGCTGTTAACCCCTAGCAGAGGAGC[CG]CTGC[CG]GGGAGCCC[CG]GGCC
[CG]C[CG]AGTCCTC[CG]GAGC[CG]GG[CG][CG]GCTCCCAGATC[CG]C[CG]GCCTTGCAGT
[CG]TGGAGCAGA[CG]GGGCTTTTCCCTT[CG]CACTTTAGGAGAGAAATTGGAGAAGCTGC[CG]GGAG
[CG]CT[CG]GCTC[CG]GCTCCAGAAGCTGAGAGGCTG[CG]GTGCAAATCC[CG]TGTCTTAGCTG
NPNT (SEQ ID NO: 23)
>hg19_dna range = chr4:106816265-106816864 5′pad = 0 3′pad = 0 strand = +
repeatMasking = none
CCTG[CG]GAG[CG]TGCACCTA[CG]CCT[CG]GGCTCCTTTCCTCCTCCACTCCCCTTTCCTGCTG
GGCACCCTGCTTTCCCTCTCCCAGAGAGGGTTTGCAACTTTTCTCCCAGGCTGGGGCT[CG]CCCTGC
TTGGCTAACCCCCAAGAGCCACTGC[CG]TCC[CG]CAG[CG]CCCCTGCCCC[CG]AGTTGCCTGCC
C[CG]CTGGGCCCC[CG]GGAGGAG[CG]GAG[CG][CG]CTCACCCTT[CG]CC[CG]GGGCTGGGA
GGG[CG]G[CG]AGT[CG]GG[CG]CA[CG][CG]CACCCCCTGCC[CG]CCCCTGG[CG]CCCCTCC
C[CG][CG]GG[CG]GTGCAGCTACCCCTGCAG[CG]CCTCCCCTAGCTAGAAGGGAG[CG]GGAGGG
GGCTC[CG]GG[CG]C[CG][CG]CAGCAGACCTGCTC[CG]GC[CG][CG][CG]CCT[CG]C[CG]
CTGTCCTC[CG]GGAG[CG]GCAGCAGTAGCC[CG]GG[CG]G[CG]AGGGCTGGGGGTTCCT[CG]A
GACTCTCAGAGGGG[CG]CCTCCCAT[CG]G[CG]CCCACCACCCCAACCTGTTCCT[CG][CG][CG]
CCACTG[CG]CTG[CG]CCCCAGGACC[CG]CTGCCCAACATGGATTTTCTCCTGG[CG]CTGGTGC
TGGTATCCT[CG]CTCTACCTGCAGG[CG]GC[CG]
OSR1 (SEQ ID NO: 24)
>hg19_dna range = chr2: 19558208-19558807 5′pad = 0 3′pad = 0 strand = +
repeatMasking = none
TGTCC[CG]ACTCCTCTCCTGC[CG][CG]GGGACTCCAAG[CG]C[CG]GACA[CG][CG]GGAG
[CG]AG[CG]CTCAGCAGCCC[CG]GATCCTGCA[CG]C[CG]GGGA[CG]GTGAGCCT[CG]CT[CG]
[CG]GCTCCCT[CG]CTGGGTCTGAACCAGGAGCAGCTGAGAA[CG]TGC[CG]GGGAGAGGC[CG]AG
CTC[CG]ACTCACTCATCCCTTAGCAC[CG]AGC[CG]CCTACTTATTTTAATCCACCACCCTCCCTC
CTCCACCACCCCCACCCC[CG]CCCCTTGCCTCAGGATTCCTCCCCTCCCTCCCCTGCCCCCCAACCC
TGCCCTG[CG]CCTG[CG]CAATGAAAGACAAAGTTTTTGCTGACC[CG]GGCAGCAGACTTTGGCAG
CTGCTCT[CG]CTTGGACC[CG]GCCTCTCCCAACCTG[CG]TCCCCTCCTCTTCAGTCC[CG][CG]
[CG]ACCCCTGCTGCCTTTTTAGGTCCAGAG[CG]GCA[CG]CCTGG[CG]CACTGGAGGAAA[CG]G
TCCCTAGCAGATGAGAG[CG]GGCTGGCAC[CG]CA[CG]TTT[CG]AGGTCCTATACTTGGGCTCCT
AGGACACAGCC[CG]TGCTCTTTGGGAGGAGGGAGGGAGGGGCAGTAGCTCTGTGCAC[CG]GGATCC
CCACAC
POU4F1 (SEQ ID NO: 25)
>hg19_dna range = chr13: 79177578-79178177 5 ′pad = 0 3′pad = 0 strand = +
repeatMasking = none
GG[CG]GCCC[CG]C[CG][CG]GGCTGCTGCTGCTGCTCCTGCTGCTGCCAGG[CG]CTCCCTCTGA
C[CG][CG]CAGAG[CG]C[CG][CG][CG]C[CG]GCCT[CG][CG]GTCC[CG]CTTCTC[CG]AC
AGCTCTAGCCC[CG][CG][CG]C[CG]A[CG]GGATGCACTCCTCTAACACCTGAGCCCCACTTCT
[CG][CG]GC[CG]TCC[CG]GGGAGCTCT[CG][CG]AGAGCT[CG][CG]GCCCCAC[CG][CG]CT
GACAGGCATCAGCTGTCTC[CG]TCTGTCTGA[CG][CG][CG]CTCTCCTTCT[CG]G[CG]GCCC
[CG]TG[CG]TCTG[CG][CG][CG][CG][CG][CG]CT[CG]TC[CG]GC[CG][CG]ACCAG[CG]
[CG]TGGGAGC[CG]CTCTTATAGTGACCACCAGCAAGGACAG[CG]CAGGTGATGCACCTGTAGCTA
CC[CG]GGCATG[CG]CACTGCC[CG]CCTCACCTTTCCCAC[CG]TGGTCTGGAAAGATCAGAAGGT
CCAGCTATTGTCTAAGGGTGGGCACCTTTTAGAATAAAAAAGA[CG]AGATG[CG][CG]CA[CG]CC
CCTCTATGTGTGTGTGTGTGTGTGTGTGTG[CG]TG[CG]TGTGTGTGTATGTGTGTATGTTTCTTCT
CCCTGATAGCAAGCCTTGAATATATGAGCATTACTTAGGCATTCCT
SHOX2 (SEQ ID NO: 26)
>hg19_dna range = chr3: 157823851-157824450 5′pad = 0 3′pad = 0 strand = +
repeatMasking = none
GCAGAGCCC[CG]CTCTTTTTTTCCTTCTTCTTTTTTTACTGCTCCAGCCCCCCCAATAATAACACAT
CAATGGGACAGGAGGTGGGGGAGGAGAGGGAGGAGGAGAAAGAAGAAGAAAAAGAGGAGAAGAAAGAA
GAAAGAGGAGGAGAAGTAGAAGGAGAAAAAGAAGTAAGAAGAGGAGGAGGAGGAAGAAGAGGAAGAGG
GGGAGAAGGGTGAAGAGGAGAGGGAGGAGGAGGAGGAGAAGAGAAGGGG[CG]GGGGCTGC[CG]GAG
GGAAATGGGAACTGATGCTTCCCTGC[CG]GAG[CG][CG]CTTGTTCAATGTTAAGATCTTTGACAA
TTCTTCCTG[CG]GAGAGTTCAGATCTGATAG[CG]A[CG]CTG[CG]CTTGAAGTCCCACTATTTAT
ACTTCCTAA[CG]G[CG]GCCTCTTGTTTTGAGTAAATTACCTCCCCT[CG]CAACT[CG]GAGTGAG
CCCCCTTCC[CG]GGTAGCAGT[CG][CG]CCTCTAC[CG]GGGGTTGGGGAGGGGAAAAGGAGAGAA
ACAGAGAGAGAGAGAGAGAGAGAGAGAGAGAGAGAGAGAGAGAGAGAGAGAAGAGGAGAGAGAGAGAT
TGGAGTT[CG]GG[CG]GGCTGGGGCTG
SLC10A4 (SEQ ID NO: 27)
>hg19_dna range = chr4 : 48484934-48485533 5′pad = 0 3′pad = 0 strand = +
repeatMasking = none
ATTACATTCTACTTTAAGACTGAAAATCATGCAAAAAATTGTTTGCAAATTCAAGCCAGATAT[CG]T
GTCCTGCTTACTAGAGAAT[CG]CTCATATTAAGTCAAGCACCCCTCTTAGGGCTGCAGGCTATAGGA
ACAGTTTCTGCCATGAG[CG]TGCAT[CG]ATGAGGTTATCCACC[CG]CCCCCTCCACTTTGTGTGT
CTGGGTCTCCAGATTTCATTATAAAGAGGCAGAGCCCATCAGAGAACTTGAT[CG]GCAGCCAGGAA
[CG]GTTTTTTCCTGCTCTGGTCCTTTGTCAGGTCTTTGTTC[CG]CCCCTGGCTACACT[CG]ATAAC
AGAACCACTTACTGG[CG]ATTCACAGGAGGGGAC[CG]GG[CG]GCAGGG[CG]GGG[CG]GGGCCT
G[CG]GGG[CG][CG]GGGC[CG][CG]G[CG][CG]GAGC[CG]G[CG]CAG[CG]ATTGGGTTCAC
C[CG]GGGGGC[CG][CG]CTG[CG][CG]GAGTGCCAGGCTG[CG]GG[CG]GCTGCAGACCTGGGA
G[CG]GAGAC[CG]GCC[CG]C[CG]CCCC[CG]A[CG]C[CG]C[CG]AGCA[CG]TCAG[CG]G
[CG][CG]CAGC[CG]GGGCT[CG]GAGAC[CG]A[CG]GGCAGAA[CG]A[CG]GG[CG]G[CG]ACTG
[CG]G[CG]AC[CG][CG]GGA[CG]G[CG]AGA
SPOCK3 (SEQ ID NO: 28)
>hg19_dna range = chr4: 168155589-168156188 5′pad = 0 3′pad = 0 strand = +
repeatMasking = none
CTGGCACATCACCTTGTTGTGAGCCAGCACTGTGCT[CG]CAGCTCCTGCTGGAAATGCAGCCCTGCT
CTGCCAACT[CG]CTGCTTTCTGGAGCTGCTGCA[CG][CG]C[CG]C[CG]TCT[CG]AGC[CG]TC
CACTCAGCTCCCTG[CG]CC[CG]G[CG][CG]CTCCCCCCATCTCCACCCTCCCTT[CG][CG][CG]
[CG]GATCTCCAGCTTACTCCCCTC[CG]TTCCCCCTCCCTCCC[CG]ACCTC[CG]TTTTTTTCCT
CTTCCACCTCCTCCTATCCCACCTTCCTCT[CG]CTGGATCCCAGCTACAGTTGGCTC[CG]GATAGG
A[CG]CTTGGCTGGAGCCAGAGGA[CG]AGGTAGCTCCTAGCCTAGGAATCCTTATCTGCTTTGTAAC
CTCTCCCCTCCAGAAGAGGTTCTCATGTT[CG][CG]AGACAGCCTGCTCATTCTCTGCTCCTGGATT
TAACACAAAGTGTACTATCTCT[CG]CAGTTGCCTCTGCTCCACAATTTCCAGGGTTGTCAGGCAGA
[CG]TGAGAAACAAGATCTGAGGAAAACTTTATGCATTAATAAATAAGAATACCTGGACTTCCTACCCT
GTAAAGCTCCTGAACTCAGCTCCTCCCTAAAGAAAGGCCATC
UNC5C (SEQ ID NO: 29)
>hg19_dna range = chr4: 96469987-96470586 5′pad = 0 3′pad = 0 strand = +
repeatMasking = none
T[CG]CC[CG]CAGACCTTTCCTCAT[CG]TAGACAGAGGTGTGC[CG]GGGGGAGGGGAGGGGGACA
GAGAGA[CG][CG]CAAACAGCTGAAAGCCCCACTGGGCAGAAGCTGAATC[CG]TGCAC[CG][CG]
AAGCAGTC[CG]AAGAAATAA[CG]ACAACCAAG[CG]CCACACTCCACTTTCTCCAAAGGCAGGGGC
CAAAAGGGGGAGGAGAGGAGA[CG]C[CG]AGGAGGAGTTTGAAGGTCCCTCTTTCCCAATGCCTTCT
AACTGA[CG]TCTCC[CG][CG]AG[CG]ACTGCTCTGCCCCCACTTGGGAAGTCCAGGGAGG[CG]C
AAGG[CG]GCCACCTC[CG]C[CG]CTCCCCCCAGCAGCAGGAGCAGC[CG]CAGCTC[CG]C[CG]C
A[CG]C[CG]AAGTCTGGACTGCTCTC[CG]GGTCCCCT[CG]CCTGTGAGGAACACACACAGACAC
[CG]GGGGTGGGGGTGGGGGTGGGGGTTGAAGTGGGGGGAGTTGGAGGAGGAGGGAA[CG]GAGGGAGG
AAGCTTGGGCAC[CG]TCAGG[CG]CTCTGGTCTCCAATCTCAGTGTTCTCA[CG]C[CG]G[CG]GA
GAGATGTCC[CG]GCTGT[CG]CTGCTGGGTGGCCAAGGAGGTTCACATCCTAGAGTAGGG[CG]C

As explained in more detail in the Examples below, the inventors additionally identified 37 individual CpGs amongst the 29 DMRs listed above. These additional 37 CpGs can be used in conjunction with CpGs of the 29 DMRs listed above to derive CpG panels for use in the assays described and defined herein. These additional 37 CpGs can also be used independently of the CpGs of the 29 DMRs listed above to derive CpG panels for use in the assays described and defined herein. The additional 37 CpGs are identified as the CG dinucleotides in square brackets in the sequences set out in Table 2 below. The 37 sequences harbouring these additional 37 CpGs are defined according to SEQ ID NOS: 30 to 66.

The methylation status of any one or more CpGs of the 37 CpGs defined according to SEQ ID NOS: 30 to 66 may be assessed by any suitable technique. As explained in more detail in the Examples below, one particular exemplary technique which the inventors have used is a fluorescence-based PCR technique referred to as MethyLight. These assays utilise forward and reverse PCR primers specific for the 37 sequences harbouring the additional 37 CpGs defined according to SEQ ID NOS: 30 to 66. The assays also utilise detectable probes specific for the 37 CpGs. For each one of the 37 CpGs forward and reverse PCR primers and detectable probes are set out in Table 3 below.

TABLE 2
For deriving the breast cancer index of the invention, the methylation status of each CpG in a set of test CpGs selected from the
panel of CpGs identified in Table 2 may be derived. CpG as defined in the table below refers to the CG dinucleotide motif identified in bold
and in square brackets in relation to each SEQ ID NO. The Illumina Identifier (Ilmn ID) and chromosome position of the sequence is listed.

| SEQ | | | | |
|---|---|---|---|---|
| ID | | | Illumina | |
| NO | chr | pos | ID | Forward_Sequence |
| 30 | chr15 | 41806141 | cg07621749 | ACCCCAGGACGCCAGAGGAGGAAAGGGAGGGGCGTGTGGGCAGCGAGGTCCCTACGCTTC[CG]GGAGGAGCCTTAAGGAGTCCCCACCCAGGGGTTCTAGTTTC |
| | | | | CAGCCGTCAGATGTGCCGC |
| 31 | chr16 | 56672415 | cg09137696 | CCCCAAAGCGGGGAGGGGGTGGTCCCGTGGTCCGCACCCAGGGGAGCTCAGTGGACTGTG[CG]CCTTGCCTTTCTGCTGCGCAAAGCCCAGTCCAGGTCATCAC |
| | | | | CTCGGGGGGGGCGGACTCG |
| 32 | chr4 | 48485233 | cg19283196 | ACTTGATCGGCAGCCAGGAACGGTTTTTTCCTGCTCTGGTCCTTTGTCAGGTCTTTGTTC[CG]CCCCTGGCTACACTCGATAACAGAACCACTTACTGGCGATT |
| | | | | CACAGGAGGGGACCGGGCG |
| 33 | chr4 | 9783198 | cg03045635 | CTGGCGCGCTGAGGGCGGTGAGCGAGGGCGCCAAGGCACAGGTGGGGGGGGAGTCGAGCG[CG]GAGGCTCGGGGGGGGGACGCGGGGCCTGGGAGCGGCCAGGG |
| | | | | ACCGCGGCAGCGCCTCAG |
| 34 | chr2 | 176987411 | cg09578028 | GCAGCCCCCAGTTTAGCGGACTGATTTACTCCCGGTATTGGTAAATATGATCACGTGGGC[CG]CGCGACCAATGGTGGAGGCTGCAGCCTGCGAACTAGTCGGT |
| | | | | GGCTCGGGCGCCGGCGGGG |
| 35 | chr4 | 9783196 | cg23847712 | CTCTGGCGCGCTGAGGGCGGTGAGCGAGGGCGCCAAGGCACAGGTGGGGGGGGAGTCGAG[CG]CGGAGGCTCGGGGGGCGGGACGCGGGGCCTGGGAGCGGCCA |
| | | | | GGGACCGCGGCAGCGCCTC |
| 36 | chr3 | 147127010 | cg14456683 | GGGATGTCAAGCGCTTTACAATACCTGGGATTGATGAGGCGGGCGGGCCAATGAGCTGCG[CG]CGGCGCCTCGGCGCGCCCTCCGTTGGCGCGGCGGCTGAGGG |
| | | | | GGGGGGAATGCGGGCGCA |
| 37 | chr3 | 157824150 | cg25694447 | GAAGAGAAGGGGCGGGGGCTGCCGGAGGGAAATGGGAACTGATGCTTCCCTGCCGGAGCG[CG]CTTGTTCAATGTTAAGATCTTTGACAATTCTTCCTGCGGAG |
| | | | | AGTTCAGATCTGATAGCGA |
| 38 | chr2 | 176964456 | cg03874199 | GATGTAGGCGGTGCTGAAATGACCGGCTTTGAAGAACCTGCAGGCAAAGTTTCGTCCAAT[CG]TCTGAGCCTGTCCTCTTATTCCCGGTTGTAACTAAATACTG |
| | | | | TTGCGAGCGCAGCCGAAGC |
| 39 | chr20 | 25062823 | cg15272362 | CTGGATCCCGTTTGCGGAGGGCCCAGCTTAGAGGAAGCTTTATAGGATTGGGCTGCCGGT[CG]GGCGCGGGAGGCCTGAAGGGCTCCAATCAGCTGGGCGCGGG |
| | | | | CGCGTCGCCGTTCCGGACC |
| 40 | chr4 | 46995741 | cg24154839 | ATGTATACCGCTCCACACCCTTTCGTGCCCGCGCGCTGAAGGTTCTGGGGTTCGTATCCG[CG]CGCTTGCGCTGCAAGACTCGGCAAGTTTGTTCCGACTGTAA |
| | | | | CTCCGGGGATGAGGAACGG |
| 41 | chr5 | 76934696 | cg21269843 | CCCGGAGAGTCCCCCACTCCGCCCGCCCGCTCGCCTGCGCCCGCCTGAAGGAGGCGGCGG[CG]GCAGCCCTGGCAGCTCCAGCCCGGAGCAGGAGCTAAAGCAG |
| | | | | CCTCAGCCGCCTCGGCCTC |
| 42 | chr16 | 56672525 | cg18438944 | GGGGCGGACTCGGCTGGGCGGACTCAGCGGGGGGGGCGCAGGCGCAGGGCGGGTCCTTTG[CG]TCCGGCCCTCTTTCCCCTGACCATAAAAGCAGCCGCTGGCT |
| | | | | GCTGGGCCCTACCAAGCCT |
| 43 | chr14 | 85996352 | cg01537995 | GCAACACACCCACTGGAAACGCACGTGAACAAAGCTCTCGCCCCCGGGAGCCGCTGCCTG[CG]GTTTCCTAGTCGATCCCAGCTTCTCTAGGGAGTGTCAGGCG |
| | | | | CACACAGGGTTAAGTTAGT |
| 44 | chr3 | 157824148 | cg26129769 | GAGAAGAGAAGGGGCGGGGGCTGCCGGAGGGAAATGGGAACTGATGCTTCCCTGCCGGAG[CG]CGCTTGTTCAATGTTAAGATCTTTGACAATTCTTCCTGCGG |
| | | | | AGAGTTCAGATCTGATAGC |
| 45 | chr4 | 9783246 | cg06469345 | GGGAGTCGAGCGCGGAGGCTCGGGGGGCGGGACGCGGGGCCTGGGAGCGGCCAGGGACCG[CG]GCAGCGCCTCAGTGCCAGCCTGGCGCCCGCGACTGCCTGCC |
| | | | | CCAGCCCCTCAGTGGCGGC |
| 46 | chr11 | 71955401 | cg05093169 | CCCCACCCCGAAGGGTCCCCGCCGCCGTCTTAGGGCTCAGAGGTGTGGAGCTGGGATGCG[CG]GGACCCTATCCCCGGAGTCCCTATCCTGCCCCAATCCGGAC |
| | | | | CAGACCTTCGGTCTCTACA |
| 47 | chr1 | 75594016 | cg01185626 | GAGAACGATTITACCGCCGATTTAAGCGTTCTCCTTTTTCCCCTTCGTGGCCGTTTTATT[CG]GCACTGAAATGCTGTTTAACCTCAGCAAGAGTAGGCGTGGG |
| | | | | AAGGTGGCGCTCTTCCCCA |
| 48 | chr8 | 132052870 | cg07557260 | TCTCCCGCCAGCCGGCGCAGCTTGGGTACGCAGCGGCAGTGGCTATTTGTCCTCAGGAGC[CG]CAGCGCTGTGAGCCACGCAGCCCCTTCCTGGGCTCAGGCTC |
| | | | | CTTGGTTGATTCTAGGCTC |
| 49 | chr3 | 137483506 | cg10343742 | ACTGGCATGATCAGCTGAACTTTGTGGGGTCAGCGCTTCTCTCTGGCTTCCCCTCAGCGG[CG]CCAAGGCGAGGGGAGCGCAGAACCCCGGCTCAGGACGGACA |
| | | | | GACAGACGGCCAGCCGCGC |
| 50 | chr20 | 54580196 | cg25993718 | CTGGTGACCCCATCTGAGCAGCTCTCTCCTGACGTTTAACGCACCCCAAAGCAAGCGGGG[CG]CACAGCACTGGTGATTGGCAAGGTTCGAGGCCCCTCAGCCC |
| | | | | CTGAAAGCCCGTGGCGTAA |
| 51 | chr10 | 28034853 | cg17389504 | GCGCCGCCCGGGAACAGGAATAGCGAGGCCTTCTCATGTTTCCTGACTGCCGGTCCCAGC[CG]GCGAACATCCTGCGGGCGCGGTATCCACGTTCCCGGGCGGG |
| | | | | TGGAGAGGAAGCGTCCGCG |
| 52 | chr20 | 25062860 | cg03111498 | CTTTATAGGATTGGGCTGCCGGTCGGGCGCGGGAGGCCTGAAGGGCTCCAATCAGCTGGG[CG]CGGGCGCGTCGCCGTTCCGGACCCCGCGAGATGGGGACGCC |
| | | | | ACCAGTTCCCCTCCCCTCG |
| 53 | chr2 | 182545562 | cg19711579 | TCTTCCCGGTCCAGCCAAGAGCCTGGAACCACGTGACCTGCCCATTTGTATGCCGCGGAG[CG]CTCCATTCCGGCCCCTTTGTGGCCAGAAGAAAGTGGCCCAT |
| | | | | CTGTCGCCAGTTAGAGACT |
| 54 | chr7 | 116963492 | cg01830294 | TGGCAAGAGATGTCTGGGGATGAGGTGAGGCGGGGAAGGGGGAACATCGCTGCATGGTTA[CG]AGCACACGCGAATACAGGAACCTTTAGGGAAGCTCCAGGCC |
| | | | | AAAGAGCATCCGTGGGCTC |
| 55 | chr20 | 25062777 | cg02631468 | AGTCCCGGCCGGTCATGGTTCCTTAGCAAGCAAGGCGCGAGCCTCTCTGGATCCCGTTTG[CG]GAGGGCCCAGCTTAGAGGAAGCTTTATAGGATTGGGCTGCC |
| | | | | GGTCGGGCGCGGGAGGCCT |
| 56 | chr8 | 109095991 | cg22600043 | TCCTAAAGCCAGAAACGTCCCGGTTTCCCTGAGGTCCTAAAGAACGTTGACAGCCAACAG[CG]CGCCTAACTTGGAGCGAATCCTCTTCGGGCTTTCCAGAGTG |
| | | | | CGGGGGATAGATAAAGAGT |
| 57 | chr2 | 176987404 | cg14991487 | AATCACAGCAGCCCCCAGTTTAGCGGACTGATTTACTCCCGGTATTGGTAAATATGATCA[CG]TGGGCCGCGCGACCAATGGTGGAGGCTGCAGCCTGCGAACT |
| | | | | AGTCGGTGGCTCGGGCGCC |
| 58 | chr13 | 79177877 | cg17576288 | ACGCGCGCTCTCCTTCTCGGCGGCCCCGTGCGTCTGCGCGCGCGCGCGCTCGTCCGGCCG[CG]ACCAGCGCGTGGGAGCCGCTCTTATAGTGACCACCAGCAAG |
| | | | | GACAGCGCAGGTGATGCAC |
| 59 | chr4 | 168155888 | cg05158197 | ACCTCCTCCTATCCCACCTTCCTCTCGCTGGATCCCAGCTACAGTTGGCTCCGGATAGGA[CG]CTTGGCTGGAGCCAGAGGACGAGGTAGCTCCTAGCCTAGGA |
| | | | | ATCCTTATCTGCTTTGTAA |
| 60 | chr2 | 19558507 | cg04690395 | ACCCCCGCCCCTTGCCTCAGGATTCCTCCCCTCCCTCCCCTGCCCCCCAACCCTGCCCTG[CG]CCTGCGCAATGAAAGACAAAGTTTTTGCTGACCCGGGCAGC |
| | | | | AGACTTTGGCAGCTGCTCT |
| 61 | chr11 | 20620824 | cg08530317 | AAGAATAGACAGTGATTTAACCCCCTCCTTCCTCCTCTACAGCCCGATCCACTCTGTTGG[CG]GCTGCGCCTTGCCTTGGGTGCAGTGGGACGTGTCCCTCCCA |
| | | | | TGTTTCCCTTAATTAGGAG |
| 62 | chr4 | 106816564 | cg08961408 | AGGGCGGCGAGTCGGGCGCACGCGCACCCCCTGCCCGCCCCTGGCGCCCCTCCCCGCGGG[CG]GTGCAGCTACCCCTGCAGCGCCTCCCCTAGCTAGAAGGGAG |
| | | | | CGGGAGGGGGCTCCGGGCG |
| 63 | chr14 | 85996364 | cg16800165 | CTGGAAACGCACGTGAACAAAGCTCTCGCCCCCGGGAGCCGCTGCCTGCGGTTTCCTAGT[CG]ATCCCAGCTTCTCTAGGGAGTGTCAGGCGCACACAGGGTTA |
| | | | | AGTTAGTTCCCTCCCTGGT |
| 64 | chr3 | 134369974 | cg00060320 | GAGGGCGCTCCCCCGCCGCTGGAGCCGCAGGGCGCTGCTTTTCGCTGACTCTGCCAAACA[CG]CCATTAGGGCCCGCCTGGGAGGGCGCTACCCTAATGAAGCG |
| | | | | GCTCTAAGTCACCTTTGAA |
| 65 | chr17 | 46692422 | cg25116388 | CACCCCCCACCCCCCAAACAAAAATATACCACCACTTAAAGAGGTCCTCGCTTTACATTT[CG]AAGAAGGGATGCCAGTTCATAAACAGTTTTCGGTGATTTAC |
| | | | | TTCCCTGCAAATGAGTTGT |
| 66 | chr4 | 96470286 | cg11723848 | CTTCTAACTGACGTCTCCCGCGAGCGACTGCTCTGCCCCCACTTGGGAAGTCCAGGGAGG[CG]CAAGGCGGCCACCTCCGCCGCTCCCCCCAGCAGCAGGAGCA |
| | | | | GCCGCAGCTCCGCCGCACG |

TABLE 3
Forward and reverse primers and probes designed for the methylated versions of specific regions of 18 genes for use in MethyLight
Reactions.

| | | SEQ ID | | SEQ ID | | |
|---|---|---|---|---|---|---|
| | | NO: of | | NO: of | | |
| | | forward | | reverse | | SEQ ID NO: |
| Gene | Forward Primer 5′ to 3′ | primer | Reverse Primer 5′ to 3′ | primer | Probe (5′ 6-FAM-3′ BHQ-1) | of probe |
| DRD5 | GCGGGCGTTAGGTTGGTATT | 85 | CCCTACACTCTAACGCGCTAAAA | 103 | AGGCGTTGTCGCGGTTTTTGGTC | 67 |
| HOXD9 | GCGGATTGATTTATTTTCGGTATT | 87 | CACCGACTAATTCGCAAACTACA | 105 | AAATATGATTACGTGGGTCGCGCGATT | 69 |
| FLRT2 | TGGGCGTTGGAAGTTAGTTACG | 86 | CACTCCTAAAAAAACTAAAATCGACTAAA | 104 | AGTTTTCGTTTTCGGGAGTCTTGTTTGC | 68 |
| MT1A | CGGTTGGGCGGATTTAGC | 88 | CCCAACAACCAACGACTACTT | 106 | TAGGGCGGGTTTTTTGCGTTCGG | 70 |
| VSX1 | GGGAATTGGTGGCGTTTTTA | 89 | ACTACCGATCGAACGCGAAA | 107 | CGCGTTCGCGTTTAGTTGATTGGAG | 71 |
| HOXD12 | CGGTTGCGTTCGTAATAGTATTTAGT | 90 | CCGACTTTAAAAAACCTACAAACAAA | 108 | TCGGGAATAAGAGGATAGGTTTAGACGATTGGA | 72 |
| LHX8 | TGGTCGTTTTATTCGGTATTGAAA | 91 | CTTCCGCTAAACAACTAAAAAATACCTAA | 109 | TTAGTAAGAGTAGGCGTGGGAAGGTGGCG | 73 |
| PHOX2A | GGATAGGGTTTCGCGTATTTTAGTT | 92 | CCCAAACACCGCCCAAA | 110 | TATATTTTTGAGTITTAAGACGGCGGCGGG | 74 |
| ZIC1 | GCGTTTTATAATATTTGGGATTGATG | 93 | CATTAATACGCCCGCATTCC | 111 | CGGGCGGGTTAATGAGTTGCGC | 75 |
| RSPO2 | CGTTTCGGTTTTTTTGAGGTTTT | 94 | CGTCTCCCCAACTACTCTITATCTATC | 112 | TTTGGAGCGAATTTTTTTCGGGTTTTTTAGAGT | 76 |
| SOX14 | CGTTGGGATTGGTATGATTAGTTG | 95 | CCGTCTATCTATCCGTCCTAAACC | 113 | TTTTTTAGCGGCGTTAAGGCGAGGG | 77 |
| OTP | GTTTTTGTTTCGGGTTGGAGTT | 96 | ATCAACGCCCGAAAAATCC | 114 | TTGTCGTCGTCGTTTTTTTTAGGCGG | 78 |
| WNT2 | TGTATGGTTACGAGTATACGCGAATAT | 97 | AACCTCGACAATCCGAAACG | 115 | TTAAAGAGTATTCGTGGGTTTTCGGAGCGT | 79 |
| NEUROD1 | GAGCGTTTCGCGGTATATAAATG | 98 | CCCGACATACGCCATATAATCTT | 116 | TAGGTTACGTGGTTTTAGGTTTTTGGTTGGATCG | 80 |
| CBLN4 | GAGCGCGAAGCGGGTAT | 99 | GCGAATTACGCATAAACCAA | 117 | CGCGTTTTATTTATAGGAGCGTAGCGTTCG | 81 |
| ADCY8 | TTTTGCGTGTTGTTTTTTCGTT | 100 | GCTACGACTCCTAAAAACAAATAACCA | 118 | CGGCGTAGTTTGGGTACGTAGCGGT | 82 |
| SLC6A5 | GGAAATATGGGAGGGATACGTTT | 101 | TAACCCCCTCCTTCCTCCTCTA | 119 | TAAGGTAAGGCGTAGTCGTTAATAGAGTGGATCGG | 83 |
| GABRA4 | GCGCGTTGAAGGTTTTGG | 102 | CCCCGAAATTACAATCGAAACA | 120 | TAAATTTGTCGAGTTTTGTAGCGTAAGCGCG | 84 |

### Cancer-Related CPGs for Analysis

In any of the assays described herein, in a sample which has been taken from an individual, the sample comprises a population of DNA molecules.

The assay of the invention comprise determining in the population of DNA molecules in the sample the methylation status of a panel of one or more CpGs selected from within a panel of one or more Differentially Methylated Regions (DMRs) defined by SEQ ID NOs 1 to 29, wherein selected CpGs in each DMR are denoted by CG. A breast cancer index value (WID-Breast-Index) is then derived based on the methylation status of the one or more CpGs in the panel, which may subsequently be used to assess the presence, absence or development of breast cancer in the individual based on the breast cancer index value.

In any of the assays described herein, the step of determining the methylation status of the one or more CpGs in the panel may comprise determining the methylation status of one or more CpGs within any one or more DMRs or within any combination of two or more DMRs defined by SEQ ID NOs 1 to 29, wherein selected CpGs in each DMR are denoted by CG. The DMRs are selected from the group consisting of:
a) DMR #1 (SEQ ID NO: 1);
b) DMR #2 (SEQ ID NO: 2);
c) DMR #3 (SEQ ID NO: 3);
d) DMR #4 (SEQ ID NO: 4);
e) DMR #5 (SEQ ID NO: 5);
f) DMR #6 (SEQ ID NO: 6);
g) DMR #7 (SEQ ID NO: 7);
h) DMR #8 (SEQ ID NO: 8);
i) DMR #9 (SEQ ID NO: 9);
j) DMR #10 (SEQ ID NO: 10);
k) DMR #11 (SEQ ID NO: 11);
l) DMR #12 (SEQ ID NO: 12);
m) DMR #13 (SEQ ID NO: 13);
n) DMR #14 (SEQ ID NO: 14);
o) DMR #15 (SEQ ID NO: 15);
p) DMR #16 (SEQ ID NO: 16);
q) DMR #17 (SEQ ID NO: 17);
r) DMR #18 (SEQ ID NO: 18);
s) DMR #19 (SEQ ID NO: 19);
t) DMR #20 (SEQ ID NO: 20);
u) DMR #21 (SEQ ID NO: 21);
v) DMR #22 (SEQ ID NO: 22);
w) DMR #23 (SEQ ID NO: 23);
x) DMR #24 (SEQ ID NO: 24);
y) DMR #25 (SEQ ID NO: 25);
z) DMR #26 (SEQ ID NO: 26);
aa) DMR #27 (SEQ ID NO: 27);
bb) DMR #28 (SEQ ID NO: 28); and
cc) DMR #29 (SEQ ID NO: 29).

The step of determining the methylation status of a panel of one or more CpGs comprises determining a breast cancer index value of each one of the one or more CpGs within any one of the DMRs of a) to cc), or within any combination of two or more DMRs of a) to cc).

The step of determining the methylation status of a panel of one or more CpGs comprises determining the methylation status of a panel of one or more CpGs comprises determining a breast cancer index value of each one of the one or more CpGs within:
a. four, five, six, seven, eight, or nine or more of a) to cc);
b. four, five, six, seven, eight, or nine or more of a) to r);
c. the four DMRs defined by a) to d);
d. the five DMRs defined by a) to e);
e. the six DMRs defined by a) to f);
f. the seven DMRs defined by a) to g);
g. the eight DMRs defined by a) to h); or
h. the nine DMRs defined by a) to i).

The panel of one or more CpGs may comprise two or more CpGs of the DMR(s), three or more CpGs of the DMR(s), four or more CpGs of the DMR(s) or all CpGs of the DMR(s).

The step of determining the methylation status of the one or more CpGs in the panel comprises determining the methylation status of one or more CpGs within any two or more DMRs defined by SEQ ID NOs 1 to 29, wherein selected CpGs in each DMR are denoted by CG, preferably the DMRs are defined by a) to r), and even more preferably wherein the two or more DMRs comprise at least the DMRs defined by a) and b).

The step of determining the methylation status of the one or more CpGs in the panel comprises determining the methylation status of one or more CpGs within any three or more DMRs defined by SEQ ID NOs 1 to 29, wherein selected CpGs in each DMR are denoted by CG, preferably wherein the DMRs are defined by a) to r), and even more preferably wherein the three or more DMRs comprise at least the DMRs defined by a) to c).

The step of determining the methylation status of the one or more CpGs in the panel comprises determining the methylation status of one or more CpGs within any four or more DMRs defined by SEQ ID NOs 1 to 29, wherein selected CpGs in each DMR are denoted by CG, preferably wherein the DMRs are defined by a) to r), and even more preferably wherein the four or more DMRs comprise at least the DMRs defined by a) to d) or comprise b), d), g) and n).

The step of determining the methylation status of the one or more CpGs in the panel comprises determining the methylation status of one or more CpGs within any five or more DMRs defined by SEQ ID NOs 1 to 29, wherein selected CpGs in each DMR are denoted by CG, preferably wherein the DMRs are defined by a) to r), and even more preferably wherein the five or more DMRs comprise at least the DMRs defined by a) to e) or comprise a), b), d), g) and n).

The step of determining the methylation status of the one or more CpGs in the panel comprises determining the methylation status of one or more CpGs within any six or more DMRs defined by SEQ ID NOs 1 to 29, wherein selected CpGs in each DMR are denoted by CG, preferably wherein the DMRs are defined by a) to r), and even more preferably wherein the six or more DMRs comprise at least the DMRs defined by a) to f) or comprise a) b), d), g), 1) and n).

The step of determining the methylation status of the one or more CpGs in the panel comprises determining the methylation status of one or more CpGs within any seven or more DMRs defined by SEQ ID NOs 1 to 29, wherein selected CpGs in each DMR are denoted by CG, preferably wherein the DMRs are defined by a) to r), and even more preferably wherein the seven or more DMRs comprise at least the DMRs defined by a) to g) or comprise a) b), c), d), g), 1) and n).

The step of determining the methylation status of the one or more CpGs in the panel comprises determining the methylation status of one or more CpGs within any eight or more DMRs defined by SEQ ID NOs 1 to 29, wherein selected CpGs in each DMR are denoted by CG, preferably wherein the DMRs are defined by a) to r), and even more preferably wherein the eight or more DMRs comprise at least the DMRs defined by a) to h) or comprise a) b), c), d), g), m), n) and o).

The step of determining the methylation status of the one or more CpGs in the panel comprises determining the methylation status of one or more CpGs within any nine or more DMRs defined by SEQ ID NOs 1 to 29, wherein selected CpGs in each DMR are denoted by CG, preferably wherein the DMRs are defined by a) to r), and even more preferably wherein the nine or more DMRs comprise at least the DMRs defined by a) to i) or comprise a) b), d), f), g), m), n), o) and p).

The step of determining the methylation status of the one or more CpGs in the panel comprises determining the methylation status of one or more CpGs within any ten or more DMRs defined by SEQ ID NOs 1 to 29, wherein selected CpGs in each DMR are denoted by CG, preferably wherein the DMRs are defined by a) to r), and even more preferably wherein the ten or more DMRs comprise at least the DMRs defined by a) to j).

The step of determining the methylation status of the one or more CpGs in the panel comprises determining the methylation status of one or more CpGs within any eighteen or more DMRs defined by SEQ ID NOs 1 to 29, wherein selected CpGs in each DMR are denoted by CG, preferably wherein the DMRs are defined by a) to r).

The step of determining the methylation status of the one or more CpGs in the panel comprises determining the methylation status of one or more CpGs within any twenty five or more DMRs defined by SEQ ID NOs 1 to 29, wherein selected CpGs in each DMR are denoted by CG, preferably wherein the DMRs are defined by a) to y).

The step of determining the methylation status of the one or more CpGs in the panel comprises determining the methylation status of one or more CpGs within all of the DMRs defined by SEQ ID NOs 1 to 29, wherein selected CpGs in each DMR are denoted by CG.

In any of the assays described herein, the assay involves a step of determining in the population of DNA molecules in the sample the methylation status of a panel of one or more CpGs selected from within a panel of one or more Differentially Methylated Regions (DMRs) defined by SEQ ID NOs 1 to 29, wherein selected CpGs in each DMR are denoted by CG. The panel of one or more CpGs may comprise two or more CpGs identified within a panel of one or more of the DMR(s), three or more CpGs identified within a panel of one or more of the DMR(s), four or more CpGs identified within a panel of one or more of the DMR(s), five or more CpGs identified within a panel of one or more of the DMR(s), six or more CpGs identified within a panel of one or more of the DMR(s), seven or more CpGs identified within a panel of one or more of the DMR(s), eight or more CpGs identified within a panel of one or more of the DMR(s), nine or more CpGs identified within a panel of one or more of the DMR(s), ten or more CpGs identified within a panel of one or more of the DMR(s), or all of the CpGs identified within a panel of one or more of the DMR(s). Preferably, the panel of one or more CpGs comprises at least three CpGs. Even more preferably, the panel of one or more CpGs comprises at least four CpGs.

In any of the assays described and defined herein, the assay may involve a step of determining in the population of DNA molecules in the sample the methylation status of a panel of one, more than one or all of the CpGs within a DMR panel comprising a combination of Differentially Methylated Regions (DMRs), wherein selected CpGs in each DMR are denoted by CG, and wherein the DMR panel comprises:
1 DMR #1 (SEQ ID NO: 1); DMR #3 (SEQ ID NO: 3); DMR #2 (SEQ ID NO: 2); DMR #4 (SEQ ID NO: 4); DMR #6 (SEQ ID NO: 6); DMR #7 (SEQ ID NO: 7); DMR #15 (SEQ ID NO: 15); and DMR #17 (SEQ ID NO: 17); and/or
2 DMR #1 (SEQ ID NO: 1); DMR #3 (SEQ ID NO: 3); DMR #4 (SEQ ID NO: 4); DMR #6 (SEQ ID NO: 6); DMR #7 (SEQ ID NO: 7); DMR #14 (SEQ ID NO: 14); DMR #15 (SEQ ID NO: 15); and DMR #17 (SEQ ID NO: 17); and/or
3 DMR #1 (SEQ ID NO: 1); DMR #2 (SEQ ID NO: 2); DMR #4 (SEQ ID NO: 4); DMR #6 (SEQ ID NO: 6); DMR #7 (SEQ ID NO: 7); DMR #14 (SEQ ID NO: 14); DMR #15 (SEQ ID NO: 15); and DMR #17 (SEQ ID NO: 17); and/or
4 DMR #1 (SEQ ID NO: 1); DMR #3 (SEQ ID NO: 3); DMR #2 (SEQ ID NO: 2); DMR #4 (SEQ ID NO: 4); DMR #6 (SEQ ID NO: 6); DMR #7 (SEQ ID NO: 7); DMR #14 (SEQ ID NO: 14); DMR #15 (SEQ ID NO: 15); and DMR #17 (SEQ ID NO: 17); and/or
5 DMR #1 (SEQ ID NO: 1); DMR #3 (SEQ ID NO: 3); DMR #4 (SEQ ID NO: 4); DMR #6 (SEQ ID NO: 6); DMR #7 (SEQ ID NO: 7); DMR #13 (SEQ ID NO: 13); DMR #14 (SEQ ID NO: 14); DMR #15 (SEQ ID NO: 15); and DMR #17 (SEQ ID NO: 17); and/or
6 DMR #3 (SEQ ID NO: 3); DMR #4 (SEQ ID NO: 4); DMR #6 (SEQ ID NO: 6); DMR #7 (SEQ ID NO: 7); DMR #15 (SEQ ID NO: 15); and DMR #17 (SEQ ID NO: 17); and/or
7 DMR #1 (SEQ ID NO: 1); DMR #3 (SEQ ID NO: 3); DMR #4 (SEQ ID NO: 4); DMR #6 (SEQ ID NO: 6); DMR #7 (SEQ ID NO: 7); DMR #15 (SEQ ID NO: 15); and DMR #17 (SEQ ID NO: 17); and/or
8 DMR #1 (SEQ ID NO: 1); DMR #2 (SEQ ID NO: 2); DMR #4 (SEQ ID NO: 4); DMR #6 (SEQ ID NO: 6); DMR #7 (SEQ ID NO: 7); DMR #15 (SEQ ID NO: 15); and DMR #17 (SEQ ID NO: 17); and/or
9 DMR #3 (SEQ ID NO: 3); DMR #2 (SEQ ID NO: 2); DMR #4 (SEQ ID NO: 4); DMR #6 (SEQ ID NO: 6); DMR #7 (SEQ ID NO: 7); DMR #15 (SEQ ID NO: 15); and DMR #17 (SEQ ID NO: 17); and/or
10 DMR #1 (SEQ ID NO: 1); DMR #2 (SEQ ID NO: 2); DMR #4 (SEQ ID NO: 4); DMR #6 (SEQ ID NO: 6); DMR #7 (SEQ ID NO: 7); DMR #14 (SEQ ID NO: 14); DMR #15 (SEQ ID NO: 15); and DMR #16 (SEQ ID NO: 16); and/or
11 DMR #1 (SEQ ID NO: 1); DMR #2 (SEQ ID NO: 2); DMR #4 (SEQ ID NO: 4); DMR #6 (SEQ ID NO: 6); DMR #7 (SEQ ID NO: 7); DMR #13 (SEQ ID NO: 13); DMR #14 (SEQ ID NO: 14); DMR #15 (SEQ ID NO: 15); and DMR #16 (SEQ ID NO: 16); and/or
12 DMR #1 (SEQ ID NO: 1); DMR #2 (SEQ ID NO: 2); DMR #4 (SEQ ID NO: 4); DMR #6 (SEQ ID NO: 6); DMR #7 (SEQ ID NO: 7); DMR #14 (SEQ ID NO: 14); DMR #15 (SEQ ID NO: 15); DMR #16 (SEQ ID NO: 16); and DMR #18 (SEQ ID NO: 18); and/or
13 DMR #4 (SEQ ID NO: 4); DMR #6 (SEQ ID NO: 6); DMR #7 (SEQ ID NO: 7); DMR #14 (SEQ ID NO: 14); and DMR #15 (SEQ ID NO: 15); and/or
14 DMR #1 (SEQ ID NO: 1); DMR #3 (SEQ ID NO: 3); DMR #2 (SEQ ID NO: 2); DMR #4 (SEQ ID NO: 4); DMR #7 (SEQ ID NO: 7); DMR #13 (SEQ ID NO: 13); DMR #14 (SEQ ID NO: 14); and DMR #15 (SEQ ID NO: 15); and/or 15 DMR #1 (SEQ ID NO: 1); DMR #3 (SEQ ID NO: 3); DMR #4 (SEQ ID NO: 4); DMR #7 (SEQ ID NO: 7); DMR #14 (SEQ ID NO: 14); and DMR #15 (SEQ ID NO: 15); and/or
16 DMR #1 (SEQ ID NO: 1); DMR #2 (SEQ ID NO: 2); DMR #4 (SEQ ID NO: 4); DMR #6 (SEQ ID NO: 6); DMR #7 (SEQ ID NO: 7); DMR #12 (SEQ ID NO: 12); and DMR #15 (SEQ ID NO: 15); and/or
17 DMR #1 (SEQ ID NO: 1); DMR #3 (SEQ ID NO: 3); DMR #2 (SEQ ID NO: 2); DMR #4 (SEQ ID NO: 4); DMR #7 (SEQ ID NO: 7); DMR #14 (SEQ ID NO: 14); DMR #15 (SEQ ID NO: 15); and DMR #16 (SEQ ID NO: 16); and/or
18 DMR #1 (SEQ ID NO: 1); DMR #3 (SEQ ID NO: 3); DMR #4 (SEQ ID NO: 4); DMR #6 (SEQ ID NO: 6); DMR #7 (SEQ ID NO: 7); DMR #12 (SEQ ID NO: 12); and DMR #15 (SEQ ID NO: 15); and/or
19 DMR #1 (SEQ ID NO: 1); DMR #3 (SEQ ID NO: 3); DMR #2 (SEQ ID NO: 2); DMR #4 (SEQ ID NO: 4); DMR #7 (SEQ ID NO: 7); DMR #14 (SEQ ID NO: 14); and DMR #15 (SEQ ID NO: 15); and/or
20 DMR #1 (SEQ ID NO: 1); DMR #4 (SEQ ID NO: 4); DMR #7 (SEQ ID NO: 7); DMR #12 (SEQ ID NO: 12); and DMR #14 (SEQ ID NO: 14); and/or
21 DMR #4 (SEQ ID NO: 4); DMR #5 (SEQ ID NO: 5); DMR #6 (SEQ ID NO: 6); DMR #7 (SEQ ID NO: 7); DMR #12 (SEQ ID NO: 12); and DMR #14 (SEQ ID NO: 14); and/or
22 DMR #1 (SEQ ID NO: 1); DMR #2 (SEQ ID NO: 2); DMR #4 (SEQ ID NO: 4); DMR #7 (SEQ ID NO: 7); DMR #14 (SEQ ID NO: 14); and DMR #15 (SEQ ID NO: 15); and/or
23 DMR #1 (SEQ ID NO: 1); DMR #2 (SEQ ID NO: 2); DMR #4 (SEQ ID NO: 4); DMR #7 (SEQ ID NO: 7); DMR #12 (SEQ ID NO: 12); and DMR #14 (SEQ ID NO: 14); and/or
24 DMR #4 (SEQ ID NO: 4); DMR #7 (SEQ ID NO: 7); DMR #14 (SEQ ID NO: 14); and DMR #15 (SEQ ID NO: 15); and/or
25 DMR #2 (SEQ ID NO: 2); DMR #4 (SEQ ID NO: 4); DMR #7 (SEQ ID NO: 7); DMR #14 (SEQ ID NO: 14); and DMR #15 (SEQ ID NO: 15); and/or
26 DMR #1 (SEQ ID NO: 1); DMR #3 (SEQ ID NO: 3); DMR #2 (SEQ ID NO: 2); DMR #4 (SEQ ID NO: 4); DMR #7 (SEQ ID NO: 7); DMR #12 (SEQ ID NO: 12); and DMR #14 (SEQ ID NO: 14); and/or
27 DMR #1 (SEQ ID NO: 1); DMR #3 (SEQ ID NO: 3); DMR #4 (SEQ ID NO: 4); DMR #7 (SEQ ID NO: 7); DMR #12 (SEQ ID NO: 12); and DMR #14 (SEQ ID NO: 14); and/or
28 DMR #2 (SEQ ID NO: 2); DMR #4 (SEQ ID NO: 4); DMR #7 (SEQ ID NO: 7); and DMR #14 (SEQ ID NO: 14); and/or
29 DMR #1 (SEQ ID NO: 1); DMR #2 (SEQ ID NO: 2); DMR #4 (SEQ ID NO: 4); DMR #7 (SEQ ID NO: 7); DMR #12 (SEQ ID NO: 12); DMR #13 (SEQ ID NO: 13); and DMR #14 (SEQ ID NO: 14); and/or 30 DMR #3 (SEQ ID NO: 3); DMR #4 (SEQ ID NO: 4); DMR #7 (SEQ ID NO: 7); DMR #12 (SEQ ID NO: 12); and DMR #14 (SEQ ID NO: 14); and/or
31 DMR #3 (SEQ ID NO: 3); DMR #2 (SEQ ID NO: 2); DMR #4 (SEQ ID NO: 4); DMR #7 (SEQ ID NO: 7); DMR #12 (SEQ ID NO: 12); and DMR #14 (SEQ ID NO: 14); and/or
32 DMR #1 (SEQ ID NO: 1); DMR #4 (SEQ ID NO: 4); DMR #7 (SEQ ID NO: 7); DMR #12 (SEQ ID NO: 12); DMR #13 (SEQ ID NO: 13); and DMR #14 (SEQ ID NO: 14); and/or
33 DMR #4 (SEQ ID NO: 4); DMR #7 (SEQ ID NO: 7); DMR #12 (SEQ ID NO: 12); and DMR #14 (SEQ ID NO: 14); and/or
34 DMR #1 (SEQ ID NO: 1); DMR #3 (SEQ ID NO: 3); DMR #4 (SEQ ID NO: 4); DMR #7 (SEQ ID NO: 7); and DMR #12 (SEQ ID NO: 12); and/or DMR #1 (SEQ ID NO: 1); DMR #3 (SEQ ID NO: 3); DMR #4 (SEQ ID NO: 4); DMR #7 (SEQ ID NO: 7); DMR #12 (SEQ ID NO: 12); DMR #13 (SEQ ID NO: 13); and DMR #14 (SEQ ID NO: 14); and/or
36 DMR #1 (SEQ ID NO: 1); DMR #3 (SEQ ID NO: 3); DMR #4 (SEQ ID NO: 4); DMR #7 (SEQ ID NO: 7); DMR #12 (SEQ ID NO: 12); DMR #14 (SEQ ID NO: 14); and DMR #16 (SEQ ID NO: 16); and/or
37 DMR #1 (SEQ ID NO: 1); DMR #4 (SEQ ID NO: 4); DMR #7 (SEQ ID NO: 7); and DMR #14 (SEQ ID NO: 14); and/or
38 DMR #1 (SEQ ID NO: 1); DMR #2 (SEQ ID NO: 2); DMR #4 (SEQ ID NO: 4); DMR #7 (SEQ ID NO: 7); and DMR #14 (SEQ ID NO: 14); and/or
39 DMR #1 (SEQ ID NO: 1); DMR #3 (SEQ ID NO: 3); DMR #4 (SEQ ID NO: 4); DMR #7 (SEQ ID NO: 7); DMR #12 (SEQ ID NO: 12); DMR #13 (SEQ ID NO: 13); DMR #14 (SEQ ID NO: 14); and DMR #16 (SEQ ID NO: 16); and/or 40 DMR #1 (SEQ ID NO: 1); DMR #4 (SEQ ID NO: 4); DMR #7 (SEQ ID NO: 7); and DMR #12 (SEQ ID NO: 12); and/or
41 DMR #1 (SEQ ID NO: 1); DMR #2 (SEQ ID NO: 2); DMR #4 (SEQ ID NO: 4); DMR #7 (SEQ ID NO: 7); and DMR #12 (SEQ ID NO: 12).

In any of the assays described herein, the step of determining the methylation status of the one or more CpGs in the panel may comprise determining the methylation status of any one or more CpGs within any one or more of the sequences identified by SEQ ID NOs 67 to 84. The sequences identified by SEQ ID NOs 67 to 84 are set out in Table 3 In any of the assays described herein, the step of determining the methylation status of the one or more CpGs in the panel may comprise determining the methylation status of any one or more CpGs within any one or more of the sequences identified by SEQ ID NOs 67 to 84. The assay may comprise determining the methylation status of any one or more CpGs within any two or more, three or more, four or more, five or more, six or more, seven or more, eight or more, nine or more, ten or more, eleven or more, twelve or more, thirteen or more, fourteen or more, fifteen or more, sixteen or more, seventeen or more, or all eighteen of the sequences identified by SEQ ID NOs 67 to 84.

Wherein the assay comprises determining the methylation status of any one or more CpGs within four or more of the sequences identified by SEQ ID NOs 67 to 84, the four or more sequences preferably comprise SEQ ID NOs 68, 70, 73 and 80.

Wherein the assay comprises determining the methylation status of any one or more CpGs within five or more of the sequences identified by SEQ ID NOs 67 to 84, the five or more sequences preferably comprise SEQ ID NOs 67, 68, 70, 73 and 80, or even more preferably comprise SEQ ID NOs 70, 72, 73, 80 and 81.

Wherein the assay comprises determining the methylation status of any one or more CpGs within six or more of the sequences identified by SEQ ID NOs 67 to 84, the six or more sequences preferably comprise SEQ ID NOs 67, 68, 70, 73, 78 and 80.

Wherein the assay comprises determining the methylation status of any one or more CpGs within seven or more of the sequences identified by SEQ ID NOs 67 to 84, the seven or more sequences preferably comprise SEQ ID NOs 67, 68, 69, 70, 73, 78 and 80.

Wherein the assay comprises determining the methylation status of any one or more CpGs within eight or more of the sequences identified by SEQ ID NOs 67 to 84, the eight or more sequences preferably comprise SEQ ID NOs 67, 68, 69, 70, 73, 79, 80 and 81.

Wherein the assay comprises determining the methylation status of any one or more CpGs within nine or more of the sequences identified by SEQ ID NOs 67 to 84, the nine or more sequences preferably comprise SEQ ID NOs 67, 68, 70, 72, 73, 79, 80, 81 and 82.

In any of the assays described herein, the step of determining the methylation status of the one or more CpGs in the panel may comprise determining the methylation status of one or more CpGs identified at nucleotide positions 61 to 62 in SEQ ID NOs 30 to 66.

The assay may comprise determining the methylation status of any one or more, two or more, three or more, four or more, five or more, six or more, seven or more, eight or more, nine or more, ten or more, fifteen or more, twenty or more, twenty five or more, thirty or more, thirty five or more, or all thirty seven CpGs identified at nucleotide positions 61 to 62 in SEQ ID NOs 30 to 66. The thirty seven CpGs identified at nucleotide positions 61 to 62 in SEQ ID NOs 30 to 66 are set out in Table 2.

Wherein the assay comprises determining the methylation status of any one or more CpGs identified at nucleotide positions 61 to 62 in SEQ ID NOs 30 to 66, the one or more CpGs may optionally comprises at least 5 CpGs selected from the CpGs identified in SEQ ID NOs 30 to 66, and preferably the at least 5 CpGs comprises at least the CpGs identified in SEQ ID NOs 30 to 34 and identified at nucleotide positions 61 to 62.

Wherein the assay comprises determining the methylation status of any one or more CpGs identified at nucleotide positions 61 to 62 in SEQ ID NOs 30 to 66, the one or more CpGs may optionally comprises at least 10 CpGs selected from the CpGs identified in SEQ ID NOs 30 to 66, and preferably the at least 10 CpGs comprises at least the CpGs identified in SEQ ID NOs 30 to 39 and identified at nucleotide positions 61 to 62.

Wherein the assay comprises determining the methylation status of any one or more CpGs identified at nucleotide positions 61 to 62 in SEQ ID NOs 30 to 66, the one or more CpGs may optionally comprises at least 15 CpGs selected from the CpGs identified in SEQ ID NOs 30 to 66, and preferably the at least 15 CpGs comprises at least the CpGs identified in SEQ ID NOs 30 to 44 and identified at nucleotide positions 61 to 62.

Wherein the assay comprises determining the methylation status of any one or more CpGs identified at nucleotide positions 61 to 62 in SEQ ID NOs 30 to 66, the one or more CpGs may optionally comprises at least 20 CpGs selected from the CpGs identified in SEQ ID NOs 30 to 66, and preferably the at least 20 CpGs comprises at least the CpGs identified in SEQ ID NOs 30 to 49 and identified at nucleotide positions 61 to 62.

Wherein the assay comprises determining the methylation status of any one or more CpGs identified at nucleotide positions 61 to 62 in SEQ ID NOs 30 to 66, the one or more CpGs may optionally comprises at least 25 CpGs selected from the CpGs identified in SEQ ID NOs 30 to 66, and preferably the at least 25 CpGs comprises at least the CpGs identified in SEQ ID NOs 30 to 54 and identified at nucleotide positions 61 to 62.

Wherein the assay comprises determining the methylation status of any one or more CpGs identified at nucleotide positions 61 to 62 in SEQ ID NOs 30 to 66, the one or more CpGs may optionally comprises at least 30 CpGs selected from the CpGs identified in SEQ ID NOs 30 to 66, and preferably the at least 30 CpGs comprises at least the CpGs identified in SEQ ID NOs 30 to 59 and identified at nucleotide positions 61 to 62.

Wherein the assay comprises determining the methylation status of any one or more CpGs identified at nucleotide positions 61 to 62 in SEQ ID NOs 30 to 66, the one or more CpGs may optionally comprises at least 35 CpGs selected from the CpGs identified in SEQ ID NOs 30 to 66, and preferably the at least 35 CpGs comprises at least the CpGs identified in SEQ ID NOs 30 to 64 and identified at nucleotide positions 61 to 62.

Wherein the assay comprises determining the methylation status of any one or more CpGs identified at nucleotide positions 61 to 62 in SEQ ID NOs 30 to 66, the one or more CpGs may optionally comprises the 37 CpGs selected from the CpGs identified in SEQ ID NOs 30 to 66, and preferably the 37 CpGs consist of the CpGs identified in SEQ ID NOs 30 to 66 and identified at nucleotide positions 61 to 62.

The invention also provides a variety of assays, each comprising any 1, 2, 3, 4, 5, 6, 7, 8, 9, 10 or more (or any range derivable therein) of a variety of steps and in no particular order, including methods of the following: measuring in a sample; analyzing a sample; assessing a sample; evaluating a sample; measuring nucleic acids in a sample; assessing nucleic acids in a sample; detecting nucleic acids in a sample; measuring methylation in nucleic acids in a sample; analyzing nucleic acids in a sample; assessing nucleic acids in a sample; measuring methylation at one or more CpG dinucleotides in a sample; detecting methylation at one or more CpG dinucleotides in a sample; assaying methylation at one or more CpG dinucleotides in a sample; assessing methylation at one or more CpG dinucleotides in a sample; measuring a methylation status in a sample; assaying a methylation status in a sample; detecting methylation status in a sample; determining methylation status in a sample; identifying methylation status in a sample; measuring one or more DNA methylation markers in a sample; assessing one or more DNA methylation markers in a sample; detecting one or more DNA methylation markers in a sample; measuring the presence of methylation at one or more markers in a sample; detecting the presence of methylation at one or more markers in a sample; assessing the presence of methylation at one or more markers in a sample; assaying the presence of one of more markers in a sample; measuring one or more DNA methylation markers in a sample but excluding the measuring of one or more other DNA methylation markers in the sample; assessing one or more DNA methylation markers in a sample but excluding the assessing of one or more other DNA methylation markers in the sample; analyzing one or more DNA methylation markers in a sample but excluding the analyzing of one or more other DNA methylation markers in the sample; detecting one or more DNA methylation markers in a sample but excluding the detecting of one or more other DNA methylation markers in the sample; measuring methylation status in nucleic acids from a sample from tissue from an individual other than tissue from the individual suspected of, or at risk for, being cancerous; detecting methylation status in nucleic acids from a sample from tissue from an individual other than tissue from the individual suspected of, or at risk for, being cancerous; analyzing methylation status in nucleic acids from a sample from tissue from an individual other than tissue from the individual suspected of, or at risk for, being cancerous; assessing methylation status in nucleic acids from a sample from tissue from an individual other than tissue from the individual suspected of, or at risk for, being cancerous; measuring methylation at one or more CpG dinucleotides in a sample but excluding the measuring of methylation at one or more CpG dinucleotides in the sample; assessing methylation at one or more CpG dinucleotides in a sample but excluding the assessing of methylation at one or more CpG dinucleotides in the sample; analyzing methylation at one or more CpG dinucleotides in a sample but excluding the analyzing of methylation at one or more CpG dinucleotides in the sample; detecting methylation at one or more CpG dinucleotides in a sample but excluding the detecting of methylation at one or more CpG dinucleotides in the sample; measuring methylation at one or more CpG dinucleotides in nucleic acids from a sample from tissue from an individual other than tissue from the individual suspected of, or at risk for, being cancerous; detecting methylation at one or more CpG dinucleotides in nucleic acids from a sample from tissue from an individual other than tissue from the individual suspected of, or at risk for, being cancerous; analyzing methylation at one or more CpG dinucleotides in nucleic acids from a sample from tissue from an individual other than tissue from the individual suspected of, or at risk for, being cancerous; assessing methylation at one or more CpG dinucleotides in nucleic acids from a sample from tissue from an individual other than tissue from the individual suspected of, or at risk for, being cancerous; treating an individual for cancer when the individual has been determined to have a methylation status at one or more methylation markers; treating an individual for cancer when the individual has been determined to have methylation at one or more CpG dinucleotides;
wherein any of the aforementioned methods, or any other methods encompassed by the disclosure, may comprise any one or more of the following method steps:
measuring methylation status, wherein the measuring identifies the methylation status of one or markers from nucleic acids in a sample;
measuring methylation status, wherein the measuring identifies the presence of one or more markers from nucleic acids in a sample;
measuring the presence of one or more methylation markers from a sample;
providing DNA from a sample;
providing nucleic acids from a sample;
determining whether one or more methylation markers from nucleic acids from a sample are methylated;
measuring whether one or more methylation markers from nucleic acids from a sample are methylated;
performing a sequencing step on nucleic acids from a sample;
determining a sequence of nucleic acids from a sample;
performing bisulphite conversion on one or more markers;
performing bisulphite conversion on one or more CpG dinucleotides;
hybridizing DNA to an array comprising probes capable of determining methylated versus non-methylated markers;
hybridizing DNA to an array comprising probes capable of determining methylated versus non-methylated CpG dinucleotides;
hybridizing DNA to an array comprising probes capable of discriminating between methylated and non-methylated markers;
hybridizing DNA to an array comprising probes capable of discriminating between methylated and non-methylated CpG dinucleotides;
performing an amplification step on sequence from nucleic acids from a sample; performing an amplification step on sequence from nucleic acids using methylation-specific primers;
performing amplification of sequence comprising one or more regions suspected of having methylation or in need of determination of a methylation status;
performing PCR on sequence comprising one or more regions suspected of having methylation or in need of determination of a methylation status;
performing a capturing step;
performing a binding step;
performing a purification step;
performing a capturing step comprising binding of polynucleotides comprising one or more methylation markers to binding molecules specific to the one or more methylation markers and collecting complexes thereof;
stratifying the grade of a cancer;
determining the risk of cancer;
determining the risk of recurrence of cancer;
obtaining a sample from an individual;
obtaining DNA from a sample from an individual;
administering a treatment to an individual;
providing DNA from a sample;
determining whether one or more methylation markers from a panel of methylation markers comprises a specific sequence; and/or
obtaining data that identifies whether each one of a group of methylation markers from a panel comprises a specific sequence.

Moreover, in some aspects of the invention, an individual who is administered a therapy or treatment has been subjected to any of the methods and steps described herein.

Described herein are assays that utilise a statistically robust panel of one or more CpGs whose methylation status can be determined to provide a reliable prediction of the presence or development of breast cancer in an individual. By determining the methylation status of each CpG within the panel of one or more CpGs, a breast cancer index value may be derived thus enabling stratification of individuals according to their risk of developing breast cancer or of having breast cancer with statistically robust sensitivity and specificity. The skilled person would understand that the methylation status of each CpG within a panel of one or more CpGs can be determined by any suitable means in order to thereby derive the breast cancer index value. Any one method, or a combination of methods, may be used to determine the methylation status of each CpG within a panel of one or more CpGs.

Various exemplary methods for determining the methylation status of each CpG within a panel of one or more CpGs are described herein. For example, in one method a percent methylated reference (PMR) value of a CpG may be determined. In another method the methylation β-values of a CpG may be determined. Different mechanisms may be employed to determine specific values depending on the circumstances, such as PCR-based mechanisms or array-based mechanisms.

TABLE 4
Breast cancer index value cutpoints and associated statistical parameters for various DMR (gene) combinations.

| | | Normal breast tissue from cancer free women vs | | | | | | |
|---|---|---|---|---|---|---|---|---|
| | | normal tissue surrounding breast cancer tissue | | | | | | |
| | | | Cutpoint | Sensitivity | Cutpoint | Sensitivity | Cutpoint | Sensitivity |
| | | | for | at 80% | for | at 90% | for | at 95% |
| Combination | WID-Breast | ROC | Specificity | Specificity | Specificity | Specificity | Specificity | Specificity |
| number | Gene Combinations | AUC | 80% | (%) | 90% | (%) | 95% | (%) |
| 1 | DRD5; HOXD9; FLRT2; | 0.91 | 4.82 | 78.71 | 5.43 | 57.53 | 5.86 | 37.79 |
| | MTA1; HOXD12; LHX8; | | | | | | | |
| | CBLN4; SLC6A5 | | | | | | | |
| 2 | DRD5; HOXD9; MTA1; | 0.90 | 4.80 | 78.46 | 5.44 | 57.91 | 6.06 | 38.73 |
| | HOXD12; LHX8; | | | | | | | |
| | NEUROD1; CBLN4; | | | | | | | |
| | SLC6A5 | | | | | | | |
| 3 | DRD5; FLRT2; MTA1; | 0.90 | 5.06 | 76.93 | 5.38 | 51.63 | 5.97 | 29.68 |
| | HOXD12; LHX8; | | | | | | | |
| | NEUROD1; CBLN4; | | | | | | | |
| | SLC6A5 | | | | | | | |
| 4 | DRD5; HOXD9; FLRT2; | 0.90 | 4.41 | 78.46 | 5.01 | 57.91 | 5.45 | 38.73 |
| | MTA1; HOXD12; LHX8; | | | | | | | |
| | NEUROD1; CBLN4; | | | | | | | |
| | SLC6A5 | | | | | | | |
| 5 | DRD5; HOXD9; MTA1; | 0.90 | 4.41 | 78.46 | 5.00 | 57.91 | 5.51 | 38.73 |
| | HOXD12; LHX8; WNT2; | | | | | | | |
| | NEUROD1; CBLN4; | | | | | | | |
| | SLC6A5 | | | | | | | |
| 6 | HOXD9; MTA1; HOXD12; | 0.90 | 6.05 | 78.46 | 6.77 | 57.91 | 7.43 | 38.73 |
| | LHX8; CBLN4; SLC6A5 | | | | | | | |
| 7 | DRD5; HOXD9; MTA1; | 0.90 | 5.32 | 78.46 | 5.99 | 57.91 | 6.65 | 38.73 |
| | HOXD12; LHX8; CBLN4; | | | | | | | |
| | SLC6A5 | | | | | | | |
| 8 | DRD5; FLRT2; MTA1; | 0.90 | 5.60 | 76.93 | 5.90 | 51.63 | 6.56 | 29.68 |
| | HOXD12; LHX8; CBLN4; | | | | | | | |
| | SLC6A5 | | | | | | | |
| 9 | HOXD9; FLRT2; MTA1; | 0.90 | 5.36 | 76.38 | 6.24 | 48.33 | 6.46 | 25.15 |
| | HOXD12; LHX8; CBLN4; | | | | | | | |
| | SLC6A5 | | | | | | | |
| 10 | DRD5; FLRT2; MTA1; | 0.90 | 4.38 | 76.38 | 5.55 | 48.33 | 5.72 | 25.15 |
| | HOXD12; LHX8; NEUROD1; | | | | | | | |
| | CBLN4; ADCY8 | | | | | | | |
| 11 | DRD5; FLRT2; MTA1; | 0.90 | 4.01 | 76.38 | 5.12 | 48.33 | 5.28 | 25.15 |
| | HOXD12; LHX8; WNT2; | | | | | | | |
| | NEUROD1; CBLN4; | | | | | | | |
| | ADCY8 | | | | | | | |
| 12 | DRD5; FLRT2; MTA1; | 0.90 | 4.18 | 76.38 | 5.53 | 48.33 | 5.67 | 25.15 |
| | HOXD12; LHX8; NEUROD1; | | | | | | | |
| | CBLN4; ADCY8; GABRA4 | | | | | | | |
| 13 | MTA1; HOXD12; LHX8; | 0.90 | 5.87 | 77.13 | 7.06 | 56.72 | 7.13 | 38.03 |
| | NEUROD1; CBLN4 | | | | | | | |
| 14 | DRD5; HOXD9; FLRT2; | 0.90 | 2.85 | 79.57 | 3.93 | 34.64 | 4.02 | 8.02 |
| | MTA1; LHX8; WNT2; | | | | | | | |
| | NEUROD1; CBLN4 | | | | | | | |
| 15 | DRD5; HOXD9; MTA1; | 0.90 | 3.48 | 80.37 | 4.62 | 48.73 | 4.86 | 22.24 |
| | LHX8; NEUROD1; CBLN4 | | | | | | | |
| 16 | DRD5; FLRT2; MTA1; | 0.90 | 4.71 | 77.13 | 5.82 | 56.72 | 5.89 | 38.03 |
| | HOXD12; LHX8; OTP; | | | | | | | |
| | CBLN4 | | | | | | | |
| 17 | DRD5; HOXD9; FLRT2; | 0.90 | 3.16 | 79.57 | 4.48 | 34.64 | 4.60 | 8.02 |
| | MTA1; LHX8; NEUROD1; | | | | | | | |
| | CBLN4; ADCY8 | | | | | | | |
| 18 | DRD5; HOXD9; MTA1; | 0.90 | 4.78 | 77.13 | 5.91 | 56.72 | 5.95 | 38.03 |
| | HOXD12; LHX8; OTP; | | | | | | | |
| | CBLN4 | | | | | | | |
| 19 | DRD5; HOXD9; FLRT2; | 0.90 | 3.15 | 79.57 | 4.25 | 34.64 | 4.33 | 8.02 |
| | MTA1; LHX8; NEUROD1; | | | | | | | |
| | CBLN4 | | | | | | | |
| 20 | DRD5; MTA1; LHX8; | 0.92 | 3.51 | 81.92 | 4.81 | 58.21 | 4.99 | 35.38 |
| | OTP; NEUROD1 | | | | | | | |
| 21 | MTA1; VSX1; HOXD12; | 0.90 | 5.07 | 79.57 | 5.95 | 66.09 | 6.50 | 52.99 |
| | LHX8; OTP; NEUROD1 | | | | | | | |
| 22 | DRD5; FLRT2; MTA1; | 0.90 | 3.44 | 79.57 | 4.69 | 34.64 | 4.77 | 8.02 |
| | LHX8; NEUROD1; CBLN4 | | | | | | | |
| 23 | DRD5; FLRT2; MTA1; | 0.91 | 3.09 | 81.92 | 4.22 | 58.21 | 4.39 | 35.38 |
| | LHX8; OTP; NEUROD1 | | | | | | | |
| 24 | MTA1; LHX8; NEUROD1; | 0.90 | 4.57 | 80.85 | 6.07 | 49.83 | 6.57 | 23.32 |
| | CBLN4 | | | | | | | |
| 25 | FLRT2; MTA1; LHX8; | 0.90 | 3.91 | 79.57 | 5.32 | 34.64 | 5.37 | 8.02 |
| | NEUROD1; CBLN4 | | | | | | | |
| 26 | DRD5; HOXD9; FLRT2; | 0.91 | 2.84 | 82.73 | 3.62 | 66.53 | 4.11 | 50.01 |
| | MTA1; LHX8; OTP; | | | | | | | |
| | NEUROD1 | | | | | | | |
| 27 | DRD5; HOXD9; MTA1; | 0.91 | 3.14 | 79.69 | 4.33 | 61.73 | 4.42 | 44.39 |
| | LHX8; OTP; NEUROD1 | | | | | | | |
| 28 | FLRT2; MTA1; LHX8; | 0.91 | 3.73 | 82.73 | 4.50 | 66.53 | 5.26 | 50.01 |
| | NEUROD1 | | | | | | | |
| 29 | DRD5; FLRT2; MTA1; | 0.91 | 3.01 | 80.64 | 3.78 | 56.23 | 4.04 | 33.44 |
| | LHX8; OTP; WNT2; | | | | | | | |
| | NEUROD1 | | | | | | | |
| 30 | HOXD9; MTA1; LHX8; | 0.90 | 3.56 | 79.69 | 4.59 | 66.52 | 5.15 | 53.71 |
| | OTP; NEUROD1 | | | | | | | |
| 31 | HOXD9; FLRT2; MTA1; | 0.90 | 3.14 | 80.62 | 3.96 | 64.37 | 4.67 | 48.36 |
| | LHX8; OTP; NEUROD1 | | | | | | | |
| 32 | DRD5; MTA1; LHX8; | 0.90 | 3.30 | 78.06 | 4.43 | 46.37 | 4.46 | 21.02 |
| | OTP; WNT2; NEUROD1 | | | | | | | |
| 33 | MTA1; LHX8; OTP; | 0.90 | 4.18 | 77.90 | 5.65 | 62.19 | 5.82 | 47.28 |
| | NEUROD1 | | | | | | | |
| 34 | DRD5; HOXD9; MTA1; | 0.90 | 3.51 | 79.88 | 4.67 | 62.29 | 4.98 | 45.23 |
| | LHX8; OTP | | | | | | | |
| 35 | DRD5; HOXD9; MTA1; | 0.90 | 3.08 | 79.37 | 3.91 | 57.68 | 4.11 | 37.33 |
| | LHX8; OTP; WNT2; | | | | | | | |
| | NEUROD1 | | | | | | | |
| 36 | DRD5; HOXD9; MTA1; | 0.90 | 3.42 | 78.06 | 4.64 | 46.37 | 4.74 | 21.02 |
| | LHX8; OTP; NEUROD1; | | | | | | | |
| | ADCY8 | | | | | | | |
| 37 | DRD5; MTA1; LHX8; | 0.90 | 3.80 | 78.65 | 4.97 | 62.68 | 5.09 | 47.40 |
| | NEUROD1 | | | | | | | |
| 38 | DRD5; FLRT2; MTA1; | 0.90 | 3.24 | 79.42 | 4.11 | 62.04 | 4.46 | 45.29 |
| | LHX8; NEUROD1 | | | | | | | |
| 39 | DRD5; HOXD9; MTA1; | 0.90 | 3.12 | 78.06 | 4.27 | 46.37 | 4.37 | 21.02 |
| | LHX8; OTP; WNT2; | | | | | | | |
| | NEUROD1; ADCY8 | | | | | | | |
| 40 | DRD5; MTA1; LHX8; | 0.90 | 4.06 | 78.70 | 5.64 | 53.54 | 5.78 | 31.02 |
| | OTP | | | | | | | |
| 41 | DRD5; FLRT2; MTA1; | 0.90 | 3.42 | 80.70 | 4.75 | 53.65 | 4.98 | 29.17 |
| | LHX8; OTP | | | | | | | |

| | Normal tissue surrounding breast cancer | | | | | | |
|---|---|---|---|---|---|---|---|
| | tissue vs breast Cancer tissue | | | | | | |
| | | Cutpoint | Sensitivity | Cutpoint | Sensitivity | Cutpoint | Sensitivity |
| | | for | at 80% | for | at 90% | for | at 95% |
| Combination | ROC | Specificity | Specificity | Specificity | Specificity | Specificity | Specificity |
| number | AUC | 80% | (%) | 90% | (%) | 95% | (%) |
| 1 | 0.87 | 7.51 | 75.37 | 14.70 | 63.85 | 23.93 | 53.21 |
| 2 | 0.87 | 8.58 | 75.67 | 16.25 | 63.38 | 25.17 | 51.99 |
| 3 | 0.87 | 8.65 | 75.70 | 15.81 | 65.09 | 20.55 | 55.28 |
| 4 | 0.87 | 7.87 | 75.67 | 14.46 | 63.38 | 22.50 | 51.99 |
| 5 | 0.87 | 7.70 | 76.42 | 14.48 | 66.09 | 20.43 | 56.47 |
| 6 | 0.87 | 9.33 | 75.37 | 19.52 | 63.85 | 30.48 | 53.21 |
| 7 | 0.87 | 8.21 | 75.37 | 16.78 | 63.85 | 27.18 | 53.21 |
| 8 | 0.87 | 8.30 | 74.74 | 16.28 | 62.87 | 24.16 | 51.97 |
| 9 | 0.87 | 8.35 | 75.37 | 16.76 | 63.85 | 26.29 | 53.21 |
| 10 | 0.87 | 7.59 | 74.34 | 20.89 | 61.93 | 22.74 | 50.58 |
| 11 | 0.87 | 6.81 | 74.34 | 18.60 | 61.93 | 20.33 | 50.58 |
| 12 | 0.87 | 7.37 | 74.34 | 20.43 | 61.93 | 21.59 | 50.58 |
| 13 | 0.86 | 10.99 | 73.71 | 21.45 | 62.84 | 30.78 | 52.96 |
| 14 | 0.86 | 5.59 | 74.34 | 11.97 | 64.92 | 16.82 | 56.35 |
| 15 | 0.85 | 7.08 | 73.15 | 15.80 | 62.87 | 25.35 | 53.57 |
| 16 | 0.85 | 8.79 | 71.98 | 17.93 | 59.56 | 26.75 | 48.46 |
| 17 | 0.85 | 6.16 | 71.57 | 19.63 | 58.75 | 21.64 | 47.33 |
| 18 | 0.85 | 8.75 | 70.90 | 19.16 | 57.30 | 33.89 | 45.28 |
| 19 | 0.85 | 6.29 | 72.58 | 13.59 | 62.70 | 21.89 | 53.82 |
| 20 | 0.83 | 7.29 | 74.07 | 11.41 | 67.24 | 20.13 | 61.11 |
| 21 | 0.84 | 10.34 | 71.57 | 15.73 | 60.37 | 32.60 | 50.36 |
| 22 | 0.84 | 7.08 | 74.67 | 15.22 | 66.52 | 19.08 | 59.12 |
| 23 | 0.82 | 6.43 | 73.38 | 9.56 | 66.43 | 17.05 | 60.23 |
| 24 | 0.83 | 10.34 | 72.94 | 22.39 | 64.44 | 29.62 | 56.81 |
| 25 | 0.83 | 8.40 | 72.94 | 17.98 | 64.44 | 24.03 | 56.81 |
| 26 | 0.82 | 5.73 | 68.84 | 8.74 | 58.79 | 22.74 | 49.99 |
| 27 | 0.82 | 6.33 | 69.58 | 10.14 | 59.57 | 26.26 | 50.77 |
| 28 | 0.81 | 6.85 | 70.09 | 13.65 | 61.73 | 19.79 | 54.36 |
| 29 | 0.82 | 5.61 | 72.62 | 8.28 | 65.53 | 14.83 | 59.21 |
| 30 | 0.82 | 7.34 | 69.74 | 11.88 | 60.35 | 29.80 | 52.08 |
| 31 | 0.82 | 6.48 | 69.74 | 9.96 | 60.35 | 24.92 | 52.08 |
| 32 | 0.82 | 6.18 | 73.38 | 9.61 | 66.43 | 17.03 | 60.23 |
| 33 | 0.82 | 8.79 | 72.14 | 13.90 | 64.74 | 28.49 | 58.16 |
| 34 | 0.82 | 6.26 | 68.16 | 11.41 | 57.18 | 29.89 | 47.63 |
| 35 | 0.82 | 5.51 | 68.84 | 8.78 | 58.79 | 22.83 | 49.99 |
| 36 | 0.82 | 6.23 | 68.10 | 18.88 | 58.25 | 22.48 | 49.68 |
| 37 | 0.81 | 6.70 | 70.44 | 13.62 | 62.57 | 18.85 | 55.64 |
| 38 | 0.81 | 5.80 | 70.44 | 11.12 | 62.57 | 15.38 | 55.64 |
| 39 | 0.81 | 5.52 | 67.04 | 16.55 | 56.96 | 19.68 | 48.23 |
| 40 | 0.80 | 7.42 | 68.05 | 13.38 | 58.07 | 31.80 | 49.38 |
| 41 | 0.80 | 6.32 | 67.43 | 10.94 | 57.44 | 25.55 | 48.78 |

TABLE 5
Breast cancer index value cutpoints and associated statistical
parameters for various CpG combinations.

| | Normal breast tissue from cancer free women vs | | | | | | |
|---|---|---|---|---|---|---|---|
| | normal tissue surrounding breast cancer tissue | | | | | | |
| | | Cutpoint | Sensitivity | Cutpoint | Sensitivity | Cutpoint | Sensitivity |
| | | for | at 80% | for | at 90% | for | at 95% |
| WID-Breast29 | ROC | Specificity | Specificity | Specificity | Specificity | Specificity | Specificity |
| CpG combinations | AUC | 80% | (%) | 90% | (%) | 95% | (%) |
| All 37 CpGs that are | 0.63 | 0.15 | 37.00 | 0.16 | 22.60 | 0.19 | 13.60 |
| identified in SEQ | | | | | | | |
| ID NOs 30 to 66 | | | | | | | |
| | Normal tissue surround breast cancer | | | | | | |
| | tissue vs Breast Cancer tissue | | | | | | |
| | | Cutpoint | Sensitivity | Cutpoint | Sensitivity | Cutpoint | Sensitivity |
| | | for | at 80% | for | at 90% | for | at 95% |
| WID-Breast29 | ROC | Specificity | Specificity | Specificity | Specificity | Specificity | Specificity |
| CpG combinations | AUC | 80% | (%) | 90% | (%) | 95% | (%) |
| All 37 CpGs that are | 0.94 | 0.19 | 91.70 | 0.21 | 81.10 | 0.25 | 67.95 |
| identified in SEQ | | | | | | | |
| ID NOs 30 to 66 | | | | | | | |

### Breast Cancer Index Values as Diagnostic and Risk Assessment Tools

In any of the assays described herein, the assessment of the presence, absence or development of breast cancer in an individual is based on the breast cancer index value of the individual at the time of testing.

As explained herein, using panels of the specific CpGs disclosed herein, breast cancer index values can be established which correspond with breast cancer negative samples, because they are based on values derived from breast tissue samples from individuals known to be breast cancer negative. Similarly, using panels of the specific CpGs disclosed herein, breast cancer index values can be established which correspond with breast cancer positive samples, because they are based on values derived from breast cancer tissue samples from individuals known to be breast cancer positive. A user can then apply these breast cancer index values to assess the presence, absence or development of breast cancer in any test individual whose breast cancer status is required to be tested. As also explained herein, the assays of the invention are capable of being performed with a high degree of statistical accuracy.

A skilled person will readily appreciate that a breast cancer index value provides a value that indicates a “likelihood” or “risk” or “prediction” of any of the assays of the invention correctly assessing the presence, absence or development of breast cancer in an individual. This is because the assessment is based upon a correlation between DNA methylation profiles of tissue samples and individual disease status. Nevertheless, as demonstrated by data set out in the Examples and elsewhere herein, the assays of the invention provide such correlations with high statistical accuracy, thus providing the skilled person with a high degree of confidence that the breast cancer index value which is determined for any test individual whose breast cancer status is required to be tested will provide an accurate correlation with actual disease status for the individual.

In the context of the present invention, “likelihood”, “risk” and “prediction” may be used synonymously with each other.

Any references herein to sequences, genomic sequences and/or genomic coordinates are derived based upon Homo sapiens (human) genome assembly GRCh37 (hg19), The skilled person would understand variations in the nucleotide sequences of any given sequence, particularly DMRs 1 to 29, may exist due to sequencing errors and/or variation between individuals.

The assay of the invention represents a ‘prediction’ because any breast cancer index value (WID-Breast-Index) derived in accordance with the invention is unlikely to be capable of diagnosing every individual as having or not having breast cancer with 100% specificity and 100% sensitivity. Rather, depending on the breast cancer index cutpoint threshold applied by the user for positively predicting the presence of breast cancer in an individual, the false positive and false negative rate will vary. In other words, the inventors have discovered that the assays of the invention can achieve variable levels of sensitivity and specificity for predicting the presence, absence or development of breast cancer, as defined by receiver operating characteristics, depending on the breast cancer index cutpoint threshold chosen and applied by the user. Such sensitivity and specificity can be seen from the data disclosed herein to be achievable at high proportions, demonstrating accurate and statistically-significant discriminatory capability.

Similarly, breast cancer index values which have been pre-determined to correlate with specific breast cancer phenotypes, such as the presence or absence of breast cancer, have been defined with a high level of statistical accuracy as explained further herein.

Assessing the ‘development’ of breast cancer in the context of the invention may refer to assessing whether an individual is likely or unlikely to develop breast cancer. The inventors have shown that the CpGs assayed in order to derive the breast cancer index value of the assays of the invention are representative of the cells within normal breast tissue that eventually transform to breast cancer. In more detail, the breast cancer index value derived in accordance with the present invention has been show to progressively increase in samples from normal breast tissue in healthy women, to samples from normal tissue surrounding a breast cancer lesion, to samples from the tissue of a breast cancer lesion itself, to subsequently increasing further in samples from a triple negative breast cancer lesion through increasing stages of breast cancer lesion tissue. Thus, assessing the development of breast cancer in accordance with the assays of the invention may refer to assessing an increased or decreased likelihood of breast cancer development. Assessing of the development of breast cancer in accordance with the assays of the invention may refer to assessing progression or worsening of a pre-existing breast cancer lesion in an individual. Assessment of the development of breast cancer in accordance with the assays of the invention may refer to predicting the likelihood of recurrence of breast cancer.

In any of the assays described herein, the step of assessing the presence or development of breast cancer in an individual based on a breast cancer index value may involve the application of a threshold value. Threshold values can provide a risk-based indication of an individual's breast cancer status, whether that is breast cancer positive, or breast cancer negative. Threshold values can also provide a means for identifying whether the breast cancer index value is intermediate between a breast cancer positive value and a breast cancer negative value. As explained herein, the breast cancer index value may be dynamic and subject to change depending upon genetic and/or environmental factors. Accordingly, the breast cancer index value may provide a means for assessing and monitoring breast cancer development. Breast cancer index values may therefore indicate at least a low risk or a high risk that the individual has a breast cancer positive status or has a status that is indicative of the development of breast cancer. If the breast cancer index value of an individual is determined by the assays of the invention at two or more time points, an increase or decrease in the individual's breast cancer index value may indicate an increased or decreased risk of the individual having or developing breast cancer. In any assay described herein which specifies that a cancer index value for the individual is less than a specific value, or is less than “about” a specific value, the individual may be assessed as not having cancer. The term “about” is to be understood as providing a range of +/−5% of the value.

Accordingly, any assay of the invention is an assay for assessing the presence, absence or development of breast cancer in an individual, the assay comprising:
a. providing a sample which has been taken from the individual, the sample comprising a population of DNA molecules;
b. determining in the population of DNA molecules in the sample the methylation status of a panel of one or more CpGs selected from within a panel of one or more Differentially Methylated Regions (DMRs) defined by SEQ ID NOs 1 to 29, wherein selected CpGs in each DMR are denoted by CG;
c. deriving a breast cancer index value (WID-Breast-Index) based on the methylation status of the one or more CpGs in the panel; and
d. assessing the presence, absence or development of breast cancer in the individual based on the breast cancer index value.

Such an assay may be performed in accordance with any of the methods disclosed and defined herein.

As explained further herein, any assay of the invention for assessing the presence, absence or development of breast cancer in an individual may alternatively be referred to as an assay for stratifying an individual in accordance with their breast cancer status.

Accordingly, any assay of the invention may be defined as an assay for stratifying an individual for the presence, absence or development of breast cancer, the assay comprising:
a. providing a sample which has been taken from the individual, the sample comprising a population of DNA molecules;
b. determining in the population of DNA molecules in the sample the methylation status of a panel of one or more CpGs selected from within a panel of one or more Differentially Methylated Regions (DMRs) defined by SEQ ID NOs 1 to 29, wherein selected CpGs in each DMR are denoted by CG;
c. deriving a breast cancer index value (WID-Breast-Index) based on the methylation status of the one or more CpGs in the panel; and
d. stratifying the individual for the presence, absence or development of breast cancer based on the breast cancer index value.

Such an assay may be performed in accordance with any of the methods disclosed and defined herein.

Any assay of the invention may be defined as an assay for stratifying an individual for breast cancer, the assay comprising:
a. providing a sample which has been taken from the individual, the sample comprising a population of DNA molecules;
b. determining in the population of DNA molecules in the sample the methylation status of a panel of one or more CpGs selected from within a panel of one or more Differentially Methylated Regions (DMRs) defined by SEQ ID NOs 1 to 29, wherein selected CpGs in each DMR are denoted by CG;
c. deriving a breast cancer index value (WID-Breast-Index) based on the methylation status of the one or more CpGs in the panel; and
d. stratifying the individual for breast cancer based on the breast cancer index value.

Such an assay may be performed in accordance with any of the methods disclosed and defined herein.

In any of the below-described assays, the assay may be characterised as having a ROC AUC of 0.60 or more, 0.61 or more, 0.62 or more, 0.63 or more, 0.64 or more, 0.65 or more, 0.66 or more, 0.67 or more, 0.68 or more, 0.69 or more, 0.70 or more, 0.71 or more, 0.72 or more, 0.73 or more, 0.74 or more, 0.75 or more, 0.76 or more, 0.77 or more, 0.78 or more, 0.79 or more, 0.80 or more, 0.81 or more, 0.82 or more, 0.83 or more, 0.84 or more, 0.85 or more, 0.86 or more, 0.87 or more, 0.88 or more, 0.89 or more or 0.90 or more.

The predicting of the presence, absence or development of breast cancer in an individual may particularly involve threshold PMR value being applied in order to stratify an individual as having or not having breast cancer or of having a high or low risk of breast cancer development.

In any of the assays described herein, wherein:
a. when the breast cancer index for the individual is about 2.80 to 6.00 or more the individual is classified as having breast cancer or as having a high risk of breast cancer development; or
b. when the breast cancer index for the individual is less than about 2.80 the individual is classified as not having breast cancer or as having a low risk of breast cancer development;
   preferably wherein the assay has a specificity of 80% or more, more preferably wherein the assay comprises determining mean percent methylated reference values for each CpG in the panel of one or more CpGs.

In any of the assays described herein, wherein:
a. when the breast cancer index for the individual is about 3.50 to 7.00 or more the individual is classified as having breast cancer or as having a high risk of breast cancer development; or
b. when the breast cancer index for the individual is less than about 3.50 the individual is classified as not having breast cancer or as having a low risk of breast cancer development;
   preferably wherein the assay has a specificity of 90% or more, more preferably wherein the assay comprises determining mean percent methylated reference values for each CpG in the panel of one or more CpGs.

In any of the assays described herein, wherein:
a. when the breast cancer index for the individual is about 4.00 to 7.50 or more the individual is classified as having breast cancer or as having a high risk of breast cancer development; or
b. when the breast cancer index for the individual is less than about 4.00 the individual is classified as not having breast cancer or as having a low risk of breast cancer development;
   preferably wherein the assay has a specificity of 95% or more, more preferably wherein the assay comprises determining mean percent methylated reference values for each CpG in the panel of one or more CpGs.

In any of the assays described herein, wherein:
a. CpGs whose breast cancer index value is determined are located within four or more of the DMRs of SEQ ID NOs 1 to 29, and wherein when the breast cancer index for the individual is about 3.73 or more the individual is classified as having breast cancer or as having a high risk of breast cancer development, and wherein the sensitivity of the assay is at least 82.00% and the specificity of the assay is at least 80.00%, preferably wherein CpGs whose breast cancer index value is determined are located within the DMRs of SEQ ID NOs 2, 4, 7 and 14), and more preferably wherein the breast cancer index value is the mean percent methylated reference for each of the one or more within the DMRs of SEQ ID NOs 2, 4, 7 and 14;
b. CpGs whose breast cancer index value is determined are located within four or more of the DMRs of SEQ ID NOs 1 to 29, and wherein when the breast cancer index for the individual is less than about 3.73 the individual is classified as not having breast cancer or as having a low risk of breast cancer development, and wherein the sensitivity of the assay is at least 82.00% and the specificity of the assay is at least 80.00%, preferably wherein CpGs whose breast cancer index value is determined are located within the DMRs of SEQ ID NOs 2, 4, 7 and 14, and more preferably wherein the breast cancer index value is the mean percent methylated reference for each of the one or more within the DMRs of SEQ ID NOs 2, 4, 7 and 14;
c. CpGs whose breast cancer index value is determined are located within four or more of the DMRs of SEQ ID NOs 1 to 29, and wherein when the breast cancer index for the individual is about 4.50 or more the individual is classified as having breast cancer or as having a high risk of breast cancer development, and wherein the sensitivity of the assay is at least 66.00% and the specificity of the assay is at least 90.00%, preferably wherein CpGs whose breast cancer index value is determined are located within the DMRs of SEQ ID NOs 2, 4, 7 and 14, and more preferably wherein the breast cancer index value is the mean percent methylated reference for each of the one or more within the DMRs of SEQ ID NOs 2, 4, 7 and 14;
d. CpGs whose breast cancer index value is determined are located within four or more of the DMRs of SEQ ID NOs 1 to 29, and wherein when the breast cancer index for the individual is less than about 4.50 the individual is classified as not having breast cancer or as having a low risk of breast cancer development, and wherein the sensitivity of the assay is at least 66.00% and the specificity of the assay is at least 90.00%, preferably wherein CpGs whose breast cancer index reference value is determined are located within the DMRs of SEQ ID NOs 2, 4, 7 and 14, and more preferably wherein the breast cancer index value is the mean percent methylated reference for each of the one or more within the DMRs of SEQ ID NOs 2, 4, 7 and 14;
e. CpGs whose breast cancer index value is determined are located within four or more of the DMRs of SEQ ID NOs 1 to 29, and wherein when the breast cancer index for the individual is about 5.26 or more the individual is classified as having breast cancer or as having a high risk of breast cancer development, and wherein the sensitivity of the assay is at least 50.00% and the specificity of the assay is at least 95.00%, preferably wherein CpGs whose breast cancer index value is determined are located within the DMRs of SEQ ID NOs 2, 4, 7 and 14, and more preferably wherein the breast cancer index value is the mean percent methylated reference for each of the one or more within the DMRs of SEQ ID NOs 2, 4, 7 and 14;
f. CpGs whose breast cancer index value is determined are located within four or more of the DMRs of SEQ ID NOs 1 to 29, and wherein when the breast cancer index for the individual is less than about 5.26 the individual is classified as not having breast cancer or as having a low risk of breast cancer development, and wherein the sensitivity of the assay is at least 50.00% and the specificity of the assay is at least 95.00%, preferably wherein CpGs whose breast cancer index value is determined are located within the DMRs of SEQ ID NOs 2, 4, 7 and 14, and more preferably wherein the breast cancer index value is the mean percent methylated reference for each of the one or more within the DMRs of SEQ ID NOs 2, 4, 7 and 14;
g. CpGs whose breast cancer index value is determined are located within five or more of the DMRs of SEQ ID NOs 1 to 29, and wherein when the breast cancer index for the individual is about 3.24 or more the individual is classified as having breast cancer or as having a high risk of breast cancer development, and wherein the sensitivity of the assay is at least 79.00% and the specificity of the assay is at least 80.00%, preferably wherein CpGs whose breast cancer index value is determined are located within the DMRs of SEQ ID NOs 1, 2, 4, 7 and 14, and more preferably wherein the breast cancer index value is the mean percent methylated reference for each of the one or more within the DMRs of SEQ ID NOs 1, 2, 4, 7 and 14;
h. CpGs whose breast cancer index value is determined are located within five or more of the DMRs of SEQ ID NOs 1 to 29, and wherein when the breast cancer index for the individual is less than about 3.24 the individual is classified as not having breast cancer or as having a low risk of breast cancer development, and wherein the sensitivity of the assay is at least 79.00% and the specificity of the assay is at least 80.00%, preferably wherein CpGs whose breast cancer index value is determined are located within the DMRs of SEQ ID NOs 1, 2, 4, 7 and 14, and more preferably wherein the breast cancer index value is the mean percent methylated reference for each of the one or more within the DMRs of SEQ ID NOs 1, 2, 4, 7 and 14;
i. CpGs whose breast cancer index value is determined are located within five or more of the DMRs of SEQ ID NOs 1 to 29, and wherein when the breast cancer index for the individual is about 4.11 or more the individual is classified as having breast cancer or as having a high risk of breast cancer development, and wherein the sensitivity of the assay is at least 62.00% and the specificity of the assay is at least 90.00%, preferably wherein CpGs whose breast cancer index value is determined are located within the DMRs of SEQ ID NOs 1, 2, 4, 7 and 14, and more preferably wherein the breast cancer index value is the mean percent methylated reference for each of the one or more within the DMRs of SEQ ID NOs 1, 2, 4, 7 and 14;
j. CpGs whose breast cancer index value is determined are located within five or more of the DMRs of SEQ ID NOs 1 to 29, and wherein when breast cancer index for the individual is less than about 4.11 the individual is classified as not having breast cancer or as having a low risk of breast cancer development, and wherein the sensitivity of the assay is at least 62.00% and the specificity of the assay is at least 90.00%, preferably wherein CpGs whose breast cancer index value is determined are located within the DMRs of SEQ ID NOs 1, 2, 4, 7 and 14, and more preferably wherein the breast cancer index value is the mean percent methylated reference for each of the one or more within the DMRs of SEQ ID NOs 1, 2, 4, 7 and 14;
k. CpGs whose breast cancer index value is determined are located within five or more of the DMRs of SEQ ID NOs 1 to 29, and wherein when the breast cancer index for the individual is about 4.46 or more the individual is classified as having breast cancer or as having a high risk of breast cancer development, and wherein the sensitivity of the assay is at least 45.00% and the specificity of the assay is at least 95.00%, preferably wherein CpGs whose breast cancer index value is determined are located within the DMRs of SEQ ID NOs 1, 2, 4, 7 and 14, and more preferably wherein the breast cancer index value is the mean percent methylated reference for each of the one or more within the DMRs of SEQ ID NOs 1, 2, 4, 7 and 14;
l. CpGs whose breast cancer index value is determined are located within five or more of the DMRs of SEQ ID NOs 1 to 29, and wherein when the breast cancer index for the individual is less than about 4.46 the individual is classified as not having breast cancer or as having a low risk of breast cancer development, and wherein the sensitivity of the assay is at least 45.00% and the specificity of the assay is at least 95.00%, preferably wherein CpGs whose breast cancer index value is determined are located within the DMRs of SEQ ID NOs 1, 2, 4, 7 and 14, and more preferably wherein the breast cancer index value is the mean percent methylated reference for each of the one or more within the DMRs of SEQ ID NOs 1, 2, 4, 7 and 14;
m. CpGs whose breast cancer index value is determined are located within six or more of the DMRs of SEQ ID NOs 1 to 29, and wherein when the breast cancer index for the individual is about 3.09 or more the individual is classified as having breast cancer or as having a high risk of breast cancer development, and wherein the sensitivity of the assay is at least 81.00% and the specificity of the assay is at least 80.00%, preferably wherein CpGs whose breast cancer index value is determined are located within the DMRs of SEQ ID NOs 1, 2, 4, 7, 12 and 14, and more preferably wherein the breast cancer index value is the mean percent methylated reference for each of the one or more within the DMRs of SEQ ID NOs 1, 2, 4, 7, 12 and 14;
n. CpGs whose breast cancer index value is determined are located within six or more of the DMRs of SEQ ID NOs 1 to 29, and wherein when the breast cancer index for the individual is less than about 3.09 the individual is classified as not having breast cancer or as having a low risk of breast cancer development, and wherein the sensitivity of the assay is at least 81.00% and the specificity of the assay is at least 80.00%, preferably wherein CpGs whose breast cancer index value is determined are located within the DMRs of SEQ ID NOs 1, 2, 4, 7, 12 and 14, and more preferably wherein the breast cancer index value is the mean percent methylated reference for each of the one or more within the DMRs of SEQ ID NOs 1, 2, 4, 7, 12 and 14;
o. CpGs whose breast cancer index value is determined are located within six or more of the DMRs of SEQ ID NOs 1 to 29, and wherein when the breast cancer index for the individual is about 4.22 or more the individual is classified as having breast cancer or as having a high risk of breast cancer development, and wherein the sensitivity of the assay is at least 58.00% and the specificity of the assay is at least 90.00%, preferably wherein CpGs whose breast cancer index value is determined are located within the DMRs of SEQ ID NOs 1, 2, 4, 7, 12 and 14, and more preferably wherein the breast cancer index value is the mean percent methylated reference for each of the one or more within the DMRs of SEQ ID NOs 1, 2, 4, 7, 12 and 14;
p. CpGs whose breast cancer index value is determined are located within six or more of the DMRs of SEQ ID NOs 1 to 29, and wherein when the breast cancer index for the individual is less than about 4.22 the individual is classified as not having breast cancer or as having a low risk of breast cancer development, and wherein the sensitivity of the assay is at least 58.00% and the specificity of the assay is at least 90.00%, preferably wherein CpGs whose breast cancer index value is determined are located within the DMRs of SEQ ID NOs 1, 2, 4, 7, 12 and 14, and more preferably wherein the breast cancer index value is the mean percent methylated reference for each of the one or more within the DMRs of SEQ ID NOs 1, 2, 4, 7, 12 and 14;
q. CpGs whose breast cancer index value is determined are located within six or more of the DMRs of SEQ ID NOs 1 to 29, and wherein when the breast cancer index for the individual is about 4.39 or more the individual is classified as having breast cancer or as having a high risk of breast cancer development, and wherein the sensitivity of the assay is at least 35.00% and the specificity of the assay is at least 95.00%, preferably wherein CpGs whose breast cancer index value is determined are located within the DMRs of SEQ ID NOs 1, 2, 4, 7, 12 and 14, and more preferably wherein the breast cancer index value is the mean percent methylated reference for each of the one or more within the DMRs of SEQ ID NOs 1, 2, 4, 7, 12 and 14;
r. CpGs whose breast cancer index value is determined are located within six or more of the DMRs of SEQ ID NOs 1 to 29, and wherein when the breast cancer index for the individual is less than about 4.39 the individual is classified as not having breast cancer or as having a low risk of breast cancer development, and wherein the sensitivity of the assay is at least 35.00% and the specificity of the assay is at least 95.00%, preferably wherein CpGs whose breast cancer index value is determined are located within the DMRs of SEQ ID NOs 1, 2, 4, 7, 12 and 14, and more preferably wherein the breast cancer index value is the mean percent methylated reference for each of the one or more within the DMRs of SEQ ID NOs 1, 2, 4, 7, 12 and 14;
s. CpGs whose breast cancer index value is determined are located within seven or more of the DMRs of SEQ ID NOs 1 to 29, and wherein when the breast cancer index for the individual is about 2.84 or more the individual is classified as having breast cancer or as having a high risk of breast cancer development, and wherein the sensitivity of the assay is at least 82.00% and the specificity of the assay is at least 80.00%, preferably wherein CpGs whose breast cancer index value is determined are located within the DMRs of SEQ ID NOs 1, 2, 3, 4, 7, 12 and 14, and more preferably wherein the breast cancer index value is the mean percent methylated reference for each of the one or more within the DMRs of SEQ ID NOs 1, 2, 3, 4, 7, 12 and 14; t. CpGs whose breast cancer index value is determined are located within seven or more of the DMRs of SEQ ID NOs 1 to 29, and wherein when the breast cancer index for the individual is less than about 2.84 the individual is classified as not having breast cancer or as having a low risk of breast cancer development, and wherein the sensitivity of the assay is at least 82.00% and the specificity of the assay is at least 80.00%, preferably wherein CpGs whose breast cancer index reference value is determined are located within the DMRs of SEQ ID NOs 1, 2, 3, 4, 7, 12 and 14, and more preferably wherein the breast cancer index value is the mean percent methylated reference for each of the one or more within the DMRs of SEQ ID NOs 1, 2, 3, 4, 7, 12 and 14;
u. CpGs whose breast cancer index value is determined are located within seven or more of the DMRs of SEQ ID NOs 1 to 29, and wherein when the breast cancer index for the individual is about 3.62 or more the individual is classified as having breast cancer or as having a high risk of breast cancer development, and wherein the sensitivity of the assay is at least 66.00% and the specificity of the assay is at least 90.00%, preferably wherein CpGs whose breast cancer index value is determined are located within the DMRs of SEQ ID NOs 1, 2, 3, 4, 7, 12 and 14, and more preferably wherein the breast cancer index value is the mean percent methylated reference for each of the one or more within the DMRs of SEQ ID NOs 1, 2, 3, 4, 7, 12 and 14;
v. CpGs whose breast cancer index value is determined are located within seven or more of the DMRs of SEQ ID NOs 1 to 29, and wherein when the breast cancer index for the individual is less than about 3.62 the individual is classified as not having breast cancer or as having a low risk of breast cancer development, and wherein the sensitivity of the assay is at least 66.00% and the specificity of the assay is at least 90.00%, preferably wherein CpGs whose breast cancer index value is determined are located within the DMRs of SEQ ID NOs 1, 2, 3, 4, 7, 12 and 14, and more preferably wherein the breast cancer index value is the mean percent methylated reference for each of the one or more within the DMRs of SEQ ID NOs 1, 2, 3, 4, 7, 12 and 14;
w. CpGs whose breast cancer index value is determined are located within seven or more of the DMRs of SEQ ID NOs 1 to 29, and wherein when the breast cancer index for the individual is about 4.11 or more the individual is classified as having breast cancer or as having a high risk of breast cancer development, and wherein the sensitivity of the assay is at least 50.00% and the specificity of the assay is at least 95.00%, preferably wherein CpGs whose breast cancer index value is determined are located within the DMRs of SEQ ID NOs 1, 2, 3, 4, 7, 12 and 14, and more preferably wherein the breast cancer index value is the mean percent methylated reference for each of the one or more within the DMRs of SEQ ID NOs 1, 2, 3, 4, 7, 12 and 14;
x. CpGs whose breast cancer index value is determined are located within seven or more of the DMRs of SEQ ID NOs 1 to 29, and wherein when the breast cancer index for the individual is less than about 4.11 the individual is classified as not having breast cancer or as having a low risk of breast cancer development, and wherein the sensitivity of the assay is at least 50.00% and the specificity of the assay is at least 95.00%, preferably wherein CpGs whose breast cancer index value is determined are located within the DMRs of SEQ ID NOs 1, 2, 3, 4, 7, 12 and 14, and more preferably wherein the breast cancer index value is the mean percent methylated reference for each of the one or more within the DMRs of SEQ ID NOs 1, 2, 3, 4, 7, 12 and 14;
y. CpGs whose breast cancer index value is determined are located within eight or more of the DMRs of SEQ ID NOs 1 to 29, and wherein when the breast cancer index for the individual is about 2.85 or more the individual is classified as having breast cancer or as having a high risk of breast cancer development, and wherein the sensitivity of the assay is at least 79.00% and the specificity of the assay is at least 80.00%, preferably wherein CpGs whose breast cancer index value is determined are located within the DMRs of SEQ ID NOs 1, 2, 3, 4, 7, 13, 14 and 15, and more preferably wherein the breast cancer index value is the mean percent methylated reference for each of the one or more within the DMRs of SEQ ID NOs 1, 2, 3, 4, 7, 13, 14 and 15;
z. CpGs whose breast cancer index value is determined are located within eight or more of the DMRs of SEQ ID NOs 1 to 29, and wherein when the breast cancer index for the individual is less than about 2.85 the individual is classified as not having breast cancer or as having a low risk of breast cancer development, and wherein the sensitivity of the assay is at least 79.00% and the specificity of the assay is at least 80.00%, preferably wherein CpGs whose breast cancer index value is determined are located within the DMRs of SEQ ID NOs 1, 2, 3, 4, 7, 13, 14 and 15, and more preferably wherein the breast cancer index value is the mean percent methylated reference for each of the one or more within the DMRs of SEQ ID NOs 1, 2, 3, 4, 7, 13, 14 and 15;
aa. CpGs whose breast cancer index value is determined are located within eight or more of the DMRs of SEQ ID NOs 1 to 29, and wherein when the breast cancer index for the individual is about 3.93 or more the individual is classified as having breast cancer or as having a high risk of breast cancer development, and wherein the sensitivity of the assay is at least 34.00% and the specificity of the assay is at least 90.00%, preferably wherein CpGs whose breast cancer index value is determined are located within the DMRs of SEQ ID NOs 1, 2, 3, 4, 7, 13, 14 and 15, and more preferably wherein the breast cancer index value is the mean percent methylated reference for each of the one or more within the DMRs of SEQ ID NOs 1, 2, 3, 4, 7, 13, 14 and 15;
bb. CpGs whose breast cancer index value is determined are located within eight or more of the DMRs of SEQ ID NOs 1 to 29, and wherein when the breast cancer index for the individual is less than about 3.93 the individual is classified as not having breast cancer or as having a low risk of breast cancer development, and wherein the sensitivity of the assay is at least 34.00% and the specificity of the assay is at least 90.00%, preferably wherein CpGs whose breast cancer index value is determined are located within the DMRs of SEQ ID NOs 1, 2, 3, 4, 7, 13, 14 and 15, and more preferably wherein the breast cancer index value is the mean percent methylated reference for each of the one or more within the DMRs of SEQ ID NOs 1, 2, 3, 4, 7, 13, 14 and 15;
cc. CpGs whose breast cancer index value is determined are located within eight or more of the DMRs of SEQ ID NOs 1 to 29, and wherein when the breast cancer index for the individual is about 4.02 or more the individual is classified as having a high risk of having breast cancer or a high risk of breast cancer development, and wherein the sensitivity is at least 8.00% and the specificity is at least 95.00%, preferably wherein CpGs whose breast cancer index value is determined are located within the DMRs of SEQ ID NOs 1, 2, 3, 4, 7, 13, 14 and 15, and more preferably wherein the breast cancer index value is the mean percent methylated reference for each of the one or more within the DMRs of SEQ ID NOs 1, 2, 3, 4, 7, 13, 14 and 15;
dd. CpGs whose breast cancer index value is determined are located within eight or more of the DMRs of SEQ ID NOs 1 to 29, and wherein when the breast cancer index for the individual is less than about 4.02 the individual is classified as not having breast cancer or as having a low risk of breast cancer development, and wherein the sensitivity is at least 8.00% and the specificity is at least 95.00%, preferably wherein CpGs whose breast cancer index value is determined are located within the DMRs of SEQ ID NOs 1, 2, 3, 4, 7, 13, 14 and 15, and more preferably wherein the breast cancer index value is the mean percent methylated reference for each of the one or more within the DMRs of SEQ ID NOs 1, 2, 3, 4, 7, 13, 14 and 15;
ee. CpGs whose breast cancer index value is determined are located within nine or more of the DMRs of SEQ ID NOs 1 to 29, and wherein when the breast cancer index for the individual is about 4.01 or more the individual is classified as having breast cancer or as having a high risk of breast cancer development, and wherein the sensitivity of the assay is at least 76.00% and the specificity of the assay is at least 80.00%, preferably wherein CpGs whose breast cancer index value is determined are located within the DMRs of SEQ ID NOs 1, 2, 4, 6, 7, 13, 14, 15 and 16, and more preferably wherein the breast cancer index value is the mean percent methylated reference for each of the one or more within the DMRs of SEQ ID NOs 1, 2, 4, 6, 7, 13, 14, 15 and 16;
ff. CpGs whose breast cancer index value is determined are located within nine or more of the DMRs of SEQ ID NOs 1 to 29, and wherein when the breast cancer index for the individual is less than about 4.01 the individual is classified as not having breast cancer or as having a low risk of breast cancer development, and wherein the sensitivity of the assay is at least 76.00% and the specificity of the assay is at least 80.00%, preferably wherein CpGs whose breast cancer index value is determined are located within the DMRs of SEQ ID NOs 1, 2, 4, 6, 7, 13, 14, 15 and 16, and more preferably wherein the breast cancer index value is the mean percent methylated reference for each of the one or more within the DMRs of SEQ ID NOs 1, 2, 4, 6, 7, 13, 14, 15 and 16;
gg. CpGs whose breast cancer index value is determined are located within nine or more of the DMRs of SEQ ID NOs 1 to 29, and wherein when the breast cancer index for the individual is about 5.12 or more the individual is classified as having breast cancer or as having a high risk of breast cancer development, and wherein the sensitivity of the assay is at least 48.00% and the specificity of the assay is at least 90.00%, preferably wherein CpGs whose breast cancer index value is determined are located within the DMRs of SEQ ID NOs 1, 2, 4, 6, 7, 13, 14, 15 and 16, and more preferably wherein the breast cancer index value is the mean percent methylated reference for each of the one or more within the DMRs of SEQ ID NOs 1, 2, 4, 6, 7, 13, 14, 15 and 16; or
hh. CpGs whose breast cancer index value is determined are located within nine or more of the DMRs of SEQ ID NOs 1 to 29, and wherein when the breast cancer index for the individual is less than about 5.12 the individual is classified as not having breast cancer or as having a low risk of breast cancer development, and wherein the sensitivity of the assay is at least 48.00% and the specificity of the assay is at least 90.00%, preferably wherein CpGs whose breast cancer index value is determined are located within the DMRs of SEQ ID NOs 1, 2, 4, 6, 7, 13, 14, 15 and 16, and more preferably wherein the breast cancer index value is the mean percent methylated reference for each of the one or more within the DMRs of SEQ ID NOs 1, 2, 4, 6, 7, 13, 14, 15 and 16; or
ii. CpGs whose breast cancer index value is determined are located within nine or more of the DMRs of SEQ ID NOs 1 to 29, and wherein when the breast cancer index for the individual is about 5.28 or more the individual is classified as having breast cancer or as having a high risk of breast cancer development, and wherein the sensitivity of the assay is at least 25.00% and the specificity of the assay is at least 95.00%, preferably wherein CpGs whose breast cancer index value is determined are located within the DMRs of SEQ ID NOs 1, 2, 4, 6, 7, 13, 14, 15 and 16, and more preferably wherein the breast cancer index value is the mean percent methylated reference for each of the one or more within the DMRs of SEQ ID NOs 1, 2, 4, 6, 7, 13, 14, 15 and 16; or
jj. CpGs whose breast cancer index value is determined are located within nine or more of the DMRs of SEQ ID NOs 1 to 29, and wherein when the breast cancer index for the individual is less than about 5.28 the individual is classified as not having breast cancer or as having a low risk of breast cancer development, and wherein the sensitivity of the assay is at least 25.00% and the specificity of the assay is at least 95.00%, preferably wherein CpGs whose breast cancer index value is determined are located within the DMRs of SEQ ID NOs 1, 2, 4, 6, 7, 13, 14, 15 and 16, and more preferably wherein the breast cancer index value is the mean percent methylated reference for each of the one or more within the DMRs of SEQ ID NOs 1, 2, 4, 6, 7, 13, 14, 15 and 16.

Even more preferably, in any of the assays described herein wherein the breast cancer index value of an individual is determined by:
a. CpGs whose breast cancer index value is determined are located within five or more of the DMRs of SEQ ID NOs 1 to 29, and wherein when the breast cancer index for the individual is about 3.24 or more the individual is classified as having breast cancer or as having a high risk of breast cancer development, and wherein the sensitivity of the assay is at least 79.00% and the specificity of the assay is at least 80.00%, preferably wherein CpGs whose breast cancer index value is determined are located within the DMRs of SEQ ID NOs 4, 6, 7, 14 and 15, and more preferably wherein the breast cancer index value is the mean percent methylated reference for each of the one or more within the DMRs of SEQ ID NOs 4, 6, 7, 14 and 15;
b. CpGs whose breast cancer index value is determined are located within five or more of the DMRs of SEQ ID NOs 1 to 29, and wherein when the breast cancer index for the individual is less than about 3.24 the individual is classified as not having breast cancer or as having a low risk of breast cancer development, and wherein the sensitivity of the assay is at least 79.00% and the specificity of the assay is at least 80.00%, preferably wherein CpGs whose breast cancer index value is determined are located within the DMRs of SEQ ID NOs 4, 6, 7, 14 and 15, and more preferably wherein the breast cancer index value is the mean percent methylated reference for each of the one or more within the DMRs of SEQ ID NOs 4, 6, 7, 14 and 15;
c. CpGs whose breast cancer index value is determined are located within five or more of the DMRs of SEQ ID NOs 1 to 29, and wherein when the breast cancer index for the individual is about 4.11 or more the individual is classified as having breast cancer or as having a high risk of breast cancer development, and wherein the sensitivity of the assay is at least 62.00% and the specificity of the assay is at least 90.00%, preferably wherein CpGs whose breast cancer index value is determined are located within the DMRs of SEQ ID NOs 4, 6, 7, 14 and 15, and more preferably wherein the breast cancer index value is the mean percent methylated reference for each of the one or more within the DMRs of SEQ ID NOs 4, 6, 7, 14 and 15;
d. CpGs whose breast cancer index value is determined are located within five or more of the DMRs of SEQ ID NOs 1 to 29, and wherein when breast cancer index for the individual is less than about 4.11 the individual is classified as not having breast cancer or as having a low risk of breast cancer development, and wherein the sensitivity of the assay is at least 62.00% and the specificity of the assay is at least 90.00%, preferably wherein CpGs whose breast cancer index value is determined are located within the DMRs of SEQ ID NOs 4, 6, 7, 14 and 15, and more preferably wherein the breast cancer index value is the mean percent methylated reference for each of the one or more within the DMRs of SEQ ID NOs 4, 6, 7, 14 and 15;
e. CpGs whose breast cancer index value is determined are located within five or more of the DMRs of SEQ ID NOs 1 to 29, and wherein when the breast cancer index for the individual is about 4.46 or more the individual is classified as having breast cancer or as having a high risk of breast cancer development, and wherein the sensitivity of the assay is at least 45.00% and the specificity of the assay is at least 95.00%, preferably wherein CpGs whose breast cancer index value is determined are located within the DMRs of SEQ ID NOs 4, 6, 7, 14 and 15, and more preferably wherein the breast cancer index value is the mean percent methylated reference for each of the one or more within the DMRs of SEQ ID NOs 4, 6, 7, 14 and 15;
f. CpGs whose breast cancer index value is determined are located within five or more of the DMRs of SEQ ID NOs 1 to 29, and wherein when the breast cancer index for the individual is less than about 4.46 the individual is classified as not having breast cancer or as having a low risk of breast cancer development, and wherein the sensitivity of the assay is at least 45.00% and the specificity of the assay is at least 95.00%, preferably wherein CpGs whose breast cancer index value is determined are located within the DMRs of SEQ ID NOs 4, 6, 7, 14 and 15, and more preferably wherein the breast cancer index value is the mean percent methylated reference for each of the one or more within the DMRs of SEQ ID NOs 4, 6, 7, 14 and 15.

In any of the assays described and defined herein, the assay may involve a step of determining in the population of DNA molecules in the sample the methylation status of a panel of one, more than one or all of the CpGs within a DMR panel comprising a combination of Differentially Methylated Regions (DMRs), preferably wherein the assay exhibits an ROC AUC of 0.9 or more, and wherein selected CpGs in each DMR are denoted by CG, and wherein the DMR panel comprises:
1 DMR #1 (SEQ ID NO: 1); DMR #3 (SEQ ID NO: 3); DMR #2 (SEQ ID NO: 2); DMR #4 (SEQ ID NO: 4); DMR #6 (SEQ ID NO: 6); DMR #7 (SEQ ID NO: 7); DMR #15 (SEQ ID NO: 15); and DMR #17 (SEQ ID NO: 17); optionally wherein when the breast cancer index for the individual is about 4.82 or more the individual is classified as having breast cancer or as having a high risk of breast cancer development, or wherein when the breast cancer index for the individual is less than about 4.82 the individual is classified as not having breast cancer or as having a low risk of breast cancer development, and preferably wherein the sensitivity of the assay is at least 78.50% and the specificity of the assay is at least 80.00%; and/or
2 DMR #1 (SEQ ID NO: 1); DMR #3 (SEQ ID NO: 3); DMR #4 (SEQ ID NO: 4); DMR #6 (SEQ ID NO: 6); DMR #7 (SEQ ID NO: 7); DMR #14 (SEQ ID NO: 14); DMR #15 (SEQ ID NO: 15); and DMR #17 (SEQ ID NO: 17); optionally wherein when the breast cancer index for the individual is about 4.80 or more the individual is classified as having breast cancer or as having a high risk of breast cancer development, or wherein when the breast cancer index for the individual is less than about 4.80 the individual is classified as not having breast cancer or as having a low risk of breast cancer development, and preferably wherein the sensitivity of the assay is at least 78.00% and the specificity of the assay is at least 80.00%; and/or
3 DMR #1 (SEQ ID NO: 1); DMR #2 (SEQ ID NO: 2); DMR #4 (SEQ ID NO: 4); DMR #6 (SEQ ID NO: 6); DMR #7 (SEQ ID NO: 7); DMR #14 (SEQ ID NO: 14); DMR #15 (SEQ ID NO: 15); and DMR #17 (SEQ ID NO: 17); optionally wherein when the breast cancer index for the individual is about 5.06 or more the individual is classified as having breast cancer or as having a high risk of breast cancer development, or wherein when the breast cancer index for the individual is less than about 4.80 the individual is classified as not having breast cancer or as having a low risk of breast cancer development, and preferably wherein the sensitivity of the assay is at least 76.50% and the specificity of the assay is at least 80.00%; and/or
4 DMR #1 (SEQ ID NO: 1); DMR #3 (SEQ ID NO: 3); DMR #2 (SEQ ID NO: 2); DMR #4 (SEQ ID NO: 4); DMR #6 (SEQ ID NO: 6); DMR #7 (SEQ ID NO: 7); DMR #14 (SEQ ID NO: 14); DMR #15 (SEQ ID NO: 15); and DMR #17 (SEQ ID NO: 17); optionally wherein when the breast cancer index for the individual is about 4.41 or more the individual is classified as having breast cancer or as having a high risk of breast cancer development, or wherein when the breast cancer index for the individual is less than about 4.41 the individual is classified as not having breast cancer or as having a low risk of breast cancer development, and preferably wherein the sensitivity of the assay is at least 78.50% and the specificity of the assay is at least 80.00%; and/or
5 DMR #1 (SEQ ID NO: 1); DMR #3 (SEQ ID NO: 3); DMR #4 (SEQ ID NO: 4); DMR #6 (SEQ ID NO: 6); DMR #7 (SEQ ID NO: 7); DMR #13 (SEQ ID NO: 13); DMR #14 (SEQ ID NO: 14); DMR #15 (SEQ ID NO: 15); and DMR #17 (SEQ ID NO: 17); optionally wherein when the breast cancer index for the individual is about 4.41 or more the individual is classified as having breast cancer or as having a high risk of breast cancer development, or wherein when the breast cancer index for the individual is less than about 4.41 the individual is classified as not having breast cancer or as having a low risk of breast cancer development, and preferably wherein the sensitivity of the assay is at least 78.50% and the specificity of the assay is at least 80.00%; and/or
6 DMR #3 (SEQ ID NO: 3); DMR #4 (SEQ ID NO: 4); DMR #6 (SEQ ID NO: 6); DMR #7 (SEQ ID NO: 7); DMR #15 (SEQ ID NO: 15); and DMR #17 (SEQ ID NO: 17); optionally wherein when the breast cancer index for the individual is about 6.05 or more the individual is classified as having breast cancer or as having a high risk of breast cancer development, or wherein when the breast cancer index for the individual is less than about 6.05 the individual is classified as not having breast cancer or as having a low risk of breast cancer development, and preferably wherein the sensitivity of the assay is at least 78.50% and the specificity of the assay is at least 80.00%; and/or
7 DMR #1 (SEQ ID NO: 1); DMR #3 (SEQ ID NO: 3); DMR #4 (SEQ ID NO: 4); DMR #6 (SEQ ID NO: 6); DMR #7 (SEQ ID NO: 7); DMR #15 (SEQ ID NO: 15); and DMR #17 (SEQ ID NO: 17); optionally wherein when the breast cancer index for the individual is about 5.32 or more the individual is classified as having breast cancer or as having a high risk of breast cancer development, or wherein when the breast cancer index for the individual is less than about 5.32 the individual is classified as not having breast cancer or as having a low risk of breast cancer development, and preferably wherein the sensitivity of the assay is at least 78.50% and the specificity of the assay is at least 80.00%; and/or
8 DMR #1 (SEQ ID NO: 1); DMR #2 (SEQ ID NO: 2); DMR #4 (SEQ ID NO: 4); DMR #6 (SEQ ID NO: 6); DMR #7 (SEQ ID NO: 7); DMR #15 (SEQ ID NO: 15); and DMR #17 (SEQ ID NO: 17); optionally wherein when the breast cancer index for the individual is about 5.60 or more the individual is classified as having breast cancer or as having a high risk of breast cancer development, or wherein when the breast cancer index for the individual is less than about 5.60 the individual is classified as not having breast cancer or as having a low risk of breast cancer development, and preferably wherein the sensitivity of the assay is at least 77.00% and the specificity of the assay is at least 80.00%; and/or
9 DMR #3 (SEQ ID NO: 3); DMR #2 (SEQ ID NO: 2); DMR #4 (SEQ ID NO: 4); DMR #6 (SEQ ID NO: 6); DMR #7 (SEQ ID NO: 7); DMR #15 (SEQ ID NO: 15); and DMR #17 (SEQ ID NO: 17); optionally wherein when the breast cancer index for the individual is about 5.36 or more the individual is classified as having breast cancer or as having a high risk of breast cancer development, or wherein when the breast cancer index for the individual is less than about 5.36 the individual is classified as not having breast cancer or as having a low risk of breast cancer development, and preferably wherein the sensitivity of the assay is at least 76.50% and the specificity of the assay is at least 80.00%; and/or
10 DMR #1 (SEQ ID NO: 1); DMR #2 (SEQ ID NO: 2); DMR #4 (SEQ ID NO: 4); DMR #6 (SEQ ID NO: 6); DMR #7 (SEQ ID NO: 7); DMR #14 (SEQ ID NO: 14); DMR #15 (SEQ ID NO: 15); and DMR #16 (SEQ ID NO: 16); optionally wherein when the breast cancer index for the individual is about 4.38 or more the individual is classified as having breast cancer or as having a high risk of breast cancer development, or wherein when the breast cancer index for the individual is less than about 4.38 the individual is classified as not having breast cancer or as having a low risk of breast cancer development, and preferably wherein the sensitivity of the assay is at least 76.50% and the specificity of the assay is at least 80.00%; and/or
11 DMR #1 (SEQ ID NO: 1); DMR #2 (SEQ ID NO: 2); DMR #4 (SEQ ID NO: 4); DMR #6 (SEQ ID NO: 6); DMR #7 (SEQ ID NO: 7); DMR #13 (SEQ ID NO: 13); DMR #14 (SEQ ID NO: 14); DMR #15 (SEQ ID NO: 15); and DMR #16 (SEQ ID NO: 16); optionally wherein when the breast cancer index for the individual is about 4.01 or more the individual is classified as having breast cancer or as having a high risk of breast cancer development, or wherein when the breast cancer index for the individual is less than about 4.01 the individual is classified as not having breast cancer or as having a low risk of breast cancer development, and preferably wherein the sensitivity of the assay is at least 76.50% and the specificity of the assay is at least 80.00%; and/or
12 DMR #1 (SEQ ID NO: 1); DMR #2 (SEQ ID NO: 2); DMR #4 (SEQ ID NO: 4); DMR #6 (SEQ ID NO: 6); DMR #7 (SEQ ID NO: 7); DMR #14 (SEQ ID NO: 14); DMR #15 (SEQ ID NO: 15); DMR #16 (SEQ ID NO: 16); and DMR #18 (SEQ ID NO: 18); optionally wherein when the breast cancer index for the individual is about 4.18 or more the individual is classified as having breast cancer or as having a high risk of breast cancer development, or wherein when the breast cancer index for the individual is less than about 4.18 the individual is classified as not having breast cancer or as having a low risk of breast cancer development, and preferably wherein the sensitivity of the assay is at least 76.50% and the specificity of the assay is at least 80.00%; and/or
13 DMR #4 (SEQ ID NO: 4); DMR #6 (SEQ ID NO: 6); DMR #7 (SEQ ID NO: 7); DMR #14 (SEQ ID NO: 14); and DMR #15 (SEQ ID NO: 15); optionally wherein when the breast cancer index for the individual is about 5.87 or more the individual is classified as having breast cancer or as having a high risk of breast cancer development, or wherein when the breast cancer index for the individual is less than about 5.87 the individual is classified as not having breast cancer or as having a low risk of breast cancer development, and preferably wherein the sensitivity of the assay is at least 77.00% and the specificity of the assay is at least 80.00%; and/or
14 DMR #1 (SEQ ID NO: 1); DMR #3 (SEQ ID NO: 3); DMR #2 (SEQ ID NO: 2); DMR #4 (SEQ ID NO: 4); DMR #7 (SEQ ID NO: 7); DMR #13 (SEQ ID NO: 13); DMR #14 (SEQ ID NO: 14); and DMR #15 (SEQ ID NO: 15); optionally wherein when the breast cancer index for the individual is about 2.85 or more the individual is classified as having breast cancer or as having a high risk of breast cancer development, or wherein when the breast cancer index for the individual is less than about 2.85 the individual is classified as not having breast cancer or as having a low risk of breast cancer development, and preferably wherein the sensitivity of the assay is at least 79.50% and the specificity of the assay is at least 80.00%; and/or 15 DMR #1 (SEQ ID NO: 1); DMR #3 (SEQ ID NO: 3); DMR #4 (SEQ ID NO: 4); DMR #7 (SEQ ID NO: 7); DMR #14 (SEQ ID NO: 14); and DMR #15 (SEQ ID NO: 15); optionally wherein when the breast cancer index for the individual is about 3.48 or more the individual is classified as having breast cancer or as having a high risk of breast cancer development, or wherein when the breast cancer index for the individual is less than about 3.48 the individual is classified as not having breast cancer or as having a low risk of breast cancer development, and preferably wherein the sensitivity of the assay is at least 80.50% and the specificity of the assay is at least 80.00%; and/or
16 DMR #1 (SEQ ID NO: 1); DMR #2 (SEQ ID NO: 2); DMR #4 (SEQ ID NO: 4); DMR #6 (SEQ ID NO: 6); DMR #7 (SEQ ID NO: 7); DMR #12 (SEQ ID NO: 12); and DMR #15 (SEQ ID NO: 15); optionally wherein when the breast cancer index for the individual is about 4.71 or more the individual is classified as having breast cancer or as having a high risk of breast cancer development, or wherein when the breast cancer index for the individual is less than about 4.71 the individual is classified as not having breast cancer or as having a low risk of breast cancer development, and preferably wherein the sensitivity of the assay is at least 77.00% and the specificity of the assay is at least 80.00%; and/or
17 DMR #1 (SEQ ID NO: 1); DMR #3 (SEQ ID NO: 3); DMR #2 (SEQ ID NO: 2); DMR #4 (SEQ ID NO: 4); DMR #7 (SEQ ID NO: 7); DMR #14 (SEQ ID NO: 14); DMR #15 (SEQ ID NO: 15); and DMR #16 (SEQ ID NO: 16); optionally wherein when the breast cancer index for the individual is about 3.16 or more the individual is classified as having breast cancer or as having a high risk of breast cancer development, or wherein when the breast cancer index for the individual is less than about 3.16 the individual is classified as not having breast cancer or as having a low risk of breast cancer development, and preferably wherein the sensitivity of the assay is at least 79.50% and the specificity of the assay is at least 80.00%; and/or
18 DMR #1 (SEQ ID NO: 1); DMR #3 (SEQ ID NO: 3); DMR #4 (SEQ ID NO: 4); DMR #6 (SEQ ID NO: 6); DMR #7 (SEQ ID NO: 7); DMR #12 (SEQ ID NO: 12); and DMR #15 (SEQ ID NO: 15); optionally wherein when the breast cancer index for the individual is about 4.78 or more the individual is classified as having breast cancer or as having a high risk of breast cancer development, or wherein when the breast cancer index for the individual is less than about 4.78 the individual is classified as not having breast cancer or as having a low risk of breast cancer development, and preferably wherein the sensitivity of the assay is at least 77.00% and the specificity of the assay is at least 80.00%; and/or
19 DMR #1 (SEQ ID NO: 1); DMR #3 (SEQ ID NO: 3); DMR #2 (SEQ ID NO: 2); DMR #4 (SEQ ID NO: 4); DMR #7 (SEQ ID NO: 7); DMR #14 (SEQ ID NO: 14); and DMR #15 (SEQ ID NO: 15); optionally wherein when the breast cancer index for the individual is about 3.15 or more the individual is classified as having breast cancer or as having a high risk of breast cancer development, or wherein when the breast cancer index for the individual is less than about 3.15 the individual is classified as not having breast cancer or as having a low risk of breast cancer development, and preferably wherein the sensitivity of the assay is at least 79.50% and the specificity of the assay is at least 80.00%; and/or
20 DMR #1 (SEQ ID NO: 1); DMR #4 (SEQ ID NO: 4); DMR #7 (SEQ ID NO: 7); DMR #12 (SEQ ID NO: 12); and DMR #14 (SEQ ID NO: 14); optionally wherein when the breast cancer index for the individual is about 3.51 or more the individual is classified as having breast cancer or as having a high risk of breast cancer development, or wherein when the breast cancer index for the individual is less than about 3.51 the individual is classified as not having breast cancer or as having a low risk of breast cancer development, and preferably wherein the sensitivity of the assay is at least 82.00% and the specificity of the assay is at least 80.00%; and/or
21 DMR #4 (SEQ ID NO: 4); DMR #5 (SEQ ID NO: 5); DMR #6 (SEQ ID NO: 6); DMR #7 (SEQ ID NO: 7); DMR #12 (SEQ ID NO: 12); and DMR #14 (SEQ ID NO: 14); optionally wherein when the breast cancer index for the individual is about 5.07 or more the individual is classified as having breast cancer or as having a high risk of breast cancer development, or wherein when the breast cancer index for the individual is less than about 5.07 the individual is classified as not having breast cancer or as having a low risk of breast cancer development, and preferably wherein the sensitivity of the assay is at least 79.50% and the specificity of the assay is at least 80.00%; and/or
22 DMR #1 (SEQ ID NO: 1); DMR #2 (SEQ ID NO: 2); DMR #4 (SEQ ID NO: 4); DMR #7 (SEQ ID NO: 7); DMR #14 (SEQ ID NO: 14); and DMR #15 (SEQ ID NO: 15); optionally wherein when the breast cancer index for the individual is about 3.44 or more the individual is classified as having breast cancer or as having a high risk of breast cancer development, or wherein when the breast cancer index for the individual is less than about 3.44 the individual is classified as not having breast cancer or as having a low risk of breast cancer development, and preferably wherein the sensitivity of the assay is at least 79.50% and the specificity of the assay is at least 80.00%; and/or
23 DMR #1 (SEQ ID NO: 1); DMR #2 (SEQ ID NO: 2); DMR #4 (SEQ ID NO: 4); DMR #7 (SEQ ID NO: 7); DMR #12 (SEQ ID NO: 12); and DMR #14 (SEQ ID NO: 14); optionally wherein when the breast cancer index for the individual is about 3.09 or more the individual is classified as having breast cancer or as having a high risk of breast cancer development, or wherein when the breast cancer index for the individual is less than about 3.09 the individual is classified as not having breast cancer or as having a low risk of breast cancer development, and preferably wherein the sensitivity of the assay is at least 82.00% and the specificity of the assay is at least 80.00%; and/or
24 DMR #4 (SEQ ID NO: 4); DMR #7 (SEQ ID NO: 7); DMR #14 (SEQ ID NO: 14); and DMR #15 (SEQ ID NO: 15); optionally wherein when the breast cancer index for the individual is about 4.57 or more the individual is classified as having breast cancer or as having a high risk of breast cancer development, or wherein when the breast cancer index for the individual is less than about 4.57 the individual is classified as not having breast cancer or as having a low risk of breast cancer development, and preferably wherein the sensitivity of the assay is at least 81.00% and the specificity of the assay is at least 80.00%; and/or
25 DMR #2 (SEQ ID NO: 2); DMR #4 (SEQ ID NO: 4); DMR #7 (SEQ ID NO: 7); DMR #14 (SEQ ID NO: 14); and DMR #15 (SEQ ID NO: 15); optionally wherein when the breast cancer index for the individual is about 3.91 or more the individual is classified as having breast cancer or as having a high risk of breast cancer development, or wherein when the breast cancer index for the individual is less than about 3.91 the individual is classified as not having breast cancer or as having a low risk of breast cancer development, and preferably wherein the sensitivity of the assay is at least 79.50% and the specificity of the assay is at least 80.00%; and/or
26 DMR #1 (SEQ ID NO: 1); DMR #3 (SEQ ID NO: 3); DMR #2 (SEQ ID NO: 2); DMR #4 (SEQ ID NO: 4); DMR #7 (SEQ ID NO: 7); DMR #12 (SEQ ID NO: 12); and DMR #14 (SEQ ID NO: 14); optionally wherein when the breast cancer index for the individual is about 2.84 or more the individual is classified as having breast cancer or as having a high risk of breast cancer development, or wherein when the breast cancer index for the individual is less than about 2.84 the individual is classified as not having breast cancer or as having a low risk of breast cancer development, and preferably wherein the sensitivity of the assay is at least 82.50% and the specificity of the assay is at least 80.00%; and/or
27 DMR #1 (SEQ ID NO: 1); DMR #3 (SEQ ID NO: 3); DMR #4 (SEQ ID NO: 4); DMR #7 (SEQ ID NO: 7); DMR #12 (SEQ ID NO: 12); and DMR #14 (SEQ ID NO: 14); optionally wherein when the breast cancer index for the individual is about 3.14 or more the individual is classified as having breast cancer or as having a high risk of breast cancer development, or wherein when the breast cancer index for the individual is less than about 3.14 the individual is classified as not having breast cancer or as having a low risk of breast cancer development, and preferably wherein the sensitivity of the assay is at least 79.50% and the specificity of the assay is at least 80.00%; and/or
28 DMR #2 (SEQ ID NO: 2); DMR #4 (SEQ ID NO: 4); DMR #7 (SEQ ID NO: 7); and DMR #14 (SEQ ID NO: 14); optionally wherein when the breast cancer index for the individual is about 3.73 or more the individual is classified as having breast cancer or as having a high risk of breast cancer development, or wherein when the breast cancer index for the individual is less than about 3.73 the individual is classified as not having breast cancer or as having a low risk of breast cancer development, and preferably wherein the sensitivity of the assay is at least 82.50% and the specificity of the assay is at least 80.00%; and/or
29 DMR #1 (SEQ ID NO: 1); DMR #2 (SEQ ID NO: 2); DMR #4 (SEQ ID NO: 4); DMR #7 (SEQ ID NO: 7); DMR #12 (SEQ ID NO: 12); DMR #13 (SEQ ID NO: 13); and DMR #14 (SEQ ID NO: 14); optionally wherein when the breast cancer index for the individual is about 3.01 or more the individual is classified as having breast cancer or as having a high risk of breast cancer development, or wherein when the breast cancer index for the individual is less than about 3.01 the individual is classified as not having breast cancer or as having a low risk of breast cancer development, and preferably wherein the sensitivity of the assay is at least 80.50% and the specificity of the assay is at least 80.00%; and/or 30 DMR #3 (SEQ ID NO: 3); DMR #4 (SEQ ID NO: 4); DMR #7 (SEQ ID NO: 7); DMR #12 (SEQ ID NO: 12); and DMR #14 (SEQ ID NO: 14); optionally wherein when the breast cancer index for the individual is about 3.56 or more the individual is classified as having breast cancer or as having a high risk of breast cancer development, or wherein when the breast cancer index for the individual is less than about 3.56 the individual is classified as not having breast cancer or as having a low risk of breast cancer development, and preferably wherein the sensitivity of the assay is at least 79.50% and the specificity of the assay is at least 80.00%; and/or
31 DMR #3 (SEQ ID NO: 3); DMR #2 (SEQ ID NO: 2); DMR #4 (SEQ ID NO: 4); DMR #7 (SEQ ID NO: 7); DMR #12 (SEQ ID NO: 12); and DMR #14 (SEQ ID NO: 14); optionally wherein when the breast cancer index for the individual is about 3.14 or more the individual is classified as having breast cancer or as having a high risk of breast cancer development, or wherein when the breast cancer index for the individual is less than about 3.14 the individual is classified as not having breast cancer or as having a low risk of breast cancer development, and preferably wherein the sensitivity of the assay is at least 80.50% and the specificity of the assay is at least 80.00%; and/or
32 DMR #1 (SEQ ID NO: 1); DMR #4 (SEQ ID NO: 4); DMR #7 (SEQ ID NO: 7); DMR #12 (SEQ ID NO: 12); DMR #13 (SEQ ID NO: 13); and DMR #14 (SEQ ID NO: 14); optionally wherein when the breast cancer index for the individual is about 3.30 or more the individual is classified as having breast cancer or as having a high risk of breast cancer development, or wherein when the breast cancer index for the individual is less than about 3.30 the individual is classified as not having breast cancer or as having a low risk of breast cancer development, and preferably wherein the sensitivity of the assay is at least 78.00% and the specificity of the assay is at least 80.00%; and/or
33 DMR #4 (SEQ ID NO: 4); DMR #7 (SEQ ID NO: 7); DMR #12 (SEQ ID NO: 12); and DMR #14 (SEQ ID NO: 14); optionally wherein when the breast cancer index for the individual is about 4.18 or more the individual is classified as having breast cancer or as having a high risk of breast cancer development, or wherein when the breast cancer index for the individual is less than about 4.18 the individual is classified as not having breast cancer or as having a low risk of breast cancer development, and preferably wherein the sensitivity of the assay is at least 78.00% and the specificity of the assay is at least 80.00%; and/or
34 DMR #1 (SEQ ID NO: 1); DMR #3 (SEQ ID NO: 3); DMR #4 (SEQ ID NO: 4); DMR #7 (SEQ ID NO: 7); and DMR #12 (SEQ ID NO: 12); optionally wherein when the breast cancer index for the individual is about 3.51 or more the individual is classified as having breast cancer or as having a high risk of breast cancer development, or wherein when the breast cancer index for the individual is less than about 3.51 the individual is classified as not having breast cancer or as having a low risk of breast cancer development, and preferably wherein the sensitivity of the assay is at least 80.00% and the specificity of the assay is at least 80.00%; and/or
35 DMR #1 (SEQ ID NO: 1); DMR #3 (SEQ ID NO: 3); DMR #4 (SEQ ID NO: 4); DMR #7 (SEQ ID NO: 7); DMR #12 (SEQ ID NO: 12); DMR #13 (SEQ ID NO: 13); and DMR #14 (SEQ ID NO: 14); optionally wherein when the breast cancer index for the individual is about 3.08 or more the individual is classified as having breast cancer or as having a high risk of breast cancer development, or wherein when the breast cancer index for the individual is less than about 3.08 the individual is classified as not having breast cancer or as having a low risk of breast cancer development, and preferably wherein the sensitivity of the assay is at least 79.50% and the specificity of the assay is at least 80.00%; and/or
36 DMR #1 (SEQ ID NO: 1); DMR #3 (SEQ ID NO: 3); DMR #4 (SEQ ID NO: 4); DMR #7 (SEQ ID NO: 7); DMR #12 (SEQ ID NO: 12); DMR #14 (SEQ ID NO: 14); and DMR #16 (SEQ ID NO: 16); optionally wherein when the breast cancer index for the individual is about 3.42 or more the individual is classified as having breast cancer or as having a high risk of breast cancer development, or wherein when the breast cancer index for the individual is less than about 3.42 the individual is classified as not having breast cancer or as having a low risk of breast cancer development, and preferably wherein the sensitivity of the assay is at least 78.00% and the specificity of the assay is at least 80.00%; and/or
37 DMR #1 (SEQ ID NO: 1); DMR #4 (SEQ ID NO: 4); DMR #7 (SEQ ID NO: 7); and DMR #14 (SEQ ID NO: 14); optionally wherein when the breast cancer index for the individual is about 3.80 or more the individual is classified as having breast cancer or as having a high risk of breast cancer development, or wherein when the breast cancer index for the individual is less than about 3.80 the individual is classified as not having breast cancer or as having a low risk of breast cancer development, and preferably wherein the sensitivity of the assay is at least 78.50% and the specificity of the assay is at least 80.00%; and/or
38 DMR #1 (SEQ ID NO: 1); DMR #2 (SEQ ID NO: 2); DMR #4 (SEQ ID NO: 4); DMR #7 (SEQ ID NO: 7); and DMR #14 (SEQ ID NO: 14); optionally wherein when the breast cancer index for the individual is about 3.24 or more the individual is classified as having breast cancer or as having a high risk of breast cancer development, or wherein when the breast cancer index for the individual is less than about 3.24 the individual is classified as not having breast cancer or as having a low risk of breast cancer development, and preferably wherein the sensitivity of the assay is at least 79.50% and the specificity of the assay is at least 80.00%; and/or
39 DMR #1 (SEQ ID NO: 1); DMR #3 (SEQ ID NO: 3); DMR #4 (SEQ ID NO: 4); DMR #7 (SEQ ID NO: 7); DMR #12 (SEQ ID NO: 12); DMR #13 (SEQ ID NO: 13); DMR #14 (SEQ ID NO: 14); and DMR #16 (SEQ ID NO: 16); optionally wherein when the breast cancer index for the individual is about 3.12 or more the individual is classified as having breast cancer or as having a high risk of breast cancer development, or wherein when the breast cancer index for the individual is less than about 3.12 the individual is classified as not having breast cancer or as having a low risk of breast cancer development, and preferably wherein the sensitivity of the assay is at least 78.00% and the specificity of the assay is at least 80.00%; and/or
40 DMR #1 (SEQ ID NO: 1); DMR #4 (SEQ ID NO: 4); DMR #7 (SEQ ID NO: 7); and DMR #12 (SEQ ID NO: 12); optionally wherein when the breast cancer index for the individual is about 4.06 or more the individual is classified as having breast cancer or as having a high risk of breast cancer development, or wherein when the breast cancer index for the individual is less than about 4.06 the individual is classified as not having breast cancer or as having a low risk of breast cancer development, and preferably wherein the sensitivity of the assay is at least 78.50% and the specificity of the assay is at least 80.00%; and/or
41 DMR #1 (SEQ ID NO: 1); DMR #2 (SEQ ID NO: 2); DMR #4 (SEQ ID NO: 4); DMR #7 (SEQ ID NO: 7); and DMR #12 (SEQ ID NO: 12); optionally wherein when the breast cancer index for the individual is about 3.42 or more the individual is classified as having breast cancer or as having a high risk of breast cancer development, or wherein when the breast cancer index for the individual is less than about 3.42 the individual is classified as not having breast cancer or as having a low risk of breast cancer development, and preferably wherein the sensitivity of the assay is at least 80.50% and the specificity of the assay is at least 80.00%.
42 DMR #1 (SEQ ID NO: 1); DMR #3 (SEQ ID NO: 3); DMR #2 (SEQ ID NO: 2); DMR #4 (SEQ ID NO: 4); DMR #6 (SEQ ID NO: 6); DMR #7 (SEQ ID NO: 7); DMR #15 (SEQ ID NO: 15); and DMR #17 (SEQ ID NO: 17); optionally wherein when the breast cancer index for the individual is about 5.43 or more the individual is classified as having breast cancer or as having a high risk of breast cancer development, or wherein when the breast cancer index for the individual is less than about 5.43 the individual is classified as not having breast cancer or as having a low risk of breast cancer development, and preferably wherein the sensitivity of the assay is at least 57.50% and the specificity of the assay is at least 90.00%; and/or
43 DMR #1 (SEQ ID NO: 1); DMR #3 (SEQ ID NO: 3); DMR #4 (SEQ ID NO: 4); DMR #6 (SEQ ID NO: 6); DMR #7 (SEQ ID NO: 7); DMR #14 (SEQ ID NO: 14); DMR #15 (SEQ ID NO: 15); and DMR #17 (SEQ ID NO: 17); optionally wherein when the breast cancer index for the individual is about 5.44 or more the individual is classified as having breast cancer or as having a high risk of breast cancer development, or wherein when the breast cancer index for the individual is less than about 5.44 the individual is classified as not having breast cancer or as having a low risk of breast cancer development, and preferably wherein the sensitivity of the assay is at least 58.00% and the specificity of the assay is at least 90.00%; and/or
44 DMR #1 (SEQ ID NO: 1); DMR #2 (SEQ ID NO: 2); DMR #4 (SEQ ID NO: 4); DMR #6 (SEQ ID NO: 6); DMR #7 (SEQ ID NO: 7); DMR #14 (SEQ ID NO: 14); DMR #15 (SEQ ID NO: 15); and DMR #17 (SEQ ID NO: 17); optionally wherein when the breast cancer index for the individual is about 5.33 or more the individual is classified as having breast cancer or as having a high risk of breast cancer development, or wherein when the breast cancer index for the individual is less than about 5.33 the individual is classified as not having breast cancer or as having a low risk of breast cancer development, and preferably wherein the sensitivity of the assay is at least 51.50% and the specificity of the assay is at least 90.00%; and/or
45 DMR #1 (SEQ ID NO: 1); DMR #3 (SEQ ID NO: 3); DMR #2 (SEQ ID NO: 2); DMR #4 (SEQ ID NO: 4); DMR #6 (SEQ ID NO: 6); DMR #7 (SEQ ID NO: 7); DMR #14 (SEQ ID NO: 14); DMR #15 (SEQ ID NO: 15); and DMR #17 (SEQ ID NO: 17); optionally wherein when the breast cancer index for the individual is about 5.01 or more the individual is classified as having breast cancer or as having a high risk of breast cancer development, or wherein when the breast cancer index for the individual is less than about 5.01 the individual is classified as not having breast cancer or as having a low risk of breast cancer development, and preferably wherein the sensitivity of the assay is at least 58.00% and the specificity of the assay is at least 90.00%; and/or
46 DMR #1 (SEQ ID NO: 1); DMR #3 (SEQ ID NO: 3); DMR #4 (SEQ ID NO: 4); DMR #6 (SEQ ID NO: 6); DMR #7 (SEQ ID NO: 7); DMR #13 (SEQ ID NO: 13); DMR #14 (SEQ ID NO: 14); DMR #15 (SEQ ID NO: 15); and DMR #17 (SEQ ID NO: 17); optionally wherein when the breast cancer index for the individual is about 5.00 or more the individual is classified as having breast cancer or as having a high risk of breast cancer development, or wherein when the breast cancer index for the individual is less than about 5.00 the individual is classified as not having breast cancer or as having a low risk of breast cancer development, and preferably wherein the sensitivity of the assay is at least 58.00% and the specificity of the assay is at least 90.00%; and/or
47 DMR #3 (SEQ ID NO: 3); DMR #4 (SEQ ID NO: 4); DMR #6 (SEQ ID NO: 6); DMR #7 (SEQ ID NO: 7); DMR #15 (SEQ ID NO: 15); and DMR #17 (SEQ ID NO: 17); optionally wherein when the breast cancer index for the individual is about 6.77 or more the individual is classified as having breast cancer or as having a high risk of breast cancer development, or wherein when the breast cancer index for the individual is less than about 6.77 the individual is classified as not having breast cancer or as having a low risk of breast cancer development, and preferably wherein the sensitivity of the assay is at least 58.00% and the specificity of the assay is at least 90.00%; and/or
48 DMR #1 (SEQ ID NO: 1); DMR #3 (SEQ ID NO: 3); DMR #4 (SEQ ID NO: 4); DMR #6 (SEQ ID NO: 6); DMR #7 (SEQ ID NO: 7); DMR #15 (SEQ ID NO: 15); and DMR #17 (SEQ ID NO: 17); optionally wherein when the breast cancer index for the individual is about 5.99 or more the individual is classified as having breast cancer or as having a high risk of breast cancer development, or wherein when the breast cancer index for the individual is less than about 5.99 the individual is classified as not having breast cancer or as having a low risk of breast cancer development, and preferably wherein the sensitivity of the assay is at least 58.00% and the specificity of the assay is at least 90.00%; and/or
49 DMR #1 (SEQ ID NO: 1); DMR #2 (SEQ ID NO: 2); DMR #4 (SEQ ID NO: 4); DMR #6 (SEQ ID NO: 6); DMR #7 (SEQ ID NO: 7); DMR #15 (SEQ ID NO: 15); and DMR #17 (SEQ ID NO: 17); optionally wherein when the breast cancer index for the individual is about 5.90 or more the individual is classified as having breast cancer or as having a high risk of breast cancer development, or wherein when the breast cancer index for the individual is less than about 5.90 the individual is classified as not having breast cancer or as having a low risk of breast cancer development, and preferably wherein the sensitivity of the assay is at least 51.50% and the specificity of the assay is at least 90.00%; and/or
50 DMR #3 (SEQ ID NO: 3); DMR #2 (SEQ ID NO: 2); DMR #4 (SEQ ID NO: 4); DMR #6 (SEQ ID NO: 6); DMR #7 (SEQ ID NO: 7); DMR #15 (SEQ ID NO: 15); and DMR #17 (SEQ ID NO: 17); optionally wherein when the breast cancer index for the individual is about 6.24 or more the individual is classified as having breast cancer or as having a high risk of breast cancer development, or wherein when the breast cancer index for the individual is less than about 6.24 the individual is classified as not having breast cancer or as having a low risk of breast cancer development, and preferably wherein the sensitivity of the assay is at least 48.50% and the specificity of the assay is at least 90.00%; and/or
51 DMR #1 (SEQ ID NO: 1); DMR #2 (SEQ ID NO: 2); DMR #4 (SEQ ID NO: 4); DMR #6 (SEQ ID NO: 6); DMR #7 (SEQ ID NO: 7); DMR #14 (SEQ ID NO: 14); DMR #15 (SEQ ID NO: 15); and DMR #16 (SEQ ID NO: 16); optionally wherein when the breast cancer index for the individual is about 5.55 or more the individual is classified as having breast cancer or as having a high risk of breast cancer development, or wherein when the breast cancer index for the individual is less than about 5.55 the individual is classified as not having breast cancer or as having a low risk of breast cancer development, and preferably wherein the sensitivity of the assay is at least 48.50% and the specificity of the assay is at least 90.00%; and/or
52 DMR #1 (SEQ ID NO: 1); DMR #2 (SEQ ID NO: 2); DMR #4 (SEQ ID NO: 4); DMR #6 (SEQ ID NO: 6); DMR #7 (SEQ ID NO: 7); DMR #13 (SEQ ID NO: 13); DMR #14 (SEQ ID NO: 14); DMR #15 (SEQ ID NO: 15); and DMR #16 (SEQ ID NO: 16); optionally wherein when the breast cancer index for the individual is about 5.12 or more the individual is classified as having breast cancer or as having a high risk of breast cancer development, or wherein when the breast cancer index for the individual is less than about 5.12 the individual is classified as not having breast cancer or as having a low risk of breast cancer development, and preferably wherein the sensitivity of the assay is at least 48.50% and the specificity of the assay is at least 90.00%; and/or
53 DMR #1 (SEQ ID NO: 1); DMR #2 (SEQ ID NO: 2); DMR #4 (SEQ ID NO: 4); DMR #6 (SEQ ID NO: 6); DMR #7 (SEQ ID NO: 7); DMR #14 (SEQ ID NO: 14); DMR #15 (SEQ ID NO: 15); DMR #16 (SEQ ID NO: 16); and DMR #18 (SEQ ID NO: 18); optionally wherein when the breast cancer index for the individual is about 5.53 or more the individual is classified as having breast cancer or as having a high risk of breast cancer development, or wherein when the breast cancer index for the individual is less than about 5.53 the individual is classified as not having breast cancer or as having a low risk of breast cancer development, and preferably wherein the sensitivity of the assay is at least 48.50% and the specificity of the assay is at least 90.00%; and/or
54 DMR #4 (SEQ ID NO: 4); DMR #6 (SEQ ID NO: 6); DMR #7 (SEQ ID NO: 7); DMR #14 (SEQ ID NO: 14); and DMR #15 (SEQ ID NO: 15); optionally wherein when the breast cancer index for the individual is about 7.06 or more the individual is classified as having breast cancer or as having a high risk of breast cancer development, or wherein when the breast cancer index for the individual is less than about 7.06 the individual is classified as not having breast cancer or as having a low risk of breast cancer development, and preferably wherein the sensitivity of the assay is at least 56.50% and the specificity of the assay is at least 90.00%; and/or
55 DMR #1 (SEQ ID NO: 1); DMR #3 (SEQ ID NO: 3); DMR #2 (SEQ ID NO: 2); DMR #4 (SEQ ID NO: 4); DMR #7 (SEQ ID NO: 7); DMR #13 (SEQ ID NO: 13); DMR #14 (SEQ ID NO: 14); and DMR #15 (SEQ ID NO: 15); optionally wherein when the breast cancer index for the individual is about 3.93 or more the individual is classified as having breast cancer or as having a high risk of breast cancer development, or wherein when the breast cancer index for the individual is less than about 3.93 the individual is classified as not having breast cancer or as having a low risk of breast cancer development, and preferably wherein the sensitivity of the assay is at least 34.50% and the specificity of the assay is at least 90.00%; and/or
56 DMR #1 (SEQ ID NO: 1); DMR #3 (SEQ ID NO: 3); DMR #4 (SEQ ID NO: 4); DMR #7 (SEQ ID NO: 7); DMR #14 (SEQ ID NO: 14); and DMR #15 (SEQ ID NO: 15); optionally wherein when the breast cancer index for the individual is about 4.62 or more the individual is classified as having breast cancer or as having a high risk of breast cancer development, or wherein when the breast cancer index for the individual is less than about 4.62 the individual is classified as not having breast cancer or as having a low risk of breast cancer development, and preferably wherein the sensitivity of the assay is at least 48.50% and the specificity of the assay is at least 90.00%; and/or
57 DMR #1 (SEQ ID NO: 1); DMR #2 (SEQ ID NO: 2); DMR #4 (SEQ ID NO: 4); DMR #6 (SEQ ID NO: 6); DMR #7 (SEQ ID NO: 7); DMR #12 (SEQ ID NO: 12); and DMR #15 (SEQ ID NO: 15); optionally wherein when the breast cancer index for the individual is about 5.82 or more the individual is classified as having breast cancer or as having a high risk of breast cancer development, or wherein when the breast cancer index for the individual is less than about 5.82 the individual is classified as not having breast cancer or as having a low risk of breast cancer development, and preferably wherein the sensitivity of the assay is at least 56.50% and the specificity of the assay is at least 90.00%; and/or
58 DMR #1 (SEQ ID NO: 1); DMR #3 (SEQ ID NO: 3); DMR #2 (SEQ ID NO: 2); DMR #4 (SEQ ID NO: 4); DMR #7 (SEQ ID NO: 7); DMR #14 (SEQ ID NO: 14); DMR #15 (SEQ ID NO: 15); and DMR #16 (SEQ ID NO: 16); optionally wherein when the breast cancer index for the individual is about 4.48 or more the individual is classified as having breast cancer or as having a high risk of breast cancer development, or wherein when the breast cancer index for the individual is less than about 4.48 the individual is classified as not having breast cancer or as having a low risk of breast cancer development, and preferably wherein the sensitivity of the assay is at least 34.50% and the specificity of the assay is at least 90.00%; and/or
59 DMR #1 (SEQ ID NO: 1); DMR #3 (SEQ ID NO: 3); DMR #4 (SEQ ID NO: 4); DMR #6 (SEQ ID NO: 6); DMR #7 (SEQ ID NO: 7); DMR #12 (SEQ ID NO: 12); and DMR #15 (SEQ ID NO: 15); optionally wherein when the breast cancer index for the individual is about 5.91 or more the individual is classified as having breast cancer or as having a high risk of breast cancer development, or wherein when the breast cancer index for the individual is less than about 5.91 the individual is classified as not having breast cancer or as having a low risk of breast cancer development, and preferably wherein the sensitivity of the assay is at least 56.50% and the specificity of the assay is at least 90.00%; and/or
60 DMR #1 (SEQ ID NO: 1); DMR #3 (SEQ ID NO: 3); DMR #2 (SEQ ID NO: 2); DMR #4 (SEQ ID NO: 4); DMR #7 (SEQ ID NO: 7); DMR #14 (SEQ ID NO: 14); and DMR #15 (SEQ ID NO: 15); optionally wherein when the breast cancer index for the individual is about 4.25 or more the individual is classified as having breast cancer or as having a high risk of breast cancer development, or wherein when the breast cancer index for the individual is less than about 4.25 the individual is classified as not having breast cancer or as having a low risk of breast cancer development, and preferably wherein the sensitivity of the assay is at least 34.50% and the specificity of the assay is at least 90.00%; and/or
61 DMR #1 (SEQ ID NO: 1); DMR #4 (SEQ ID NO: 4); DMR #7 (SEQ ID NO: 7); DMR #12 (SEQ ID NO: 12); and DMR #14 (SEQ ID NO: 14); optionally wherein when the breast cancer index for the individual is about 4.81 or more the individual is classified as having breast cancer or as having a high risk of breast cancer development, or wherein when the breast cancer index for the individual is less than about 4.81 the individual is classified as not having breast cancer or as having a low risk of breast cancer development, and preferably wherein the sensitivity of the assay is at least 58.00% and the specificity of the assay is at least 90.00%; and/or
62 DMR #4 (SEQ ID NO: 4); DMR #5 (SEQ ID NO: 5); DMR #6 (SEQ ID NO: 6); DMR #7 (SEQ ID NO: 7); DMR #12 (SEQ ID NO: 12); and DMR #14 (SEQ ID NO: 14); optionally wherein when the breast cancer index for the individual is about 5.95 or more the individual is classified as having breast cancer or as having a high risk of breast cancer development, or wherein when the breast cancer index for the individual is less than about 5.95 the individual is classified as not having breast cancer or as having a low risk of breast cancer development, and preferably wherein the sensitivity of the assay is at least 66.00% and the specificity of the assay is at least 90.00%; and/or
63 DMR #1 (SEQ ID NO: 1); DMR #2 (SEQ ID NO: 2); DMR #4 (SEQ ID NO: 4); DMR #7 (SEQ ID NO: 7); DMR #14 (SEQ ID NO: 14); and DMR #15 (SEQ ID NO: 15); optionally wherein when the breast cancer index for the individual is about 4.69 or more the individual is classified as having breast cancer or as having a high risk of breast cancer development, or wherein when the breast cancer index for the individual is less than about 4.69 the individual is classified as not having breast cancer or as having a low risk of breast cancer development, and preferably wherein the sensitivity of the assay is at least 34.50% and the specificity of the assay is at least 90.00%; and/or
64 DMR #1 (SEQ ID NO: 1); DMR #2 (SEQ ID NO: 2); DMR #4 (SEQ ID NO: 4); DMR #7 (SEQ ID NO: 7); DMR #12 (SEQ ID NO: 12); and DMR #14 (SEQ ID NO: 14); optionally wherein when the breast cancer index for the individual is about 4.22 or more the individual is classified as having breast cancer or as having a high risk of breast cancer development, or wherein when the breast cancer index for the individual is less than about 4.22 the individual is classified as not having breast cancer or as having a low risk of breast cancer development, and preferably wherein the sensitivity of the assay is at least 58.00% and the specificity of the assay is at least 90.00%; and/or
65 DMR #4 (SEQ ID NO: 4); DMR #7 (SEQ ID NO: 7); DMR #14 (SEQ ID NO: 14); and DMR #15 (SEQ ID NO: 15); optionally wherein when the breast cancer index for the individual is about 6.07 or more the individual is classified as having breast cancer or as having a high risk of breast cancer development, or wherein when the breast cancer index for the individual is less than about 6.07 the individual is classified as not having breast cancer or as having a low risk of breast cancer development, and preferably wherein the sensitivity of the assay is at least 50.00% and the specificity of the assay is at least 90.00%; and/or
66 DMR #2 (SEQ ID NO: 2); DMR #4 (SEQ ID NO: 4); DMR #7 (SEQ ID NO: 7); DMR #14 (SEQ ID NO: 14); and DMR #15 (SEQ ID NO: 15);
optionally wherein when the breast cancer index for the individual is about 5.32 or more the individual is classified as having breast cancer or as having a high risk of breast cancer development, or wherein when the breast cancer index for the individual is less than about 5.32 the individual is classified as not having breast cancer or as having a low risk of breast cancer development, and preferably wherein the sensitivity of the assay is at least 34.50% and the specificity of the assay is at least 90.00%; and/or
67 DMR #1 (SEQ ID NO: 1); DMR #3 (SEQ ID NO: 3); DMR #2 (SEQ ID NO: 2); DMR #4 (SEQ ID NO: 4); DMR #7 (SEQ ID NO: 7); DMR #12 (SEQ ID NO: 12); and DMR #14 (SEQ ID NO: 14); optionally wherein when the breast cancer index for the individual is about 3.62 or more the individual is classified as having breast cancer or as having a high risk of breast cancer development, or wherein when the breast cancer index for the individual is less than about 3.62 the individual is classified as not having breast cancer or as having a low risk of breast cancer development, and preferably wherein the sensitivity of the assay is at least 66.50% and the specificity of the assay is at least 90.00%; and/or
68 DMR #1 (SEQ ID NO: 1); DMR #3 (SEQ ID NO: 3); DMR #4 (SEQ ID NO: 4); DMR #7 (SEQ ID NO: 7); DMR #12 (SEQ ID NO: 12); and DMR #14 (SEQ ID NO: 14); optionally wherein when the breast cancer index for the individual is about 4.33 or more the individual is classified as having breast cancer or as having a high risk of breast cancer development, or wherein when the breast cancer index for the individual is less than about 4.33 the individual is classified as not having breast cancer or as having a low risk of breast cancer development, and preferably wherein the sensitivity of the assay is at least 61.50% and the specificity of the assay is at least 90.00%; and/or
69 DMR #2 (SEQ ID NO: 2); DMR #4 (SEQ ID NO: 4); DMR #7 (SEQ ID NO: 7); and DMR #14 (SEQ ID NO: 14); optionally wherein when the breast cancer index for the individual is about 4.50 or more the individual is classified as having breast cancer or as having a high risk of breast cancer development, or wherein when the breast cancer index for the individual is less than about 4.50 the individual is classified as not having breast cancer or as having a low risk of breast cancer development, and preferably wherein the sensitivity of the assay is at least 66.50% and the specificity of the assay is at least 90.00%; and/or
70 DMR #1 (SEQ ID NO: 1); DMR #2 (SEQ ID NO: 2); DMR #4 (SEQ ID NO: 4); DMR #7 (SEQ ID NO: 7); DMR #12 (SEQ ID NO: 12); DMR #13 (SEQ ID NO: 13); and DMR #14 (SEQ ID NO: 14); optionally wherein when the breast cancer index for the individual is about 3.78 or more the individual is classified as having breast cancer or as having a high risk of breast cancer development, or wherein when the breast cancer index for the individual is less than about 3.78 the individual is classified as not having breast cancer or as having a low risk of breast cancer development, and preferably wherein the sensitivity of the assay is at least 56.00% and the specificity of the assay is at least 90.00%; and/or
71 DMR #3 (SEQ ID NO: 3); DMR #4 (SEQ ID NO: 4); DMR #7 (SEQ ID NO: 7); DMR #12 (SEQ ID NO: 12); and DMR #14 (SEQ ID NO: 14); optionally wherein when the breast cancer index for the individual is about 4.59 or more the individual is classified as having breast cancer or as having a high risk of breast cancer development, or wherein when the breast cancer index for the individual is less than about 4.59 the individual is classified as not having breast cancer or as having a low risk of breast cancer development, and preferably wherein the sensitivity of the assay is at least 66.50% and the specificity of the assay is at least 90.00%; and/or
72 DMR #3 (SEQ ID NO: 3); DMR #2 (SEQ ID NO: 2); DMR #4 (SEQ ID NO: 4); DMR #7 (SEQ ID NO: 7); DMR #12 (SEQ ID NO: 12); and DMR #14 (SEQ ID NO: 14); optionally wherein when the breast cancer index for the individual is about 3.96 or more the individual is classified as having breast cancer or as having a high risk of breast cancer development, or wherein when the breast cancer index for the individual is less than about 3.96 the individual is classified as not having breast cancer or as having a low risk of breast cancer development, and preferably wherein the sensitivity of the assay is at least 64.50% and the specificity of the assay is at least 90.00%; and/or
73 DMR #1 (SEQ ID NO: 1); DMR #4 (SEQ ID NO: 4); DMR #7 (SEQ ID NO: 7); DMR #12 (SEQ ID NO: 12); DMR #13 (SEQ ID NO: 13); and DMR #14 (SEQ ID NO: 14); optionally wherein when the breast cancer index for the individual is about 4.43 or more the individual is classified as having breast cancer or as having a high risk of breast cancer development, or wherein when the breast cancer index for the individual is less than about 4.43 the individual is classified as not having breast cancer or as having a low risk of breast cancer development, and preferably wherein the sensitivity of the assay is at least 46.50% and the specificity of the assay is at least 90.00%; and/or
74 DMR #4 (SEQ ID NO: 4); DMR #7 (SEQ ID NO: 7); DMR #12 (SEQ ID NO: 12); and DMR #14 (SEQ ID NO: 14); optionally wherein when the breast cancer index for the individual is about 5.65 or more the individual is classified as having breast cancer or as having a high risk of breast cancer development, or wherein when the breast cancer index for the individual is less than about 5.65 the individual is classified as not having breast cancer or as having a low risk of breast cancer development, and preferably wherein the sensitivity of the assay is at least 61.00% and the specificity of the assay is at least 90.00%; and/or
75 DMR #1 (SEQ ID NO: 1); DMR #3 (SEQ ID NO: 3); DMR #4 (SEQ ID NO: 4); DMR #7 (SEQ ID NO: 7); and DMR #12 (SEQ ID NO: 12); optionally wherein when the breast cancer index for the individual is about 4.67 or more the individual is classified as having breast cancer or as having a high risk of breast cancer development, or wherein when the breast cancer index for the individual is less than about 4.67 the individual is classified as not having breast cancer or as having a low risk of breast cancer development, and preferably wherein the sensitivity of the assay is at least 26.50% and the specificity of the assay is at least 90.00%; and/or
76 DMR #1 (SEQ ID NO: 1); DMR #3 (SEQ ID NO: 3); DMR #4 (SEQ ID NO: 4); DMR #7 (SEQ ID NO: 7); DMR #12 (SEQ ID NO: 12); DMR #13 (SEQ ID NO: 13); and DMR #14 (SEQ ID NO: 14); optionally wherein when the breast cancer index for the individual is about 3.91 or more the individual is classified as having breast cancer or as having a high risk of breast cancer development, or wherein when the breast cancer index for the individual is less than about 3.91 the individual is classified as not having breast cancer or as having a low risk of breast cancer development, and preferably wherein the sensitivity of the assay is at least 57.50% and the specificity of the assay is at least 90.00%; and/or
77 DMR #1 (SEQ ID NO: 1); DMR #3 (SEQ ID NO: 3); DMR #4 (SEQ ID NO: 4); DMR #7 (SEQ ID NO: 7); DMR #12 (SEQ ID NO: 12); DMR #14 (SEQ ID NO: 14); and DMR #16 (SEQ ID NO: 16); optionally wherein when the breast cancer index for the individual is about 4.64 or more the individual is classified as having breast cancer or as having a high risk of breast cancer development, or wherein when the breast cancer index for the individual is less than about 4.64 the individual is classified as not having breast cancer or as having a low risk of breast cancer development, and preferably wherein the sensitivity of the assay is at least 46.50% and the specificity of the assay is at least 90.00%; and/or
78 DMR #1 (SEQ ID NO: 1); DMR #4 (SEQ ID NO: 4); DMR #7 (SEQ ID NO: 7); and DMR #14 (SEQ ID NO: 14); optionally wherein when the breast cancer index for the individual is about 4.97 or more the individual is classified as having breast cancer or as having a high risk of breast cancer development, or wherein when the breast cancer index for the individual is less than about 4.97 the individual is classified as not having breast cancer or as having a low risk of breast cancer development, and preferably wherein the sensitivity of the assay is at least 62.50% and the specificity of the assay is at least 90.00%; and/or
79 DMR #1 (SEQ ID NO: 1); DMR #2 (SEQ ID NO: 2); DMR #4 (SEQ ID NO: 4); DMR #7 (SEQ ID NO: 7); and DMR #14 (SEQ ID NO: 14); optionally wherein when the breast cancer index for the individual is about 4.11 or more the individual is classified as having breast cancer or as having a high risk of breast cancer development, or wherein when the breast cancer index for the individual is less than about 4.11 the individual is classified as not having breast cancer or as having a low risk of breast cancer development, and preferably wherein the sensitivity of the assay is at least 62.00% and the specificity of the assay is at least 90.00%; and/or
80 DMR #1 (SEQ ID NO: 1); DMR #3 (SEQ ID NO: 3); DMR #4 (SEQ ID NO: 4); DMR #7 (SEQ ID NO: 7); DMR #12 (SEQ ID NO: 12); DMR #13 (SEQ ID NO: 13); DMR #14 (SEQ ID NO: 14); and DMR #16 (SEQ ID NO: 16); optionally wherein when the breast cancer index for the individual is about 4.27 or more the individual is classified as having breast cancer or as having a high risk of breast cancer development, or wherein when the breast cancer index for the individual is less than about 4.27 the individual is classified as not having breast cancer or as having a low risk of breast cancer development, and preferably wherein the sensitivity of the assay is at least 46.50% and the specificity of the assay is at least 90.00%; and/or
81 DMR #1 (SEQ ID NO: 1); DMR #4 (SEQ ID NO: 4); DMR #7 (SEQ ID NO: 7); and DMR #12 (SEQ ID NO: 12); optionally wherein when the breast cancer index for the individual is about 5.64 or more the individual is classified as having breast cancer or as having a high risk of breast cancer development, or wherein when the breast cancer index for the individual is less than about 5.64 the individual is classified as not having breast cancer or as having a low risk of breast cancer development, and preferably wherein the sensitivity of the assay is at least 53.50% and the specificity of the assay is at least 90.00%; and/or
82 DMR #1 (SEQ ID NO: 1); DMR #2 (SEQ ID NO: 2); DMR #4 (SEQ ID NO: 4); DMR #7 (SEQ ID NO: 7); and DMR #12 (SEQ ID NO: 12); optionally wherein when the breast cancer index for the individual is about 4.75 or more the individual is classified as having breast cancer or as having a high risk of breast cancer development, or wherein when the breast cancer index for the individual is less than about 4.75 the individual is classified as not having breast cancer or as having a low risk of breast cancer development, and preferably wherein the sensitivity of the assay is at least 53.50% and the specificity of the assay is at least 90.00%.
83 DMR #1 (SEQ ID NO: 1); DMR #3 (SEQ ID NO: 3); DMR #2 (SEQ ID NO: 2); DMR #4 (SEQ ID NO: 4); DMR #6 (SEQ ID NO: 6); DMR #7 (SEQ ID NO: 7); DMR #15 (SEQ ID NO: 15); and DMR #17 (SEQ ID NO: 17); optionally wherein when the breast cancer index for the individual is about 5.86 or more the individual is classified as having breast cancer or as having a high risk of breast cancer development, or wherein when the breast cancer index for the individual is less than about 5.86 the individual is classified as not having breast cancer or as having a low risk of breast cancer development, and preferably wherein the sensitivity of the assay is at least 38.00% and the specificity of the assay is at least 95.00%; and/or
84 DMR #1 (SEQ ID NO: 1); DMR #3 (SEQ ID NO: 3); DMR #4 (SEQ ID NO: 4); DMR #6 (SEQ ID NO: 6); DMR #7 (SEQ ID NO: 7); DMR #14 (SEQ ID NO: 14); DMR #15 (SEQ ID NO: 15); and DMR #17 (SEQ ID NO: 17); optionally wherein when the breast cancer index for the individual is about 6.06 or more the individual is classified as having breast cancer or as having a high risk of breast cancer development, or wherein when the breast cancer index for the individual is less than about 6.06 the individual is classified as not having breast cancer or as having a low risk of breast cancer development, and preferably wherein the sensitivity of the assay is at least 38.50% and the specificity of the assay is at least 95.00%; and/or
85 DMR #1 (SEQ ID NO: 1); DMR #2 (SEQ ID NO: 2); DMR #4 (SEQ ID NO: 4); DMR #6 (SEQ ID NO: 6); DMR #7 (SEQ ID NO: 7); DMR #14 (SEQ ID NO: 14); DMR #15 (SEQ ID NO: 15); and DMR #17 (SEQ ID NO: 17); optionally wherein when the breast cancer index for the individual is about 5.97 or more the individual is classified as having breast cancer or as having a high risk of breast cancer development, or wherein when the breast cancer index for the individual is less than about 5.97 the individual is classified as not having breast cancer or as having a low risk of breast cancer development, and preferably wherein the sensitivity of the assay is at least 29.50% and the specificity of the assay is at least 95.00%; and/or
86 DMR #1 (SEQ ID NO: 1); DMR #3 (SEQ ID NO: 3); DMR #2 (SEQ ID NO: 2); DMR #4 (SEQ ID NO: 4); DMR #6 (SEQ ID NO: 6); DMR #7 (SEQ ID NO: 7); DMR #14 (SEQ ID NO: 14); DMR #15 (SEQ ID NO: 15); and DMR #17 (SEQ ID NO: 17); optionally wherein when the breast cancer index for the individual is about 5.45 or more the individual is classified as having breast cancer or as having a high risk of breast cancer development, or wherein when the breast cancer index for the individual is less than about 5.45 the individual is classified as not having breast cancer or as having a low risk of breast cancer development, and preferably wherein the sensitivity of the assay is at least 38.50% and the specificity of the assay is at least 95.00%; and/or
87 DMR #1 (SEQ ID NO: 1); DMR #3 (SEQ ID NO: 3); DMR #4 (SEQ ID NO: 4); DMR #6 (SEQ ID NO: 6); DMR #7 (SEQ ID NO: 7); DMR #13 (SEQ ID NO: 13); DMR #14 (SEQ ID NO: 14); DMR #15 (SEQ ID NO: 15); and DMR #17 (SEQ ID NO: 17); optionally wherein when the breast cancer index for the individual is about 5.51 or more the individual is classified as having breast cancer or as having a high risk of breast cancer development, or wherein when the breast cancer index for the individual is less than about 5.51 the individual is classified as not having breast cancer or as having a low risk of breast cancer development, and preferably wherein the sensitivity of the assay is at least 38.50% and the specificity of the assay is at least 95.00%; and/or
88 DMR #3 (SEQ ID NO: 3); DMR #4 (SEQ ID NO: 4); DMR #6 (SEQ ID NO: 6); DMR #7 (SEQ ID NO: 7); DMR #15 (SEQ ID NO: 15); and DMR #17 (SEQ ID NO: 17); optionally wherein when the breast cancer index for the individual is about 7.43 or more the individual is classified as having breast cancer or as having a high risk of breast cancer development, or wherein when the breast cancer index for the individual is less than about 7.43 the individual is classified as not having breast cancer or as having a low risk of breast cancer development, and preferably wherein the sensitivity of the assay is at least 38.50% and the specificity of the assay is at least 95.00%; and/or
89 DMR #1 (SEQ ID NO: 1); DMR #3 (SEQ ID NO: 3); DMR #4 (SEQ ID NO: 4); DMR #6 (SEQ ID NO: 6); DMR #7 (SEQ ID NO: 7); DMR #15 (SEQ ID NO: 15); and DMR #17 (SEQ ID NO: 17); optionally wherein when the breast cancer index for the individual is about 6.65 or more the individual is classified as having breast cancer or as having a high risk of breast cancer development, or wherein when the breast cancer index for the individual is less than about 6.65 the individual is classified as not having breast cancer or as having a low risk of breast cancer development, and preferably wherein the sensitivity of the assay is at least 38.50% and the specificity of the assay is at least 95.00%; and/or
90 DMR #1 (SEQ ID NO: 1); DMR #2 (SEQ ID NO: 2); DMR #4 (SEQ ID NO: 4); DMR #6 (SEQ ID NO: 6); DMR #7 (SEQ ID NO: 7); DMR #15 (SEQ ID NO: 15); and DMR #17 (SEQ ID NO: 17); optionally wherein when the breast cancer index for the individual is about 6.56 or more the individual is classified as having breast cancer or as having a high risk of breast cancer development, or wherein when the breast cancer index for the individual is less than about 6.56 the individual is classified as not having breast cancer or as having a low risk of breast cancer development, and preferably wherein the sensitivity of the assay is at least 29.50% and the specificity of the assay is at least 95.00%; and/or
91 DMR #3 (SEQ ID NO: 3); DMR #2 (SEQ ID NO: 2); DMR #4 (SEQ ID NO: 4); DMR #6 (SEQ ID NO: 6); DMR #7 (SEQ ID NO: 7); DMR #15 (SEQ ID NO: 15); and DMR #17 (SEQ ID NO: 17); optionally wherein when the breast cancer index for the individual is about 6.46 or more the individual is classified as having breast cancer or as having a high risk of breast cancer development, or wherein when the breast cancer index for the individual is less than about 6.46 the individual is classified as not having breast cancer or as having a low risk of breast cancer development, and preferably wherein the sensitivity of the assay is at least 25.00% and the specificity of the assay is at least 95.00%; and/or
92 DMR #1 (SEQ ID NO: 1); DMR #2 (SEQ ID NO: 2); DMR #4 (SEQ ID NO: 4); DMR #6 (SEQ ID NO: 6); DMR #7 (SEQ ID NO: 7); DMR #14 (SEQ ID NO: 14); DMR #15 (SEQ ID NO: 15); and DMR #16 (SEQ ID NO: 16); optionally wherein when the breast cancer index for the individual is about 5.72 or more the individual is classified as having breast cancer or as having a high risk of breast cancer development, or wherein when the breast cancer index for the individual is less than about 5.72 the individual is classified as not having breast cancer or as having a low risk of breast cancer development, and preferably wherein the sensitivity of the assay is at least 25.00% and the specificity of the assay is at least 95.00%; and/or
93 DMR #1 (SEQ ID NO: 1); DMR #2 (SEQ ID NO: 2); DMR #4 (SEQ ID NO: 4); DMR #6 (SEQ ID NO: 6); DMR #7 (SEQ ID NO: 7); DMR #13 (SEQ ID NO: 13); DMR #14 (SEQ ID NO: 14); DMR #15 (SEQ ID NO: 15); and DMR #16 (SEQ ID NO: 16); optionally wherein when the breast cancer index for the individual is about 5.28 or more the individual is classified as having breast cancer or as having a high risk of breast cancer development, or wherein when the breast cancer index for the individual is less than about 5.28 the individual is classified as not having breast cancer or as having a low risk of breast cancer development, and preferably wherein the sensitivity of the assay is at least 25.00% and the specificity of the assay is at least 95.00%; and/or
94 DMR #1 (SEQ ID NO: 1); DMR #2 (SEQ ID NO: 2); DMR #4 (SEQ ID NO: 4); DMR #6 (SEQ ID NO: 6); DMR #7 (SEQ ID NO: 7); DMR #14 (SEQ ID NO: 14); DMR #15 (SEQ ID NO: 15); DMR #16 (SEQ ID NO: 16); and DMR #18 (SEQ ID NO: 18); optionally wherein when the breast cancer index for the individual is about 5.67 or more the individual is classified as having breast cancer or as having a high risk of breast cancer development, or wherein when the breast cancer index for the individual is less than about 5.67 the individual is classified as not having breast cancer or as having a low risk of breast cancer development, and preferably wherein the sensitivity of the assay is at least 25.00% and the specificity of the assay is at least 95.00%; and/or
95 DMR #4 (SEQ ID NO: 4); DMR #6 (SEQ ID NO: 6); DMR #7 (SEQ ID NO: 7); DMR #14 (SEQ ID NO: 14); and DMR #15 (SEQ ID NO: 15); optionally wherein when the breast cancer index for the individual is about 7.13 or more the individual is classified as having breast cancer or as having a high risk of breast cancer development, or wherein when the breast cancer index for the individual is less than about 7.13 the individual is classified as not having breast cancer or as having a low risk of breast cancer development, and preferably wherein the sensitivity of the assay is at least 38.00% and the specificity of the assay is at least 95.00%; and/or
96 DMR #1 (SEQ ID NO: 1); DMR #3 (SEQ ID NO: 3); DMR #2 (SEQ ID NO: 2); DMR #4 (SEQ ID NO: 4); DMR #7 (SEQ ID NO: 7); DMR #13 (SEQ ID NO: 13); DMR #14 (SEQ ID NO: 14); and DMR #15 (SEQ ID NO: 15); optionally wherein when the breast cancer index for the individual is about 4.02 or more the individual is classified as having breast cancer or as having a high risk of breast cancer development, or wherein when the breast cancer index for the individual is less than about 4.02 the individual is classified as not having breast cancer or as having a low risk of breast cancer development, and preferably wherein the sensitivity of the assay is at least 8.00% and the specificity of the assay is at least 95.00%; and/or
97 DMR #1 (SEQ ID NO: 1); DMR #3 (SEQ ID NO: 3); DMR #4 (SEQ ID NO: 4); DMR #7 (SEQ ID NO: 7); DMR #14 (SEQ ID NO: 14); and DMR #15 (SEQ ID NO: 15); optionally wherein when the breast cancer index for the individual is about 4.86 or more the individual is classified as having breast cancer or as having a high risk of breast cancer development, or wherein when the breast cancer index for the individual is less than about 4.86 the individual is classified as not having breast cancer or as having a low risk of breast cancer development, and preferably wherein the sensitivity of the assay is at least 22.00% and the specificity of the assay is at least 95.00%; and/or
98 DMR #1 (SEQ ID NO: 1); DMR #2 (SEQ ID NO: 2); DMR #4 (SEQ ID NO: 4); DMR #6 (SEQ ID NO: 6); DMR #7 (SEQ ID NO: 7); DMR #12 (SEQ ID NO: 12); and DMR #15 (SEQ ID NO: 15); optionally wherein when the breast cancer index for the individual is about 5.89 or more the individual is classified as having breast cancer or as having a high risk of breast cancer development, or wherein when the breast cancer index for the individual is less than about 5.89 the individual is classified as not having breast cancer or as having a low risk of breast cancer development, and preferably wherein the sensitivity of the assay is at least 38.00% and the specificity of the assay is at least 95.00%; and/or
99 DMR #1 (SEQ ID NO: 1); DMR #3 (SEQ ID NO: 3); DMR #2 (SEQ ID NO: 2); DMR #4 (SEQ ID NO: 4); DMR #7 (SEQ ID NO: 7); DMR #14 (SEQ ID NO: 14); DMR #15 (SEQ ID NO: 15); and DMR #16 (SEQ ID NO: 16); optionally wherein when the breast cancer index for the individual is about 4.60 or more the individual is classified as having breast cancer or as having a high risk of breast cancer development, or wherein when the breast cancer index for the individual is less than about 4.60 the individual is classified as not having breast cancer or as having a low risk of breast cancer development, and preferably wherein the sensitivity of the assay is at least 8.00% and the specificity of the assay is at least 95.00%; and/or
100 DMR #1 (SEQ ID NO: 1); DMR #3 (SEQ ID NO: 3); DMR #4 (SEQ ID NO: 4); DMR #6 (SEQ ID NO: 6); DMR #7 (SEQ ID NO: 7); DMR #12 (SEQ ID NO: 12); and DMR #15 (SEQ ID NO: 15); optionally wherein when the breast cancer index for the individual is about 5.95 or more the individual is classified as having breast cancer or as having a high risk of breast cancer development, or wherein when the breast cancer index for the individual is less than about 5.95 the individual is classified as not having breast cancer or as having a low risk of breast cancer development, and preferably wherein the sensitivity of the assay is at least 38.00% and the specificity of the assay is at least 95.00%; and/or
101 DMR #1 (SEQ ID NO: 1); DMR #3 (SEQ ID NO: 3); DMR #2 (SEQ ID NO: 2); DMR #4 (SEQ ID NO: 4); DMR #7 (SEQ ID NO: 7); DMR #14 (SEQ ID NO: 14); and DMR #15 (SEQ ID NO: 15); optionally wherein when the breast cancer index for the individual is about 4.33 or more the individual is classified as having breast cancer or as having a high risk of breast cancer development, or wherein when the breast cancer index for the individual is less than about 4.33 the individual is classified as not having breast cancer or as having a low risk of breast cancer development, and preferably wherein the sensitivity of the assay is at least 8.00% and the specificity of the assay is at least 95.00%; and/or
102 DMR #1 (SEQ ID NO: 1); DMR #4 (SEQ ID NO: 4); DMR #7 (SEQ ID NO: 7); DMR #12 (SEQ ID NO: 12); and DMR #14 (SEQ ID NO: 14); optionally wherein when the breast cancer index for the individual is about 4.99 or more the individual is classified as having breast cancer or as having a high risk of breast cancer development, or wherein when the breast cancer index for the individual is less than about 4.99 the individual is classified as not having breast cancer or as having a low risk of breast cancer development, and preferably wherein the sensitivity of the assay is at least 35.50% and the specificity of the assay is at least 95.00%; and/or
103 DMR #4 (SEQ ID NO: 4); DMR #5 (SEQ ID NO: 5); DMR #6 (SEQ ID NO: 6); DMR #7 (SEQ ID NO: 7); DMR #12 (SEQ ID NO: 12); and DMR #14 (SEQ ID NO: 14); optionally wherein when the breast cancer index for the individual is about 6.50 or more the individual is classified as having breast cancer or as having a high risk of breast cancer development, or wherein when the breast cancer index for the individual is less than about 6.50 the individual is classified as not having breast cancer or as having a low risk of breast cancer development, and preferably wherein the sensitivity of the assay is at least 53.00% and the specificity of the assay is at least 95.00%; and/or
104 DMR #1 (SEQ ID NO: 1); DMR #2 (SEQ ID NO: 2); DMR #4 (SEQ ID NO: 4); DMR #7 (SEQ ID NO: 7); DMR #14 (SEQ ID NO: 14); and DMR #15 (SEQ ID NO: 15); optionally wherein when the breast cancer index for the individual is about 4.77 or more the individual is classified as having breast cancer or as having a high risk of breast cancer development, or wherein when the breast cancer index for the individual is less than about 4.77 the individual is classified as not having breast cancer or as having a low risk of breast cancer development, and preferably wherein the sensitivity of the assay is at least 8.00% and the specificity of the assay is at least 95.00%; and/or
105 DMR #1 (SEQ ID NO: 1); DMR #2 (SEQ ID NO: 2); DMR #4 (SEQ ID NO: 4); DMR #7 (SEQ ID NO: 7); DMR #12 (SEQ ID NO: 12); and DMR #14 (SEQ ID NO: 14); optionally wherein when the breast cancer index for the individual is about 4.39 or more the individual is classified as having breast cancer or as having a high risk of breast cancer development, or wherein when the breast cancer index for the individual is less than about 4.39 the individual is classified as not having breast cancer or as having a low risk of breast cancer development, and preferably wherein the sensitivity of the assay is at least 35.50% and the specificity of the assay is at least 95.00%; and/or
106 DMR #4 (SEQ ID NO: 4); DMR #7 (SEQ ID NO: 7); DMR #14 (SEQ ID NO: 14); and DMR #15 (SEQ ID NO: 15); optionally wherein when the breast cancer index for the individual is about 6.57 or more the individual is classified as having breast cancer or as having a high risk of breast cancer development, or wherein when the breast cancer index for the individual is less than about 6.57 the individual is classified as not having breast cancer or as having a low risk of breast cancer development, and preferably wherein the sensitivity of the assay is at least 23.50% and the specificity of the assay is at least 95.00%; and/or
107 DMR #2 (SEQ ID NO: 2); DMR #4 (SEQ ID NO: 4); DMR #7 (SEQ ID NO: 7); DMR #14 (SEQ ID NO: 14); and DMR #15 (SEQ ID NO: 15); optionally wherein when the breast cancer index for the individual is about 5.37 or more the individual is classified as having breast cancer or as having a high risk of breast cancer development, or wherein when the breast cancer index for the individual is less than about 5.37 the individual is classified as not having breast cancer or as having a low risk of breast cancer development, and preferably wherein the sensitivity of the assay is at least 8.00% and the specificity of the assay is at least 95.00%; and/or
108 DMR #1 (SEQ ID NO: 1); DMR #3 (SEQ ID NO: 3); DMR #2 (SEQ ID NO: 2); DMR #4 (SEQ ID NO: 4); DMR #7 (SEQ ID NO: 7); DMR #12 (SEQ ID NO: 12); and DMR #14 (SEQ ID NO: 14); optionally wherein when the breast cancer index for the individual is about 4.11 or more the individual is classified as having breast cancer or as having a high risk of breast cancer development, or wherein when the breast cancer index for the individual is less than about 4.11 the individual is classified as not having breast cancer or as having a low risk of breast cancer development, and preferably wherein the sensitivity of the assay is at least 50.00% and the specificity of the assay is at least 95.00%; and/or
109 DMR #1 (SEQ ID NO: 1); DMR #3 (SEQ ID NO: 3); DMR #4 (SEQ ID NO: 4); DMR #7 (SEQ ID NO: 7); DMR #12 (SEQ ID NO: 12); and DMR #14 (SEQ ID NO: 14); optionally wherein when the breast cancer index for the individual is about 4.42 or more the individual is classified as having breast cancer or as having a high risk of breast cancer development, or wherein when the breast cancer index for the individual is less than about 4.42 the individual is classified as not having breast cancer or as having a low risk of breast cancer development, and preferably wherein the sensitivity of the assay is at least 44.50% and the specificity of the assay is at least 95.00%; and/or
110 DMR #2 (SEQ ID NO: 2); DMR #4 (SEQ ID NO: 4); DMR #7 (SEQ ID NO: 7); and DMR #14 (SEQ ID NO: 14); optionally wherein when the breast cancer index for the individual is about 5.26 or more the individual is classified as having breast cancer or as having a high risk of breast cancer development, or wherein when the breast cancer index for the individual is less than about 5.26 the individual is classified as not having breast cancer or as having a low risk of breast cancer development, and preferably wherein the sensitivity of the assay is at least 50.00% and the specificity of the assay is at least 95.00%; and/or
111 DMR #1 (SEQ ID NO: 1); DMR #2 (SEQ ID NO: 2); DMR #4 (SEQ ID NO: 4); DMR #7 (SEQ ID NO: 7); DMR #12 (SEQ ID NO: 12); DMR #13 (SEQ ID NO: 13); and DMR #14 (SEQ ID NO: 14); optionally wherein when the breast cancer index for the individual is about 4.04 or more the individual is classified as having breast cancer or as having a high risk of breast cancer development, or wherein when the breast cancer index for the individual is less than about 4.04 the individual is classified as not having breast cancer or as having a low risk of breast cancer development, and preferably wherein the sensitivity of the assay is at least 33.55% and the specificity of the assay is at least 95.00%; and/or
112 DMR #3 (SEQ ID NO: 3); DMR #4 (SEQ ID NO: 4); DMR #7 (SEQ ID NO: 7); DMR #12 (SEQ ID NO: 12); and DMR #14 (SEQ ID NO: 14); optionally wherein when the breast cancer index for the individual is about 5.15 or more the individual is classified as having breast cancer or as having a high risk of breast cancer development, or wherein when the breast cancer index for the individual is less than about 5.15 the individual is classified as not having breast cancer or as having a low risk of breast cancer development, and preferably wherein the sensitivity of the assay is at least 53.50% and the specificity of the assay is at least 95.00%; and/or
113 DMR #3 (SEQ ID NO: 3); DMR #2 (SEQ ID NO: 2); DMR #4 (SEQ ID NO: 4); DMR #7 (SEQ ID NO: 7); DMR #12 (SEQ ID NO: 12); and DMR #14 (SEQ ID NO: 14); optionally wherein when the breast cancer index for the individual is about 4.67 or more the individual is classified as having breast cancer or as having a high risk of breast cancer development, or wherein when the breast cancer index for the individual is less than about 4.67 the individual is classified as not having breast cancer or as having a low risk of breast cancer development, and preferably wherein the sensitivity of the assay is at least 48.50% and the specificity of the assay is at least 95.00%; and/or
114 DMR #1 (SEQ ID NO: 1); DMR #4 (SEQ ID NO: 4); DMR #7 (SEQ ID NO: 7); DMR #12 (SEQ ID NO: 12); DMR #13 (SEQ ID NO: 13); and DMR #14 (SEQ ID NO: 14); optionally wherein when the breast cancer index for the individual is about 4.46 or more the individual is classified as having breast cancer or as having a high risk of breast cancer development, or wherein when the breast cancer index for the individual is less than about 4.46 the individual is classified as not having breast cancer or as having a low risk of breast cancer development, and preferably wherein the sensitivity of the assay is at least 21.00% and the specificity of the assay is at least 95.00%; and/or
115 DMR #4 (SEQ ID NO: 4); DMR #7 (SEQ ID NO: 7); DMR #12 (SEQ ID NO: 12); and DMR #14 (SEQ ID NO: 14); optionally wherein when the breast cancer index for the individual is about 5.82 or more the individual is classified as having breast cancer or as having a high risk of breast cancer development, or wherein when the breast cancer index for the individual is less than about 5.82 the individual is classified as not having breast cancer or as having a low risk of breast cancer development, and preferably wherein the sensitivity of the assay is at least 47.50% and the specificity of the assay is at least 95.00%; and/or
116 DMR #1 (SEQ ID NO: 1); DMR #3 (SEQ ID NO: 3); DMR #4 (SEQ ID NO: 4); DMR #7 (SEQ ID NO: 7); and DMR #12 (SEQ ID NO: 12); optionally wherein when the breast cancer index for the individual is about 4.98 or more the individual is classified as having breast cancer or as having a high risk of breast cancer development, or wherein when the breast cancer index for the individual is less than about 4.98 the individual is classified as not having breast cancer or as having a low risk of breast cancer development, and preferably wherein the sensitivity of the assay is at least 45.00% and the specificity of the assay is at least 95.00%; and/or
117 DMR #1 (SEQ ID NO: 1); DMR #3 (SEQ ID NO: 3); DMR #4 (SEQ ID NO: 4); DMR #7 (SEQ ID NO: 7); DMR #12 (SEQ ID NO: 12); DMR #13 (SEQ ID NO: 13); and DMR #14 (SEQ ID NO: 14); optionally wherein when the breast cancer index for the individual is about 4.11 or more the individual is classified as having breast cancer or as having a high risk of breast cancer development, or wherein when the breast cancer index for the individual is less than about 4.11 the individual is classified as not having breast cancer or as having a low risk of breast cancer development, and preferably wherein the sensitivity of the assay is at least 37.50% and the specificity of the assay is at least 95.00%; and/or
118 DMR #1 (SEQ ID NO: 1); DMR #3 (SEQ ID NO: 3); DMR #4 (SEQ ID NO: 4); DMR #7 (SEQ ID NO: 7); DMR #12 (SEQ ID NO: 12); DMR #14 (SEQ ID NO: 14); and DMR #16 (SEQ ID NO: 16); optionally wherein when the breast cancer index for the individual is about 4.74 or more the individual is classified as having breast cancer or as having a high risk of breast cancer development, or wherein when the breast cancer index for the individual is less than about 4.74 the individual is classified as not having breast cancer or as having a low risk of breast cancer development, and preferably wherein the sensitivity of the assay is at least 21.00% and the specificity of the assay is at least 95.00%; and/or
119 DMR #1 (SEQ ID NO: 1); DMR #4 (SEQ ID NO: 4); DMR #7 (SEQ ID NO: 7); and DMR #14 (SEQ ID NO: 14); optionally wherein when the breast cancer index for the individual is about 5.09 or more the individual is classified as having breast cancer or as having a high risk of breast cancer development, or wherein when the breast cancer index for the individual is less than about 5.09 the individual is classified as not having breast cancer or as having a low risk of breast cancer development, and preferably wherein the sensitivity of the assay is at least 47.50% and the specificity of the assay is at least 95.00%; and/or
120 DMR #1 (SEQ ID NO: 1); DMR #2 (SEQ ID NO: 2); DMR #4 (SEQ ID NO: 4); DMR #7 (SEQ ID NO: 7); and DMR #14 (SEQ ID NO: 14); optionally wherein when the breast cancer index for the individual is about 4.46 or more the individual is classified as having breast cancer or as having a high risk of breast cancer development, or wherein when the breast cancer index for the individual is less than about 4.46 the individual is classified as not having breast cancer or as having a low risk of breast cancer development, and preferably wherein the sensitivity of the assay is at least 45.50% and the specificity of the assay is at least 95.00%; and/or
121 DMR #1 (SEQ ID NO: 1); DMR #3 (SEQ ID NO: 3); DMR #4 (SEQ ID NO: 4); DMR #7 (SEQ ID NO: 7); DMR #12 (SEQ ID NO: 12); DMR #13 (SEQ ID NO: 13); DMR #14 (SEQ ID NO: 14); and DMR #16 (SEQ ID NO: 16); optionally wherein when the breast cancer index for the individual is about 4.37 or more the individual is classified as having breast cancer or as having a high risk of breast cancer development, or wherein when the breast cancer index for the individual is less than about 4.37 the individual is classified as not having breast cancer or as having a low risk of breast cancer development, and preferably wherein the sensitivity of the assay is at least 21.00% and the specificity of the assay is at least 95.00%; and/or
122 DMR #1 (SEQ ID NO: 1); DMR #4 (SEQ ID NO: 4); DMR #7 (SEQ ID NO: 7); and DMR #12 (SEQ ID NO: 12); optionally wherein when the breast cancer index for the individual is about 5.78 or more the individual is classified as having breast cancer or as having a high risk of breast cancer development, or wherein when the breast cancer index for the individual is less than about 5.78 the individual is classified as not having breast cancer or as having a low risk of breast cancer development, and preferably wherein the sensitivity of the assay is at least 31.00% and the specificity of the assay is at least 95.00%; and/or
123 DMR #1 (SEQ ID NO: 1); DMR #2 (SEQ ID NO: 2); DMR #4 (SEQ ID NO: 4); DMR #7 (SEQ ID NO: 7); and DMR #12 (SEQ ID NO: 12); optionally wherein when the breast cancer index for the individual is about 4.98 or more the individual is classified as having breast cancer or as having a high risk of breast cancer development, or wherein when the breast cancer index for the individual is less than about 4.98 the individual is classified as not having breast cancer or as having a low risk of breast cancer development, and preferably wherein the sensitivity of the assay is at least 29.00% and the specificity of the assay is at least 95.00%.

The predicting of the presence, absence or development of breast cancer in an individual may particularly involve threshold PMR value being applied in order to stratify an individual as having or not having breast cancer or of having a high or low risk of breast cancer development.

In any of the assays described herein wherein the breast cancer index value of an individual is determined by assaying a panel that comprises one or more CpGs identified at nucleotide positions 61 to 62 in SEQ ID NOs 30 to 66:
a. the panel of one or more CpGs may comprise at least 5 of the CpGs that are identified in SEQ ID NOs 30 to 66, and optionally wherein the at least 5 CpGs comprise the CpGs identified in SEQ ID NOs 30 to 34;
b. the panel of one or more CpGs may comprise at least 10 of the CpGs that are identified in SEQ ID NOs 30 to 66, and optionally wherein the at least 10 CpGs comprise the CpGs identified in SEQ ID NOs 30 to 39;
c. the panel of one or more CpGs may comprise at least 20 of the CpGs that are identified in SEQ ID NOs 30 to 66, and optionally wherein the at least 20 CpGs comprise the CpGs identified in SEQ ID NOs 30 to 49;
d. the panel of one or more CpGs may comprise at least 30 of the CpGs that are identified in SEQ ID NOs 30 to 66, and optionally wherein the at least 30 CpGs comprise the CpGs identified in SEQ ID NOs 30 to 59; or
e. the panel of one or more CpGs may comprise at least the 37 CpGs that are identified in SEQ ID NOs 30 to 66; and optionally wherein the at least 37 CpGs comprise the CpGs identified in SEQ ID NOs 30 to 66.

In any of these aforementioned particular assays, wherein:
a) when the breast cancer index for the individual is about 0.15 or more the individual is classified as having breast cancer or as having a high risk of breast cancer development; or
b) when the breast cancer index for the individual is less than about 0.15 the individual is classified as not having breast cancer or as having a low risk of breast cancer development;
   preferably wherein the assay has a specificity of 80% or more, more preferably wherein the assay comprises determining methylation β-values for each CpG in the panel of one or more CpGs.

In any of these aforementioned particular assays, wherein:
a) when the breast cancer index for the individual is about 0.16 or more the individual is classified as having breast cancer or as having a high risk of breast cancer development; or
b) when the breast cancer index for the individual is less than about 0.16 the individual is classified as not having breast cancer or as having a low risk of breast cancer development;
   preferably wherein the assay has a specificity of 90% or more, more preferably wherein the assay comprises determining methylation β-values for each CpG in the panel of one or more CpGs.

In any of these aforementioned particular assays, wherein:
a) when the breast cancer index for the individual is about 0.19 or more the individual is classified as having breast cancer or as having a high risk of breast cancer development; or
b) when the breast cancer index for the individual is less than about 0.19 the individual is classified as not having breast cancer or as having a low risk of breast cancer development;
   preferably wherein the assay has a specificity of 95% or more, more preferably wherein the assay comprises determining methylation β-values for each CpG in the panel of one or more CpGs.

In any of these aforementioned particular assays, wherein:
a) the breast cancer index for the individual is about 0.23 to 0.40 or more the individual is assessed as having breast cancer or as having a high risk of breast cancer development; or
b) when the breast cancer index for the individual is about 0.12 or less the individual is classified as not having breast cancer,
and more preferably wherein the assay comprises determining methylation (3-values for each CpG in the panel of one or more CpGs.

In the assays described herein, wherein the determination of the methylation status of the one or more CpGs in the panel comprises determining methylation β-values for each CpG in the panel of one or more CpGs, preferably the sensitivity of the assay is at least 76% and the specificity of the assay is at least 91%.

In any of the assays described herein, the predicting of the presence, absence or development of breast cancer in an individual is may be based on based on breast cancer index threshold values that may stratify an individual. For example, depending on the breast cancer index value of an individual, the individual may be considered as not having breast cancer, as having breast cancer, or as at risk of having breast cancer or developing breast cancer. Thus, by utilising any panel of CpGs in accordance with the assays of the invention, stratifying an individual as being at risk of developing breast cancer may be in view of an individual having a breast cancer index value as being within a particular breast cancer index range. The further stratifying of an individual may particularly involve threshold PMR value being applied in order to stratify an individual as not having breast cancer, as having breast cancer, or as at risk of having breast cancer or developing breast cancer. In any of the assays described herein, wherein when the breast cancer index value of an individual is determined by PMR analysis the individual may be stratified as:
(i) not having breast cancer if their breast cancer index value is 3.5 or lower;
(ii) having breast cancer if their breast cancer index value is 8 or higher; or
(iii) at risk of having breast cancer or developing breast cancer if their breast cancer index value is greater than 3.5 but less than 8.

Any of the methods of treatment and/or methods of further screening described herein may be administered to the individual depending on their breast cancer index value.

In other assays of the invention, In any of the assays described herein, the predicting of the presence, absence or development of breast cancer in an individual is preferably based on the breast cancer index value. The predicting of the presence, absence or development of breast cancer in an individual may particularly involve methylation 3-value thresholds being applied in order to stratify an individual as having a high or low risk of having breast cancer or of breast cancer development. The individual assayed in accordance with the invention described herein may be further stratified as at risk of developing breast cancer. In any of the assays described herein wherein the breast cancer index value of an individual is determined by methylation (3-value and the individual is stratified as:
(i) not having breast cancer if their breast cancer index value is 0.1 or lower;
(ii) having breast cancer if their breast cancer index value is 0.3 or higher; or
(iii) at risk of having breast cancer or developing breast cancer if their breast cancer index value is greater than 0.1 but less than 0.3.

Any of the methods of treatment and/or methods of further screening described herein may be administered to the individual depending on their breast cancer index value.

Assays according to the present invention utilise a statistically robust panel of one or more CpGs whose methylation status can be determined to provide a reliable prediction of the presence or development of breast cancer in an individual. By determining the methylation status of each CpG within the panel of one or more CpGs, a breast cancer index value may be derived thus enabling stratification of individuals according to their risk of developing breast cancer or of having breast cancer with statistically robust sensitivity and specificity.

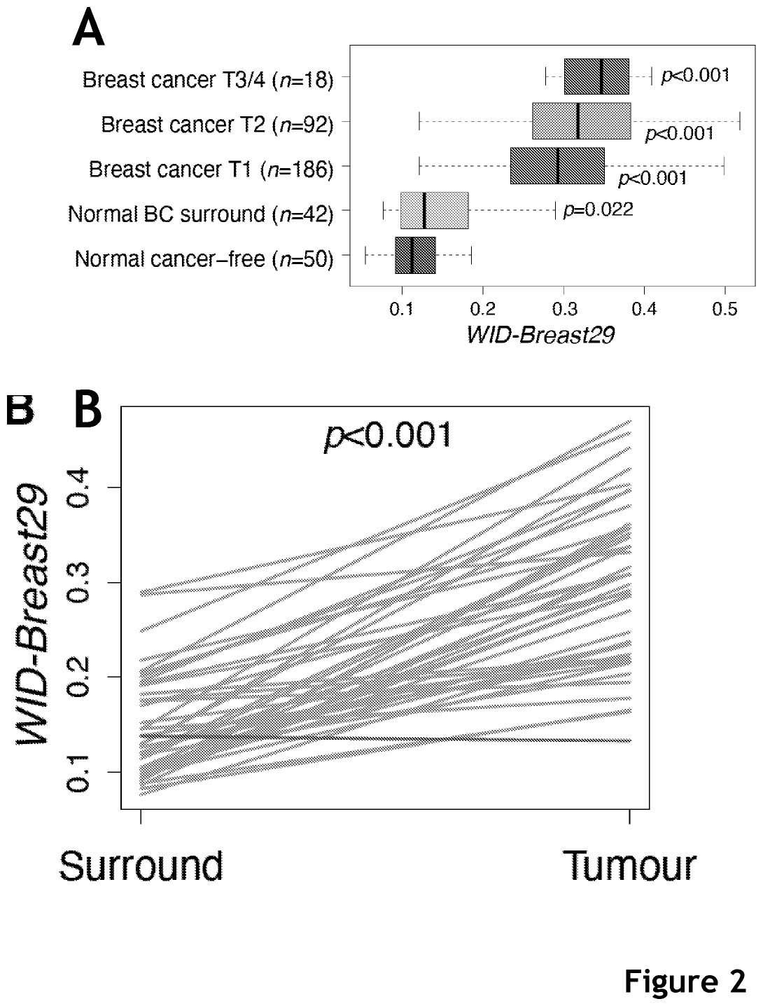

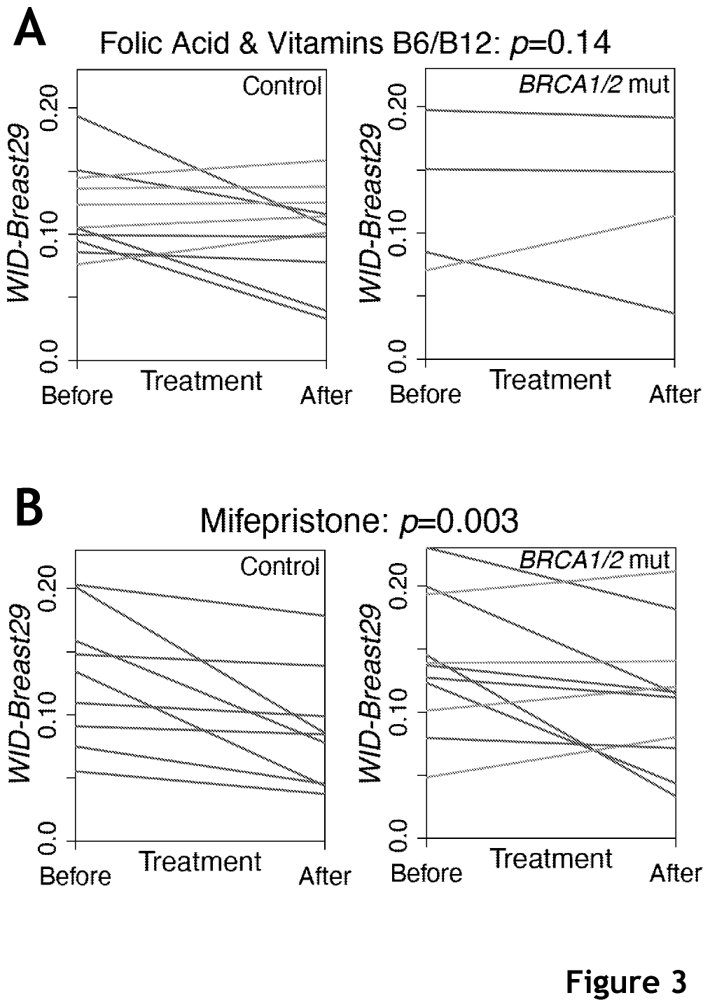

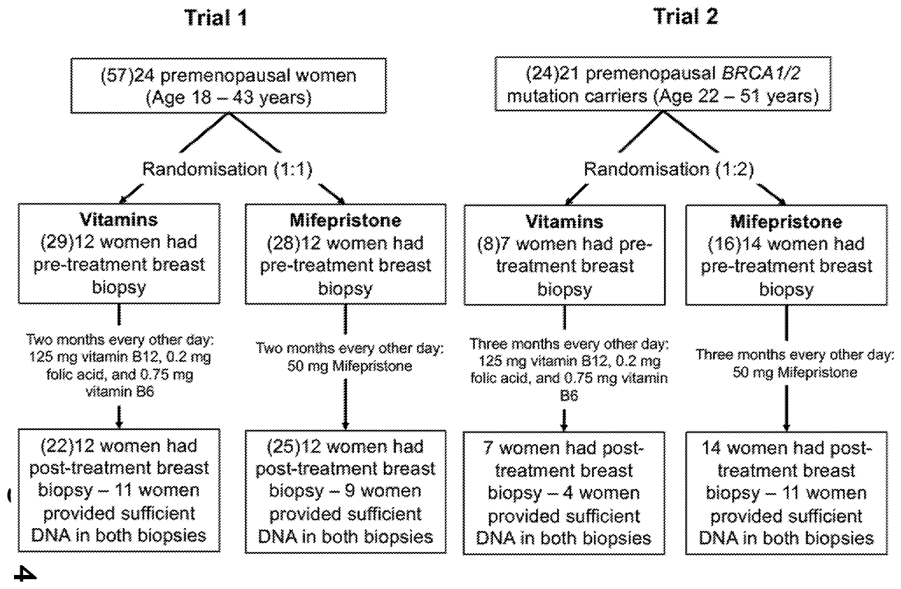

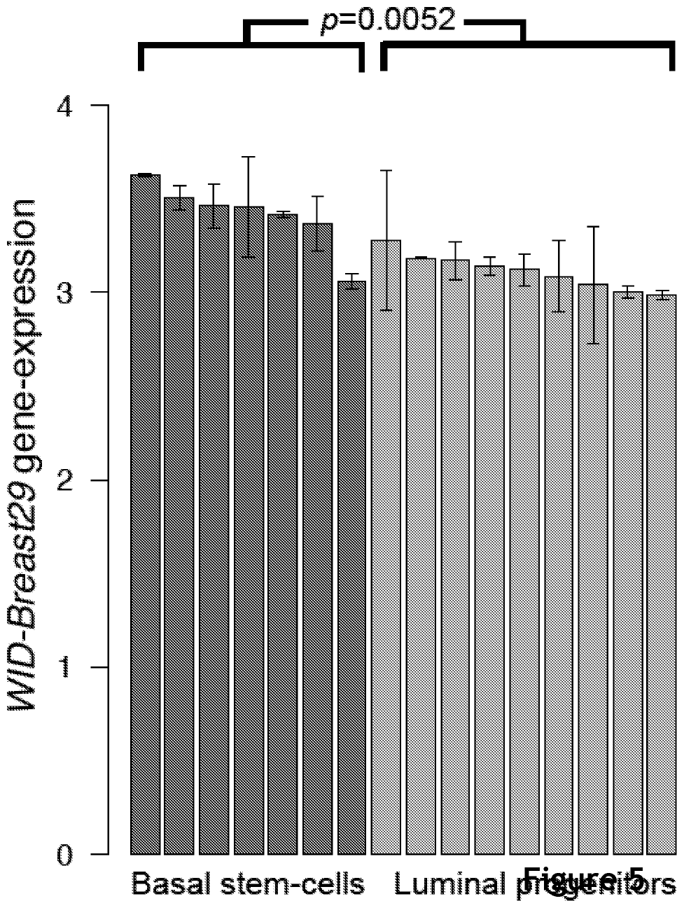

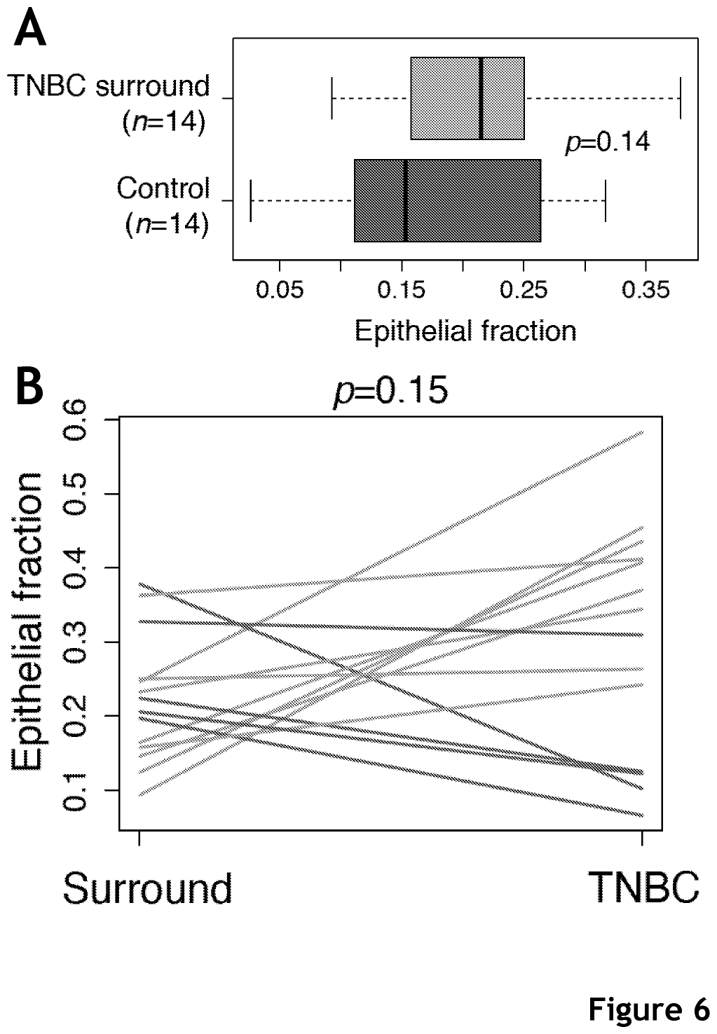

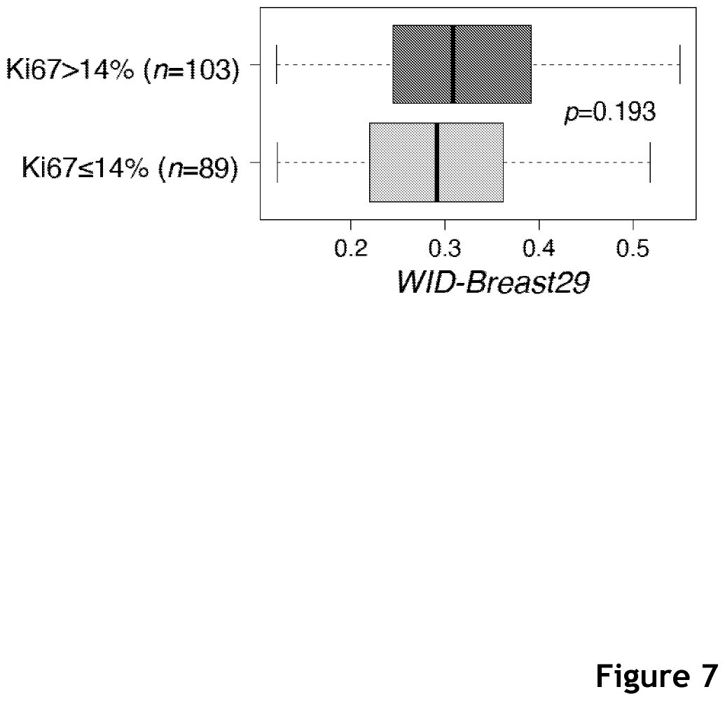

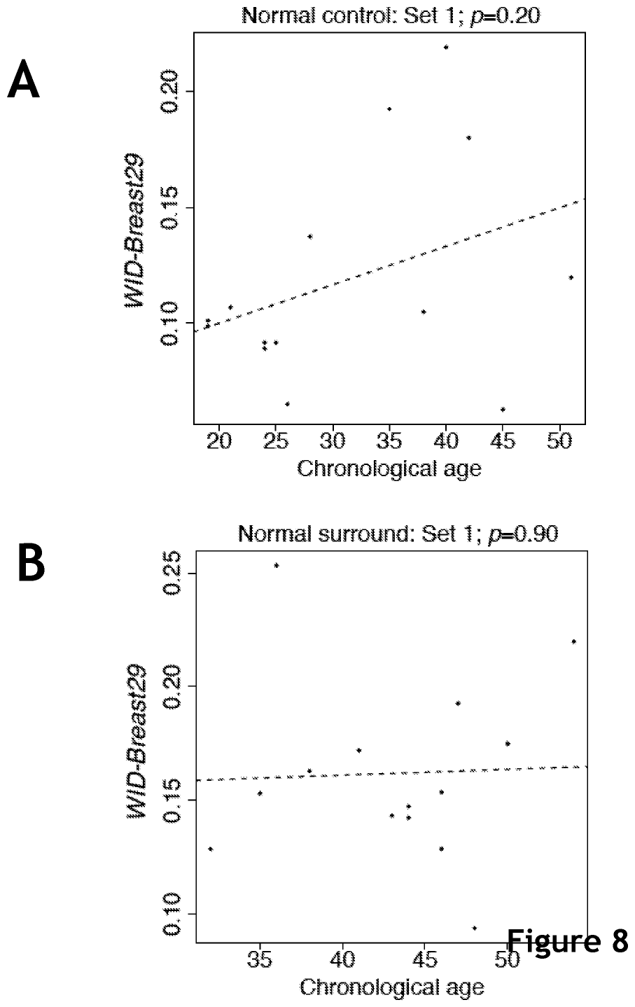

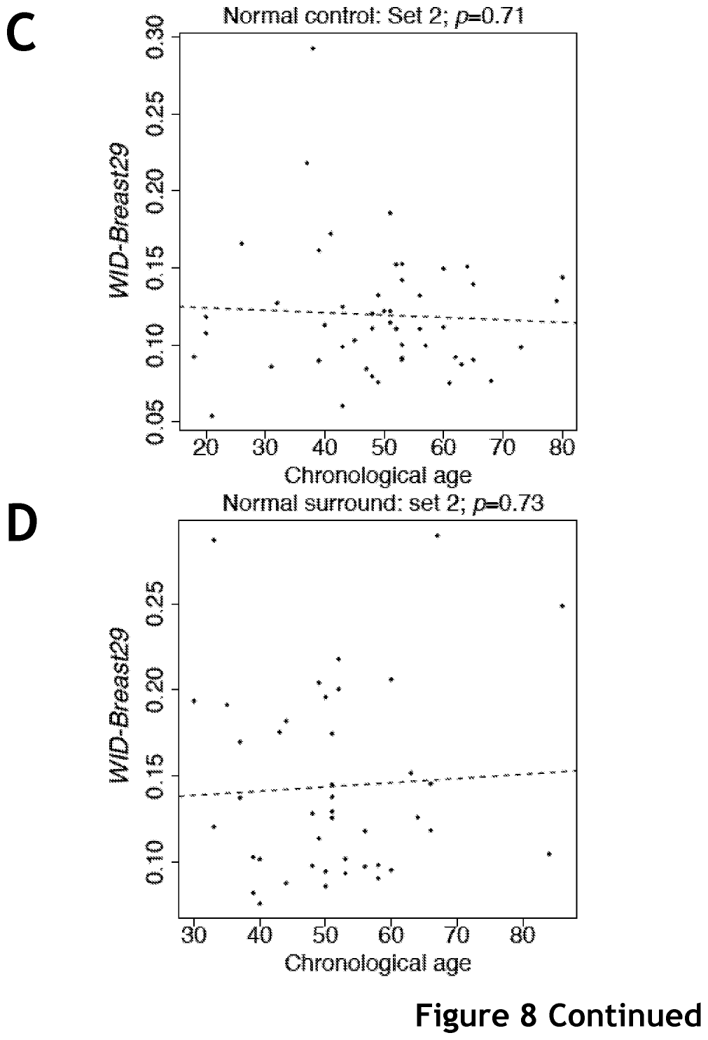

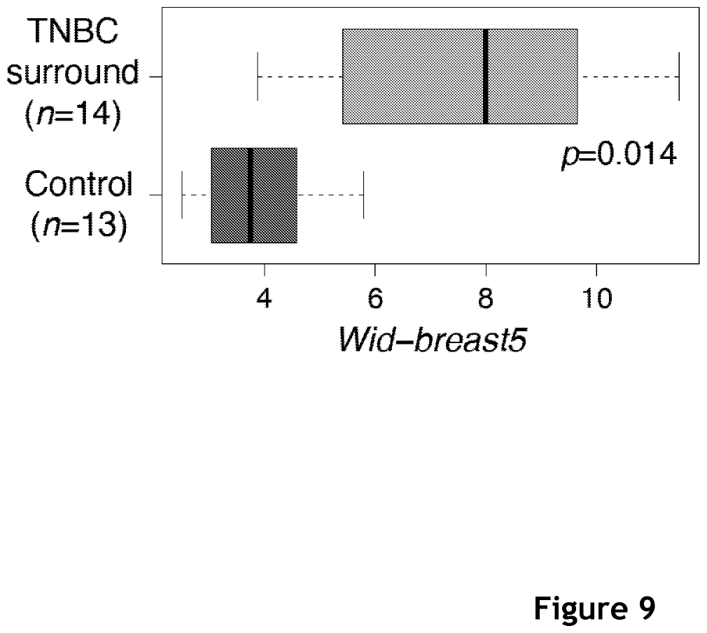

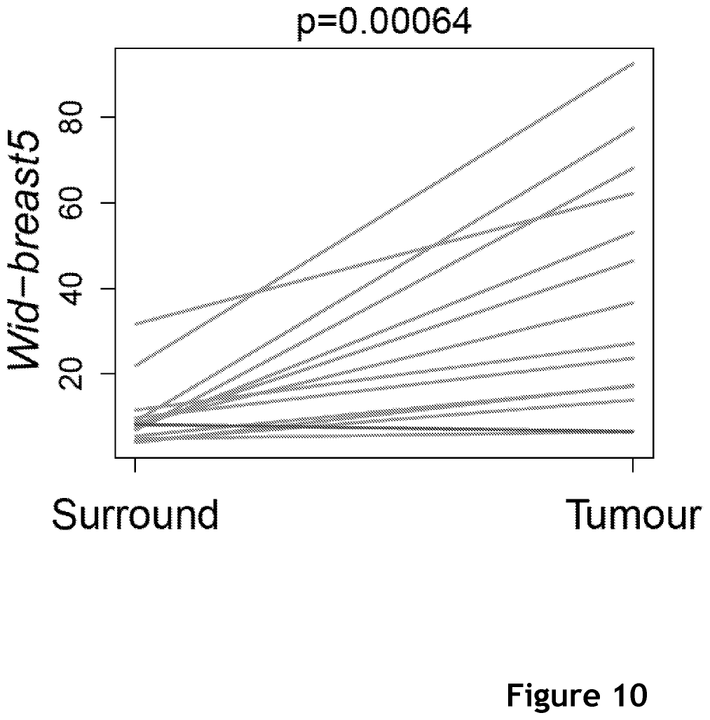

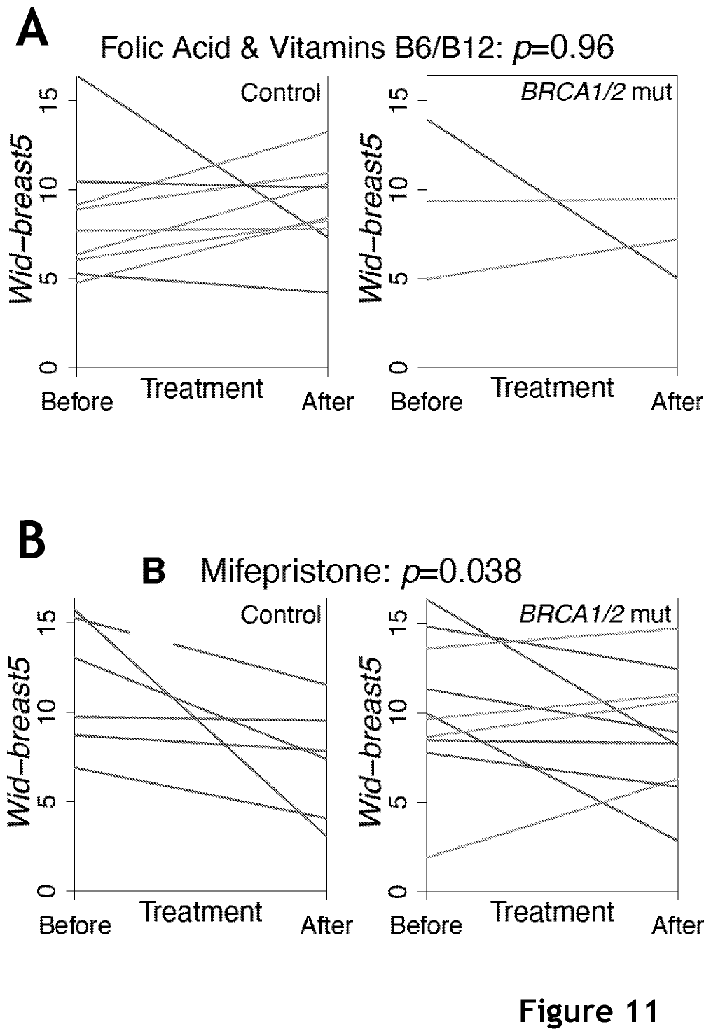

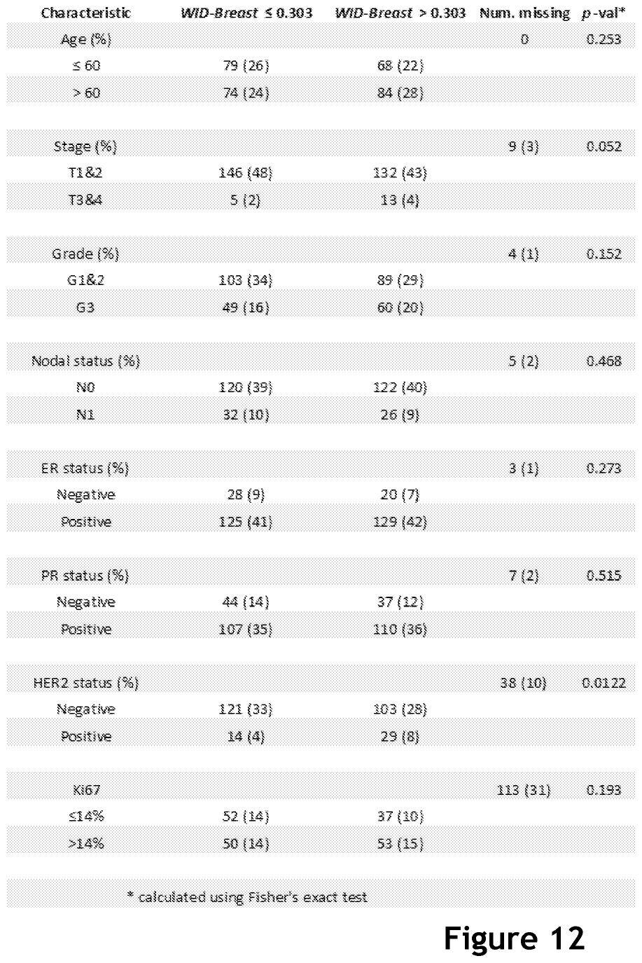

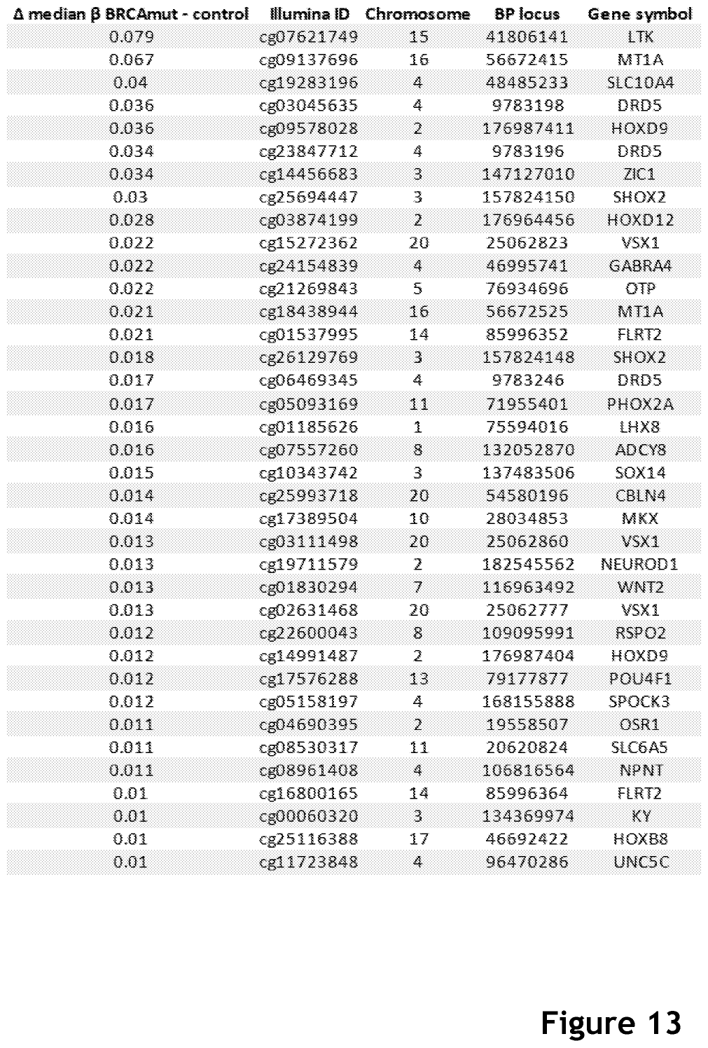

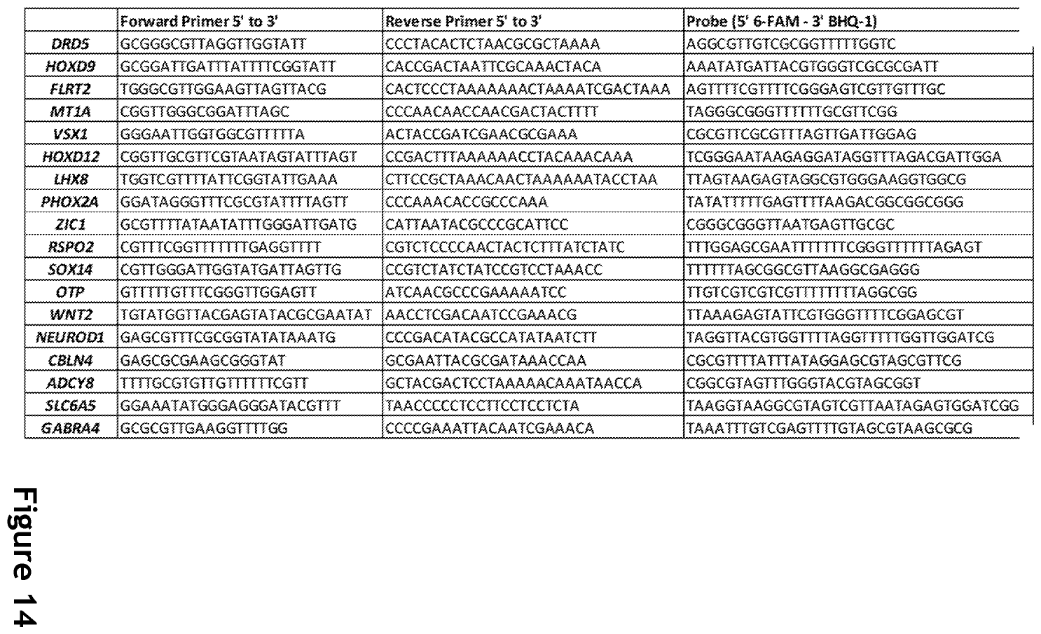

The breast cancer index thresholds for identifying individuals having breast cancer or at risk of developing breast cancer are provided via the patient data provided in the exemplary embodiments of the invention shown in the Examples (see particularly FIG. 2A, FIG. 9, and Tables 4 and 5).

In any of the assays described herein, the sensitivity and specificity of the breast cancer index threshold values vary depending on the number of CpGs comprised within the set, and specifically what CpGs are comprised within the set. Table 4 exemplifies this assertion, however the data of Table 4 is demonstrative that any combination of the CpGs found within the DMRs defined according to the invention remains capable of predicting the presence or development of breast cancer in an individual with a high degree of sensitivity and specificity.

### Relationship Between Breast Cancer Index Value and Determining Methylation Status of CpGs

In view of the observations described herein (see Examples), the inventors derived a breast cancer index based on an analysis of methylation status (DNAme; as described above) for use in assays for assessing the presence or development of breast cancer in an individual.

Any of the assays described herein involve deriving a breast cancer index value based on the methylation of status of a panel of one or more CpGs assayed in a sample provided from an individual, as described and defined herein.

The breast cancer index value may be derived by any suitable means.

The inventors have identified specific CpGs, as described and defined herein, which may be used to form a panel of CpGs whose methylation status is determined in order to establish breast cancer index values in accordance with the assays described and defined herein. Using these panels the inventors have demonstrated that it is possible to derive a breast cancer index value which correlates with and is indicative of normal breast tissue, i.e. tissue which is breast cancer negative. Accordingly, breast cancer can be assessed to be absent in the individual. Using these panels the inventors have demonstrated that it is possible to derive a breast cancer index value which correlates with and is indicative of breast cancer tissue, i.e. tissue which is breast cancer positive. Accordingly, breast cancer can be assessed to be present in the individual. As explained herein, the inventors have shown that using panels of the CpGs that have been identified it can be shown that the DNA methylation profile of breast tissue, as indicated by the breast cancer index value, is dynamic and subject to change on a continuum from breast cancer negative to breast cancer positive. As such, using panels of the CpGs that have been identified it is possible to establish a breast cancer index value scale that can be used to assess the presence, absence or development of breast cancer in an individual.

As described herein, the inventors have used certain methods for determining the methylation status of specific CpGs in the population of DNA molecules in the sample. For example, in one method a percent methylated reference (PMR) value of a CpG may be determined. In another method the methylation β-values of a CpG may be determined. Different mechanisms may be employed to determine specific values depending on the circumstances, such as PCR-based mechanisms or array-based mechanisms.

As will be apparent to a skilled person, in the assays of the invention the steps of determining the methylation status of specific CpGs in the population of DNA molecules in the sample are not limited to any one specific methodology. As the skilled person will appreciate, because the breast cancer index value is based on the methylation status of CpGs, and since the methylation status of CpGs can be represented by values which may be specific to a specific methodology, e.g. percent methylated reference (PMR) value or methylation β-value, then the range of breast cancer index values which define breast cancer negative and breast cancer positive samples may be dependent upon the methodology used to determine the methylation status of CpGs. Nevertheless, a user may readily reproduce and implement the assays of the invention using any suitable methodology for determining the methylation status of CpGs, provided that the same methodology is used consistently. Moreover, the user can readily establish, de novo, breast cancer index values which define breast cancer negative and breast cancer positive samples by determining the methylation status of CpGs in panels constituting the specific CpGs disclosed herein from known breast cancer negative and breast cancer positive patient samples. Once such breast cancer index values are established using the CpGs identified herein, a user may use these values as a basis for assessing the presence, absence or development of breast cancer in any test individual whose breast cancer status is to be determined. Accordingly, breast cancer index values according to the present invention are not limited to specific methods of determination of methylation status of CpGs. On the contrary, the skilled person will appreciate that breast cancer index values can be established which reflect the intrinsic capabilities of the CpGs identified herein to correlate methylation status with breast cancer disease status.

Accordingly, the breast cancer index value may be derived by assessing the methylation status of the one or more CpGs in the panel in a sample provided from an individual by any suitable means.

The step of determining the methylation status of each CpG in the panel of one or more CpGs may be achieved by determining a percent methylated reference (PMR) value of each one of the one or more CpGs. The step of determining the methylation status of each CpG in the panel of one or more CpGs may be achieved by determining the methylation β-value of each one of the one or more CpGs.

Preferably, the step of determining the methylation status of each CpG in the panel of one or more CpGs comprises:
(i) determining a percent methylated reference (PMR) value of each one of the one or more CpGs within any one of the DMRs of SEQ ID NOs 1 to 29, or within any combination of two more DMRs of SEQ ID NOs 1 to 29 in accordance with the invention; and/or
(ii) determining methylation β-values for each one of the CpGs in the panel.

Determining a PMR value for any given one or more CpGs within a sample to be assayed is a procedure well understood in the art. In the assays described herein, the skilled person may undertake a PMR analysis in any way deemed suitable. Likewise, determining methylation β-values for each one of any given CpG in a panel to be assayed is a procedure well understood in the art. In the assays described herein, the skilled person may undertake a methylation 3-value analysis in any way deemed suitable.

### Assessment of Methylation Status of CpGs

Methylation of DNA is a recognised form of epigenetic modification which has the capability of altering the expression of genes and other elements such as microRNAs. In cancer development and progression, methylation may have the effect of e.g. silencing tumor suppressor genes and/or increasing the expression of oncogenes. Other forms of dysregulation may occur as a result of methylation. Methylation of DNA occurs at discrete loci which are predominately dinucleotides consisting of a CpG motif, but may also occur at CHH motifs (where H is A, C, or T). During methylation, a methyl group is added to the fifth carbon of cytosine bases to create methylcytosine.

Methylation can occur throughout the genome and is not limited to regions with respect to an expressed sequence such as a gene. Methylation typically, but not always, occurs in a promoter or other regulatory region of an expressed sequence such as enhancer elements. Most typically, the methylation status of CpGs is clustered in CpG islands, for example CpG islands present in the regulatory regions of genes, especially in their promoter regions.

Typically, an assessment of DNA methylation status involves analysing the presence or absence of methyl groups in DNA, for example methyl groups on the 5 position of one or more cytosine nucleotides. Preferably, the methylation status of one or more cytosine nucleotides present as a CpG dinucleotide (where C stands for Cytosine, G for Guanine and p for the phosphate group linking the two) is assessed.

A variety of techniques are available for the identification and assessment of CpG methylation status, as will be outlined briefly below. The assays described herein encompass any suitable technique for the determination of CpG methylation status.

Methyl groups are lost from a starting DNA molecule during conventional in vitro handling steps such as PCR. To avoid this, techniques for the detection of methyl groups commonly involve the preliminary treatment of DNA prior to subsequent processing, in a way that preserves the methylation status information of the original DNA molecule. Such preliminary techniques involve three main categories of processing, i.e. bisulphite modification, restriction enzyme digestion and affinity-based analysis. Products of these techniques can then be coupled with sequencing or array-based platforms for subsequent identification or qualitative assessment of CpG methylation status.

Techniques involving bisulphite modification of DNA have become the most common assays for detection and assessment of methylation status of CpG dinucleotides. Treatment of DNA with bisulphite, e.g. sodium bisulphite, converts cytosine bases to uracil bases, but has no effect on 5-methylcytosines. Thus, the presence of a cytosine in bisulphite-treated DNA is indicative of the presence of a cytosine base which was previously methylated in the starting DNA molecule. Such cytosine bases can be detected by a variety of techniques. For example, primers specific for unmethylated versus methylated DNA can be generated and used for PCR-based identification of methylated CpG dinucleotides. DNA may be amplified, either before or after bisulphite conversion. A separation/capture step may be performed, e.g. using binding molecules such as complementary oligonucleotide sequences. Standard and next-generation DNA sequencing protocols can also be used.

In other approaches, methylation-sensitive enzymes can be employed which digest or cut only in the presence of methylated DNA. Analysis of resulting fragments is commonly carried out using mircroarrays.

Affinity-based techniques exploit binding interactions to capture fragments of methylated DNA for the purposes of enrichment. Binding molecules such as anti-5-methylcytosine antibodies are commonly employed prior to subsequent processing steps such as PCR and sequencing.

Olkhov-Mitsel and Bapat (2012) provide a comprehensive review of techniques available for the identification and assessment of biomarkers involving methylcytosine.

For the purposes of assessing the methylation status of the CpG-based biomarkers characterised and described herein, any suitable assay can be employed.

Assays described herein may comprise determining methylation status of CpGs by bisulphite converting the DNA. Preferred assays involve bisulphite treatment of DNA, including amplification of the identified CpG loci for methylation specific PCR and/or sequencing and/or assessment of the methylation status of target loci using methylation-discriminatory microarrays.

Amplification of CpG loci can be achieved by a variety of approaches. Preferably, CpG loci are amplified using PCR. A variety of PCR-based approaches may be used. For example, methylation-specific primers may be hybridized to DNA containing the CpG sequence of interest. Such primers may be designed to anneal to a sequence derived from either a methylated or non-methylated CpG locus. Following annealing, a PCR reaction is performed and the presence of a subsequent PCR product indicates the presence of an annealed CpG of identifiable sequence. In such assays, DNA is bisulphite converted prior to amplification. Such techniques are commonly referred to as methylation specific PCR (MSP)

In other techniques, PCR primers may anneal to the CpG sequence of interest independently of the methylation status, and further processing steps may be used to determine the status of the CpG. Assays are designed so that the CpG site(s) are located between primer annealing sites. This assay scheme is used in techniques such as bisulphite genomic sequencing, COBRA, Ms-SNuPE. In such assay, DNA can be bisulphite converted before or after amplification.

Small-scale PCR approaches may be used. Such approaches commonly involve mass partitioning of samples (e.g. digital PCR). These techniques offer robust accuracy and sensitivity in the context of a highly miniaturised system (pico-liter sized droplets), ideal for the subsequent handling of small quantities of DNA obtainable from the potentially small volume of cellular material present in biological samples, particularly urine samples. A variety of such small-scale PCR techniques are widely available. For example, microdroplet-based PCR instruments are available from a variety of suppliers, including RainDance Technologies, Inc. (Billerica, MA; http://raindancetech.com/) and Bio-Rad, Inc. (http://www.bio-rad.com/). Microarray platforms may also be used to carry out small-scale PCR. Such platforms may include microfluidic network-based arrays e.g. available from Fluidigm Corp. (www.fluidigm.com).

Following amplification of CpG loci, amplified PCR products may be coupled to subsequent analytical platforms in order to determine the methylation status of the CpGs of interest. For example, the PCR products may be directly sequenced to determine the presence or absence of a methylcytosine at the target CpG or analysed by array-based techniques.

Any suitable sequencing techniques may be employed to determine the sequence of target DNA. In the assays of the present invention the use of high-throughput, so-called “second generation”, “third generation” and “next generation” techniques to sequence bisulphite-treated DNA can be used.

In second generation techniques, large numbers of DNA molecules are sequenced in parallel. Typically, tens of thousands of molecules are anchored to a given location at high density and sequences are determined in a process dependent upon DNA synthesis. Reactions generally consist of successive reagent delivery and washing steps, e.g. to allow the incorporation of reversible labelled terminator bases, and scanning steps to determine the order of base incorporation. Array-based systems of this type are available commercially e.g. from Illumina, Inc. (San Diego, CA; http://www.illumina.com/).

Third generation techniques are typically defined by the absence of a requirement to halt the sequencing process between detection steps and can therefore be viewed as real-time systems. For example, the base-specific release of hydrogen ions, which occurs during the incorporation process, can be detected in the context of microwell systems (e.g. see the Ion Torrent system available from Life Technologies; http://www.lifetechnologies.com/). Similarly, in pyrosequencing the base-specific release of pyrophosphate (PPi) is detected and analysed. In nanopore technologies, DNA molecules are passed through or positioned next to nanopores, and the identities of individual bases are determined following movement of the DNA molecule relative to the nanopore. Systems of this type are available commercially e.g. from Oxford Nanopore (https://www.nanoporetech.com/). In an alternative assay, a DNA polymerase enzyme is confined in a “zero-mode waveguide” and the identity of incorporated bases are determined with florescence detection of gamma-labeled phosphonucleotides (see e.g. Pacific Biosciences; http://www.pacificbiosciences.com/).

In other assays sequencing steps may be omitted. For example, amplified PCR products may be applied directly to hybridization arrays based on the principle of the annealing of two complementary nucleic acid strands to form a double-stranded molecule. Hybridization arrays may be designed to include probes which are able to hybridize to amplification products of a CpG and allow discrimination between methylated and non-methylated loci. For example, probes may be designed which are able to selectively hybridize to an CpG locus containing thymine, indicating the generation of uracil following bisulphite conversion of an unmethylated cytosine in the starting template DNA. Conversely, probes may be designed which are able to selectively hybridize to a CpG locus containing cytosine, indicating the absence of uracil conversion following bisulphite treatment. This corresponds with a methylated CpG locus in the starting template DNA.

Following the application of a suitable detection system to the array, computer-based analytical techniques can be used to determine the methylation status of a CpG. Detection systems may include, e.g. the addition of fluorescent molecules following a methylation status-specific probe extension reaction. Such techniques allow CpG status determination without the specific need for the sequencing of CpG amplification products. Such array-based discriminatory probes may be termed methylation-specific probes.

Any suitable methylation-discriminatory microarrays may be employed to assess the methylation status of the CpGs described herein. One particular methylation-discriminatory microarray system is provided by Illumina, Inc. (San Diego, CA; http://www.illumina.com/). In particular, the Infinium MethylationEPIC BeadChip array and the Infinium HumanMethylation450 BeadChip array systems may be used to assess the methylation status of CpGs for predicting cancer development as described herein. Such a system exploits the chemical modifications made to DNA following bisulphite treatment of the starting DNA molecule. Briefly, the array comprises beads to which are coupled oligonucleotide probes specific for DNA sequences corresponding to the unmethylated form of a CpG, as well as separate beads to which are coupled oligonucleotide probes specific for DNA sequences corresponding to the methylated form of an CpG. Candidate DNA molecules are applied to the array and selectively hybridize, under appropriate conditions, to the oligonucleotide probe corresponding to the relevant epigenetic form. Thus, a DNA molecule derived from a CpG which was methylated in the corresponding genomic DNA will selectively attach to the bead comprising the methylation-specific oligonucleotide probe, but will fail to attach to the bead comprising the non-methylation-specific oligonucleotide probe. Single-base extension of only the hybridized probes incorporates a labeled ddNTP, which is subsequently stained with a fluorescence reagent and imaged. The methylation status of the CpG is determined by calculating the ratio of the fluorescent signal derived from the methylated and unmethylated sites.

Infinium HumanMethylation450 BeadChip array systems can be used to interrogate CpGs in the assays described herein. Alternative or customised arrays could, however, be employed to interrogate the cancer-specific CpG biomarkers defined herein, provided that they comprise means for interrogating all CpG for a given assay, as defined herein.

Techniques involving combinations of the above-described assays may also be used. For example, DNA containing CpG sequences of interest may be hybridized to microarrays and then subjected to DNA sequencing to determine the status of the CpG as described above.

In the assays described above, sequences corresponding to CpG loci may also be subjected to an enrichment process if desired. DNA containing CpG sequences of interest may be captured by binding molecules such as oligonucleotide probes complementary to the CpG target sequence of interest. Sequences corresponding to CpG loci may be captured before or after bisulphite conversion or before or after amplification. Probes may be designed to be complementary to bisulphite converted DNA. Captured DNA may then be subjected to further processing steps to determine the status of the CpG, such as DNA sequencing steps.

Capture/separation steps may be custom designed. Alternatively a variety of such techniques are available commercially, e.g. the SureSelect target enrichment system available from Agilent Technologies (http://www.agilent.com/home). In this system biotinylated “bait” or “probe” sequences (e.g. RNA) complementary to the DNA containing CpG sequences of interest are hybridized to sample nucleic acids. Streptavidin-coated magnetic beads are then used to capture sequences of interest hybridized to bait sequences. Unbound fractions are discarded. Bait sequences are then removed (e.g. by digestion of RNA) thus providing an enriched pool of CpG target sequences separated from non-CpG sequences. Template DNA may be subjected to bisulphite conversion and target loci amplified by small-scale PCR such as microdroplet PCR using primers which are independent of the methylation status of the CpG. Following amplification, samples may be subjected to a capture step to enrich for PCR products containing the target CpG, e.g. captured and purified using magnetic beads, as described above. Following capture, a standard PCR reaction is carried out to incorporate DNA sequencing barcodes into CpG-containing amplicons. PCR products are again purified and then subjected to DNA sequencing and analysis to determine the presence or absence of a methylcytosine at the target genomic CpG.

CpG biomarker loci defined herein by SEQ ID NOs 30 to 66 are identified e.g. by Illumina® identifiers (IlmnID) (see Table 2). These CpG loci identifiers refer to individual CpG sites used in the commercially available Illumina® Infinium Methylation EPIC BeadChip kit and Illumina® Infinium Human Methylation450 BeadChip kit. The identity of each CpG site represented by each CpG loci identifier is publicly available from the Illumina, Inc. website under reference to the CpG sites used in the Infinium Methylation EPIC BeadChip kit and the Infinium Human Methylation450 BeadChip kit.

To complement evolving public databases to provide accurate CpG loci identifiers and strand orientation, Illumina® has developed a method to consistently designate CpG loci based on the actual or contextual sequence of each individual CpG locus. To unambiguously refer to CpG loci in any species, Illumina® has developed a consistent and deterministic CpG loci database to ensure uniformity in the reporting of methylation data. The Illumina® method takes advantage of sequences flanking a CpG locus to generate a unique CpG locus cluster ID. This number is based on sequence information only and is unaffected by genome version. Illumina's standardized nomenclature also parallels the TOP/BOT strand nomenclature (which indicates the strand orientation) commonly used for single nucleotide polymorphism (SNP) designation.

Illumina® Identifiers for the Infinium MethylationEPIC BeadChip and Infinium Human Methylation450 BeadChip system are also available from public repositories such as Gene Expression Omnibus (GEO) (http://www.ncbi.nlm.nih.gov/geo/).

By assessing the methylation status of a CpG it is meant that a determination is made as to whether a given CpG is methylated or unmethylated. In addition, it is meant that a determination is made as to the degree to which a given CpG site is methylated across a population of CpG loci in a sample.

CpG methylation status may be measured indirectly using a detection system such as fluorescence. A methylation-discriminatory microarray may be used. When calculating the degree of methylation of a given CpG, the Illumina® definition of beta-values may be used. The Illumina® methylation beta-value of a specific CpG site is calculated from the intensity of the methylated (M) and unmethylated (U) alleles, as the ratio of fluorescent signals β=Max(M,0)/[Max(M,0)+Max(U,0)+100]. On this scale, 0<β<1, β values of 1 or close to 1 indicate 100% methylation whereas values of 0 or close to 0 indicate 0% methylation.

The methylation status of any one or more CpGs of the 37 CpGs defined according to SEQ ID NOS: 30 to 66 may be assessed by any suitable technique. As explained in more detail in the Examples below, one particular exemplary technique which the inventors have used is a methylation discriminatory array, such as an Illumina InfiniumMethylation EPIC BeadChip. These assays utilise probes directed to methylated and unmethylated CpGs at a given locus.

Another exemplary technique which the inventors have used to determine the methylation status of any one or more CpGs is a fluorescence-based PCR technique referred to as MethyLight. These assays utilise forward and reverse PCR primers specific for sequences encompassing any one or more CpGs within the 29 DMRs listed above. The methylation status of one or more of the 37 CpGs defined according to SEQ ID NOS: 30 to 66 may therefore be determined by MethyLight analysis. The assays also utilise detectable probes for specific regions within the 29 DMRs depending on the one or more CpGs that are to be assayed. The detectable probes are typically designed such that they hybridise only to methylated forms of the one or more CpGs to be assayed. Exemplary forward and reverse PCR primers and detectable probes that target regions within eighteen of the 29 DMRs are set out in Table 3 below.

### Bioinformatic Tools and Statistical Metrics for CpG-Based Assays

Software programs which aid in the in silico analysis of bisulphite converted DNA sequences and in primer design for the purposes of methylation-specific analyses are generally available and have been described previously.

In risk models for predicting cancer, a receiver-operating-characteristic (ROC) curve analysis is often used, in which the area under the curve (AUC) is assessed. Each point on the ROC curve shows the effect of a rule for turning a risk/likelihood estimate into a prediction of the presence, absence or development of cancer in an individual. The AUC measures how well the model discriminates between case subjects and control subjects. An ROC curve that corresponds to a random classification of case subjects and control subjects is a straight line with an AUC of 50%. An ROC curve that corresponds to perfect classification has an AUC of 100%.

In any of the methods described herein, the 95% confidence interval for the ROC AUC may be between 0.60 and 1.

In any of the methods described herein, the interval may be defined as a range having as an upper limit any number between 0.60 and 1. The upper limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99 or 1.00.

In any of the methods described herein, the interval may be defined as a range having as a lower limit any number between 0.60 and 1. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99 or 1.00.

In any of the methods described herein, the interval range may comprise any of the above lower limit numbers combined with any of the above upper limit numbers as appropriate.

Preferably, the upper limit number is 1. Thus, the 95% confidence ROC AUC interval may be defined as a range having an upper limit of 1 and as a lower limit any number between 0.60 and 1. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99 or 1.00.

The upper limit number may be 0.99. Thus, the 95% confidence ROC AUC interval may be defined as a range having an upper limit of 0.99 and as a lower limit any number between 0.60 and 0.99. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98 or 0.99.

The upper limit number may be 0.98. Thus, the 95% confidence ROC AUC interval may be defined as a range having an upper limit of 0.98 and as a lower limit any number between 0.60 and 0.98. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97 or 0.98.

The upper limit number may be 0.97. Thus, the 95% confidence ROC AUC interval may be defined as a range having an upper limit of 0.97 and as a lower limit any number between 0.60 and 0.97. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96 or 0.97.

The upper limit number may be 0.96. Thus, the 95% confidence ROC AUC interval may be defined as a range having an upper limit of 0.96 and as a lower limit any number between 0.60 and 0.96. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95 or 0.96.

The upper limit number may be 0.95. Thus, the 95% confidence ROC AUC interval may be defined as a range having an upper limit of 0.95 and as a lower limit any number between 0.60 and 0.95. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94 or 0.95.

The upper limit number may be 0.94. Thus, the 95% confidence ROC AUC interval may be defined as a range having an upper limit of 0.94 and as a lower limit any number between 0.60 and 0.94. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93 or 0.94.

The upper limit number may be 0.93. Thus, the 95% confidence ROC AUC interval may be defined as a range having an upper limit of 0.93 and as a lower limit any number between 0.60 and 0.93. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92 or 0.93.

The upper limit number may be 0.92. Thus, the 95% confidence ROC AUC interval may be defined as a range having an upper limit of 0.92 and as a lower limit any number between 0.60 and 0.92. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91 or 0.92.

The upper limit number may be 0.91. Thus, the 95% confidence ROC AUC interval may be defined as a range having an upper limit of 0.91 and as a lower limit any number between 0.60 and 0.91. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90 or 0.91.

The upper limit number may be 0.90. Thus, the 95% confidence ROC AUC interval may be defined as a range having an upper limit of 0.90 and as a lower limit any number between 0.60 and 0.90. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89 or 0.90.

The upper limit number may be 0.89. Thus, the 95% confidence ROC AUC interval may be defined as a range having an upper limit of 0.89 and as a lower limit any number between 0.60 and 0.89. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88 or 0.89.

The upper limit number may be 0.88. Thus, the 95% confidence ROC AUC interval may be defined as a range having an upper limit of 0.88 and as a lower limit any number between 0.60 and 0.88. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87 or 0.88.

The upper limit number may be 0.87. Thus, the 95% confidence ROC AUC interval may be defined as a range having an upper limit of 0.87 and as a lower limit any number between 0.60 and 0.87. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86 or 0.87.

The upper limit number may be 0.86. Thus, the 95% confidence ROC AUC interval may be defined as a range having an upper limit of 0.86 and as a lower limit any number between 0.60 and 0.86. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85 or 0.86.

The upper limit number may be 0.85. Thus, the 95% confidence ROC AUC interval may be defined as a range having an upper limit of 0.85 and as a lower limit any number between 0.60 and 0.85. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84 or 0.85.

The upper limit number may be 0.84. Thus, the 95% confidence ROC AUC interval may be defined as a range having an upper limit of 0.84 and as a lower limit any number between 0.60 and 0.84. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83 or 0.84.

The upper limit number may be 0.83. Thus, the 95% confidence ROC AUC interval may be defined as a range having an upper limit of 0.83 and as a lower limit any number between 0.60 and 0.83. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82 or 0.83.

The upper limit number may be 0.82. Thus, the 95% confidence ROC AUC interval may be defined as a range having an upper limit of 0.82 and as a lower limit any number between 0.60 and 0.82. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81 or 0.82.

The upper limit number may be 0.81. Thus, the 95% confidence ROC AUC interval may be defined as a range having an upper limit of 0.81 and as a lower limit any number between 0.60 and 0.81. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80 or 0.81.

The upper limit number may be 0.80. Thus, the 95% confidence ROC AUC interval may be defined as a range having an upper limit of 0.80 and as a lower limit any number between 0.60 and 0.80. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79 or 0.80.

The upper limit number may be 0.79. Thus, the 95% confidence ROC AUC interval may be defined as a range having an upper limit of 0.79 and as a lower limit any number between 0.60 and 0.79. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78 or 0.79.

The upper limit number may be 0.78. Thus, the 95% confidence ROC AUC interval may be defined as a range having an upper limit of 0.78 and as a lower limit any number between 0.60 and 0.78. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77 or 0.78.

The upper limit number may be 0.77. Thus, the 95% confidence ROC AUC interval may be defined as a range having an upper limit of 0.77 and as a lower limit any number between 0.60 and 0.77. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76 or 0.77.

The upper limit number may be 0.76. Thus, the 95% confidence ROC AUC interval may be defined as a range having an upper limit of 0.76 and as a lower limit any number between 0.60 and 0.76. The lower limit number may be 0.60, 0.61, 0.62, 0.63, 0.64, 0.65, 0.66, 0.67, 0.68, 0.69, 0.70, 0.71, 0.72, 0.73, 0.74, 0.75 or 0.76.

The upper limit number may be 0.75. Thus, the 95% confidence ROC AUC interval may be defined as a range having an upper limit of 0.75 and as a lower limit any number between 0.60 and 0.75. The lower limit number may be 0.60, 0.61, 0.62, 0.63, 0.64, 0.65, 0.66, 0.67, 0.68, 0.69, 0.70, 0.71, 0.72, 0.73, 0.74 or 0.75.

The upper limit number may be 0.74. Thus, the 95% confidence ROC AUC interval may be defined as a range having an upper limit of 0.74 and as a lower limit any number between 0.60 and 0.74. The lower limit number may be 0.60, 0.61, 0.62, 0.63, 0.64, 0.65, 0.66, 0.67, 0.68, 0.69, 0.70, 0.71, 0.72, 0.73 or 0.74.

The upper limit number may be 0.73. Thus, the 95% confidence ROC AUC interval may be defined as a range having an upper limit of 0.73 and as a lower limit any number between 0.60 and 0.73. The lower limit number may be 0.60, 0.61, 0.62, 0.63, 0.64, 0.65, 0.66, 0.67, 0.68, 0.69, 0.70, 0.71, 0.72 or 0.73.

The upper limit number may be 0.72. Thus, the 95% confidence ROC AUC interval may be defined as a range having an upper limit of 0.72 and as a lower limit any number between 0.60 and 0.72. The lower limit number may be 0.60, 0.61, 0.62, 0.63, 0.64, 0.65, 0.66, 0.67, 0.68, 0.69, 0.70, 0.71 or 0.72.

The upper limit number may be 0.71. Thus, the 95% confidence ROC AUC interval may be defined as a range having an upper limit of 0.71 and as a lower limit any number between 0.60 and 0.71. The lower limit number may be 0.60, 0.61, 0.62, 0.63, 0.64, 0.65, 0.66, 0.67, 0.68, 0.69, 0.70 or 0.71.

The upper limit number may be 0.70. Thus, the 95% confidence ROC AUC interval may be defined as a range having an upper limit of 0.70 and as a lower limit any number between 0.60 and 0.70. The lower limit number may be 0.60, 0.61, 0.62, 0.63, 0.64, 0.65, 0.66, 0.67, 0.68, 0.69 or 0.70.

### Methods of Treatment and Diagnosis

The invention also encompasses the performance of one or more treatment steps following a positive classification of breast cancer based on any of the methods described herein. Said treatments may be considered “therapeutic” treatments.

The invention also encompasses the performance of one or more treatment steps following a negative classification of breast cancer or prediction of an individual being at risk of breast cancer development based on any of the methods described herein. Said treatments may be considered “risk prevention”, “preventative” or “prophylactic” treatments.

The invention also encompasses the performance of one or more treatment steps following a negative classification of breast cancer or prediction of an individual being at risk of breast cancer development based on any of the methods described herein, in an individual that harbours one or more mutations that predispose the individual to an increased risk of developing breast cancer, such as a BRCA1 and/or a BRCA2 mutation.

The invention thus encompasses a method of treating a cancer patient comprising administering chemotherapy, radiation, immunotherapy or any cancer therapy described herein to the patient determined to have a cancer index value which indicates that the patient has is positive for cancer based on any of the assays described herein, preferably wherein the cancer is breast cancer.

The invention thus encompasses a method of treating and/or preventing breast cancer in an individual comprising:
a. determining the breast cancer status of an individual by assessing the presence, absence or development of breast cancer in an individual comprising any one of the assays described herein;
b. stratifying the individual according to their breast cancer status; and
c. administering one or more treatments to the individual based on their breast cancer status.

The invention thus encompasses a method of treating and/or preventing breast cancer in an individual comprising:
a. determining the breast cancer status of an individual by assessing the presence, absence or development of breast cancer in an individual comprising:
   i. providing a sample which has been taken from the individual, the sample comprising a population of DNA molecules;
   ii. determining in the population of DNA molecules in the sample the methylation status of a panel of one or more CpGs selected from within a panel of one or more Differentially Methylated Regions (DMRs) defined by SEQ ID NOs 1 to 29, wherein selected CpGs in each DMR are denoted by CG;
   iii. deriving a breast cancer index value (WID-Breast-Index) based on the methylation status of the one or more CpGs in the panel; and
   iv. predicting the presence or development of breast cancer in the individual based on the breast cancer index value;
b. stratifying the individual according to their breast cancer status; and
c. administering one or more treatments to the individual based on their breast cancer status.

The invention thus encompasses a method of treating and/or preventing breast cancer in an individual comprising:
a. determining the breast cancer status of an individual by assessing the presence, absence or development of breast cancer in an individual comprising:
   i. providing a sample which has been taken from the individual, the sample comprising a population of DNA molecules;
   ii. determining in the population of DNA molecules in the sample the methylation status of a panel of one or more CpGs selected from within a panel of one or more Differentially Methylated Regions (DMRs) defined by SEQ ID NOs 1 to 29, wherein selected CpGs in each DMR are denoted by CG, and wherein the step of determining the methylation status of a panel of one or more CpGs comprises determining a percent methylated reference value of each one of the one or more CpGs within any one of the DMRs of SEQ ID NOs 1 to 29, or within any combination of two or more DMRs of SEQ ID NOs 1 to 29;
   iii. deriving a breast cancer index value (WID-Breast-Index) based on the methylation status of the one or more CpGs in the panel; and
   iv. predicting the presence or development of breast cancer in the individual based on the breast cancer index value;
b. stratifying the individual according to their breast cancer status; and
c. administering one or more treatments to the individual based on their breast cancer status.

The invention thus encompasses a method of treating and/or preventing breast cancer in an individual comprising:
a. determining the breast cancer status of an individual by assessing the presence, absence or development of breast cancer in an individual comprising:
   i. providing a sample which has been taken from the individual, the sample comprising a population of DNA molecules;
   ii. determining in the population of DNA molecules in the sample the methylation status of a panel of one or more CpGs selected from within a panel of one or more Differentially Methylated Regions (DMRs) defined by SEQ ID NOs 1 to 29, wherein selected CpGs in each DMR are denoted by CG, and wherein the step of determining the methylation status of a panel of one or more CpGs comprises determining methylation β-values for each one of the CpGs in the panel;
   iii. deriving a breast cancer index value (WID-Breast-Index) based on the methylation status of the one or more CpGs in the panel; and
   iv. predicting the presence or development of breast cancer in the individual based on the breast cancer index value;
b. stratifying the individual according to their breast cancer status; and
c. administering one or more treatments to the individual based on their breast cancer status.

In any of the methods of treatment encompassed by the invention, the step of predicting the presence, absence or development of breast cancer in an individual may involve determining in DNA derived from cells in the sample the methylation status of CpGs in any panel of one or more CpGs selected from within one or more DMRs defined by SEQ ID NOs 1 to 29, wherein selected CpGs in each DMR are denoted by CG, in accordance the with the assays of the invention described herein.

In any of the methods of treatment encompassed by the invention, the step of predicting the presence or development of breast cancer in an individual may involve deriving a breast cancer index value.

In any of the methods of treatment encompassed by the invention, the step of predicting the presence or development of breast cancer in an individual may involve the use of any one of the arrays described herein.

In any of the methods of treatment encompassed by the invention, the step of stratifying the individual may involve applying any one of the thresholds according to any one of the assays of the invention described herein.

The step of administering one or more treatments may comprise different treatment steps depending on the stratification of an individual on the basis of their risk of having breast cancer or on the basis of risk of breast cancer development.

Particularly the amount of an invasiveness of the treatments administered may vary dependent on the stratification of an individual on the basis of their risk of having breast cancer or on the basis of their risk of breast cancer development. The treatments administered to the individual may comprise any treatments considered suitable by a person skilled in the art.

For example, wherein the individual is stratified as not having breast cancer or as having a low risk of breast cancer development, the individual may be subjected to treatment according to their risk, for example, preventative therapy and/or routine screening. Routine screening methods are known in the art. Routine screening may, for example, comprise one or more mammography screenings. Particularly, mammography screening may be performed about once every one, about once every two, about once every three, about once every four, or about once every five years. Preferably, mammography screening may be performed about once every three years. Preventative therapy may comprise antagonists of the progesterone and/or RANKL cell signalling pathways. Antagonists of these pathways are known in the art, and any suitable progesterone and/or RANKL cell signalling pathway antagonist may be used.

Preferably, the preventative therapy comprises administration of one or more doses of one or more selective progesterone receptor modulators (SPRMs) and/or RANKL inhibitors. Even more preferably, the one or more SPRMs comprises Mifeprestone.

Wherein the individual is stratified as having breast cancer or having a high risk of breast cancer development, the individual may be subjected to treatment according to their risk. Exemplary treatments are as follows:
a. intensified screening, optionally comprising one or more mammography scans and/or breast MRI scans;
b. administration of one or more of Mifeprestone, Aromatase inhibitors, Denosumab, selective estrogen receptor modulators (SERMs) and SPRMs; and/or
c. bilateral mastectomy.

Wherein the individual is stratified as high risk and the individual is subjected to intensified screening and/or administration of one or more suitable doses of one or more of Mifepristone, Aromatase inhibitors, Denosumab, “selective estrogen modulators” (SERMs) and “selective progesterone receptor modulators” (SPRMs). SERMs may include Anordin, Bazedoxifene, Broparestrol, Clomifene, Cyclofenil, Lasofoxifene, Ormeloxifene, Ospemifene, Raloxifene, Tamoxifen. Preferably, the SERMs include Tamoxifen, Bazedoxifene and Raloxifene. Preferably, the SPRMs include Mifepristone, Ulipristal, Asoprisnil, Proellex, Onapristone, Asoprisnil and Lonaprisan. The intensified screening may comprise one or more mammography scans and/or breast MRI scans. Any of the methods of treatment described herein, wherein the individual is stratified as “moderate” risk, the one or more treatments to the individual may function as ‘preventative’ treatments. Particularly, any one of the treatments described herein may be administered to an individual stratified as at least moderate risk as a measure of preventing manifestation of breast cancer in said individual.

Wherein the individual is stratified as high risk and the individual is subjected to intensified screening and/or administration of one or more suitable doses of one or more of Mifeprestone, Aromatase inhibitors, Denosumab, SERMS, SPRMs and/or bilateral mastectomy. SERMs may include Anordin, Bazedoxifene, Broparestrol, Clomifene, Cyclofenil, Lasofoxifene, Ormeloxifene, Ospemifene, Raloxifene, Tamoxifen. Preferably, the SERMs include Tamoxifen, Bazedoxifene and Raloxifene. Preferably, the SPRMs include Mifepristone, Ulipristal, Asoprisnil, Proellex, Onapristone, Asoprisnil and Lonaprisan.

Wherein the individual is stratified as being at risk of having breast cancer or developing breast cancer, the individual may be subjected to further screening methods as described herein. If the further screening methods result in a negative breast cancer diagnosis, one or more preventative therapies may be administered such as routine screening as described herein, and preventative therapies described herein. Particularly, preventative therapies include administration of one or more doses of one or more selective progesterone receptor modulators (SPRMs) and/or RANKL inhibitors, preferably wherein the SPRMs comprise mifepristone.

In any one of the methods of treatment described herein, the method may further comprise genetic and/or expression profiling of any panel of genes known in the art as being associated with breast cancer. For example, the methods described herein may further comprise genetic and/or expression profiling of any one or more of the genes comprised within the MammaPrint™ test (Cardoso et al, N Engl J Med, 2016; 375:717-729). For any panel of genes known in the art as being associated with breast cancer, the skilled person would be aware of what genetic and/or expression profiles would be considered to be abnormal. Furthermore, the skilled person would be aware of treatments in the art that are known to be efficacious with respect to specific abnormalities observed in profiling any panel of genes known in the art as being associated with breast cancer. For example, upon observing one or more mutations in one or both of the BRCA1 and BRCA2 genes the skilled person would consider administering platinum-based treatments to the individual.

Wherein the individual is predicted as not having breast cancer, the individual may be subjected to risk-prevention treatments. Particularly, for example, if the individual has one or more genetic mutations that predispose an individual to an increased risk of developing breast cancer, the individual may be subjected to risk-prevention treatments. Risk-prevention treatments may comprise any suitable treatment. For example, a risk prevention treatment may be administering one or more doses of mifepristone. In any of the methods described herein, the individual may not harbour breast cancer, but may harbour one or more genetic mutations that pre-dispose the individual to breast cancer such as one or more mutations in the BRCA genes. Other mutations may include any mutations in the art that are considered to pre-dispose individuals to breast cancer. In any of the methods of treatment described herein, the individual may not harbour breast cancer but may harbour one or more genetic mutations that pre-dispose the individual to breast cancer, and this individual may be subjected to any of the methods of monitoring described herein. For example, in any of the methods described herein, the individual does not harbour breast cancer and harbours one or more mutations that predispose the individual to an increased risk of developing breast cancer, and wherein one or more treatments administered to the individual comprises one or more doses of mifepristone.

Other exemplary treatments comprise one or more surgical procedures, one or more chemotherapeutic agents, one or more cytotoxic chemotherapeutic agents one or more radiotherapeutic agents, one or more immunotherapeutic agents, one or more biological therapeutics, one or more anti-hormonal treatments or any combination of the above following a positive diagnosis of cancer.

Cancer treatments may be administered to an individual having breast cancer or at risk of breast cancer development, in an amount sufficient to prevent, treat, cure, alleviate or partially arrest breast cancer or one or more of its symptoms. Such treatments may result in a decrease in severity, and/or decreased breast cancer index value, of breast cancer symptoms, or an increase in frequency or duration of symptom-free periods. A treatment amount adequate to accomplish this is defined as “therapeutically effective amount”. Effective amounts for a given purpose will depend on the severity of breast cancer and/or the individual's breast cancer index value as well as the weight and general state of the individual. As used herein, the term “individual” includes any human, preferably wherein the human is a woman. As used herein, “treatment” is to be considered synonymous with “therapeutic agent”.

The following therapeutic agents may be administered to an individual based on their breast cancer risk alone or in combination with any other treatment described herein. The therapeutic agent may be directly attached, for example by chemical conjugation, to an antibody. Methods of conjugating agents or labels to an antibody are known in the art. For example, carbodiimide conjugation (Bauminger & Wilchek (1980) Methods Enzymol. 70, 151-159) may be used to conjugate a variety of agents, including doxorubicin, to antibodies or peptides. The water-soluble carbodiimide, 1-ethyl-3-(3-dimethylaminopropyl) carbodiimide (EDC) is particularly useful for conjugating a functional moiety to a binding moiety. Other methods for conjugating a moiety to antibodies can also be used. For example, sodium periodate oxidation followed by reductive alkylation of appropriate reactants can be used, as can glutaraldehyde cross-linking. However, it is recognised that, regardless of which method of producing a conjugate of the invention is selected, a determination must be made that the antibody maintains its targeting ability and that the functional moiety maintains its relevant function.

A cytotoxic moiety may be directly and/or indirectly cytotoxic. By “directly cytotoxic” it is meant that the moiety is one which on its own is cytotoxic. By “indirectly cytotoxic” it is meant that the moiety is one which, although is not itself cytotoxic, can induce cytotoxicity, for example by its action on a further molecule or by further action on it. The cytotoxic moiety may be cytotoxic only when intracellular and is preferably not cytotoxic when extracellular.

Cytotoxic chemotherapeutic agents are well known in the art. Cytotoxic chemotherapeutic agents, such as anticancer agents, include: alkylating agents including nitrogen mustards such as mechlorethamine (HN2), cyclophosphamide, ifosfamide, melphalan (L-sarcolysin) and chlorambucil; ethylenimines and methylmelamines such as hexamethylmelamine, thiotepa; alkyl sulphonates such as busulfan; nitrosoureas such as carmustine (BCNU), lomustine (CCNU), semustine (methyl-CCNU) and streptozocin (streptozotocin); and triazenes such as decarbazine (DTIC; dimethyltriazenoimidazole-carboxamide); Antimetabolites including folic acid analogues such as methotrexate (amethopterin); pyrimidine analogues such as fluorouracil (5-fluorouracil; 5-FU), floxuridine (fluorodeoxyuridine; FUdR) and cytarabine (cytosine arabinoside); and purine analogues and related inhibitors such as mercaptopurine (6-mercaptopurine; 6-MP), thioguanine (6-thioguanine; TG) and pentostatin (2′-deoxycoformycin). Natural Products including vinca alkaloids such as vinblastine (VLB) and vincristine; epipodophyllotoxins such as etoposide and teniposide; antibiotics such as dactinomycin (actinomycin D), daunorubicin (daunomycin; rubidomycin), doxorubicin, bleomycin, plicamycin (mithramycin) and mitomycin (mitomycin C); enzymes such as L-asparaginase; and biological response modifiers such as interferon alphenomes. Miscellaneous agents including platinum coordination complexes such as cisplatin (cis-DDP) and carboplatin; anthracenedione such as mitoxantrone and anthracycline; substituted urea such as hydroxyurea; methyl hydrazine derivative such as procarbazine (N-methylhydrazine, MIH); and adrenocortical suppressant such as mitotane (o,p′-DDD) and aminoglutethimide; taxol and analogues/derivatives; and hormone agonists/antagonists such as flutamide and tamoxifen.

A cytotoxic chemotherapeutic agent may be a cytotoxic peptide or polypeptide moiety which leads to cell death. Cytotoxic peptide and polypeptide moieties are well known in the art and include, for example, ricin, abrin, Pseudomonas exotoxin, tissue factor and the like. Methods for linking them to targeting moieties such as antibodies are also known in the art. Other ribosome inactivating proteins are described as cytotoxic agents in WO 96/06641. Pseudomonas exotoxin may also be used as the cytotoxic polypeptide. Certain cytokines, such as TNFα and IL-2, may also be useful as cytotoxic agents.

Certain radioactive atoms may also be cytotoxic if delivered in sufficient doses. Radiotherapeutic agents may comprise a radioactive atom which, in use, delivers a sufficient quantity of radioactivity to the target site so as to be cytotoxic. Suitable radioactive atoms include phosphorus-32, iodine-125, iodine-131, indium-111, rhenium-186, rhenium-188 or yttrium-90, or any other isotope which emits enough energy to destroy neighbouring cells, organelles or nucleic acid. Preferably, the isotopes and density of radioactive atoms in the agents of the invention are such that a dose of more than 4000 cGy (preferably at least 6000, 8000 or 10000 cGy) is delivered to the target site and, preferably, to the cells at the target site and their organelles, particularly the nucleus.

The radioactive atom may be attached to an antibody, antigen-binding fragment, variant, fusion or derivative thereof in known ways. For example, EDTA or another chelating agent may be attached to the binding moiety and used to attach 111In or 90Y. Tyrosine residues may be directly labelled with 1251 or 131I.

A cytotoxic chemotherapeutic agent may be a suitable indirectly-cytotoxic polypeptide. In a particularly preferred embodiment, the indirectly cytotoxic polypeptide is a polypeptide which has enzymatic activity and can convert a non-toxic and/or relatively non-toxic prodrug into a cytotoxic drug. With antibodies, this type of system is often referred to as ADEPT (Antibody-Directed Enzyme Prodrug Therapy). The system requires that the antibody locates the enzymatic portion to the desired site in the body of the patient and after allowing time for the enzyme to localise at the site, administering a prodrug which is a substrate for the enzyme, the end product of the catalysis being a cytotoxic compound. The object of the approach is to maximise the concentration of drug at the desired site and to minimise the concentration of drug in normal tissues. In a preferred embodiment, the cytotoxic moiety is capable of converting a non-cytotoxic prodrug into a cytotoxic drug.

In any of the methods of treatment described herein, the one or more treatments that the individual is subjected to may be repeated on one or more occasions. The one or more treatments may be repeated at regular intervals. The repetitive nature of the treatment administration may depend on the particular treatment being administered. Some treatments may require repetitive administration at greater frequency than others. The skilled person would be aware of the frequency of administration required for therapies known in the art. The one or more treatments may be repeated weekly, two weekly, three weekly, four weekly, monthly, three monthly, six monthly, yearly, two yearly, three yearly, four yearly, or five yearly.

### Methods of Monitoring

The invention also provides methods of monitoring the risk of the presence or development of breast cancer in an individual.

“Monitoring” in the context of the present invention may refer to longitudinal assessment of an individual's risk of having breast cancer or risk of breast cancer development. This longitudinal assessment may be carried out according to the assays of the invention described herein. This longitudinal assessment may involve performance of the assays of the invention described herein to predict the presence or development of breast cancer in an individual at more than one time point over the course of an undetermined time window. The time window may be any period of time whilst the individual is still living. The time window may persist for the lifetime of the individual. The time window may persist until the individual's risk of having breast cancer or risk of breast cancer development falls below a certain level. The level may be a particular breast cancer index value e.g. a breast cancer index value.

The invention thus encompasses a method of monitoring for the presence, absence or development of breast cancer in an individual, the method comprising:
a. assessing the presence or absence of breast cancer in an individual or assessing breast cancer development in an individual to establish a breast cancer status for the individual by performing any one of the assays of the invention described herein at a first time point;
b. assessing the presence or absence of breast cancer in the individual or assessing breast cancer development in the individual to establish a breast cancer status for the individual by performing any one of the assays of the invention described herein at one or more further time points, preferably wherein the breast cancer status of the individual in steps a and b are assessed using the same assay; and
c. monitoring any change in the breast cancer status of the individual by observing the individual's breast cancer index between time points.

The invention also encompasses a method of monitoring for the presence, absence or development of breast cancer in an individual, the method comprising:
a. assessing the presence or absence of breast cancer in an individual or assessing breast cancer development in an individual to establish a breast cancer status for the individual by performing an assay at a first time point, comprising:
   i. providing a sample which has been taken from the individual, the sample comprising a population of DNA molecules;
   ii. determining in the population of DNA molecules in the sample the methylation status of a panel of one or more CpGs selected from within a panel of one or more Differentially Methylated Regions (DMRs) defined by SEQ ID NOs 1 to 29, wherein selected CpGs in each DMR are denoted by CG;
   iii. deriving a breast cancer index value (WID-Breast-Index) based on the methylation status of the one or more CpGs in the panel; and
   iv. assessing the presence, absence or development of breast cancer in the individual based on the breast cancer index value;
b. assessing the presence or absence of breast cancer in the individual or assessing breast cancer development in the individual to establish a breast cancer status for the individual by performing the assay of steps a(i) to a(iv) or by performing any one of the assays of the invention described herein at one or more further time points; and
c. monitoring any change in the breast cancer status of the individual by observing the individual's breast cancer index between time points.

The invention also encompasses a method of monitoring for the presence, absence or development of breast cancer in an individual, the method comprising:
a. assessing the presence or absence of breast cancer in an individual or assessing breast cancer development in an individual to establish a breast cancer status for the individual by performing an assay at a first time point, comprising:
   i. providing a sample which has been taken from the individual, the sample comprising a population of DNA molecules;
   ii. determining in the population of DNA molecules in the sample the methylation status of a panel of one or more CpGs selected from within a panel of one or more Differentially Methylated Regions (DMRs) defined by SEQ ID NOs 1 to 29, wherein selected CpGs in each DMR are denoted by CG, and wherein the step of determining the methylation status of a panel of one or more CpGs comprises determining a percent methylated reference value of each one of the one or more CpGs within any one of the DMRs of SEQ ID NOs 1 to 29, or within any combination of two or more DMRs of SEQ ID NOs 1 to 29;
   iii. deriving a breast cancer index value (WID-Breast-Index) based on the methylation status of the one or more CpGs in the panel; and
   iv. assessing the presence, absence or development of breast cancer in the individual based on the breast cancer index value;
b. assessing the presence or absence of breast cancer in the individual or assessing breast cancer development in the individual to establish a breast cancer status for the individual by performing the assay of steps a(i) to a(iv) or by performing any one of the assays of the invention described herein at one or more further time points; and
c. monitoring any change in the breast cancer status of the individual by observing the individual's breast cancer index between time points.

The invention also encompasses a method of monitoring for the presence, absence or development of breast cancer in an individual, the method comprising:
a. assessing the presence or absence of breast cancer in an individual or assessing breast cancer development in an individual to establish a breast cancer status for the individual by performing an assay at a first time point, comprising:
   i. providing a sample which has been taken from the individual, the sample comprising a population of DNA molecules;
   ii. determining in the population of DNA molecules in the sample the methylation status of a panel of one or more CpGs selected from within a panel of one or more Differentially Methylated Regions (DMRs) defined by SEQ ID NOs 1 to 29, wherein selected CpGs in each DMR are denoted by CG, and wherein the step of determining the methylation status of a panel of one or more CpGs comprises determining methylation β-values for each one of the CpGs in the panel;
   iii. deriving a breast cancer index value (WID-Breast-Index) based on the methylation status of the one or more CpGs in the panel; and
   iv. assessing the presence, absence or development of breast cancer in the individual based on the breast cancer index value;
b. assessing the presence or absence of breast cancer in the individual or assessing breast cancer development in the individual to establish a breast cancer status for the individual by performing the assay of steps a(i) to a(iv) or by performing any one of the assays of the invention described herein at one or more further time points; and
c. monitoring any change in the breast cancer status of the individual by observing the individual's breast cancer index between time points.

In any of the methods of monitoring encompassed by the invention, the steps of predicting the presence, absence or development of breast cancer in an individual may involve determining in DNA derived in the sample the methylation status of CpGs in any panel of one or more CpGs selected from within one or more DMRs defined by SEQ ID NOs 1 to 29, wherein selected CpGs in each DMR are denoted by CG, in accordance the with the assays of the invention described herein.

In any of the methods of monitoring described herein, the steps of assessing the presence, absence or development of breast cancer in an individual based on a breast cancer index value may involve the application of threshold values. Threshold values can provide an indication of an individual's risk of having breast cancer or an individual's risk of breast cancer development. For example, breast cancer index values may indicate a high or low risk of harbouring or developing breast cancer. In any of the methods of monitoring encompassed by the invention, the step of predicting the presence, absence or development of breast cancer in an individual involves deriving a breast cancer index value.
individual involves deriving a cancer index value.

The invention further encompasses a method of measuring methylation in a patient at multiple time points comprising (a) assessing the presence, absence or development of cancer in an individual by performing any one of the assays of the invention described herein at a first time point; (b) assessing the presence, absence or development of cancer in the individual by performing any one of the assays of the invention described herein at one or more further time points, and (c) detecting differential methylation status between (a) and (b).

In any of the methods of monitoring described herein, the individual may already harbour breast cancer. The individual may not have breast cancer. The individual may not harbour breast cancer. The individual may not harbour breast cancer but may harbour one or more genetic mutations that predispose the individual to an increased risk of breast cancer development e.g. the individual may harbour one or more mutations in a BRCA gene. Other mutations may include any mutations in the art that are considered to pre-dispose individuals to breast cancer. In any of the methods of monitoring described herein, the individual may not harbour breast cancer but may harbour one or more genetic mutations that pre-dispose the individual to breast cancer, and this individual may be subjected to any of the methods of monitoring described herein in order to determine their risk of having breast cancer or of developing breast cancer. For example, in any of the methods described herein, the individual does not harbour breast cancer and harbours one or more mutations that predispose the individual to an increased risk of developing breast cancer, and wherein one or more treatments are administered to the individual in accordance with any of the methods of treatment described herein as a method of prophylaxis. In any of the methods described herein, the individual does not harbour breast cancer and harbours one or more mutations that predispose the individual to an increased risk of developing breast cancer, and wherein one or more treatments are administered to the individual in accordance with any of the methods of treatment described herein as a method of prophylaxis, and wherein the one or more treatments administered to the individual comprises one or more doses of SPRMs e.g. comprising one or more doses of mifepristone. Preferably the one or more prophylactic treatments administered to the individual comprises one or more doses of mifepristone.

In any of the methods of monitoring described herein, depending on the risk of the presence or development of breast cancer in the individual, one or more treatments are administered to the individual according to any one of the methods of treatment encompassed by the invention and described herein. Different treatments may be administered depending on the stratification of an individual on the basis of their risk of having breast cancer or on the basis of their risk of breast cancer development. The method may further comprise administration of one or more treatments according to the methods of treatment described herein.

The breast cancer index value may change between any two or more time points. For this reason, longitudinal monitoring of an individual's breast cancer index value could be of particular benefit to the assessment of, for example, breast cancer progression, prevention of recurrence of breast cancer, breast cancer treatment efficacy, or breast cancer efficacy.

In any of the methods of monitoring described herein, the one or more further time points may be any suitable time point. Preferably the one or more further time points may of suitable distance apart for sufficiently frequent screening in order to predict any particularly early onset cases of presence or development of breast cancer in an individual. Preferably the one or more further time points may be of suitable distance apart for assessing the efficacy of one or more treatments. Preferably the one or more further time points may be of suitable distance apart for predicting whether an individual remains free of cancer after a successful course of treatment. The one or more further time points may be about monthly, about two monthly, about three monthly, about four monthly, about five monthly, about six monthly, about seven monthly, about eight monthly, about nine monthly, about ten monthly, about eleven monthly, about yearly, about two yearly, or more than two yearly.

In any of the methods of monitoring described herein, changes may be made to the one or more treatments wherein a positive or negative responses to the one or more treatments are observed. Treatments may be changed in accordance with the methods of treatments described herein. Treatments may particularly be changed if the individual's risk stratification, based on their breast cancer index value, changes.

In any of the methods of monitoring encompassed by the invention, the step of predicting the presence or development of breast cancer in an individual may involve the use of any one of the arrays described herein.

### Biological Samples

The assays described herein are preferably performed on samples of tissue from the breast of the individual.

Preferably, any of the assays described herein for assessing the presence, absence or development of breast cancer in an individual comprises providing a sample which has been taken from the individual. Preferably the individual is a woman.

In any of the assays described herein, the assay may or may not encompass the step of obtaining the sample from the individual. In assays which do not encompass the step of obtaining the sample from the individual, a sample which has previously been obtained from the individual is provided.

The sample may be provided directly from the individual for analysis or may be derived from stored material, e.g. frozen, preserved, fixed or cryopreserved material.

In any of the assays described herein, the sample may be self-collected or collected by any suitable medical professional.

In any of the assays described herein, the sample from the individual may be a sample from the breast. In any of the assays described herein, the sample from the individual may be from non-cancerous breast tissue. In any of the assays described herein, the sample may be derived from breast tissue comprising epithelial cells.

Samples of biological material may include breast biopsy samples, breast solid tissue samples, breast aspirates such as nipple fluid aspirates and fine needle aspirates.

Any of the assays described herein, the sample may comprise cells. The sample may comprise genetic material such as DNA and/or RNA.

Any of the assays described herein may involve providing a biological sample from the patient as the source of patient DNA for methylation analysis.

Any of the assays described herein may involve obtaining patient DNA from a biological sample which has previously been obtained from the patient.

Any of the assays described herein may involve obtaining a biological sample from the patient as the source of patient DNA for methylation analysis. The sample may be self-collected or collected by any suitable medical professional. Procedures for obtaining a biological sample include biopsy.

Methods for sample isolation and for the subsequent extraction and isolation of DNA from such cell or tissue samples in preparation for assessing DNA methylation, are well known to those skilled in the art. In the context of the assays or methods described herein, the entirety of a sample may be used, or alternatively cells may be concentrated or cell types may be fractionated in order to only apply subsets of one or more cell types to the present assays or methods. Any suitable methods of concentration or fractionation may be used.

### Types of Cancers

The methods described herein may be applied to any breast cancer.

The breast cancer may be a primary breast cancer lesion. The breast cancer may be a secondary breast cancer lesion. The breast cancer may be a metastatic breast cancer lesion.

The breast cancer may be a ductal carcinoma in situ or an invasive ductal carcinoma such as tubular type invasive ductal carcinoma (IDC), medullary type IDC, mucinous type IDC, papillary type IDC or cribriform type IDC.

The breast cancer may be an invasive carcinoma such as a pleomorphic carcinoma, carcinoma with osteoclast giant cells, carcinoma with choriocarcinoma features, carcinoma with melanotic features. The invasive breast carcinoma may be an invasive lobular carcinoma, tubular carcinoma, invasive cribriform carcinoma, medullary carcinoma, mucinous carcinoma and other tumours with abundant mucin such as mucinous carcinoma, cystadenocarcinoma and columna cell mucinous carcinoma, signet ring cell carcinoma. The invasive breast carcinoma may be a neuroendocrine tumour such as solid neuroendocrine carcinoma (carcinoid of the breast), atypical acarcinoid tumour, small cell/oat cell carcinoma, large cell neuroendocrine carcinoma. The invasive breast carcinoma may be an invasive papillary carcinoma, invasive micropapillary carcinoma, apocrine carcinoma, metaplastic carcinomas such as pure epithelial metaplastic carcinomas including squamous cell carcinoma, adenocarcinoma with spindle cell metaplasia, adenosquamous carcinoma, mucoepidermoid carcinoma, mixed epithelial/mesenchymal metaplastic carcinomas, matrix-producing carcinoma, spindle cell carcinoma, carcinosarcoma, squamous cell carcinoma of mammary origin, metaplastic carcinoma with osteoclastic giant cells. The invasive breast carcinoma may be a lipid-rich carcinoma, secretory carcinoma, oncocytic carcinoma, adenoid cystic carcinoma, acinic cell carcinoma, glycogen-rich clear cell carcinoma, sebaceous carcinoma, inflammatory carcinoma, bilateral breast carcinoma.

The breast cancer may be a mesenchymal breast tumour. The mesenchymal tumour may include sarcoma. The mesenchymal breast tumour may be a hemangioma, angiomatosis, Hemangiopericytoma, Pseudoangiomatous stromal hyperplasia, Myofibroblastoma, Fibromatosis (aggressive), Inflammatory myofibroblastic tumor, Lipoma Angiolipoma, Granular cell tumour, Neurofibroma, Schwannoma, Angiosarcoma, Liposarcoma, Rhabdomyosarcoma, Osteosarcoma, Leiomyoma, Leiomyosarcoma.

The breast cancer may be a malignant lymphoma such as Non-Hodgkin lymphoma.

The breast cancer may be a metastatic tumour in which the primary lesion originated in a tissue other than the breast.

The breast cancer may be a precursor breast cancer lesion. The precursor breast cancer lesion may be a Lobular neoplasia, lobular carcinoma in situ, Intraductal proliferative lesions, Usual ductal hyperplasia, Flat epithelial hyperplasia, Atypical ductal hyperplasia, Ductal carcinoma in situ, Microinvasive carcinoma, Intraductal papillary neoplasms, Central papilloma, Peripheral papilloma, Atypical papilloma, Intraductal papillary carcinoma, Intracystic papillary carcinoma.

The breast cancer may be a myoepithelial breast cancer lesion. The myoepithelial breast cancer lesion be myoepitheliosis, adenomyoepithelial adenosis, adenomyoepithelioma, malignant myoepithelioma.

The breast cancer may be a fibroepithelial breast tumour. The fibroepithelial breast tumour may be a fibroadenoma, phyllodes tumour, periductal stromal sarcoma, mammary hamartoma.

The breast cancer may be Paget's disease of the nipple.

### Arrays and Kits

The invention also encompasses arrays capable of discriminating between methylated and non-methylated forms of CpGs as defined herein; the arrays may comprise oligonucleotide probes specific for methylated forms of CpGs as defined herein and oligonucleotide probes specific for non-methylated forms of CpGs as defined herein. In any of the arrays described herein, the array may comprise oligonucleotide probes specific for a methylated form of each CpG in a CpG panel and oligonucleotide probes specific for a non-methylated form of each CpG in the panel; wherein the panel consists of at least 5 CpGs selected from the CpGs identified within the DMRs defined by SEQ ID NOs 1 to 29 and denoted by CG.

The panel may consist of at least 10 CpGs selected from the CpGs identified within the DMRs defined by SEQ ID NOs 1 to 29 and denoted by CG, preferably wherein the CpGs are identified within the DMRs defined by SEQ ID NOs 1 to 18 and denoted by CG.

The panel may consist of at least 20 CpGs selected from the CpGs identified within the DMRs defined by SEQ ID NOs 1 to 29 and denoted by CG, preferably wherein the CpGs are identified within the DMRs defined by SEQ ID NOs 1 to 18 and denoted by CG.

The panel may consist of at least 50 CpGs selected from the CpGs identified within the DMRs defined by SEQ ID NOs 1 to 29 and denoted by CG, preferably wherein the CpGs are identified within the DMRs defined by SEQ ID NOs 1 to 18 and denoted by CG.

The panel may consist of all of the CpGs identified in SEQ ID NOs 67 to 84.

The panel may consist of all of the CpGs identified in SEQ ID NOs 1 to 18 and denoted by CG.

In some embodiments the array is not an Infinium MethylationEPIC BeadChip array or an Illumina Infinium HumanMethylation450 BeadChip array.

Separately or additionally, in some embodiments the number of CpG-specific oligonucleotide probes of the array is 482,000 or less, 480,000 or less, 450,000 or less, 440,000 or less, 430,000 or less, 420,000 or less, 410,000 or less, or 400,000 or less, 375,000 or less, 350,000 or less, 325,000 or less, 300,000 or less, 275,000 or less, 250,000 or less, 225,000 or less, 200,000 or less, 175,000 or less, 150,000 or less, 125,000 or less, 100,000 or less, 75,000 or less, 50,000 or less, 45,000 or less, 40,000 or less, 35,000 or less, 30,000 or less, 25,000 or less, 20,000 or less, 15,000 or less, 10,000 or less, 5,000 or less, 4,000 or less, 3,000 or less or 2,000 or less.

The CpG panel may comprise any set of CpGs defined in the assays of the invention described herein.

The arrays of the invention may comprise one or more oligonucleotides comprising any set of CpGs defined in the assays of the invention, wherein the one or more oligonucleotides are hybridized to corresponding oligonucleotide probes of the array.

The invention also encompasses a process for making a hybridized array described herein, comprising contacting an array according to the present invention with a group of oligonucleotides comprising any set of CpGs defined in the assays of the invention.

Any of the arrays as defined herein may be comprised in a kit. The kit may comprise any array as defined herein together with instructions for use.

The invention further encompasses the use of any of the arrays as defined herein in any of the assays for determining the methylation status of CpGs for the purposes of predicting the presence or development of breast cancer in an individual.

The following Examples serve to illustrate but not to limit the invention.

### EXAMPLES

In the Examples described herein, WID-Breast or WID-Breast-Index is a breast cancer index value as described herein (WID=women's risk identification for breast cancer index).

In the Examples described herein, WID-Breast29 is a breast cancer index value where the index value has been determined by assaying the methylation status of all 37 CpGs defined by SEQ ID NOs: 30 to 66 that reside within 29 gene promoter regions in a given sample from an individual. The 29 gene promoter regions may be defined by SEQ ID NOs: 1 to 29.

WID-Breast5 is a breast cancer index value where the index value has been determined by assaying the methylation status of all of the CpGs within five sub-regions within defined gene promoter regions (Table 4; MTA1, HOXD12, LHX8, NEUROD1, CBLN4). The 5 sub regions are respectively defined by SEQ ID NOs 70, 72, 73, 80 and 81 (Table 3), each of which respectively reside within the gene promoter regions defined in the sequences set out in SEQ ID NOS: 4, 6, 7, 14 and 15 (Table 1).

These indices were assessed in healthy and cancerous breast tissue and subsequently tested its effectiveness as an intermediate surrogate end-point in clinical trials of Mifepristone and vitamins by comparing breast biopsies taken before and after the intervention.

### Materials and Methods

### Patients, Tissue Samples and Data-Sets

The inventors used three sets of samples:

Set 1: to establish and validate the WID-Breast29 test. Cancer-free normal breast-tissue samples from a control population who had no family or personal history of breast cancer (n=14, average age at cosmetic surgery 31 years) and from BRCA1/2 mutation carriers (n=14, average age at surgery 36 years); triple negative breast cancer (TNBC) biopsies and healthy breast tissue samples taken from sites surrounding the TNBC in the same individuals (n=14, average age at surgery 43 years).

Set 2: to independently validate the WID-Breast29 test. Cancer-free breast tissue samples (n=50, average age at surgery 49 years), and breast cancer samples (n=305, average age at surgery 60 years, of which 18 were stage T3/4, 109 were grade 3, 58 were node-positive, 254 were ER-positive, 217 were PR-positive, 43 were HER2-positive, and 103 were Ki67>14%); a subset of the breast cancer volunteers additionally provided adjacent healthy breast tissue samples (n=42, average age at surgery 51 years).

Set 3: to assess the performance of the WID-Breast29 test monitoring preventive measures in real time. The samples were obtained from two clinical trials (FIG. 4) as follows.

Trial 1 was “Mifepristone treatment prior to insertion of a levonorgestrel releasing intrauterine system for improved bleeding control—a randomised controlled trial” (1) (EudraCT number: 2009-009014-40; Regional ethical review board at Karolinska Institutet permit 2009/144-31/4). The primary objective was to assess the effect of mifepristone pre-treatment on the initial bleeding pattern after insertion of a levonorgestrel releasing intrauterine system. An included secondary objective was to study the effect of mifepristone on breast tissue.

Trial 2 was “The Effect of a Progesterone Receptor Modulator on Breast Tissue in Women with BRCA-1 and -2 Mutations” (ClinicalTrials.gov Identifier: NCT01898312; regional ethical review board at Karolinska Institutet permit 2012/729 31/1). The core objective of this ongoing study is to assess the safety and effect of treatment with mifepristone on epithelial cell proliferation in human breast tissue in women with BRCA-1 or -2 mutations prior to protective mastectomy.

Study subjects for both trials were healthy premenopausal women aged 18 and above with regular menstrual cycles lasting 25-35 days and with no contraindications to mifepristone. The main exclusion criteria were: use of any hormonal or intrauterine contraception and pregnancy or breastfeeding two months prior to the study; a history of breast cancer or other malignancies and adnexal abnormality upon transvaginal ultrasound examination. All women were instructed to use barrier contraceptive methods throughout the duration of the study. After signing an informed consent, study subjects in both trials were randomised 1:1 or 1:2 into the vitamin arm (Triobe®—used as a comparator in these studies) or mifepristone arm in Trial 1 and Trial 2, respectively. One group (i.e. 16 BRCA carriers and 28 controls) was treated with 50 mg mifepristone (one quarter of 200 mg Mifegyne®, Exelgyn, Paris, France) every other day for two months in Trial 1 (n=28) and three months in Trial 2 (n=16) starting on the first day of the menstrual cycle. As mifepristone is only available in 200 mg tablets in Sweden, a study nurse divided the tablets into 4 parts (each 50 mg) and instructed the study subjects to take one part every other day. The other group (i.e. 8 BRCA carriers and 29 controls) received Triobe® tablets which are visually identical to mifepristone and were also divided into 4 parts [one quarter of TrioBe® contains 0.125 mg cyanocobalamin (Vitamin B12), 0.2 mg folic acid (Vitamin B9), and 0.75 mg Pyridoxinhydrochlorid (Vitamin B6)]. Tablets were dispensed for two weeks at a time. Core needle aspiration biopsies were collected during the luteal phase before treatment and at the end of treatment during the luteal phase (vitamin treatment) or corresponding time of the cycle (mifepristone treatment). The biopsies were collected under ultrasound guidance from the upper outer quadrant of one breast using a 14 Gauge needle with an outer diameter of 2.2 mm. The end-of-treatment breast biopsy was taken from the same area. From Trial 1, 12 women from each of the vitamin and mifepristone groups were included in this study; of these, 11 and 9 women, respectively, had provided sufficient DNA from the pre- and post-treatment biopsies for subsequent processing and analysis. In Trial 2, 7/8 and 14/16 women in the vitamin and mifepristone groups, respectively, had provided sufficient pre-treatment biopsies, of which 4 and 11, respectively, later also provided a post-treatment sample with sufficient DNA yield (FIG. 4).

### DNA Methylation Analysis

DNA samples were normalized to a concentration of 25 ng/μl. The inventors bisulfite-modified 500 ng of tissue DNA using the DNA methylation Lightning Mag Prep Kit from Zymo Research (cat number D5047) on a Hamilton Star liquid handling platform. The inventors eluted 15 μl of the bisulfite converted DNA, which was then subjected to methylation analysis on the Illumina InfiniumMethylation EPIC BeadChip (Illumina, CA, USA) at UCL Genomics, according to the manufacturer's standard protocol. The resulting DNA methylation data were background-corrected and normalized using the BMIQ method 21. Probes were then removed if they had <95% coverage across samples, and any remaining probes with detection p-value<0.05 were replaced by k-NN imputation, with k=5.

### Methylight Analysis

Sodium bisulfite conversion of genomic DNA and the MethyLight assay were performed as previously described (A. Jones et al., Role of DNA methylation and epigenetic silencing of HAND2 in endometrial cancer development. PLoS Med 10, e1001551 (2013)). Briefly, primers sets and probes, designed specifically for bisulfite-converted DNA have been used: a methylated set for the genes of interest and a reference set, collagen 2A1 (COL2A1), to normalize for input DNA and account for bisulfite conversion rate. Specificity of the reactions for methylated DNA has been confirmed separately using SssI-treated human DNA. The percentage of fully methylated molecules at a specific locus has been calculated by dividing the GENE:COL2A1 ratio of a sample by the GENE:COL2A1 ratio of SssI-treated human white blood cell DNA and multiplied by 100. The abbreviation PMR (Percentage of Methylated Reference) indicates this measurement. A complete list of all MethyLight primers and probes is provided in FIG. 14 and Table 3.

### Breast Cell Gene-Expression Profiles

The 37 CpGs used for the WID-Breast29 index are located within 29 gene promoter sequences. When reference is made to the ‘WID-Breast29 genes’ in these Examples, reference is being made to the 29 genes downstream of the 37 WID-Breast29 index CpGs.

In order to assess the expression-levels of WID-Breast29 genes, the inventors studied gene expression profiles from (i) basal epithelial cells highly enriched for bipotent as well as myoepithelial clonogenic activity in vitro and (ii) luminal progenitors similarly enriched by cells with luminal clonogenic activity in vitro. For both these sets, microarray gene expression profiles (obtained using the Agilent SurePrint G3 array) are already publicly available (GEO accession number GSE37223, with quantile normalisation applied, hence the inventors did no further normalisation). The WID-Breast29 CpGs were represented by 29 genes on this array.

### Statistical Analysis

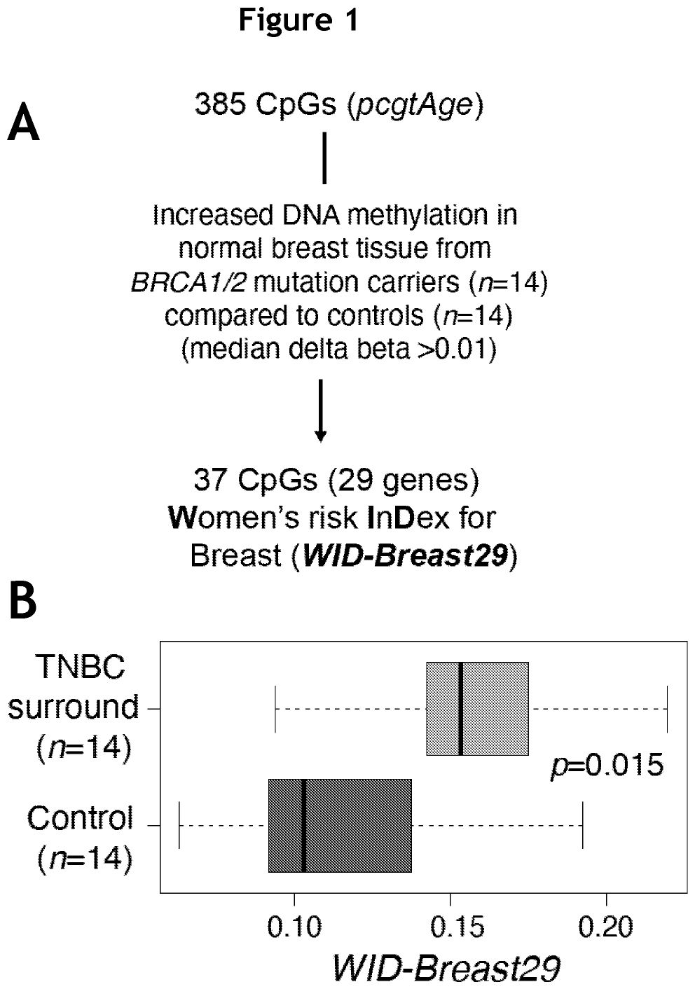

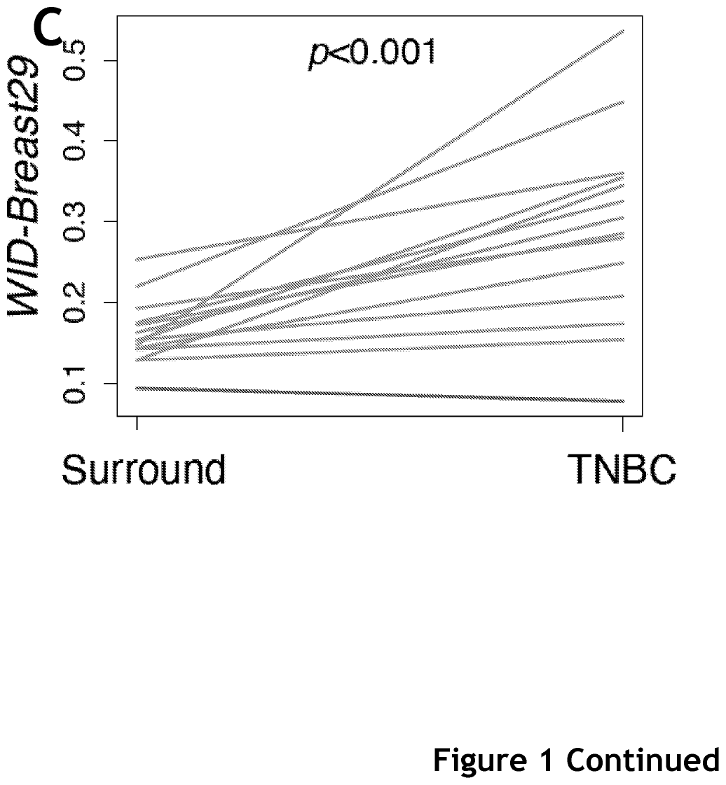

The WID-Breast29 was defined by selecting a subset of the 385 CpG loci (Z. Yang et al., Correlation of an epigenetic mitotic clock with cancer risk. Genome Biol 17, 205 (2016)) (a measure of ‘biological mitotic age’) (FIG. 1A). Specifically, the inventors selected those CpG loci which showed increasing methylation levels in normal breast tissue of BRCA1/2 mutation carriers (n=14) compared to normal breast tissue from controls (n=14). The inventors assessed this increase by the difference in median methylation levels of the samples in these groups for each CpG locus. Reasoning that CpG loci with only very small increases in median DNAme (DNA methylation) level are probably less informative, the inventors selected only CpGs with median delta beta >0.01, comparing BRCA1/2 mutation carriers with controls. The result was 37 CpG loci that passed this threshold (FIG. 13).

The WID-Breast29 breast cancer index is defined as the mean methylation level of these 37 CpGs in a sample.

The WID-Breast5 is defined as the mean methylation level (i.e. percent methylated reference) of CpGs identified within 5 methylight TaqMan probes, each targeting a region within the promotor sequences of the genes MTA1, HOXD12, LHX8, NEUROD1 and CBLN4 in a sample.

The main analyses (FIGS. 1-3) of Sets 1-3 were designed around the t-test (with n>10), as follows. Comparing triple negative breast cancer with the surrounding normal tissue (from the same women), the paired-sample t-test was used (FIG. 1; Sets 1-2). Comparing tumour or normal tissue surrounding the tumour with normal breast tissue from cancer-free women, the non-paired sample t-test was used (FIG. 2; Sets 1-2). Comparing samples from the same women before and after treatment, the paired-sample t-test was used (FIG. 3; Set 3). To compare the WID-Breast29 with various clinical factors, samples were divided into two groups partitioned by the median WID-Breast in the group, and Fisher's exact test was carried out on the resulting 2×2 contingency tables. All analyses were performed using the R software, version 3.6.

### Example 1

In order to establish the WID-Breast29 breast cancer index, the inventors selected 37 CpGs from the 385 pcgtAge (Z. Yang et al., Correlation of an epigenetic mitotic clock with cancer risk. Genome Biol 17, 205 (2016)) CpGs that showed higher methylation levels in the normal breast tissue of BRCA1/2 mutation carriers compared with cancer-free women (FIG. 1A). Assessing expression-levels of the 29 genes with promoter regions associated with the 37 loci of the WID-Breast29 CpGs showed that the expression level in luminal progenitor cells is significantly lower compared to basal epithelial cells (FIG. 5). These lower expression levels are expected to correspond to higher promoter methylation levels in luminal cells, supporting the view that the higher methylation levels of the WID-Breast29 CpGs in breast tissue records cell replication of luminal progenitor cells which are deemed to be the cells-of-origin for aggressive breast cancers.

To further assess whether the WID-Breast29 represents the epigenome of those cells within currently-normal breast tissue that eventually transforms to highly aggressive breast cancer, the inventors analysed TNBC tissue and the paired surrounding normal breast tissue (Set 1, Methods). The WID-Breast29 was significantly higher in the normal breast tissue surrounding TNBC compared with normal breast tissue from cancer-free healthy women (FIG. 1). In 94% (13/14) of the breast cancer patients, the WID-Breast29 was higher in the TNBC tissue compared to the paired normal surrounding breast tissue (FIG. 1C). Importantly, the proportion of epithelial cells did not significantly differ between normal breast from controls and women with TNBC or between normal surrounding and actual TNBC tissue (FIG. 6), again supporting the view that the WID-Breast29 is reflective of the specific cells-of-origin for breast cancer. These data therefore evidence that the assays of the invention are capable of predicting not only the presence or absence of breast cancer, but also development of breast cancer, wherein ‘development’ may refer to the progression/worsening of a pre-existing breast cancer in an individual or the increasing risk of occurrence of breast cancer in an individual which does not have breast cancer.

In order to assess whether the WID-Breast29 also behaves similarly in non-TNBC, the inventors analysed another set of normal breast tissue from cancer-free women (n=50) and normal breast tissue surrounding non-TNBC (n=42), as well as from a large range of non-TNBCs (n=305) (24) (Set 2, Methods). Again, the WID-Breast29 was substantially higher in normal breast tissue surrounding the cancerous non-TNBC breast tissue, and increased further in the actual cancerous tissue (FIG. 2A-B), thus suggesting that WID-Breast 29 is reflective of the cells-of-origin for breast cancer and that the WID-Breast29 may predict not only the presence or absence of breast cancer, but also the development of breast cancer. See also Table 5 for associated statistical metrics.

The WID-Breast29 tended to be higher in more advanced and HER2 positive breast cancers, but showed no association with grade, nodal involvement or hormone receptor status (FIG. 12). Interestingly, neither the actual proliferation rate in the breast cancer as assessed by Ki67 expression (FIG. 12, FIG. 7), nor the chronological age in the various sets of normal breast tissue (FIG. 8) were associated with the WID-Breast29 in these bulk-tissue samples.

In order to assess the potential of the WID-Breast29 as a surrogate measure of efficacy of preventive breast cancer therapeutic strategies, the inventors assessed the WID-Breast29 in breast biopsies from 15 and 20 women before and after 2-3 month treatment with either vitamins or mifepristone, respectively (Set 3, FIG. 3); each woman provided a breast biopsy before and after taking the study medication. Amongst the 15 women in the vitamin group, 9 (60%) exhibited a lower WID-Breast29 after treatment, which was non-significant (FIG. 3A). Amongst the 20 women in the mifepristone group, 16 (80%) exhibited a highly significant reduction in the WID-Breast29 (p=0.003; FIG. 3B). Importantly, whereas all 9 control volunteers (100%) showed a reduction of the WID-Breast29 after mifepristone treatment, this was not the case for 4/11 (36%) of the BRCA mutant volunteers, which might indicate an adoption of progesterone independence and a lack of response to mifepristone with a high likelihood of subsequent progression to cancer.

In this study, the inventors have developed the WID-Breast29 test based on 37 CpGs associated with 29 genes, and showed it is effective as a surrogate measure of cancer risk in normal breast tissue. The WID-Breast29 was defined based on mitotic age, and appears to be specifically aggravated in the normal breast tissue of as-yet cancer-free BRCA1/2 mutation carriers. Furthermore, the WID-Breast29 is elevated in the normal breast tissue surrounding cancers, and was found to score highest in actual breast cancer. These findings are complemented by the observation that several of the WID-Breast29 genes are linked to breast carcinogenesis (e.g. ZIC1, GABRA4, OTP, FLRT2, NEUROD1 and UNC5C), and that these (and other WID-Breast29) genes display lower expression in luminal progenitor cells (considered the cell-of origin of aggressive cancers). Taken together, these findings suggest that the WID-Breast29 can be used to determine the presence of breast cancer and can be used to assess the risk of breast cancer development through assaying normal breast tissue. Mifepristone is a synthetic progesterone receptor modulator acting as a progesterone receptor antagonist, and is currently in clinical use for pregnancy termination and as an emergency contraceptive. It is comprehensively studied in treatment of fibroids, endometriosis and as a long term contraceptive. Furthermore, mifepristone has been shown to exert an antiproliferative effect in breast tissue in healthy women and has also previously been shown to reduce mammary cancer development in mice. Likewise, antagonising RANKL (which is upregulated by progesterone) has shown impressive mammary cancer preventive effects in rodents. Denosumab is a fully humanised monoclonal antibody that binds RANKL and has been shown in three premenopausal volunteers to reduce breast epithelial proliferation. However, in postmenopausal breast cancer patients, denosumab does not appear to impact on the incidence of contralateral breast cancer. Given that progesterone (i) increases RANKL in the breast and (ii) is associated with low levels of the systemic RANKL-antagonist OPG, antagonising progesterone activity appears to be more potent than blocking RANKL activity alone.

Change in mammographic breast density is proven as a good predictor of response to tamoxifen in preventive studies. However, molecular markers (including the WID-Breast29) which are assessed directly in breast tissue have three essential advantages for premenopausal high risk women: (i) They can be measured frequently (e.g. every 2-3 months). (ii) The dynamic of the WID-Breast29 in individual volunteers reflects cancer presence and risk of development in real time and individual adjustments to preventive measures can be made ad hoc. (iii) They do not require repeated exposure to x-rays. Importantly, unlike markers which indicate proliferation (e.g. Ki-67), DNA-based markers (e.g. WID-Breast29) reflect the proportion of cells at risk in an organ and not just any cells which are proliferating at the time of the assessment.

The inventors' findings that (i) the WID-Breast29 increased in almost all cancers for which normal surrounding tissue was available (which is not the case for the epithelial cell fraction; FIG. 6) and (ii) there was no correlation between the proliferation marker Ki-67 and the WID-Breast29 (FIG. 7) support the view that the WID-Breast29 is a specific marker for those breast epithelial cells which are prone to neoplasmic transformation and breast cancer development.

### Example 2

In order to assess whether (i) a lower number of genes and (ii) an alternative technique to assess DNAme which also takes the methylation status of a larger number of neighbouring CpGs into account the inventors did the following: The 29 regions (see Table 1 detailing 600 basepairs of each region; SEQ ID NOs 1 to 29) on which the WID-Breast29 is based on were further narrowed down to 18 by considering two factors. Firstly, regions were ranked according to mean promoter methylation (calculated across all probes marked on the Illumina EPIC manifest as “UCSC_RefGene_Group” as “TSS200”). Only genes/regions where the mean promoter methylation showed an increase from normal breast (from cancer-free women) to surround normal breast tissue to triple negative breast cancer tissue were then considered. Secondly, a combination analysis of all 29 regions was conducted and regions with the highest proportion of representation in the top 1000 combinations were selected (only genes with significant (p<0.01) mean promoter methylation when comparing normal to surround and surround to triple negative breast cancer were considered). Methylight reactions were designed and real-time PCR conducted for the top 18 regions (FIG. 14). Following Methylight analysis, Percent Methylated Reference (PMR) was calculated and the inventors generated an index based on average PMR value over different combinations of genes and ranking based on AUC (only gene combination with AUC>0.9 comparing normal and surround were considered). The combinations which lead to a ROC AUC>0.9 when comparing normal breast tissue from cancer-free women with normal breast tissue surround breast cancer and a ROC AUC>0.8 when comparing normal breast tissue surround breast cancer with triple negative breast cancer are provided in Table 4 (in this Table the inventors also provide the specific PMR cutpoints of the WID-Breast index depending on the gene combination at a specificity of 80%, 90% and 95% and the resulting sensitivity). The highest ranking combination of 5 genes only (i.e. MTA1, HOXD12, LHX8, NEUROD1, CBLN4—marked in bold in the attached Table 4) was then selected for further analysis. The WID-Breast5 is defined as the mean methylation level (i.e. PMR) of a specific selection of 5 genes (MTA1, HOXD12, LHX8, NEUROD1, CBLN4) in a sample. FIG. 10 shows the difference of the WID-Breast5 between the normal breast tissue from cancer-free women and the normal tissue surrounding breast cancer.

In order to assess the potential of the WID-Breast5 as a surrogate measure of efficacy of preventive breast cancer therapeutic strategies, the inventors assessed the WID-Breast5 in breast biopsies from 12 and 16 women before and after 2-3 month treatment with either vitamins or mifepristone, respectively (Set 3, FIG. 11); each woman provided a breast biopsy before and after taking the study medication. Amongst the 12 women in the vitamin group, 4 (33%) exhibited a lower WID-Breast5 after treatment, which was non-significant (FIG. 3A). Amongst the 16 women in the mifepristone group, 12 (75%) exhibited a highly significant reduction in the WID-Breast5 (p=0.038; FIG. 11B). Importantly, whereas all 6 control volunteers (100%) showed a reduction of the WID-Breast5 after mifepristone treatment, this was not the case for 6/10 (60%) of the BRCA mutant volunteers, which might indicate an adoption of progesterone independence and a lack of response to mifepristone with a high likelihood of subsequent progression to cancer.

### Discussion

In conclusion, the inventors have established and validated a novel epigenetic test, the WID-Breast29, based on a 37 CpG (29 gene) DNA methylation signature (as well as the WID-Breast5 consisting of 5 genes) that is capable of determining the presence of breast cancer in an individual, in addition to monitoring real-time biological responses to preventive breast cancer therapies and therapies aimed at treating breast cancer. The WID-Breast is capable of indicating cancer risk in the breast and can be modulated by exposure to potential cancer-preventive drugs such as mifepristone.

The assays presented herein establish risk monitoring tools and provide surrogate markers for use in real-time monitoring of individuals, allowing for individualised cancer prevention—a strategy which has shown to be highly successful in reducing death in the cardiovascular disease setting.

1. A method comprising assaying from a sample from an individual:
the methylation status of a panel of one or more CpGs selected from within a panel of one or more Differentially Methylated Regions (DMRs) defined by SEQ ID NOs 1 to 29, wherein selected CpGs in each DMR are denoted by CG.

2. A method according to claim 1, wherein the step of assaying the methylation status of the one or more CpGs in the panel comprises assaying the methylation status of one or more CpGs within any one DMR, or within any combination of two or more DMRs, wherein DMRs are selected from the group consisting of:
a) DMR #1 (SEQ ID NO: 1);
b) DMR #2 (SEQ ID NO: 2);
c) DMR #3 (SEQ ID NO: 3);
d) DMR #4 (SEQ ID NO: 4);
e) DMR #5 (SEQ ID NO: 5);
f) DMR #6 (SEQ ID NO: 6);
g) DMR #1 (SEQ ID NO: 7);
h) DMR #8 (SEQ ID NO: 8);
i) DMR #9 (SEQ ID NO: 9);
j) DMR #10 (SEQ ID NO: 10);
k) DMR #11 (SEQ ID NO: 11);
l) DMR #12 (SEQ ID NO: 12);
m) DMR #13 (SEQ ID NO: 13);
n) DMR #14 (SEQ ID NO: 14);
o) DMR #15 (SEQ ID NO: 15);
p) DMR #16 (SEQ ID NO: 16);
q) DMR #17 (SEQ ID NO: 17);
r) DMR #18 (SEQ ID NO: 18);
s) DMR #19 (SEQ ID NO: 19);
t) DMR #20 (SEQ ID NO: 20);
u) DMR #21 (SEQ ID NO: 21);
v) DMR #22 (SEQ ID NO: 22);
w) DMR #23 (SEQ ID NO: 23);
x) DMR #24 (SEQ ID NO: 24);
y) DMR #25 (SEQ ID NO: 25);
z) DMR #26 (SEQ ID NO: 26);
aa) DMR #27 (SEQ ID NO: 27);
bb) DMR #28 (SEQ ID NO: 28); and
cc) DMR #29 (SEQ ID NO: 29).

3. A method according to claim 2, wherein:
i) the step of assaying the methylation status of a panel of one or more CpGs comprises assaying the methylation status of each one of the one or more CpGs within any one of the DMRs of a) to cc), or within any combination of two or more DMRs of a) to cc), optionally wherein the panel of one or more CpGs comprises two or more CpGs of the DMR(s), three or more CpGs of the DMR(s), four or more CpGs of the DMR(s) or all CpGs of the DMR(s); or
ii) the step of assaying the methylation status of a panel of one or more CpGs comprises assaying the methylation status of each one of the one or more CpGs within:
a. four, five, six, seven, eight, or nine or more of a) to cc);
b. four, five, six, seven, eight, or nine or more of a) to r);
c. the four DMRs defined by a) to d);
d. the five DMRs defined by a) to e);
e. the six DMRs defined by a) to f);
f. the seven DMRs defined by a) to g);
g. the eight DMRs defined by a) to h); or
h. the nine DMRs defined by a) to i), and optionally
wherein the panel of one or more CpGs comprises two or more CpGs of the DMR(s), three or more CpGs of the DMR(s), four or more CpGs of the DMR(s) or all CpGs of the DMR(s); or
iii) the panel of one or more CpGs comprises two or more CpGs of the DMR(s), three or more CpGs of the DMR(s), four or more CpGs of the DMR(s) or all CpGs of the DMR(s).

4. (canceled)

5. (canceled)

6. (canceled)

7. (canceled)

8. (canceled)

9. (canceled)

10. A method according to claim 1, wherein:
i) the step of assaying the methylation status of the one or more CpGs in the panel further comprises or additionally comprises assaying the methylation status of each CpG within one or more of the sequences identified by SEQ ID NOs 67 to 84; and/or
ii) the step of assaying the methylation status of a panel of one or more CpGs further comprises additionally assaying the methylation status of one or more CpGs identified at nucleotide positions 61 to 62 in SEQ ID NOs 30 to 66, optionally wherein the one or more CpGs identified at nucleotide positions 61 to 62 in SEQ ID NOs 30 to 66 comprises:
a. at least 5 CpGs, and preferably wherein the at least 5 CpGs comprise the CpGs identified in SEQ ID NOs 30 to 34;
b. at least 10 CpGs, and preferably wherein the at least 10 CpGs comprise the CpGs identified in SEQ ID NOs 30 to 39;
c. at least 20 CpGs, and preferably wherein the at least 20 CpGs comprise the CpGs identified in SEQ ID NOs 30 to 49;
d. at least 30 CpGs, and preferably wherein the at least 30 CpGs comprise the CpGs identified in SEQ ID NOs 30 to 59;
e. at least 37 CpGs, and preferably wherein the at least 37 CpGs comprise the CpGs identified in SEQ ID NOs 30 to 66.

11. (canceled)

12. (canceled)

13. A method according to claim 1, wherein the panel comprises one or more CpGs identified at nucleotide positions 61 to 62 in SEQ ID NOs 30 to 66.

14. A method according to claim 13, wherein:
a. the panel of one or more CpGs comprises at least 5 of the CpGs that are identified in SEQ ID NOs 30 to 66, and optionally wherein the at least 5 CpGs comprise the CpGs identified in SEQ ID NOs 30 to 34; b. the panel of one or more CpGs comprises at least 10 of the CpGs that are identified in SEQ ID NOs 30 to 66, and optionally wherein the at least 10 CpGs comprise the CpGs identified in SEQ ID NOs 30 to 39; c. the panel of one or more CpGs comprises at least 20 of the CpGs that are identified in SEQ ID NOs 30 to 66, and optionally wherein the at least 20 CpGs comprise the CpGs identified in SEQ ID NOs 30 to 49; d. the panel of one or more CpGs comprises at least 30 of the CpGs that are identified in SEQ ID NOs 30 to 66, and optionally wherein the at least 30 CpGs comprise the CpGs identified in SEQ ID NOs 30 to 59;
e. the panel of one or more CpGs comprises at least the 37 CpGs that are identified in SEQ ID NOs 30 to 66; and optionally wherein the at least 37 CpGs comprise the CpGs identified in SEQ ID NOs 30 to 66.

15. (canceled)

16. (canceled)

17. (canceled)

18. A method according to claim 1, wherein the step of assaying the methylation status of each CpG in the panel of one or more CpGs comprises:
a. performing a sequencing step to determine the sequence of each CpG; b. hybridising DNA to an array comprising probes capable of discriminating between methylated and non-methylated forms of the CpGs and applying a detection system to the array so as to determine the methylation status of each CpG; and/or
c. performing a PCR step using methylation-specific primers, wherein the methylation status of the CpG is determined by the presence or absence of a PCR product, and optionally wherein the step of assaying the methylation status of a panel of one or more CpGs further comprises determining a β value of each CpG.

19. (canceled)

20. A method of treating and/or preventing breast cancer in an individual, the method comprising administering one or more treatments to the individual determined to have cancer by the step of performing the method of claim 1 on a sample from the individual.

21. A method according to claim 20, wherein the treatment is a preventative therapy, and therapy comprises administration of one or more doses of one or more selective progesterone receptor modulators (SPRMs) and/or RANKL inhibitors, and optionally wherein the one or more SPRMs comprises Mifepristone.

22.-41. (canceled)

42. A method according to claim 20, further defined as comprising the step of stratifying the individual according to their risk of having breast or according to their risk of breast cancer.

43. A method according to claim 42, wherein the individual is stratified as not having breast cancer and the individual is subjected to routine screening, optionally wherein the routine screening comprises a mammography about once every three years.

44. A method according to claim 42, wherein the individual is stratified as having breast cancer or as having a high risk of breast cancer development and the individual is subjected to one or more treatments according to their risk, wherein the one or more treatments comprise any of:
a. intensified screening, optionally comprising one or more mammography scans and/or breast MRI scans;
b. administration of one or more of mifeprestone, aromatase inhibitors, denosumab, selective estrogen receptor modulators (SERMs) and SPRMs; and
c. bilateral mastectomy.

45. A method according to claim 42, wherein the one or more treatments that the individual is subjected to are on a repeated monthly, three monthly, six monthly, yearly or two yearly basis following an initial administration.

46. A method of assaying methylation in an individual at multiple time points, the method comprising: (a) performing the assay according to claim 1 at a first time point; (b) performing the assay according to claim 1 at one or more further time points; and (c) detecting differential methylation status between (a) and (b).

47. A method according to claim 46, wherein the further time points are monthly, three monthly, six monthly, yearly or two yearly basis following an initial assessment; and/or wherein one or more treatments are administered to the individual according to claim 12.

48. A method according to claim 1, wherein:
a. the sample from the individual is a sample of breast tissue; and/or
b. the sample is a sample of breast tissue comprising epithelial cells; and/or
c. the cancer is ductal carcinoma in situ; an invasive ductal carcinoma such as tubular type invasive ductal carcinoma (IDC), medullary type IDC, mucinous type IDC, papillary type IDC, cribriform type IDC; invasive lobular carcinoma, inflammatory breast cancer, lobular carcinoma in situ, male breast cancer, luminal A breast cancer, luminal B breast cancer, triple-negative/basal-like breast cancer, HER2-enriched breast cancer, normal-like breast cancer, Paget's Disease of the nipple, Phyllodes tumours of the breast, or metastatic breast cancer.

49. An array for discriminating between methylated and non-methylated forms of CpGs; the array comprising oligonucleotide probes specific for a methylated form of each CpG in a CpG panel and oligonucleotide probes specific for a non-methylated form of each CpG in the panel; wherein the panel comprises at least 5 CpGs selected from the CpGs identified in SEQ ID NOs 1 to 29 and denoted by CG, optionally provided that the array is not an Infinium MethylationEPIC BeadChip array or an Infinium HumanMethylation450, and/or provided that the number of CpG-specific oligonucleotide probes of the array is 482,000 or less, 480,000 or less, 450,000 or less, 440,000 or less, 430,000 or less, 420,000 or less, 410,000 or less, or 400,000 or less, optionally wherein the panel comprises any panel of CpGs defined in a method of assaying from a sample from an individual the methylation status of a panel of one or more Differentially Methylated Regions (DMRs) defined by SEQ ID NOs 1 to 29, wherein selected CpGs in each DMR are denoted by CG, wherein DMRs are selected from the group consisting of:
a) DMR #1 (SEQ ID NO: 1);
b) DMR #2 (SEQ ID NO: 2);
c) DMR #3 (SEQ ID NO: 3);
d) DMR #4 (SEQ ID NO: 4);
e) DMR #5 (SEQ ID NO: 5);
f) DMR #6 (SEQ ID NO: 6);
g) DMR #7 (SEQ ID NO: 7);
h) DMR #8 (SEQ ID NO: 8);
i) DMR #9 (SEQ ID NO: 9);
j) DMR #10 (SEQ ID NO: 10);
k) DMR #11 (SEQ ID NO: 11);
l) DMR #12 (SEQ ID NO: 12);
m) DMR #13 (SEQ ID NO: 13);
n) DMR #14 (SEQ ID NO: 14);
o) DMR #15 (SEQ ID NO: 15);
p) DMR #16 (SEQ ID NO: 16);
q) DMR #17 (SEQ ID NO: 17);
r) DMR #18 (SEQ ID NO: 18);
s) DMR #19 (SEQ ID NO: 19);
t) DMR #20 (SEQ ID NO: 20);
u) DMR #21 (SEQ ID NO: 21);
v) DMR #22 (SEQ ID NO: 22);
w) DMR #23 (SEQ ID NO: 23);
x) DMR #24 (SEQ ID NO: 24);
y) DMR #25 (SEQ ID NO: 25);
z) DMR #26 (SEQ ID NO: 26);
aa) DMR #27 (SEQ ID NO: 27);
bb) DMR #28 (SEQ ID NO: 28); and
cc) DMR #29 (SEQ ID NO: 29).

50. A hybridized array, wherein the array is obtainable by hybridizing to an array according to claim 49 to a group of oligonucleotides comprising any panel of CpGs defined in a method of assaying from a sample from an individual the methylation status of a panel of one or more Differentially Methylated Regions (DMRs) defined by SEQ ID NOs 1 to 29, wherein selected CpGs in each DMR are denoted by CG, wherein DMRs are selected from the group consisting of:
a) DMR #1 (SEQ ID NO: 1);
b) DMR #2 (SEQ ID NO: 2);
c) DMR #3 (SEQ ID NO: 3);
d) DMR #4 (SEQ ID NO: 4);
e) DMR #5 (SEQ ID NO: 5);
f) DMR #6 (SEQ ID NO: 6);
g) DMR #7 (SEQ ID NO: 7);
h) DMR #8 (SEQ ID NO: 8);
i) DMR #9 (SEQ ID NO: 9);
j) DMR #10 (SEQ ID NO: 10);
k) DMR #11 (SEQ ID NO: 11);
l) DMR #12 (SEQ ID NO: 12);
m) DMR #13 (SEQ ID NO: 13);
n) DMR #14 (SEQ ID NO: 14);
o) DMR #15 (SEQ ID NO: 15);
p) DMR #16 (SEQ ID NO: 16);
q) DMR #17 (SEQ ID NO: 17);
r) DMR #18 (SEQ ID NO: 18);
s) DMR #19 (SEQ ID NO: 19);
t) DMR #20 (SEQ ID NO: 20);
u) DMR #21 (SEQ ID NO: 21);
v) DMR #22 (SEQ ID NO: 22);
w) DMR #23 (SEQ ID NO: 23);
x) DMR #24 (SEQ ID NO: 24);
y) DMR #25 (SEQ ID NO: 25);
z) DMR #26 (SEQ ID NO: 26);
aa) DMR #27 (SEQ ID NO: 27);
bb) DMR #28 (SEQ ID NO: 28); and
cc) DMR #29 (SEQ ID NO: 29).

51. A process for making the hybridized array according to claim 49, comprising contacting an array comprising oligonucleotide probes specific for a methylated form of each CpG in a CpG panel and oligonucleotide probes specific for a non-methylated form of each CpG in the panel; wherein the panel consists of one or more CpGs selected from within a panel of one or more Differentially Methylated Regions (DMRs) defined by SEQ ID NOs 1 to 29, wherein selected CpGs in each DMR are denoted by CG, wherein DMRs are selected from the group consisting of:
a) DMR #1 (SEQ ID NO: 1);
b) DMR #2 (SEQ ID NO: 2);
c) DMR #3 (SEQ ID NO: 3);
d) DMR #4 (SEQ ID NO: 4);
e) DMR #5 (SEQ ID NO: 5);
f) DMR #6 (SEQ ID NO: 6);
g) DMR #7 (SEQ ID NO: 7);
h) DMR #8 (SEQ ID NO: 8);
i) DMR #9 (SEQ ID NO: 9);
j) DMR #10 (SEQ ID NO: 10);
k) DMR #11 (SEQ ID NO: 11);
l) DMR #12 (SEQ ID NO: 12);
m) DMR #13 (SEQ ID NO: 13);
n) DMR #14 (SEQ ID NO: 14);
o) DMR #15 (SEQ ID NO: 15);
p) DMR #16 (SEQ ID NO: 16);
q) DMR #17 (SEQ ID NO: 17);
r) DMR #18 (SEQ ID NO: 18);
s) DMR #19 (SEQ ID NO: 19);
t) DMR #20 (SEQ ID NO: 20);
u) DMR #21 (SEQ ID NO: 21);
v) DMR #22 (SEQ ID NO: 22);
w) DMR #23 (SEQ ID NO: 23);
x) DMR #24 (SEQ ID NO: 24);
y) DMR #25 (SEQ ID NO: 25);
z) DMR #26 (SEQ ID NO: 26);
aa) DMR #27 (SEQ ID NO: 27);
bb) DMR #28 (SEQ ID NO: 28); and
cc) DMR #29 (SEQ ID NO: 29),
with a group of oligonucleotides comprising any panel of CpGs defined in the method of assaying from a sample from an individual the methylation status of a panel of one or more Differentially Methylated Regions (DMRs) defined by SEQ ID NOs 1 to 29, wherein selected CpGs in each DMR are denoted by CG, wherein DMRs are selected from the group consisting of:
a) DMR #1 (SEQ ID NO: 1);
b) DMR #2 (SEQ ID NO: 2);
c) DMR #3 (SEQ ID NO: 3);
d) DMR #4 (SEQ ID NO: 4);
e) DMR #5 (SEQ ID NO: 5);
f) DMR #6 (SEQ ID NO: 6);
g) DMR #7 (SEQ ID NO: 7);
h) DMR #8 (SEQ ID NO: 8);
i) DMR #9 (SEQ ID NO: 9);
j) DMR #10 (SEQ ID NO: 10);
k) DMR #11 (SEQ ID NO: 11);
l) DMR #12 (SEQ ID NO: 12);
m) DMR #13 (SEQ ID NO: 13);
n) DMR #14 (SEQ ID NO: 14);
o) DMR #15 (SEQ ID NO: 15);
p) DMR #16 (SEQ ID NO: 16);
q) DMR #17 (SEQ ID NO: 17);
r) DMR #18 (SEQ ID NO: 18);
s) DMR #19 (SEQ ID NO: 19);
t) DMR #20 (SEQ ID NO: 20);
u) DMR #21 (SEQ ID NO: 21);
v) DMR #22 (SEQ ID NO: 22);
w) DMR #23 (SEQ ID NO: 23);
x) DMR #24 (SEQ ID NO: 24);
y) DMR #25 (SEQ ID NO: 25);
z) DMR #26 (SEQ ID NO: 26);
aa) DMR #27 (SEQ ID NO: 27);
bb) DMR #28 (SEQ ID NO: 28); and
cc) DMR #29 (SEQ ID NO: 29).
